SEQUENCE LISTING (4 sequences)

SEQ ID NO: 1 - DNA, 267235 bp, Homo sapiens
ggggtagccaggcagggccgtccacaggagcaccagggccgcactggctgtgtcccactgccaaaggctgcaaacaattcccacagacccagggccagggcctgtgaaccaagaaccccagtctatctttgtctcaggttgccaggcctcctggaaagcccgaagtcctgggggcagctgtgtccagcagacgctgcacccagcagtcttgggggttgggctgacccgagagagtgggcattgctggtgcccagaccccaggagagggggtggccagcatgagggtcacagtgccagggcatggctggggtcaagccacactcagggccagtgtatgcctggacctaggggcaccaggcaggggggcacagccaggaggcaggtgtgggatgcatctcagggtgcctgactctaggcataaaaggcgctggtttctaggcccagctgcctgctgtgggttattaaaggggaaggaccctcccctgcagagttgcgcttgcagcctcgtctcccagaaggcatggccagccctgagccacctcagcccgacaccaggaggggtgatgtgcactcgtgtcctcggcctgggagagtgtgtgtcctgcaggcaggcgtgtgtgtgtggtggagtgtgtgtgtgcgtggcctgaagcccggggctccgtgtattgcagagtgcctcgggcccgggcctggccttcgtcgtcttcacggagaccgacctccacatgccgggggctcctgtgtgggccatgctcttcttcgggatgctgttcaccttggggctatcgaccatgttcgggaccgtggaggcggtcatcacacccctgctggacgtgggggtcctgcctagatgggtccccaaggaggccctgactggtgagcgcacagctccgccgccctggaggacccgtccccagcatctgactgtccactcccgcccgctgtccagacgcccctcctggatggagagcgcaaggggccaagcctgagttcagggaaaggctgagccaggctactccttgctgacagccatcaacggagagccgagggtcgagggagggtcagggctgcccctcccccacggccccggaggccacatcccccatctggagcaggaaagcaggtccttcctgctccttgagcacaatacaggaaagagactggggagggcagggatggagggtgggaagccgagagaagtctccagcccaggtgccctggccctccccgctgtccgagggcaggtctgcctggctggcttcctcctgaccctccccacacctccactccccatccccttaccccccacacccctttcccactgccccagggctggtctgcctggtctgcttcctctccgccacctgcttcacgctgcagtctgggaactactggctggagattttcgacaattttgccgcttccccgaacctgctcatgttggcctttctcgaggttgtgggtgtcgtttatgtttatggaatgaaacggtgagctgccgccccgccgagtgctcctctgggacccaccagggtgggacagggaatggctgccgggcacaggttcaacccgcagctgcccagaccgccgagaatgttctgccctggggagctgccgaggcaccagagggtgcaggttggaccccagttagggtccgatcctcggcttggagtgggtggaccttccacagaccatgtgaagcctgagcccagcatctggtgcaggttctgcgatgacattgcgtggatgaccgggaggcggcccagcccctactggcggctgacctggagggtggtcagtcccctgctgctgaccatctttgtggcttacatcatcctcctgttctggaagccactgagatacaaggcctggaaccccaaatacgtaggtccttccggtgggaacctgggaagtcctgggacccctcgggcttggtctggcccctacggtgccatccggtgcccgacgacccatgcggtgcacattcacgtgctagtgacaaggtggcgtggtcacttctgcctggcaaactgggaagccagggatgtgaacaggatgggctttgtgcagcgcccgagtgcccgctgggatcagaaatggcatgggggtgaccatgggcaagcgcctggtcagtggagtgaggacctgagcagtgcccagagcccagggaccccacagggccctggagctcctccttggggatgtggcttcatcctccctgcctgtgtcctgcccatgtggcatggggacgggcagccggacaacggcccattcctggtctgtgacaagagaggttgtgcacagacccctactgccacccagtgggtcagtttggctggaggccgtttccacacaaaagcctcccgaggagagatcagagtgccagccacactccgaaaacgcctcaggggctccaagcagcgccagcccctgacataccatctgagtccccactggtggccgctcaaaacctcaccatgccaccccttctcgggaatgtggacgccactcctctccaaatgtgtcttggaaagtccctccgtagcagaacaccaagggcaggtgaaggaggagtgaccatgtatttttgaagaatttccacggtacctgcagccgggtgggtggtgccacccagtgtataggggagaaagctggggacacggcctccaacgacgcccaatgggttgaggccatgtagcccctctggttccagaacacaacacctaggcctacctcggggaggccccagccccaggagaagctcagcccaggtccccaaactcctggcccggaggggcggtggtgagcggccacacggcctggccactgctctggccgtgccttttccattcccatccaagtccgggtgggcaggtggctcttgccccttggattgaggagcacgggggtcagcctcacggcccagggtggccggcgccagctaatgaggctgtggtcccgcaggagctgttcccctcgcgtcaggagaagctctacccgggctgggcgcgcgccgcctgtgtgctgctgtccttgctgcccgtgctgtgggtcccggtggccgcgcttgctcagctgctcacccggcggaggcggacgtggagggacagggacgcgcgcccagacacggacatgcgcccggacacggacacgcgcccagacacggacatgcgcccggacacggacatgcgctgaagccggccggagcggggcctgcatgggcgggtctgtgggggggcttggcctgatggtgggcggggccccgcccacagggccgaccccaatacaccagcgactcaaccttgatgccgctgtgtacgtgtggttactgctttcctgacgcccgggcagcgctgctgcttggggcccgcccctagggaggtctcgcagggccccggcatccctgctgagcccgcggcctgggcgtcctgcagcggctgaggtccctgcgcttggccgggctggctcagggatcttcgcaggtgcggggctcccggtctcacagactctgaacctagcctggctggtctctggggaagagcggccgcccgggtggctcagggggccgcctgcaccccggctccaggtcccaggcgggctgtccccctacagctcgcaggcggcaggggtacccaggacagatgcttcctcccgcctgggagcgggggcctggggtctggagtctgcggtctggctgacccttcctgaaatttctgagaaacactgtaggttgccacttttattttcccaaactgtcaaactgtattaaggaggtaccaaaaggctcctgctgtcccggggcagaggggcgacaggcaggagctcgctcacgtggggcagtcacccgcgccccgggcgccaagcgcactgtgcagcccccgtccagcccgccctgctccagagtaccccggctccaggcgccacgcgcgtgcttccctcctccctctccctctgttccctccgccctcctcctccctctttcctccccatccttttccctctcctccttcccctgcaacagcaccacggagccagggatcaggtctctgactcgccacacggagggaggccccgcaggtcctggcttcctgccgccgggaggggccctgtgaagacccaaggcacctgctcccgctttggaaaagggcgcctggccaggcagcactgtccgtggtgctgaaaaatcccagcatcaaggcgcggccgcggtgccaagcgtccattacacgcattcttgttgggaacccgtccttgaccaaggccagcccagagtaaacaaaagcacgtgcaccgctacccacctgcagataagctgagtgctaaacatgcagttaacgccagtgcaccccacccaagcgcatcctgagcccacagtgggggaagctgcccacctaaacgtgtcctgagcccacggcaggggaagctgcccacctaagcgtgtcccgagcccacggcagggggggagctgcccacctaagcgtgtcctgagcccacggcaggggaagctgcccacctaagagtgtcccgagcccacggcaggggaagctgcccacctaagcgtgtcccaagcccacagcagaggggtgctgcacttcgtccctccgtcctgcagttgtgatgccccccacaccggtggtaaggcagggggacttgaggatgaacatgtcacaaagcgagtcagtccactgtgagataaggctgctgcagaggacagtctggcatcccccagttcagcctcacaatcttgccacaactccatgccttgggcctaatagccagcctcttccattgtaactgacacaaacaaactcaaaccaaatggccttcaccagctctcagaactggccaagttcagggtgggcttcaggccctactggacccaggagttcacaggatccatgggactctctccccacactgttcagcttttttaagtagggcgtctctgtgaccattccaaaatccttttggttcatgatctgggtgagatgctcccccttcagagtctagacacgaaattgcagggaaaagtgattgcagagcctggagggtggggtggagggaggggtgcttccccaaaggaaagcctccagcagccaaagcaacagctgcgttccatggtgtcacccactcacagagcctggagggtggagtggagggaggggtggttccccaaaggaaagcctccagaagccaaagcaacagctgcgttccatggtgtcaccaactcacagacgcaaaaggtagagtaacctgctcagaagcaaacactgccaagggcgtgtgtccccaaaggcagctcttgggacaccttccccaactcaccatagggcctgcaagtcctgtcgttgacccggacgagaaaaacaggcagctgttggcagcaggggtgctgacaagccactcagacttatccgaggttacattttgcgtcaataccatacttagaaaataactaggatgtgagcaggtgccccctgctcccaccctggatcagacttttgggtgtagcacatcctaacgagggctggggctctgcccaacactctgtatggggggagctagaagcagcatctatagccccaccctgaagcttcagagtccttgtccagctgacgaaaagggaaggggtggggggctgggacagaggacacagtggacgctcagccctccagaaaatgccacaccacccatgtgcctgatgcagccatggataaaagtgggtagaaaggaggagggacagaggtggcccaataggtgatgacaggtggacagacggacgccacaaagggaggcagaggtggtctgtcccatcacattgccaaggttgttcagggacaggggctgaggcctcatgggctgtggaggtacccgtgcaccatcgagggagggggagacaagggccaggcggcgagagcagaactggagggactgaccctgcgtggccacgccccatgctcaacaccttcaacttgtccctcacccttgggaaaaacattccaggggcgggtgtctcagactggccagtgtcgggacaggaacaaaatgctcctatcacctgaccagaagtggctggaatagaggatctgtagctccacaggggctctccccttccaggggccaccgtgttgaactggaaataaagccggctgcaaatgaggccagccggccccagagatggacacagctcccatgaccatggtggtcacatgaggacaggctgtcccgtgagcgggttcctggcagagggtgaccccaaggctgctttgccaggcacagaggagtcagggagcacttgatgggaaaacgaggttcagggcccagttttcaaaagcctgatggggctcatcctcaggggagagtctgaggtcaccaagaaccaggggagaatgcgtgaaggacataagtgcagaaggagaagtgaggcgggaggtcatggagggcgcagtcagcactggagctgccccaggactcacccacccagctccaagggaagcagccccttccagcaagggtgggcctggacctgacagagaggaccccgctgccacggccagggagcatgagtctcccgtccacactgggggctggggacttaccactccaggtggcgagcccggggcttcgtgctgtgtcagaggagaaagccagccaggccccctccggcaagactcggttctttcatgcctgcatgaggctgaggtgaggcccaggcctgtcgtgatgctcgctctctggggccgcagaaatgggaccatggaacctgtgcatccagatggcaccttggagacctgtccgaccctcctgcaggccacagcggagtgcccaacaccaggctccacaaacctgggtgacctgcactcgccgggggccctggtcaaagctggatccagggtgaagaggctccatcccttgcccgtcagtgtgaggactccgccagcggcctcggccattggagctggatgggcacaggagccctgctgccccagcacacacatttcatcatgaccccaggtgaccagcacgcagggaggctggcagccccactgtctctgttcctttctccctctcggcagcaactccacctgagctgtcccacaggatccccaggggtgggctgagagtcactctgagaactgtccctgtattcatttccctgtactgctattccatttgctgctgtgacgaatcactgcagcttagtggctgaacacaaacgcatcatcttacagctctggaggccggaaagctgaaacaggtcccactggcctgaaatcgaggcgtgggcagggccgtgtgccctcaggaccggggaggattgcttgcttttccagcatggagcggctgcccccattccttggctggggaccccctcctccatctcaaagccagcagtggccagtggtccttctcacgtcccctctctgactctcccctgcctccctctctcacttctagggacccttgtggccatatcaggcccaccagataatccaggatgaccttaagatcggctgactggcagccgtgattccacctgcagcctccaccagcctctgccttgcgggtgacacattcacaggttccaggagaaggacgtgggcatctttggggaggggctgtaattgtgcctgccacaagtgcctggggcttctgaaacccaccaaagtttggcaagccccctgcacagcatccttcccaggtgggcacctggcaccaacatcgacggttacagcaggtgcaggaccggcaggagcgtggggctgaggcaggaaaacaaccactccctttcaggggtcctggctggtgtcacccacagcctccacccttgcctgcttctcctccctttctgctttgaactcactcgctccatacacgcttgtctgtggaaggaagctgcttgagatgaagttcaggcctaaggaagtccaaagagctggggtttttctttccctttaaaactttgttattttcattttgctctgagatttgacagtctacctagcacctagcaggcctccttctccttctttcttccttcttccttcttcttctctttccttcttccttcttcttctcttctccttctccttctttttttttttttttttttgagacagggtctcactctgttgcccaggctggagtgcgatggtacaatcacagctcactgcagccatgacctcccgggctcaagcgatcctcccactcagcctcccgagcagctgggactacagatgctggaccccacgcccggataactttttgcattttttgtagagatggggtctccctatgttacccatgctggtctcaaactcctggcctcagacaatccttctgcctcggcttcccaaactactgggatgacaggtgtggaagccactggacctgaccttacatagtggtttttaaaagtgccaggcaagggcagtggctcacacctgtaatcccagcactctgggaagctgaggccggaggatcacttgagcctgggggtttgcggctacagcgagccatgactgtgccactgcactaacaacctgggtgacagagtagacaccatctctaataataataataataataaataaaaacagacaaataaagtaggtgcttagtgagttaagccatatcaaagcaaataccttttttaagggtctaatactttagcaaaaaatataagtaatcctgagtaaaatttcaacaaaaaacacccggagaaacaggaatctggagctttgtagaagatggatagctgaacaatggcctgacctgttggtctgtgattcaaacccaggtgtgcctgagcagcggcctctgctcctgggagtccccgccctgttctgtccaacctcccactgaggtcgtccgcccacggcgcccggggccgctcccttccagtttgcagggcaaaggcgcccccaggccggctgccaagagcccctccttcctcacagctcctatacgggggctccaggcaccagagtgcatttacgacacagtctgtggacttgggcccaccataaacacattaggctcggtcactcagacattctctgaagcagcagcaataaatgacgccatatccagtggccataagcccccaccccgacaccaggcacgtccgggctgcactcaaaccagaagccctgtccaaggtgcactcagtaggacccggccataccataaggccccaccagggacctgctggaaggctctggagccactcacgcagggccctcggtttcgtgtggcccaagccaacttctgaggaggggctgtttcttcaccccaagactgcgtccctctgagccgcccctcctgttctctgcaatccttccagttccttctgcctgaggaaggacgtatgtccagctccactgcgagcctggcacccgccctgggaggtaagtctggatgagactaggagataagaggataagagatgagactgggagatggtaagaggatgacagatgagactgggagataagaggaagacagatgagactgggagatgataagaggatgagagatgagactgggagatgataagaggatgagagagcatcaccagccccaccctgaagcctcctgtgtccacaccctccactgccccaccctacccacccttcagagatcagaatcctggtctactcttaagagaatggccagaggcggaatgtgactttcaacctgagttggggcgttcaggaggaggaatcctggattttatacaggatggtgtcatgcccctgccccccacctccatgaaagctgatggcccagaggatgtgcaccagggaagcacatccggatccacggctgcaggtgggaggcaggggaggagggggcccagccaggcttccccatcttccccgccacccagaccagccccccccatcacccctgccaccccagctggggcccccatcatccctgccaccctggccaggccctccatcatccccgccgcccaagctgggcccccagcatccctgctgcccttcgggcctggacttactgttatgtcttccagggtgggggctcccactgtctatcccctaccctccttcccctcctgcctcacagcatcagaaacctcccaggcccagccaggccatgggcccgcactgaccacaaccccattcactcatagacttccgtaaatgcacagccaggccatgggcccgcactgaccaccaccccgttcactcacaggcttctgtaattgcatacagtggtgtctatgcagtgcacattaggattcaaacatgagatttttttcaaaactgaaaaactcatatattcagtattttactcccacagcacctccccccaatttgacccacagggacccccatccaggtgcagggtcctcgcctgtgtacagggcacacctttggtcactccaaattcccagagctcccagggtccttctcagggtctccacctggatggtgggggtggaaggcaaaggagggcagggcgaggggtgaacaatggcgaatctggggatggactattcctatgtggggagtggaagccgggctcctggtgaggaaaagctggccctggggtggagccgagcgccagcctgtggggaagtgaagacggcaggtgtgctggacactcagcccttggctggacactcgctcaggcctcagccggacactcagccttcagccggacatgcaggcctcggccaaacactcactcaggcctcagactcccagcggtgcgggcctgggtgtgggccgcccctccctccctgggacgtagagcccggcgtgacagggctgctggtgtctgctctcggcctggctgtgggcgggtggccatcagtccaggatggtcttgaagtctgagggcagtgccgggttggctgcggcctccagggcagtcagcgtcgtccccgggagcttccgactcagctgcgtctgggctgcggggccaaaatcagactccgttccagaagaggccagaggtggcatcctccaaccctcctaggacgtgtgggtggccgggcaggcagggcctgcacctcgtggccctggctggtgggaacctcagtgaagggacagacacccctccaccggggcgagcagacaggcagcccagtgacgctggagcggcggggcggggagagaacttgctcaggaccccaggagctcagggcagcagccagggaagaggctgtgagaggacacaggtcaggggtcagaaggctcccaagcgtggggagccatcgcccctgagatgggaagcaggggttcaagaagttgtggaccctgccaggctccgtggcctggggacaccagcgtttaatcacatagggctgccagcgtggctccaggcccccagggtcaggcccgggggcgtctgcacttcagcagcatctgaggctgctcgccccacacagggcgttcaaggatgacccctgggcaggtggggcccgcactggcctccacccacacttgcctgtcctgagtgaccccaggagtggcacgtaggtgacacggtgtcgagtcagcttgagcaggaatgcttggtggcacagccactgcacggcctcggagggcagagggccggcggcgcccttggcccccagcgacatccctgggggaaaacagaggctgaggagtcacaggcccagcccagctcccctcccaggagacaccggaaagctgcccacacccgacggtctgcggggtggctccatctctggctgtcccgacccaagcaccgcaggagtcacgacagaaatgttttcttcggcttctccaggtgaaatttccacaacacagaaagaacagaaagtagaagctgttaagctcaggaccgcaggttcgacatggcttgaatttcagacggatcagaaacctccctgatgctggacaaagtgtccaggggcagaggacggtgactgggaagcagaggacggcgcggggctctgtcgtggtgatacgcggcctctgctcctgagaggggtggggtagcctgggcctggcctcctcctaagcccttggcctgggtttgcacaagggccctaagtgccatggacgggagacagcccaatcccaccacagggcaaacaggagaggccaggccccccaaatcccaccatggggcaaacaggagagaccaggccccccagcgcttccccaggcagagcaaggtgggaaggatggcagctcccagacagctcctgtcccttccatcaggtgctctgctcaccccacgagagggcgggcagacgagcctgacagtggttgggttaaaccacttcctgatgcgaaaaggggtaagaacttcctaagcccagattcactcagtctcctgacacactaacaccagcaggcaggcactgctgccactgaggccaggcacctgcacatacctgcgttcttggctttcaggatggagtagcagagggaggccgtgtcagagatgacgcgcaggaaaaatgtggggttcttccaaacttgctgatgaaatgggagctgcagcacacatgcgtgaaacctgagaggatggcggacagcgtcagaggaaaggcctcctaatcagacggtgctcgtgggtgtgggcatgggcccaccggtgcctgtgtgcgtgcatgaatgcacatgcatgggtttcctcatgggcacaggtgcacacacacggatgcatgcatgcatgtctgtgtgtgtgcttgtgtgtgcgagtagctgcgtgtctgtgtgtgcacaggtacactgcgtttctgtgcaccatctgtatgaacacgcatgtggaggctgaagcagctccatcctggatgccagccccccatgctggcttctgattagcccctgttccgggaaggcctctaaggtttccagtttatccattgttccttgtgtaccagcaggtacttatcatgaatcctgcccttcgtcagacaaccttgatgtcatcgtatatcaacgtcctgcgcatcccttctgagccacccgcccctgtggtgcataaaccctgggtctggggtgatggtgtgaggacccatcatctcatctcactgacaccgagacaccgacttggcttctgtgtgtaggttaagttcctactaaatgtttctttccaagaaactgcatttgtcagtcttctttgttgttgttgagatgggctctcactctgtcacccaggctgcagtacagtggtgcaatctcagctcactgcagcttccacctcctgggctcaagtgatcctcccacttcagcctcccaagtattggaactacaggcgcacaccaccatgctcagctaatttttgtttttatttttcatagagatggggttctactatgttgctcaggctggtcttaaactcctgggctcaagggatcctctcgcctcggcctcccaaagtgctgggactacaggtgtgagccaccgcgccagccctgtcagcctccatctttggcctctcagcttcctgggactttggggcaggtgtgcacagacctggctaccatgaaacagcacatgtgcacatgtgctcatatgtgcgtgtgatcagagcccccgtgtacctggtctgaccctctgcctgagccccccgtgtacctcatctcacccagtgcctgagccccccatgtacctggcctcacccactgcctgagccccccgtgtacctcatctcacccactgcctgagccccccatgtacctggtctcactgacttcctgagccacttctggaatgacctgagatcacaccagtcaagccagcaccccggtttcagcctctcagtaactctgctgaggtgggatgtcttttccccatttagcaacaacggaagccaggcccagaatgtttttaaacaaataaataagcctaaggtcccggggttgccacacggccacgctcctgacccaggagggaaacacctagcatgggtgaggggctctcttccaaagggagaaatagccacctgctagaggtcggggcgtccaccccaagcccgtgtggaggcgcggccagcacgggccaggaaggccgagccccagcggatttgaatcagaccccgcccccagcgccacgtggaccaccacagcctcgcccactgcagcctggctgccgtgaaatcggggcccagctctccgttctcccactcctgcctcgagggaaggggattcctgggccttccacgtgcctgactctgtgtttggaaacggcccgtggcccatcgcatctcggcctcctcagggctacccaatcaagcgactacccaagggtccccacatgggtggggaaactcagccgccctgccctgagcccaggccctctacgggagctcctcggagctagagaccctggtggggaaatgaggacaagactgagctgaatatgggaggagcaggtggcaggtcacatggccctgggagtttcccaggagttttcacctgggaaaactcctctgacctcatcatggcccaaggaggccccaagcacgccagctggaccctggggtcaaccacagtgtcagacacctccagtcctcagtggcacctgtatccagcacaccccaagggtttccggaaaggagtcaagcagcttgcacgagggcccaggcacgcgtcagggagatgcaaacgagggaagatttgaggcagagagagaaggcagagaggagggcggtctgagccggacgcaccctggaactgagacagcctggtcccaagcctctggagctggtgcaaaggcggtaacatgcagctgtggctctgccctccctgccagagcatgaaggcgcaagcactcgggttaaaaaccattttcatggtacgaccttgtaatggaaaactctcccgtgagcccttgctgggtttccacgatagacgacgacctcatttcacatcaagtgtgcacggtaattcgcgcagcctcggccctgggtaacacaaatgctcacactccttctcagccctcatggggcgagggcttcggcctcctggcattcagctcccagtggctcgggaggcctgttgaggtggaggcccgggcctgtggtgcccgactgccctttggtacagccccgaggcttgtgccaccacctgccctgactccaggcagaagctggccctgtggcctccacagattggcccttccctctgtgataaagttgtcgattcttggcaggggcaccccctgaccagccaggacagggaacgttccaggagcacagccatgcccagagcacaggtgctggccgggctgtgtggggagccacagctctgctgcaccttcccgggggtggggccagcgaccctcacctgcacccacctcggccctctttgctgagaccccaaatcctccgggcatctgatctcccatctactgttcagacaccttcccagcctcctctgctaagactccccataaacatctctttaccccatgcccctgctgccctgggatggcctccatgacgtcccacggccagtgcacaggcacagtggggacacctggggccacaccggctcctaccactacccagagatggagaacaggtgctttgcacagagccacgcgaacagaactgtgcacaggcagcagcaccactgaaaacgtaagacattccttgcccctaaaacccaggagttccaaggtgaagccccgggtcagaggtgagcagagcgcggagggtccctggaggctgggcctgcaccccttggtggcggctcacctgtacgcctgcagcaggaggatcttgtagatgttggtgcacaccgtctggaggctgttcacctagagtcgccaagaaagagtgagaaacggtagaaacctctctgggattttaagtttttactttttgctttatcatccattcagatggaacaagaaagaggaacattttgacaagaaactatccctcttcccagtgaaatccggcctggccctcacccggcagctgcgaaccaccctgggcgagtcaagactctgtgtcatctgcctgcccccgaggctcggccaagacaggaaggaaccaggagagggagtggacgcaaatgcccacagagaggggaggtggacgcagatgcccacagagagggggagtggacgcggatgcccacaggagagagggagtggagtggatgtaaatgcccacaggagagggggagtggacacagacgcccacaggagagagggaatggagtggatgtaaatgcccacaggagagggggagtggacacggacgcccacaggagagagggagtggatgcagatgcccacaggagagggagtggacgcggatgcccacaggagagggggagtggagtggatgcagatgcacacaggagagggagtggacgtggatgcccacaggagatggagtggatgcagatgcccacaggagagggagtggacgcggatgcccacaggagagggagtggacgcagatgcccacaggagagggagtggacatggacgcccacaggagagagggagcagacgcagatgcccacaggagagggagtggatgcggacgcccacaggaggggggagtggacacggacgcccacaggagagagggagcggacgcagatgcccacaggagagggagtggacgcggacgcccacaggaggggggagtggacacggacgcccacaggagagagggagcagacgcagatgcccacaggagagggagtggacgcagatgcccacaggagagggagtggacatggatgcccacaggagagggggagtggacacggatgcccacaggagagagggagtggacgcagatgcccacaggagagggggagtggacacggacgcccacaggagagacgagtggatgcagatgcccacagaagagggagtggacgcggatgcccacaggagagggggagtggacacagacgcccacaggagagagggagtggatgcagatgcccacaggagagggagtggacgcggacgcccacaggaggggggagtggacacggacaaccacaggagagagggagtggacacagatgcccacaggagagggagtggatgcggatgcccacaggagagggggtgtggacgcccacaggagagggggagtggacacagaagcccacaggagagggagtggacgcggatgcccagatgggacaatgccctcagggctgcagcttcagagcctcagcccaccaaggcctgggaccccgtctcatcctgtaaggagtggcactggtgctgaggacctagcggggtgaacacgaggacccctcaatacatgttatgtgcacacatgctactcacactgcacacccacacgcatgcacctcacgtgaatgcccacacgactgtgcacgaacacacatgcatgtcaggtgtgcattcgcatgtgcacacacttgtgcccttgcacacctgtgcacacactgccatgccttcatgtacacacatgcacacatgtatgtgctcacgtgtatatgcgtacatgtgcactcttacgtgcagccagtcaccatcagccttgcaggcacgcgcgcacacacacacacacatacttgcgcacacacctcctgaactctgaactctgtgctgaccatcagcctgctcacctgcaaatccagaaacaggctgtgacacttcagccgcaagaccccaaagagtttgcgacgcatgttcctcccagccttgaagccgcggttgaaggtgagactggctctgatggaggtccgggcatagctgagacacaggggggaatgtcagacacaggtgcctgccccacacccagccccctcctgcaaagcttgctccaaagagcctcctgccttcctcccgcttccttgtcctgcctggggcatgcgctgcagcccgagggggctgggtgctcactccatggactccaaatgctacatgccatccagactctgcatgaccaaactccaccaggacctggctagtgatgttgagtgtatccaaacctcgcacggccgtgtttgtgcactcatcatctgccctgatgagatctgcacataactcagccaaaaacacataataaactgtggagccctggctcccgggcgtccctcgagtgaacacaggtgtgactcaggttttttgtgggagaacactgagttgggcgtggggaatctcactgtgtttgaatctcagggatggaagagccaggtggcaccagctaaacagggctccagtgagtgcacgtggcacacatggtgtctccagaggggcaggatggaggcatcccaccgatgtcctccagggcctcaccaccactcactgcccatgtatgtccatgtccaaagatgaacacacatgacatccacggcaggctcctggggtgtgttcttttacatccagctttgaacactgctacattgaaataaacgttttgtgccattaaacattcttcaaaaattctttgaaaaatgtttaatggcagagcaagttttgtgatgcataatatctccttagccctgtatttgggcaggttcgtgacttgctgtggcaagtccccctgcctggaacactggaaatggtaaataagacccgtgataatgtctggtcacttcagaagtggaattactgggaagacacaggacattgtgaggacttccatgcaaacaagctgctttccagaaaagtctttccagcttgcctcaacctgatgctggctcttggcaagcccagaaaaggagccagcatgagggatggggacaggtctcgccatcagtttcacgtgcgtttctttgagggatgggtctcgctgtcagtttcacatgcatttctttgacgctgctggtaggaccttcccaagtgtgcatcagacgtccatttcttctttcacagccatccccaacacgaccttgggctccggctcaccctgagcctatgggtctgacttgtgtggggtcctcgggcagagcaagggccccggagcaggaggccaggctgcaatcatgcatgccctttgccagctgggtgaccaagggcggctgctcccctggagcctttcctcctctacctctcaggtggttaggagaacaaagtgagaggatgaatgtgcaggacctcacagggcgccttaaataaatcacgtgcacaaacgctccagggaatggccattttcactgtggctcaggcaagctgagtttctctgcagtgaaagcaatgcccgttgcctcactggtttcatccgcctccctgtgtgtccttccccaacaccaaaccagggaacgttcacctcccttccctttgagtcttttgttttatttctgttgctggtgttgtttgcatgtgtctcctcttctacctagaatctgctctggtatatgctgtaaagacacaacttatttgcttccagtcagttttctcagaaaaaaaaattacacacacactctcttttccatcagctgtgccacataccacattaacacacgtttctgagagatagggcttccgttttgttctattgttctgtccatcagttctgaccacattctaccatctgagctgtggtttgggagactaaaatctggacaagaaaattcccatcttaccacttttcctttttcacttttcttaatcattttgcctgttttcttctcccagatgcccttcaagcctctgctgtatacttcagacacagaggatgtgagttcaccctgagtatggcgaaccactgcctccactgctgagcttttagagccacctctgcaagcacacactctgaccaagtggcctttgggagcacagaatattctggcattggacccacatctgatgacctgatgcccacccatcctgcactagcctctgtggctttgcaatgtgtttagagagaacccattttcctgatttgtttgtctgtcctttgtccttcaataacattcactgatgctgtgccagaggctggaaccaggggtgcctgtcctaaaaaatacatttcatggggagcaaaataagcagtggacagttcacagctgactggtatggccagcactgcagaggtgcctgggagaggcgaagaggtgctgggagcccaggaaatgtggaaatcatgcagacaaagccagtggaggccaggaccaggtaaggctgtgagagggaagcagggactgtggggagcacaggagaccggcatccttgtggggagggagctgcaacagggacatgctgggcaccatgcacaagggtgtctgcacctgccactcccccatcatgagggcgtctgcacctgctgctcccccatcacgagggtgtccacacctgctgctcccccatgaagcaaaccccagggagcattctggaaagaggcaaagtccagatctggactgttaactcagaacgacaggaccccagtttaaacctctggtttaacaaccatccaccacttatttctcttcccatgaaggtgtgcctttcagaggaaggcacccccagttccggcctctagcacagcaccagtaaatattatcgaaggacagaataaaaaaacacctgtccactatggttttggaatgctgatacttttttttttttttttaagatgaagtctcactctgttgcccaggctggagtgcagtggtgtgatctcggctcactgcaacctctgcctcctgggttcaaatgattctcctgcctcagcctcccgcatagcggggactacaggcgtgtgccaccaggccaggctaatttttgtattttttaagtagaaatggaatttcactatgttggttaggttggcctcaaactcctggcctcaagtgatccacccacctcggcctcccaaagtgctgggattataggtgtgagccaccttgccctgcctggaatgttgatactttttattcataaaagaaacagcacagatttctgcatctgtgtccatcttcaattcatttgtgtctgagcctgagacggggccttgagctctcgggctctggggaaacaagaccccagaattttgtagagaccccccccacaccatggccctgtcccctgatgggtcacacgtgatcatgaactcatggcatcctgtagactgtggatgagccttggagtgtggggctcacagcggagagactcacgcccaaaagggccctgaagtctctgggttcggagagactcacgcccagcagggccccagggtctcgggttcagagggactcatgcccagcagggcccctgtgtttcgggtttggagagactcacgcccagcagggcccgggcgtcttgggttcggatccagactgctttctgaagacacctcagtgcacccaggtctggtgcaccatttccttttaccaaaatagcacagaaaactgctcccgttgtgagcaccctcactcccacagaaagatgcatttctgctcagccccagatgggacgaggggcaccctgtggcctctgaccctttgggattggcagtcgcctgccccacacggaagcagaggtggacgcaacggccctgcagcagcacctgccccagccgggcacaggctccacttccggccaggtgcgctcacctggagtagtcgctctgcacctccagggtccgggtatccagcagcaggccgcaccaggggaataggccgtgggccggcatctgaacaaaagccgtgccacccagggcctcgtcttctacagggaagttcaccactgtcttccgcaagttcaccacgcagccatactcagggacacctcggaccagggtcctaaggcagaggggcaatgtcagccccaggatgcggggccgtcacccaggaggtaacctgacacccttgttaaatgctttggaaaaccccagagaagtggtgatttggagcagggtgctgggcctggcaggagctctgaggagcctggacccagccctgctccagacttcggggtgctttccctgtctcccgggcaggacaggtaggtgagcatgcaagaacctggcctggacccgggacagccaggactcagatgggaggtgcagccccagtggcctctgtgatggtccatctcatgtgtccccatggcaacaccacaatcccgttatagactcatctgtggtccggggcagactaaaatctggacaagaaattgccaccatcccaccttcttttaagacgtttctcaacgactttgcctgttttctcctagatgccctgaaggcatctgctgtgctttagacaaaggggagggttctgagttcctgctcaggcgcgggacctggctgggctgtgagctgggcaacaccagtcgtcagcttcacgtcaaggaggttccctcgccgagtcatgagcctcgctcacccagctcagggagcccccctgtgcccggtagcctctgctctggcctgggcatccccttcccttcctgcggcctctgctgtggccccgggcgtccccctgcccttcctgtggcctggccctgggacctcggccagctgcgcagctcccctggtgcattcccagctccctgtggccaccccttcctgttacatggctgccgactcatcagagacagagttctgcccccggtgggaccccaacatacactctgagccacctccctggccagcaccctcaggaacttgccacacagaagcagcgttccccgtctgctctgtcccctcagcagcctcaggactcctgagccctcagttgcttgggacctgggctccccccgagtgaatcttagtggatttaaatgtgcacagcctgctgtgctccgggctgcggcacaaggaacgccaggcatacccccagcccagggcggccctggagggtggacctggagggtggacttggagggcgggtccaaatgggttttcacaaacacagcccagcaaagcacctgaaaccacccctggcggccacattttctgagggttccctttacagctttcaaggtttctcgtccctcgtcaaatcgcactttaaaacaagcccaccggctctgtggggcgcccttgagggggaccacattggacaatcccctgtgctgaggccaggtcccctgtgcctgattttagtttgattcacaaaaatcaactctgaaacccactcctagccatctccgtggttcaagacagcagcactcagcaaatccaaattctcccaccgatcccaaacaaccccacgcaggaacgtgaccgcagcgaacgctgtgggccagctcactcagcagctcctgctctccgcaggctcaggtgcaccctggggaggaggctgccgcctcggccctgcaggccctgtgcacagctcctgggggtctcactggggccccaggagccgccactcttgactttccaaagagcagcaggagccaggtcaccgcctgcactcaccacgtgtgtaacctgggtgcgtctctgcaggaccagggcctgcacggtacccactccctcccgaaggtgcccctgcgcccccaggcctgggtgcacggccaagcgcctctgctcttgcggatccagcaccggcagagagagaggacttggcagagacaacgctgcggtgccaggggcaggacacggggggctcaactgcaaagcccacaggctgtggaggtccccacagacacacggcacgggcctcacctgaggaaggttttcgcgtgggtgaggtgaggtgtcaccaacaagaaatcatccaccaaacgcaggagcagcctaaaataagggaaaatacacagcaaggttaactttacactttttacgtagtatctgtctacgagggactgaatgtcaaacaatttacacagccataaataaagtaacattctccaaagcggttaaatgaaaaacttaaatttctttccaacagacgagctctctacagttaactgctacagcaattcctcattcattataagtactcagtgtttaccatcagcttgtgcaattctgtgccagctgaagacgaacgaaggccatcaggcgcggccccacttctcagactcttagggaaaaataactgaagacggtaagaaagctggggctgggaacctcgccccctctgagccacgtggtgccctttaccagcatggcagctgtggtcctcagcatgatccgagacccagatcctatccacaggaagctgggcatggtgcactgtgtgaccctcaggcccaagcccagcgccccatgaccatgggccagaggtatcctgtcccagagggctgaccagtgtgccgccagctcgaggacactttcctccaaatcccagagcaagttgtgtgtacaagaaatttttgtttctgttacataaacttatttcaatctagaacttgaaccttaatgaagaagaaccttgatccactgtacatgctataattggagctagagtttcaaaagatgcacaggaaaaacccggagtcttcgagagaaatggctgtgtggcctgcaggcttcctgacgcatccttcactaactgtctgcttgttcagtgatactattttcataaaaaaataactgctgtccacatcctctccagcaccagaacacttttacactgttggtgggagtgtaaactagttcaaccattgtggaagacagtgtggtgattcctcaaggatctagaactagaaataccatttgacccagccatcccattactgggtatatacccaaaggattataaatcatgctgctataaagacacatgcacacgtatgtttattgcggcactattcacagtagcaaagacttgcaaccaacccaaatgtccatcgatgatagactggattaagaaaatgtggcacatatacaccacggaatactatgcagtcataaaaaaggatgagttcatgtcctttgtagggacatggacgaagctggaaaccatcactctgagcaaactattgcaaggaaagaaaaccaaacaccacatgttctcactcacaggtgggaactgaacaatgagaacacttggacacagggtggggaacatcacacaccagggcctgtcatgggatggggggagtggggagggatagcattaggagatatacctaatgtaaatgatgagttaatgggtgcagcacaccaacatggcacatgtatacatatgtaactaacctgcacgtcgtgcacatgtaccctagaacttaaagtattaaaaaaaaaaaagtaactgctgtcaataaaagccagctgagttgaaccacattaggttggcacgatatttaggtgtgtgcgtccttgggtgtagaccccatgcaagtgggatgggcaatgggctgaagaggcgcccagtccaggccacctgtcgagggcctgctgggagatgtggggcctcaggctgcaccagccccatctccccaaggaccctaacggaacctgggtgacgtgacagaagctggtgtgcttcctcggtgttgaattcacatgctcatcatgcagggcagcggccctcccgcctgctgagccctgaggcctctggacatggccgctggacagagcctgcgtggagcccgcagtcctcaggctgtgcaacccctcccgtgcggcttcataccaagaaggggctgcaaccttgtctggttcctcaagacagagcagtcatggtctccagagcaccaggaatattaacactgaatgcatcaaaagcaaatcaacccccacccaagcccccctggggaagaggaggcctcacccgtcccgccgaatccccgcaaacagcttgttctccatgtcgccgtagcacaggctgcagagcagcgtggagaggatggagccctgcgggatcccctggcactggacgtaggacctggggcgggaagacacaggtgagagacgggcagggcatgtgctggacatgcgtacactcaaaccgagccacacacagacacagaacctcatacacacaaacgacacgtctaccattcagccggcaaacacctcagaaacataccagatgagacctctgaacaaacaatccctgctccccactctcaccatgcactggggcggctgaaacagatccagccacaggtgtgaccacatccttgctctgaatcatcaaggttttctttcattaaggtttcttaaatcttagagtttattcacagaaccagacacttgacccggaatgaatgcttaaccggactccttttttttttttcaaggaatttgtccacggtgaaagactcatgaccctacatgtagccgctgcgcgccaacggatacaaccttgcccctagtttcagcatgaccaacgagaagcagaaactctccaaacaaggatgccaagggtgcgttttcagacaaacagtgagagcagaatagccccgtggaacctaaaggtgcacacagaaggcatggctgccacgcgaccctcagccatccccagagcccacaggacgccactcctgttgctaagagacgcttgcagcctactgcaaaaacaagacctgttattttcgggaagcgctataggtggtcaccttaaaaagaggctttccggctgggcgtgatggctcacacctgtaatcccagcactttgggaggccgaggtgggtggatcacctgaggtcaggagttcgagaccagcctggccaacatggcaaaaacccctctctactaaaaatacaaaaattagccaggcgcagggggtgggtgcctgtaatctcagctgctcgggaggctgaggcaggagaaatgctggaacccgggaggcggaggttgcagtgagttgagatcgcgccactgcactcaagcccaggcagacaacagtgacgttccgtctcaaaaaaaaaaaaagaaagaaaagaaaagaggctttccttgctggtgcagatatcacagtgatgggcaagaggactgcactttttgcacagaagcacacgcaccaactctaggagtccggccagcccagcgagtcaacgcaagcagatacgcccctgtgagcacatgggatgggaaactctctctgttcttagtttttggggcaacaggttttgaagacgtttttattcagaggccaaactggaccacgatggggactagagcttcgggcctgtccgtgtcctagggacaggacagacacagtcacctcctgtctgtcagggcatgaggacgcagtcatcacctcacattccccaaagccctgccacacgtggacggtacgtgacttgatccatcttaataaaattcacgaccacgaaggaggcccgtggtggcttctctggggaggtgactttgtcagcgaattctgtgggcctgggggccgcttacacacatgtgcacacgtgtccctcggccaaaattcactctgctgccatgtgcaatttgtgtttttccagaacgtgtgtactacttaatatttctaattatactttaataaatattataattgctgataccattagtgattacttataatgcaataattaatagttatatttttaaataaaagcttccgaagctgtgcacagtcctttatccgctggagacgatcccagagagaggcctccacgttccacctgactgcggatgaccttgctgatgaccttgagtgtgcacgaatttcctccgtgctaccactctccccaggcacacacagggtcgcaggcagatgcctgccgggaggtgtgtggttttacttaaaatccagaggacgtgatgtggcaccaggcactgtggctctaccaccgtgagtgtggctcacccagtggctgtgtgacggcagctctcccaggccgcctgccccatggccaccacaggccccccgagaggagcaaactacacggtcaaccccgcactttccagatgggaaatctagtcacagagagaagagcccgcgccctcgcgggggtcacacgacagggacaggaatgagaatcggataaaatccttttgtatcgggagagagagatacaagcgtgtgagagccgggtgtccagcgtcagtagtaacttggagctgacaagctgcaggctcaaacgctcggcgcacacctgacccagacacaccccccgcccctcgtcctccacgcaggagcaactccacaccccgcttgccatttccaggcctcgtgtggcacctgctccgctccggctgccgcaagccgagccatgtttccggggcctcgggagcctgcagcccaggagccggagggggcgggggccagaaaaggagactctggtggccccgggcagaagggcagtggggaaggggcaggagagaggtgagcagaagccctgccagcccgcccagccacccgcagggcaatggcacctggccacctgactcacttgcccctgatgcgcacggcgtggtggcacatgaagcgtaggaagacgtcgaagaggccactgctggcctcattcagggaggagctctgcgaaagcagacgggagacacatgggagtgagccggtgggtgctgagacaggcaggaccacgtggccaagaccctccgtcccttttggggtcagtgtttgtgatgaagtgcggggctgggctggaatgcagggccatcgtgggctggccgggccgagtctctgcagacacacatgggcttaggcagagtgtgtgacacagaatgtctcctctgggccacctgtgggtccagctgggctcacacccacacagcccaccgtggcccggatggcatatgtgatccaggagttgctgcccagctgccgggcacagggtctgggtcccagcagcccaggccacctccacccccaccctgggagccaaggaaattcgcccagaggtctctgagcctcatgcggtggacacgactgtcccctagagccgagcaagtgctaacaggcatctgcaaagagccacagggccacgtggacagaccacatctgcaaagggccacagggccacgtggacagacccagcactgagaggcccgctcagcggggcgtggctcaaacacggcccgtcctcctaagaagggggacacgtttcctttggcctcagacccagcactgaggggccagctgggcaggacacggctcaaacatggcctgccctcctaaaggagggggacacctttcctttggcctcaagatcaaaactggcagagaagacatttagcaaatcatgaacaaaggcactgcggaactggagagaggccgcgcagtcaaacgtgagcaggtgcctgaatgcagcagctgtcgcagcctggcatggcccagctcagatttcgccaaggcacacagctcatcatgcccccatcacagctcattccccccactgccccccagggccaacagtctgtccggtcatgagcccagtgattgcccaggggaggacccactgctgggagtccgtgcccaaccctgcagggcagtgcccagacctgctcgatgacgacggcatccctcagcgggctggtctcctgcaggtgagccacgaactgtcgcatgtacggctggaggtctgtcaaggtagagacctgccggcagaggagagggcatgagccacaaatgtggcctgccccggccagagctgggcacttgtttcttccgatcaggacgtgtggacctgcggccaagcccgatggcgggatgaagcccagagagcgcctgggaagcttctgtttgggaaacgggggccggggggccgaggacgcaaagtagctacagacacacctgggactccacctgcagctaccacacatcagacccctgtgaccgatcaccatccacagtcaccacatcagaccccacgaccgccatccacagtcaccacatcagaccctacgaccgccatccacagtcaccacatcagaccccacgaccgccatccacagtcaccacacatcagaccccacgaccgccatccacagtcaccacacatcagaccccacgaccgccatccacagtcaccacacatcagaccccacgaccgccatccacagtcaccacacatcagaccccacgaccgccatccacagtcaccacatcagaccccacgaccgccatccacagtcaccacacatcagaccccacgaccgccatccacagtcaccacatcagaccccacgaccgccatccacagtcaccacacatcagaccccacgaccgccatccacagtcaccacacatcagacccctgtgaccgatcgccatccagtcaccacacatcagacccccgggaccaaccgccatccacagtcaccacacatcagaccccacgaccgccatccacagtcaccacacatcagacccctgtgaccgatcaccatccacagtcaccacacatcagaccccacaaccgccatccacagtcaccacacatcagaccccacgaccgccatccacagtcaccacatcagaccccacgaccgccatccacagtcaccacacatcagaccccacgaccgccatccacagtcaccacacatcagaccccacgaccgccatccacagtcaccacacatcagaccccacgaccgccatccacagtcaccacatcagaccccacgaccgccatccacagtcaccacacatcagaccccacgaccgccatccacagtcaccacatcagaccccacgaccgccatccacagtcaccacacatcagaccccacgaccgccatccacagtcaccacacatcagacccctgtgaccgatcgccatccacagtcaccacacatcagacccccgggaccaaccgccatccacagtcaccacacatcagaccccacgaccgccatccacagtcaccacatcagaccccacgaccgccatccacagtcaccacatcagaccccacgaccgccatccacagtcaccacacatcagacccccgggaccaaccgccatccacagtcaccacacatcagaccccacgaccgccatccacagtcaccacatcagaccccacgaccgccatccacagtcaccacatcagaccccacgaccgccatccacagtcaccacacatcagacccctgtgaccaaccgccatccacagtcaccacacatcagacccccgtgaccgactgccatccacagtcacgtggcacaagtggcactcgagctcccgggcctctccagcccagtgctggagggattgcagggatgcaggtgcagccacagcacaggcaggagaagagtctgaagacgcgggagagagaccggcacccccacctcctattctgcagactcccccaggacgcttgtcatttactacgggacctgctaagcccctgcgtccccaagacaggatgtgagcaggcacgtgacacacactaacacggacacaggaggcctcgagcttgtgaggcatcaaaagacgcgcacggtgactctgtgcttcagcatgttccccagcccccaggagctggcggcagggccgtgggctaaaaccacatcgagtacgattcaaccctgagaaccaacctggaaggcagtaacaggaaggtacatggggcctgcacccttcccaacccaggagcaggcaacacgcccacaaatagcccatgggtctgaggtcggcggaaagcatcacagaaatccgtaattattctggactcaacacagaaaagccaatatatcaacactgacgagaggctgctgaagcaggtctttagcacagggtccccaaccattgtggtaccagggactggtttcgtggaagacgatttttccacaggacagcagtggggaaggggatggttttgggagcaaacggttccacctcagatcatcaggcattagattcccataaggagcacagaatctagacccctctcacgcacagttctcagtagggtgtgtgcgcctctgagcaccgcatgccactgctgatctgagaggggcggggctcaggcgggaacgctggctccccacaccccacctagcctcccgctgtgcgccgggttcctaacaggccccagaccggtgccactccacagccaggggcctcgggatccctgctttagagggaaaacggcaacttcagatgcttgtttagaaaagaaagttcaaacatccataatcgaaacttttccttcaaggagccggaaaaacaacaataaacaaagcctaaaggaaatggaaggaggccatcgataaacagcagacaccaattacgcaggaaacagccgggaaatcagcacacgtgaaactcttctgtgaagagagtcatgaaatggaaagacccctagcgagacctggagaaggaaaacggaggttgtcagcaccaaggctgcaggcagctatcgctgcagatctcacggaagctgcaggaagcgagtctcggccgggcgcggcggttcacacctgtcatcccagcactttgggaggccgaggtgggaggatcacctgaggtcaggagttcgagaccagcctggccaacatggagaaacctcgtctctactaaaattacaaaaattagtcagcatggtggtgggcgcctataattccagctacttgggaggctgaggcaagagaattgcttgaactcaggaagcacaggttgcagtgagccaagatcacgccaaaccactccagcctgggcaaaaaagagcgagactctgttatcaaaaaaaaaaaaaaagaaagaaaaaaagaaagaaagcaagcctccaactcgcaggggaattccaaggacggagcgtagccagggccacatccaggccaatgaacacaaggaggcctggaagccctgcccaccggccacacaccacagcaggcaccacacgaccccagagtggaagaaacagaacagggagcccaggggaggcctccacacccctgacttactataaacccgaccatagttcaaaggaagatatggtctcctcaataaagctgttggagcacctgcagacccactacaggaagaaagctcgccttgaactctgtgttaccccactcataaaaaccaatcccacagatccccacctatcccacacatgaaaaccaatcccacagatctccacctaccccacacatgaaaaccaactccacagatccccacctaccccacacatgaaaaccaatcccacagatccccacctaccccacacatgaaaaccaatcccacagatccccacctaccccacacatgaaaaccaatcccacagatctccacctaccctacacatgaaaaccaactccacagatccccacctacccccacacataaaaaccaactccacagatccccacctaccccacacatgaaaaccaatcccacagatccccacctaccccacacatgaaaaccaatcccacagatccccacctaccccacacatgaaaaccaatcccacagatccccacctaccccacggatgaaaaccaattcacagatccccacctaccccacacataaaaaccaatcccacaggtccccacctaccccacacatagaaaccaatcccacagatccccacctaccccacggatgaaaaccaattcacagatctccacctacaccacacatgaaaaccaactccacagatccccacctatcccacacatgaaaaccaatcccacagatccccacctaccccacggatgaaaaccaattcacagatccccacctaccccacgcataaaaaccaatcccacagatccccacctaccccacggatgaaaaccaattcacatatccccacctaccccacacataaaaaccaatcccacagatccccacctaccccacacaagaaaaccaatcccacagatccccacctaccccgcacatgaaaaccaatcccacctaccccacacataaaaaccaatcccacagatccccacctaccccacggatgaaaaccaattcacagatctccacctacaccacacatgaaaaccaatcccacagatccccacctaccccacacatgaaaaccaattccaccaatccccacccaggtttgcaagctgacacagtaaagactgttctaaaaggaggcgcagaagcaacgtcaccacaacctcaataccttgagttcctgcagagaacacaaacagctttagccacaaacagctgataaagtggactgcgttactggactgtgttagtggactgcgatagaagtacaatcttccagtcctcaaaaggcttgacttgagagcacaaaagcaagacacacacggcacagagattcacagtgcacgcatgtgatgccacacttgcagcagaacacacaaagagctcctgcaagccgatgatggcaggcaggcgccccatacagagcggggcaggacggacagccacctcccgcaagagccccggggcggccagcacgggaacactgtgggcacgtgggcatcaagagagccccctgcacaatcccacgaggcggcttcgacaccttcaggccagcggccaccggaggcatcgccctcacaagcagccagccggggtgtcactgtcacccccctggaaactcccacaacatccctaaagcacgtctgtgacccagcaaccccacccctggaattcacccagaggcgagggcctctgacccctgggacactcccagaacccagcaggctcacagcaggcgcagccgtgcagtccgcacccaggagcctccacagcagcgggaaggatacatccacgtggcgagttccacaacggatgccattcatggacagaacgcacagctgaggcgttcagcatagctgaggctcgaaccccaaaacccacctcctgcctgatctcactcacatggagaccgggagagggagaccatggaggtcgaggtcggaacgagggtcacggtgggggcggtagcttccgctgcagcggggatgtgggggggggtctcctgggctctggtgactttctacacccgggcacaggtgggcggtggggacgggggggtctcctgggctctagtgactttctacacctgggcacaggtggtggccatcagctttccatgagggtaaaattctgctgagatttgcaagtagctgcgatagctcaagaagtcagagaaagaggtccttgtgggtttccacatgtgcctctccccagaccgaagctccagggatacttcttcacctctgcaccccggcctggcacgactttgagggcttacccctccagcgtgctcatgaccactgggtgggaacccaagcccagggcccctttcacccctgccgagccccaaggcccccggccgctctgggaggagtgtgtcccggggagcgaggcctgaatcagccttcctatatctcatggagccggcataggccagactggggtcctgggggtgccaggggtccaggaaggtaccaccagcatccctactctcctctcaaacctggcttacagtctccaaggcccagccctgtgacaggacaggggcacccatgctagaagtgaggaccctgaatgtggagctccccaaaccagcacccgagagcgtgaggacaatggtgcggaccatgcctggaggaggaaggagtaaggccaaggggccacacacacccatgccatagacacgcatatgtcacggacacatgcacacacacacaccacaaataggcacatcacagacatgcagcacacagcatgaacatgcacgtgtcacagacacacacacatgcacaccacaggcacacacaccatggacacacacgtgccacacacacacacaccacaaatatgcacgccacagatacatgcaccacgacacacacgtgccacacacgcatatcacagatgtgtaaatcatggaagtacctccaccacagaaacgcatcacagacacgactgcattctagacacattcatatcccagagacacacatcctggacacgactatcacacgtgaaccttacgtggctcttgaaggccttgcggacgtgcccatgggcggccttctggaccacggcataccgacgcacgcagtacgtgttctggggtttgatgatgctggcgatgacctccgtgagcctgtcctgggggatggtgtcgtacgcgcccgtcacatccacctgtgtgagtggaggcgaggagactgacagtggccacgcagaaactcagacatcacctctgccctcagggcctggcctggcggtgtcccccactctctctctgacccccaccactccagaccccaagggcagggcgggctgtgtcccctctctgagcctcaggacaggagacccctggctcaggactggggtgcaaggcaccggggcctggtggctgagccgttgcggttccttctctgacggaaactggaatccagtgggaccctccttgggaataggagcccgggcagtcagggtgcacagtcggccccatgtgctgcaggaaaggctgaaggcctccaccctaggtgccaggtgtgtcctcaacagtgacagggtcacctgcactccctgcggcccacccaggagtacctcctccacccaacatgaggtgccaatcaggcagccactcccaaggtccagcagggctgctcacgggggtccccggcacccaccttgacaaagtacagctcaggcggcgggtcctgggcccgcacacgcagcacgaaggtgcgccaggccctgtggatatcgtccaggcccagcacagaggcgcccaggaggccggggcgccgcgcccgctcgtagttgagcacgctgaacagtgccttcaccctcgaggtgagacgctcggcctggcggggacagcatgggagacagtcaggaaagtggatccggccaagtgcccaccccaccatagggacccaggggagaagcagcccctccccagtgtccccagccacccagacccgggacctagaacccctcccagcttcctcagaccctgtttgaaacgggttcctggccgcatgtgtgttgcacacgggatcctcatgccacacctctgtccacctcaccccacactctcctcagatgacggggtcaccgcagccaccgcagccacaggggtggggtgcaggagccgtggggcaaggtccaggatcctcagagcccggtgggctcctggcagtcgtggcctggtccccgggccccgtaagcggaagcgcacggaggctgcgcaccacagcaggacacggatccaggacctcgggccgtgctgcatccaggccctggcccggctgcttcttgtggtcctcagagcctgtccctgccccaggagctccccctacacccccacgctgcttttctggaggcaccggcacctcggccacctcactgtgcgcacaaatgggttggcgccgtgccatggccctggctgtgtcccgtggcccctcctccgggcccttcatctaagctgataccaaatgtggggctcaaacgcacttctgtttaaaaaggaagttaaaccaaagcacagccaccctcttttctctgcggaacgttctggctcccacgacgtagtccatgttcacaatcggccgcagcccgtcaggcttggggatgaagcggagtctggacgtcagcagggcgggcctggcttcccgatgctgcctgacctctgcttccgacagctcccgcagctgcaccctcttcaagtgctgtctgcaatagagagcccctcaggaggcttgctcagccagacaacagactagggggaagctcacgggaagccacaagcccccaccgactcagtgagggctcagggcacccacggcagcacacgctgaaggccatgcccggggccacgtccacccatgccagccagacgcctctgagagcccctctacttgcagggcacctggacacctggtcctatagtcaaccctggggacgcccagtccggcggtcaacccccagggtgactgtcccaccatcagcaccagggagcctggtcccaggctgcatctggggccacgtgaacccatgcccctccacacaggatgcctgtccctctggtacagccactggggtagcccaaggctggccccaggcaggacccccaggtagaaagagccacatcccttcagatggagagggcgggagaggctgctcagggcagctcagccaaggagcagaatcagctctcatgagcccaccagtctcctgacacctctgccctttggccttttcccccaaaaccacccatgggggcacgggggctggagcggccacacctggacctgggcatctgtggctgacagcctggcactccgtggacactggggccgcgggtgctccggagctggtgccctgaccgcaggccgcaggcccagcagagtagaaaaggcacggtaccctgtccagggaggaggggcctccaaggccccccaggcagaacaacagtcccagtagtgacaaggagcatcttggggaccaccacgtctgtctgctccttccggtcaccccaattaaattctttccaaaatactggtcaaatgaccatgttgggtcgcacaccattaaatacgtgtaaagtctaagtaaggtgcattgtgtattaacttgttcccacagggaaagccctgaccctcccccagatgcaaggaaatggccccagcaccacgtcggggtccccagcccagaggctgtgttacctctgacgaggtctgctgccagctccctctgcagacatcctgatttggggcatggggtgtcacaaggccccccagggactcgagtgtgcaggggagctggggaagccctgatcttctgactaaagccctccacagtcagcctgggcgggacacggagaagctaataccatggcctgggctcagccaggctggcatctcccagaccagcgctcccgtccctcactgatgcaaatccagcttgaatctgtcacaatctcacactctcatcagccgaacattgcagacaaaatctcacagccattcaaatgcagccacctagaagttagctcatggtggaactctcctatccccaggtgctccctgcacaggtgctccctgcagtgagtcgccaagtgggtgggattttatcctctcatgacctaaatccaatagattgaagattcatacagaacagcatgaattaaaagagaatcgggggctgggcacggtggcttagcctgtaaacccagcactttgggaggccaaggtgggtggatcacctgaggtcatgagttcgagaccagcctggccaacatggtgaaaccccgtctctcctaaaagtacaaaaattagccaggcgtggtggcaggtgcctgtaatcccagctacttaggaggctgaggctggtgaatcgcttaaacccaggaggtggaggttgcagtgagccaagattatgccactgcactccagcctgggcaacagagtgagactccatctcaaaaaagaaaaaaaaaagagagagagaatcagacccacatcccagggaaaggcaaggaggctagtggtagtgtttccatctaatttaaatcccttctgctttacaaaccctagagaccacttggcacaaactcctgattttacaagtaagagaacgtgagctccaggcagtcagagccttgcacagaatccacttggaccaggcctgtgacggggcccctggctcccagcccagagctgcacagccagggaagcacaggcaagtggagacaggcgcatgctgaggccgggctggtgttccaggacttcgagaagcagaggcctggcgtggggatacagtacctgattccaatgctttgcaacttgctccagacactcttccggtagaaaaagagcctgttcttttgaaacgtggtctccgtgacataaaagaaagacctgagcagctcgacgacgtacacactcatcagccagtgcaggaacttggccaggatctcctcacgcagacggtgctctgcggccggaacacagccaaccccttaaacgagaaggacatgccacatccagatcaccgagggcctggtgacctcaccccggacctgcaccatccggacaccgcacatccagctcaccaagggcctggcgacctcaccccagacctgcaccatccggacacggcacatccagcccaccaagggcctggcgacctcaccctggacctgcaccattcggacacggggacaccgcatatccagctcaccgagggcctggcgaactcaccccggacctgcagcatccagacaccacacatccagctcaccgcagggcctggcgaactcaccccggacctgcaccatccagacaacacacatacagcccaccgcagggcctggcgacctcactccggacctgcaccatccgtacactgcacatccagctcaccgcagggcctggcgacctcacgccagacctgcaccatccggatacagcacatccagctcaccgcagggcctggcgacctcaccccggacctgcaccatccggatacagcacatccagctcacagcagggcctggcgacctcaccccagacctgcaccatccggacaccacacatccagctaaccgcagggcctggtgacctcaccccggacctgcaccatccgacaccgcatatccagctaaccgcagggcctggcgacctcacgccagacctgcaccatccggatacagcacatccagctcactgcagggcctggcgacctcaccccggacctgcaccatctggatacagcacatccagctcacagcagggcctggcgacctcaccccggacctgcaccatccagacaccgcacatccagctaaccgcagggcctggtgacctcaccccggacctgcaccatccggacaccgcatatccagctaaccgcagggcctggcgacctcaccccggacctgcaccatccggacaccccacatccagctcaccgaaggccttggctacctcaccccggacctgcaccatccagacaccgcacatccagctcacagcagggcctggcgacctcacgccggacctgcaccatccggacaccacacatccagcccaccaagggcctggcgacctcaccccagacatgcaccatgcagacaccgcacatccagctcaccaaaggcctggcgacctcaccccagacctgcaccatccggacacggcacatccagcccaccaagggcctagcgacctcaccctggacctgcaccatccggacacggggacaccgcacatccagctcaccgagggcctggcgaactcaccccggacctgcagcaaccggacactgcacaaccagctcaccgcagggcctggtgacctcaccccagacctgcaccatccggatacagcacatccagctcacagcagggcctggcgacctcaccccggacctgcaccatccggatacagcacatccagctcacagcagggcctggcaacctcaccccggacctgcatcatccggactccatacctccagctcaccgagggcctggcgaccacaccccagacctgcaccatccagacgccgcacatccagctcacagcagggcctggcgacctcaccccggacctgcatcatccggactccatacctccagctcaccgagggcctggcgaccacaccccagacctgcaccatccagacgccgcacatccagctcacagcagggcctggcgacctcaccccggacctgcatcatccggactccatacctccagctcaccgagggcctggagacctcacccctgacctgcaccatccggacaccgcatatccagctcacagcagggtctggcgacctcaccccggaccggcaccatccggactccatacatccagctcaccgagggcctggcgacctcactccagacctgcaccatccagacaccgcacatccagctcaccgcagggtctggcgacctcaccccggacctgcaccatctggacactgcacatccagctcaccgagggcctggcgacctcactccagacctgcaccatccagacaccacacatccagctcacagcagggcctggcgacctcaccccagacctgcaccatccagacaccacacatccagctcacagcagggcctggtgacctcacaccggacctgcagcatccggacaccccacatccaactcacagcagggcctggcaacctcaccccagacctgcaccatccggacaccgcatatccagctcaccgcacggtctggcaccgtctggcgacctcactccagacctgcaccatccagacaccgcacacccagctcaccgcagggcctggtgacctcaccccagacctgcaccatccagacaccacacatccagctcacagcagggcctggcgacctcaccccggacctgcatcatccggactccatacctccagctcaccgagggcctggagacctcacccctgacctgcaccatccagacaccacacatccagctcacagcagggcctggcgacctcaccccggacctgcatcatccggactccatacctccagctcaccgagggcctggagacctcacccctgacctgcaccatccagacaccacacatccagctcacagcagggcctggcgacctcatcccagacgtgcaccatccggacactgcacatccagctcactgagggcctggcgacctcatcccggacctgcaccatctggtacaacagccatggccatgggaagcccagcaggtccaggccgagatggccacgcacgggacctacacacgggatcccgcagacaaagctcaaaaaatgtcaaatgtcctgtgagtgatgctcccaccaataacaccctaaaatgatgaggtgtggacatgctgggcttcagtaaaatctattattacattaatttcaccttttcttttttacttttttaatgtggctactagaatttttttttttttttttttttttgagacagagtctcgctgtgttgccaggctggagtgcagtggtgcaatgtcggctcactgcaacttccaactcccaggttcaagcaattctcctgcctcagcctcccgagtagctgggattacaggcacccgccaccacgcccagctaatttttgtgtttttagtacagatggggtttcatcatgttggccaggatggtctcgacctcctgacctcgtgatccacccgccttggcctcccaaagtgctgggattacaggcgtgagccaccgcgcccagcctagaaattttaaaattatccacatggctcacgtcatgctcctgttgggcgacaacggctcggagcctcatcctttgtcctggttcccaagcagaagggaggaagcagacactcccccgccaggcagcaccgtcagagctggcgccacaccgcaggtttgcgcgatttcaaactcgacaccgcggagccaccagaccccgacatgcctggggttttccaggctgaacccaggccgagcggacgttgctgtcactcactggctaaggaacgctggccacgtggtgctccagacactcacgggccaagatgaccgccctcctcgtgagtctccacatcttcatctgtgcatcataagcagaggtccccggacgtcgctagatgccatggaggccagcacaggtaaagctggggtttaccagcaataacaagacagaagaaccacgcaaaggacaaggaaggggaccccagacggcgggcctgagacagaagacagacggggaacaaaggaggaaaagcagggcgggggcaaagctacagaaacactcaacacggaaaacaatattaataatgcttagcttgtgggggtgtcaagatgcctgagatagaactaagtcagcagggaaaacagcacacaggctggggggtggccagagctaaaatacactaagggtcttattgctcaggacaagggtagcaatgtttcagacttcattttaaaagatcaaggtatgccgggcacggtggcttgcacctgtaatcccagcactgtgggaggccaaggcgggcagatcacttgaggtcaggagtttgagaccagcctggccaacatggtgaaagcctatctctactaaaaatacaaaaattagccaggcgtggtggtgggcgcctgtaatcccagctactcatgaggttgaggaatgagaactgcttgaaccgaggaggcagaggttgcagtgagccgagattgtgccattacactccagcctgggtgacagagtgagactgtctcaataaacaaaagcaaacaacgacaataaaaagatcagggggtaacactaagcaaagagaaataaaagcaaatgcccagtgaccaggaggcaacgagaggatcaacacacactcggcaggaaacgcacatggcaacccaagaggtggtgagaaacaagggaacgaggacatgcaacaggcaagtggagaatcagagtgcaccaggcgggtctctagtgacaaaaaaatgaaaataaatcaggttatccagttaaacaacgactgtcagactggattggaaattcacctacatgctgttaacaggacatacccacactataagaatataaaagtgttgaaaattaggccagacatggtggctcatgcctgtaattcagcactttgggaagccgaggtagatggatcccttgagtccaagagtttgagaccagcctgggcaacacagcaagaccctgtctccagaaaaaatttaaaaattagccaggtgtagcggtgcgtgtctgcagttccagctattcaggaggctgaggtgagaggacggctcaggccagaaggtaaaagttacactgacctgtgatcacaccactgcactcaccctgggcgacagaccaagactctgtctcaaaaaaaaaaaatatatatatatatatataaattaaaggcagaaaacagacacagcaggaaaatactaatcaaaagaagcccagtgtggcagtactaagagcaatttaaaaaaagaattactagattactagagacatagaagtctccaaaagattcactcatcaggaagataaaaattctaccacgtgcatgcacctaacaatgcctcacataaatgctaccaaacgagaagaaatgaacagacccatcccccaggtgagggactatggcctccttcttggaatttcatgcatctagaaggagacagaagctttgcactggaccttaataagctggatatagtgaacatgcatgcccactattgacaggatgtcaaatttacaatggacagttctggaaaccatgcacacactagatcacaaagcaaggctccgcaaacttcaaataattggtattatacagaccactacacaactgagttagaaatcaataataaaatgataacttaaaatactctacgtatcttgatactctacatatcttgaacataaacagtgttcaaataactcactgctcaaagaaatcacagaataagctaaagaatacttaaaagtcactgatatcaaaatgatttcacgtcaaaacacagcgtgcacaaagaaaaaaatcacagaataagaatacttaaaagaagtattaacactttgaagtattttaagaacacttgaaagtggctgatgttgagattacttcacatcaaaacacagggtgcaggtaaggcagcacttagagggaaaggcatgactaaactaagaagctagaaaaggaatgaaagaataaaccaagaaaaagcatacaatggagagatagaaacgtcagaggtaaatcagtgaaattcaaaactaagacccaagagggaagtctgacgaaggctggcggagacaccggccctccacgtgggtctcagagcaggagacagacagagacactccaacctgtggctggacgtcaatccatgtgagggggcgactggaggcagagcccagcctaagcaatgagcggcaggtgcccagaataaggtgacaagcgttgccacccacccaagggggccaagcagaggggcagcttgggagaaaacaggacaagtgagggggctggggtgacttgctttccttcagggcacagagaaccctttctgggatgtctccagcaacacgtggcagcagacacaggcagcccagagtccagcctcggcatgagacaagctgggagaggagtattggcagcatgcatgtggcgggcacgtggtgagcaagccaggcccaacagaggcaagcggacctgaagctgtggctgcagtgcctggcacgcgggaggcgagagcctgcagtcccgtgtccccgtagcaaaatggaacggagaggtgaccaagaagagccgagcatcagaaaagataggcttggggaccagcactgcgcctgcaccatctacccaggcaatgggcaaccggcgcagctgtggctatagaaagagcaaacattcaggagcaagctcaagtgagcaaacgccaaggacacctggggacagagcctcactcaccctacacgagacagggacacccagggacagcgcctcactcaccctgcacctgagagggacacccgggggccgcaactcactcaccctacacgtgacagggacacccggggacggcgcctcactcaccctacacgtgacagggacacccggggatggcgcctcactcaccctacacctgagagggacacccagggacggcgcctcactcaccctacacgtgacagagacacccggggacagtgcctcactcaccctacacgtgacagggacacccggggacggggcctcactcaccctgcacgtgacagggacacccggggacggcgcctcactcaccctacacgtgacagggacacccggggacggcgcctcactcaccctacacgtgacagggacacccggggacggcgcctcactcaccctacacgtgacagggacacccggggacggcgcctcactcaccctacacgtgacagggacacccggggacggcgcctcactcaccctgcacgtgacagggacacgcggggacggcgcctcactcaccctgcacgtgacagggacacccgggggcctcgcgtcaatcaccctgcacgtgacagggacacccggggacggcgcctcactcaccctgcacgtgacagggacacccggggacagtgcctcattcaccctacacgtgacagggacacccggggaccgcgcctcactcaccctgaacgtgacagggacacccggggacagtgcctcactcaccctgcaagtgacagggacacccgggggccgcgcctcactcaccctgcacgtgacagggacacccgggggccgtgcctcactcaccctgcacgtgacagggacacccgggggccgcgcctcactcaccctgcacgtgacagggacacccgggggccgcgcctcactcaccctgcacgtgacagggacacccgggggccgcgcctcactcaccctacacgtgacagggacacccgggggccgcgcctcactcaccctgcacgtgacagggacacccgggggccgcgcctcactcaccctacacgtgacagggacacccggggacagtgcctcactcaccctacacgtgacagtgacacccggggaccgcgcctcactcaccctacacgtgacagggacacccgggggccgtgcctcactcaccctgaacgtgacagggacacccggggacggcgcctcactcaccctacacgtgacagggacacccggggacggcacctcactcaccctacacgtgacagggacacccggggacggcgcctcactcaccctgcacgtgacagggacacgcggggacggcgcctcactcaccctgcacgtgacagggacacccgggggcctcgcgtcaatcaccctgcacgtgacagggacacccggggacagcgcctcactcaccctgcacgtgacagggacacccggggacggtgcctcactcaccctacacgtgacagggacacccggggaccgcgcctcactcaccctgaacgtgacagggacacccggggacagtgcctcactcaccctacacgggacagggacacccggggaccgtgcctcactcaccctgcacgtgacagggacacccggggaccgcgcctcactcaccctgcacgtgacagggacacccggggaccgcgcctcactcaccctgcacgtgacagggacacccggggaccgcgcctcactcaccctgcacgtgacagggacacccgggggccgcgcctcactcaccctgcacgtgacagggacacccgggggccgcgcctcactcaccctgcacgtgacagggacacccgggggccgtgcctcactcaccctgcacgtgacagggacacccggggacggcgcctcactcaccctgcacgggacagggacacctggggaccgcgcctcactcaccctgcacgggacagggacacccggggacagtgcctcactcaccctacacgtgacagggacacctggggaccgcgcctcactcaccctgcacgtgacagggacacccggggacagtgcctcactcaccctatacctgggagggacacccagggacggtgcctcactcaccctacacgtgacagggacacctggggccgcgcctcactcaccctacacctgagagggacacccggggacagcgcctcactcaccctacacctgggagggacacccagggacggtgcctcactcaccctacacgtgacagggacacccggggaccgcgcctcactcaccctgcacgtgacagggacacccggggacagcgcctcactcaccctgcacgggacagggacacccggggaccacgcctcactcaccctacacgtgacagggacacccggggacggcgcctcactcaccctacacctgagagggacacccggggacagtgcctcactcaccctacacgtgacagggacacccggggaccgcgcctcactcaccctgcacgtgacagggacacccggggacagtgcctcactcaccctgcacgggacagggacacccgggggccacacctcaatcacgctgcacgggacagggacacccgggggccgcgcctcactcaccctacacgtgacagggacacctgggggcaacgcctcactcaccctgcacgtgacagggacacccggggaccacgcctcactccctgcataagccaggggcagattgtgacctcatctgaagtcagagaacagcaatgacaggcagagtcctgatcagagaactcaaactctcctcaacgaaggaagctggagcacaaaaagcaaaactgggttgcatgacgcttatctgactcggcgtggtccacctgagccgcagcaggtgtgaggcagctgccgttcgatgggtagggacttccagtcacgcaagacgcagcatttcaagcaacctgctgtaaacaccgccgagttagcaattctgcactgtacacagaaaacggtgttaggagtgccaatctcatgttatatgacttttgccaccataaaaagaaaaaaagaaaaaaaagagccccaagaaggtcaccctccttgtctgcatggccggaagtcttacatgtcttgggagtttgtggggagggggtgaaatcgggacttcttctagctgccacggtagggcctgggagcactgggagccaaaagggggctggagcggaggttcctcaacatcaaatccagaaaaacagggtggggacacggcagggcccagcagcaccatcccctgaacacccacaaacactgtcccttcctcagcaggtggagccatctgctgtcctctgctcccatgtggccctcttcatacctaaagatgggaccaggatctgtgctggagaacagtcttatctccctccctctaccctgtcctggcacaatcaacgaacactttttttttttaaagacagagtttcactcttgtcgcccaggctggagtacaatggcacaatctcagctcactgcagcctccgcctcctgggttcaagtgattctcctgcctcagcctcccaagtagctgggattacaggcacacaccaccatgcccagctaatttttgtatttttagtacagatagggtttcaccatgttggtcaggctggtctcaaactcctgacctcaggtgatccacctgcctcagcttcccaaagtgctgggattaccggcgtgagccaccgcacctggccgtcaacacacaattaaatcttaaacacaaacctgcatattggctgaccacgtgcacctgcaaaacccttacctcccacccccaggaagagggggttctcgtccccacctctcattcccacccttgaaattgcgaagaggattataggtaacctgcaggcaccctcgccagagcgtctgtgcttccagacacttctccccattgccggcaacccggctccactgccgcgcccagcctcctctgttcactgctctggcctcggcgcctggaaaccgcgtgtccatcaaaacgtgaaggtgaacctcgtaagtttatgcaaactggacaggagggagagcagaggcagagatcaccgtgtccactcgacgtcctgagcgaaaagccacgtgtgcccacgtgacgatggagacaggaggaccagggctctgcctgcccccttttctgagcccctactgcattcagctctggggcctgggccctcgacggccaccacctcctcacctgggctcctgcgcagccaagcgcagtcccgcacgctcatcttccacgtcagctcctgcagcgagagcttggcatgcttccccagggagatgaacttcttggtgttcctgaggaagcggcgttcgttgtgcctggagccccagaggcctgggggcaccagccggcgcaggcaggcccgcacgaagccgtacacctgccaggggctgctgtgctggcggagcagctgcaccaggcgacgggggtctgtgtcctcctcctcgggggccgccacagagccctggggcttctcccgggcacagacaccggctgctggggtgaccgcagctcgcagcgggcagtgcgtcttgaggagcaccccgtaggggcactgcgcgtggttcccaagcagctccagaaacaggggccgcatttgccagtagcgctggggcaggcggggcaacctgcggggagtccctggcatccagggcctggaacccagaaagatggtctccacgagcctccgagcgccagtcaggctgggcctcagagagctgagtaggaaggagggccgcagctgctccttgtcgcctgaggagtagaggaagtgcttggtctcggcgtacaccgggggacaaggcgtgtcccagggacgtggtggccgcgatgtggatggggggcccgcgtggtgctggcggcccacggatgggtgggagtggcgcgtgccagagagcgcaccctccaaagaggtggcttcttcggcgggtctggcaggtgacaccacacagaaaccacggtcactcggtccacgcgtcctgcccgggtgggcccaggacccctgcccaacgggcgtccgctccggctcaggggcagcgccacgcctgggcctcttgggcaacggcagacttcggctggcactgcccccgcgcctcctcgcacccggggctggcaggcccagggggaccccggcctccctgacgctatggttccaggcccgttcgcatcccagacgccttcggggtccactagcgtgtggcgggggccgggcctgagtggcagcgccgagctggtacagcggcggcccgcacacctggtaggcgcagctgggagccaccagcacaaagagcgcgcagcgtgccagcaggtgaaccagcacgtcgtcgcccacgcggcgcagcagcagcccccacgccccgctcccccgcagtgcgtcggtcaccgtgttgggcaggtagctgcgcacgctggtggtgaaggcctcgggggggcccccgcgggccccgtccagcagcgcgaagccgaaggccagcacgttcttcgcgccgcgctcgcacagcctctgcagcactcgggccaccagctccttcaggcaggacacctgcgggggaagcgccctgagtcgcctgcgctgctctccgcatgtcgctggttccccccggccgccctcaaccccagccggacgccgaccccggggaggcccacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctcggcagcggggagcgcgcggcatcgcgggggtggccggggccagggcttcccacgtgcgcagcaggacgcagcgctgcctgaaactcgcgccgcgaggagagggcggggccgcggaaaggaaggggaggggctgggagggcccggagggggctgggccggggacccgggaggggtcgggacggggcggggtccgcgcggaggaggcggagctggaaggtgaaggggcaggacgggtgcccgggtccccagtccctccgccacgtgggaagcgcggtcctgggcgtctgtgcccgcgaatccactgggagcccggcctggccccgacagcgcagctgctccgggcggacccgggggtctgggccgcgcttccccgcccgcgcgccgctcgcgctcccagggtgcagggacgccagcgagggccccagcggagagaggtcgaatcggcctaggctgtggggtaacccgagggaggggccatgatgtggaggccctgggaacaggtgcgtgcggcgaccctttggccgctggcctgatccggagacccagggctgcctccaggtccggacgcggggcgtcgggctccgggcaccacgaatgccggacgtgaaggggaggacggaggcgcgtagacgcggctggggacgaacccgaggacgcattgctccctggacgggcacgcgggacctcccggagtgcctccctgcaacacttccccgcgacttgggctccttgacacaggcccgtcatttctctttgcaggttctcaggcggcgaggggtccccaccatgagcaaaccaccccaaatctgttaatcacccaccggggcggtcccgtcgagaaagggtgggaaatggagccaggcgctcctgctggccgcgcaccgggcgcctcacaccagccacaacggccttgaccctgggccccggcactctgtctggcagatgaggccaacatctggtcacatcccgcccgcacagggtggagggcaacctcggggtccaggcacctggctccaagcctcggactgcagagctaggaggcccgacttccagcccagcagtagaagccacacggccactggtcccctccagacctggggccccggcacaaccgcaggacagctgaggacttcccaggaatccagactccgggttgctcaagtttggatctaaggggcgagaaacttctgggtctcccgaggccttgcagggatgctgtagctgaggtcggcaaacactgaaatgctaacaaacgcaaccttaaatgtaacctttcctactttcagaaactgccggaggaaattgctttatttatggagctagcatttgaacaggcctcgcaccctccctgggctgtcacgctcgctggaggttagcctcgtcttgtaaatacttaggattacaggtcgctcttctagaaatccccttagtgatccctaagcctttttaaagggctgtgtttgtgaattgtctctgccactagggcaaaggggcggtttggaaaatttgttccaacaaaagttaagttgtagcttacactggttctctgcagagaagccaacatagaaaacacaattttaaaagagggaagagaagaaatggaagcagaagattatgctggagtaattaacaccatgtgcatggcgaggaaacgcctcccggcattcaatgaagatcgctgatacccagaagacaccccagtattatgggtgcagttagtgtgtctttgaaaagctgatgatgtcttagtcatcacagtgtaaaacatcaagagtgttctaacaacaataaaaaaattctatcattggcttaaaacaccacaacacttgagtggggtgagcttcctacctcagacccagatgtttctaaacagagtaaattctgagctgggcatggtggctcacacctgtaatcccagcactttaggaggcagaggcaggtggatcatctgaggtcagaagttcgagaccagcctggccaacatggtgaaccccacccccacccccgtctctactaaaaatacaaaaaattagctgggcatggtggcgggtgcctgtaatcccagccaaatgggaggcggaagcaggagaatcacttgaacctgggaggcagaggctgcagtaagccaagatcgcgccattgcactccctccagcctgagcaacaagagtgaaactgcctctcaaaacaaaacaaacaaaccaacaaatgaaacagtggattcagtaccagcagtgaaaaataacaaagtatcattccttcctccacaggagtgagaggaactcctctgcccctggggggggatagggaaggggctcaatcccagtagagtaggagggatggactttaatttctacatgtttcagcacacactgagacattgagattcaggacagtgtccacactcgcgccctgattctacatcaggtttctactggcagatggcaaccccactgggacctgaagcctgcagcctcccagctgcccctgcagtgggtcccatgggataacaggtggtcacagaaactgtgaatatgtcaagagacggtgtatccccagtctacgaagaattacagaaaatatcaaaggagggtaactcctgagtaaaggggaaattgaactccatgaactccttaccagtgggtgtgacttgagcccagggaagttttaaaatggttaaataggccaggtgcagtggctcatgcctgtaatcccagcactttgggaggctgaggtgggcggatcacctgaggtcaggattttgagatcagcctgaccaacatggtgaaagcccatctctactaaaaatacaaaattagccgggtgtggtggtgcatgcctgaaatcccagctacttgggaggctgaggcacgagaattgcttgaacccaggagacggaggttgcagtgagccaagatcatgccgctgcactccagcctgggcaacaagagcataactctgtctcagaaagtaagtaaataaaataaaatagctaaataaaatcactcgtgcctgccctgcccttgcccggaggcacttcagccctgtccttggatgtttctgtaaacctttgttttgaaataatcctagattcacagtgcatgggtttcctgttgcttctgcaaatccccacacacttcatgctagaagggcagcgcgagtgccctcctatggcacggaggccggaggtccacgatgggccctggtgggctaagggccagccgtgccttcctgctgcaggctccgtggagggtcccttccccgctttttccagcttctggaggcgcttgcaggcctgggctcgaggccctccctcccttttcagcctcagactgactgcctccatcgtcctgaggctgcccccctctgaccctcctgcccgcctcttcccagaacccctggattacttttcgggcctgcaggtagtcccgaaggatcccagacctcagggtcctcacggagatcacacctgcaaaggccctggggccgggcgggttctgtccacaggtttgggggaatcaggacgtgggcatctctagagcattatcctgcccaggacactcgggcatggcaaagatggcacagcctccgggttcccccatccccactcccctatggttagtctcccataactgagccaataccatcagaaccaggaaactggctttagtaaaaggtgcgtgcagtagcccccagctgccgcccccaccgtccttaacccccctaaccactcccctgccatggggtgaaattctttctttttgagcatgttgcgtacgtatcccatggaggtggcagttttgactcagcgtaattcccttgagatgcctgcaggctttataaacggcatgttcctcttcactgtgaggaactgtccaccgtgcggatggacccgtttgtttaaccagtttgttaaaggacattttagcagggatttccctgctttgtagctattaaaataaaacagcgggggtcggtcgtggacgggagtgtgtacatgatgtgcgtagtcacgtgaattcccagggctgtggggcaaatgtccaggggtgccaatgctgggtcttttgggaagaggatgcttagtgcttgtggaaactgccactctgtgttccagagggaggggggcagaaaagtgctgggcagagaagggcgggtccctggcgggggctccatccccaggcctgtgcccgtggacctaggtgaggacaggcactcctactttcacacccaaatgttgcatttcccaagagcgccctggcccaccacgcccccgtcctatgcctataaaaacccccagaccctagcgggcagagacacacgcagctggacgtggaaaggagcacagaaacagaagaacacaccggcaggccccggcagatgacaggagacggggagcgcctggaaatggaggtcagcgtgcagccggacgccaggaacctgcatcgggggccacgagccaggttcaggctcagaggagccagtctgggaggcgcagaatctcctcccttcaacgcatgtgagtgttggggaaggagacagacaccccatgtgaggaggaaccgtgcgagctacgtcgctgagacaagacacagtgggacaggtgagacagtagcgctggaacacggcccacccagggtttaggaagctgctggagaagctgaggcctggaggtggagggtgtggagacagggctggcctcagtgtggcaccccaggctgagtctccagggcagatggcgccctcgggaagcagcaaccaggccctagaatctgggggagtgcctgccagggtctactagtctagaggaacagactgttcagccgctgttgtcaccccagcctggtggctgaaagcagcctcatctctcctggactcttaatatatcaggggtgtgggccgtgctctgtgggcctagtaaaagagtggtgtttgtactgagtgttgtcatggcttgtcacacccgaagaagttgctggaatcattcaatccttgggggtgaagatgaataatgatggaaggaacggtccccatcagcagagtcgtgtaatttacatggccatttcctgcacaccacttcattacccttgacccacccagggaagcaggcaggcaagggtcatgttcgtttccacgggttcagagctccgtgacttcctgggttagtgtgggtgggcccaggaagccggtatggggtcctgagtcacacgcagcgtggccgccttcgcgttgcccaatgtggcagctcccagggaggacggtcagtggtcctgctgtcagcctcagcctcccaggccggggactctctccccggtggccttgccgtgctcatagccctgccgcccagtcctctcctcgtgtactttcccttgcaccctaagccaccttatgaaaaccaataaaaagtacttagggcgaaaaatccctctgaacatggttattaagtccttggaccttggcaaacatagctcagttcctgttttacgcctttggatttctgcactcctgaatgttatttgatgaaatttaagaaaatattaagataaaatccaaatccattcggatgtggaaaggttgaatttactcatggaattaagtctttgaatcccgccgtcgctgtccgcccccagccacatcccagtttgttattaagccaggctcagactcctctctccagggcgggggtctgtggagccctttgcaagagggtcctcagcacgggctcggagaagctccacgggggtcactctgtcccgatcagaaaccgcgagggcaagagagggagagaggaaagaggtaaatgaccgcagagcgggcgtctggttttcccttgagaagggtgcttccatgactttctgttttaaagaaggcacctaactcaagcttccctcagaatacaccttggtcactgcttcaggtgaaaaaaagatttaagccatacttcgaggtgccttgactcctctccctctggcacacgcccgggtgggcgccatagctgctggcctgggtttgagcaggaggcttctgactccttgcattcccgctggccgccttttccagatggtatccagagcaatctttgcaaagcgtaaataagggcacttcatgcgctgctcaccaacgaggcaggcttctcatcgccccttcccatccgagctttcctgtcctcgggtcatcaggcctctgccttgcacctgggtcttggccagagacccttgaagattgaagtggctcaggtccacagagccacctgcttcctgccaggtactctaattcttctggaggaaatcaccaccatttttcgtttcaggattcatacagattaagtttaaccaaacacgaacacacaatcgaaaatctccaagcacacaagaaaaaaagcaccgtgaatgagaggacggtgtgatcagacgtgccaattctgcagctcgattttagagatatctgtaattattggtaattaatagagattacctttcaccattattagcagttgttttagggcttatagaatgcctctttaacttctcccagcctgtctttggttaaaattaagtcacttacagagagtatgagcgtctaacaacagtaacttccactgttccctcctcacatttctgccatcgtggccataacttttacttcttcagaagctagaaaccccacatacattgttcttattttagattaagtggttaattaccttttataattgttttaatttttaacttctaatatagtagagtatatatttatacggtacataaaaattacaattttaatcttcttttttttgtttttgttttgagatggagtcttgctctgtcacccaggctgcagtgcagtggcatgatctcggctcactgcaacctccgcctcccaggttcaagcaattctcctgtctcagcctcctgagtagttgggactacaggtatgtgccaccacgcctggctaatttttgtatctttagtagagacagggtttcatcatggtggccagactggtttcaaactcctgacctcaggtgatccaccgcctcggcctcccaaagtgctgggattatggacatgagccaccgtgcctggccattaaaattttaatttttaatatagtagggtttatatctatagcatacatgagatattttgatacaggcatacaatgtgtaataatcacatcagggtaaatggggtatctgtcaccttcaagcatttatcctttctttgtgttacaaacaacccaattatactcttttagttattttaaaacatacaataaattattgttgactctagtcaccctgtcccccacccctctagaaccaccattctcctctatttctatgtcaattgttttaatttttgctcccacaagtaagggagaacatgaaaagtttgtctttctgtgcctggcttatttcacttaacatactgaccagtgccccccatgctgttgcaactgacaggatctcattcttttttatggctgcgtagtactccattgtgtatgtgcaccacattttctttttccattcgtctgttgatggacactgaggttgcttccaaaccttggctgctatgaacagtgctgcagtaaatgtgggagtggagatagcttgttgatacactgattcccttttttggagtatatacctagcaatgggattgctgtatggtagctttatttttagttttttgaggaactttcaaactgttctccatagtggtagcaatttccattcccaccaacagtgtccaagggtttccttttatccacatcctcactggcatttgttattgcctgtcttttgaataaaagccattttaactggggtgagatgatctctcatagtagttttgatttgcatttctctgatgatcagtggtgttgagcacattttcagatgcctgtttgccatttgtatgacttattttgagaaatgtctacccagatcttttgcttatttttgaaaatcagcttattaggtgtttttcttatagagtagtttgagctccttatatatactggttattaattccttgttagatgaatagtttgcaaatatattctcccattctgtgggttgtctcttctctttgttgattgtttcctttgctgggcagaagctttttaacttgatatgatcccatttgtctgtttttgctttggttgcctgtgcctgcagggaattactcaagaagtctttgcccactccaatgtcctggagagtttctccggtgttttcttttagtagttgcaaagtttgaggttttagatttaagcctttcatccattttgatttgcttttgtatatggtgagagacagagttctagtattgttattttgcatatggatatccagttcctccagcaccatttattgaagagattatcttttccccaaagtatgttcttggcacctttgtcaaaaatgagttcactgtagatgcatggatttgtttctggattctctattctgttcctctggtccatgtctcatctgtttttatgccagtaccatgtagtttgggttactatagctttgtagtataatttgaagtcaggtaatgtgatgcttccagttttgttctttttggtcaggatagctttggctattctgggtcttttgtggttctgtacaaatcttagaattgtttcttctatttctgtgaagaacgtcattagtattttaatagggattgcattgaatttgtaaattgctttgggtagtatggacattttaacagtatttattcttccaatctatgaacatggaatatcttttctttttttgtgccctcttcaatttcttttatcaatattttatagttttcattgtagagatctttcacttctttggttaattcctagctgtttaattttatttgtagctgcttttttggtttctttttcagattgtttgctgttggaatatagaaatactactgatttttgtacattgattttgtatcctgctgtaccaaatttcagctctaatagttttttagtggtgtcttaagtcttttccagatataagatcatatcatctgcaaacaggataatttgacttcttcctttccagtttggatgccttttatttctttctcttatctgattgctctagctaggacttctagtactatgttgaatcacagtggtgaaagtgggcccttgtcattttccagatcttagaggaaaggctttcagtttttcctcataatactacctcggtctgtcatatagggcttttattatgttgaaatatgtttcttcttcttcctctttcccttccccttccccttccacttctcctccacctcctccttctcctcctcctcccttctccctcccttctcccttctccttctccttcttcttattcagtggggtcttgttctgttgtctaggctggagtacagtgacatgttcatggctcactgcagcctcgacctcctaggcttaagtgattctcccacctcagcctccagactagctgggactacaggcatatgccatcatgcctggctaattttttaaaaaagttttttgtagaccaggtctcattttgttgcccaggctagtctcaaactcctgggcccaagcaatcatcccaccttggcctcccagagtgctgggatcgcaggtgtgagccactgtgcctggcctgtatgtttcttgtatacctggttttttgagggtttttatcatgaagggtgcagaattttatcaaatgctttttctgcataaagtgaaataatcatatagtttttgtacttcattttgttgggatgatgtatcacattgattgatttgcatatgttgaaccatccttccatccctggaataaatcccacttgatcacaatgaatgatctttcgaaggtgttgtttaatttggtttgctagtgttttgttgaggatttttgtatcagtattcatcagggatattggcctgtagttttctttctttctgtttttttgtttgtttgtttttgatgtgtttttgtctggttttggaatcagggtaatactggccatgtagaatgagtttggaagtattctttcctctatttttcagaatagtttgagtaggattggaattcattcttctttaaatgattggtaaaatccagcagggaagccattgggtcctgggcttctctttgctaggagactttttattatggctttaatcccattattgttattggtctgtttgggctttggatttcttcatggttcaatcttggtaggttgtatgcatctaggaatttatctattttttctaggtttttcaatttattagtgtacagttgctcatagtagcctctagtgatcctttgaatttctgtggtatcagttgtaatgtctcttttttcatctctgactttatttatttaggtcttctttccttttttcttagtctgggcaaaggtttgtcaattttgtttatcttttcaaaaaaccaacttttaattttgttgatcttttgtattgttatctttcagtttcatttatttctgctctgatctttattatttcttttcttttaccaattttaggtttagtttgctcttgcttttctagttctttaaggtgcatcattaggttgtatttttaagtttttctacttttttttttttcttgagacagggtttcactcttgttacccaggctggagtccaatggcgcaatcccggctcaccgcaacctccacctcctgggttcaagcggttatcctgcctcagcctcccgagtagctgggattacaggcatgtgccaccatgcccagctaattttgtatttttagtagcgacaaggtttctctgtgttggtcaggctggtctcgaactcctgacctcaggcgatctgcccgccttggcctcccaaagtgctgggattacaggcatgagccactgcgcctggcttttctacttttttgatgtagctgcttatagctataaacttccttcttagcactgcttttgctgtataccgtgggttttgagaggttatgtttccgttatcatttgcttaaataaatttttcaatttcctttttaatttcttcattgactcactggttattcagaagtatattgtttaattttcatgtgtttgtatagttttcaaaattcctcttgttgttgatttctggttttgttccactgtggccagagaagatgctttatattatttcaatttttttttttttttttgagacagggtctcattctgtcaccaaggctggtgtgtggtgttgcaatcatggctcaccgtagccttgaactcttgggctcaagtgatcctcccacttcagcctcctaagtagctgggactacaggcacacaccactatgcctggctaattaaaaaaaaaagtttgtagagacagggtctcactattttgcccaggctggtctcaaactcctgtactcaagagatcctcccacctcaccctccccaaagtgctgtaattacaagcataagccatcatgcctggctcaatttttttgaatttttaatggcttgttctgtggcctaatatatggtctatccttgagaatgatccatatgctgaggaaaaaatgcatattctgcaactactggatgaaatgtcttgtaaacatcaattaggtacatttgttctatattatagattaagtccaattttttggttgattttctgtctggatgatctgtccaatgctaaaagcggggtgaagtctccagctattattgtatggggatctctctctctccttagttctaataatatttgctgtatatatctgggtgtcccagtgttgggcgcatataaattcacaattgttatatctttttgctttattattatataattactttgtctctttttgtactttttgtcttgaaatctattttgtctgatataagtgtagctagttctgttctttttttggtttccatttgcatagaatatttttccttccttttattttcagtctatgtgtccctttataggtgaagtatattcttgtaggcaacagattattgagtcttgtttttttcgtccattcagccactctatgtcttttgattggagagtttagtccatttacacccagcattattattgataagtcaggaattactcctgctgttttgttgtttgttttctggttgtttcatggtcttctcttccttctttctttccttcctgtgttccttttagtgaaggtaatttcttctggtggcatgatttaatttattggtttttatttttggtgtatctgttatatgtttttttatttgaggttaccatgagacttatatgtgccatgagatttggaaaggtgcccagccaaaagtttttttatatatttatatatatatgtatatacgtatatacatttatatatatacgtatatacgtatatacatttatatatatacgtatatacgtatatacatgtatatacgtatatacgtatatacatgtatatacgtatatacgtatatacatgtatatacgtacgtatatacatgtatatacgtatatacgtatatacatgtatatacgtatatatgtttatatatgtatatatttgtatatacttgtatatttatatatttatatatgtatatatatttatatatgtatatttatatatgtatatatttatatatgtatatttatatatgtatatttatatatgtgtatatatatttatatatgtatatgtatatttatatatgtatatatgtatatttatatatgtatatatttatatatgtatatttatatatgtatatatttatatatgtatatttatatatgtatttatatatgtatatttatatatgtatatatttatatatgtatatttatatatgtatttatatatgtatatttatatatgtatatatttatatatgtaaatatattatatatgtatatatatgtatatagatttatatatgtgtatatatgtatatatgtatatagatttatatatgtgtatatatatttatatatgtatatatatgtgcatatatatttatgtatatgtattttgtttgtttgtttttgagatggagtttcactctgtcaccaaggctggagtgcactggtgcaatcttggctcaccacctctgcctcccagttcaagcaattctcctgccttagcctcctgagtacctgggattacaggcatgtgtcaccacacccagctaatttttgtgtttttagtacagatggggtttcgctatattggccaggctggtctcgaactcctgaccttaagcgatccacccaccttggcctcccaaagtgctgggattacaggtgtgagccaccaagcctggcctaaaatatgttttgaaatatactttcagtgggtaaaaaacattttaatgtagcattttttttccttttggttttaaaaaatgtcttttgtcttgtgtttcctacaagaaatctgtattgttttcgttgtttcttggcctgtcattaatctcttttcctctgatactcttaagatattctctttgttgctggttttcagcaatttgactgtgatataacttttaatttcttcctgtttcttgtgctttgttttgttgagcttcttggatctgtggttttatgtttttcctcatatttgaaaaacttttagacattatttcttaaagtattttccccctgcctctgctcctacagggcctctgcttacacatatgtaaggccgatggaacttctgctgaagctcactgatgcccttttcttttcgtttctttatcttttttttcctgtgtttcacttcatgtagtttctgttgctgtgtgttcaaatccattaatcttttcttcggcaatgcctcatcttccattgatctaatccaatgtatttttcttctcagacattacactttttgtctgtagatgtttgacaaggatcttttgctatcttccatgtctctgccttaacatcttaatctttcctctagcgacttgaacacgcggaataaaggtgatcaacgggtttaatgtccttttctactaattctgtcatgtctgtcgctccccggtaagctctgggtggttttcttcccattgcaggaattgtttccttgcttcctcgcatgcctggtaatatggatgcacgcagtgtgcgtttcatcttgtttggtgatgtatatttttgtgttcctaaaaatattgtttagttttttcctgggaaacagttacattccttggaaaaactcgatccttctgaatcctgtgcttgggctccagtaagtgggaccagggcaggggttgatccacgaggaaggttttttctccgctactgaggccagactcttctgaatacgccagtcaataccttgtgaattgagcttttctaactcacacggtattgactggagtactccaaagagtcatcatgccaagataattgacgtttgggctgggcgtggtggctcacgcctgtagtcccatcactttgggaggctgaggtaggtggatcaggatcacttgagcccaggagtttgagaccagcctgggcaacatggtgcaactctgtctctacaaaaaatataaaaattagccacatggggtggcatgtgcctgtggtcccagctacttgggaggctgaggtgagaggatcacctgtgcctgggaaggtcatagctgcggtaagccaaggtcgcacccctgtactccagccttggtgacagtgagaccctgtctctgaggccaggctctactgagtactccagtcaatacctggtgaattagggctgggggcggtggctcatgcctgtaatcccagcactttgggaggctgagatgggtggatcacttgaggccaagagtttgagaccagcctggtcaacatggtgaaaccctgtctctactaaaaatacaaaaattagccaggcatggtagcatgcacctgtactcccagctactcaggaggttgaggcaggagaattgcttgaacccgggaggaagacattgcagtgagtcaagatcgcgccactgcactccagccttagcaatagagtaagaccttgtctcaaaaacaaacgaacaaaaacaaaaacaaaacaaaacaaataacaacaaaaaaacctggtgaattagaagcttttctgctctggctaattagaaccggcactattgctgtgggaacaccattccttccaatcctattgggttgttcttatttggaccttttcctgtaatccagtcctttttctgacacacatgccgaccagccatcagccacatatcctgaagcccagtttccacacagttcactcctccagtgctttctcagacctcagccaccttggcctccctggtctccagctccatctcctcgccccagggagacgaccaggtccagcctcaggtgggcaccattgcccggacagtcttcccaggtaagcgattgtaggcttgccgcatttggctcctgccttttgaggttcaccttctttccttggctgagcttcaggatctttagggctgtgatttcatacattttgtctagtctattgttggttgtgttgtcatgtcaaatgggaggacgaatcccgttcctgttgcttcattttgtccagaagtgaaagttcctagaacaaggctaaagattaatgagctcattaaccagctctaaaggtatgaaatgaaatatgaaaatagaccaatgaatgtacaaagaaacaagtaatggatctgagcagaaattagtgaaggagaaaacaaaatatcaacaatcagcaaagctaaacatttaacatttactcttctaagaaaaactaataataatgaaaatgacaaaaacaggtcattccagaaaaataaagatacaagtaactatgtaagtgatgcaggacacgcaagccccaaagtggggcttagcaggtgagggttcttggcttcactcaaggatgagccagtggtagtgttaccagtggaggttgtccaggttcttggtgttttgaactaagaattggacaaaatccacaaagcaatgaaaaaaaaaagcacagatttattaaaacaaaagtacactccacagagtgggagtgggctcaagcaagtggcccaagagcccaggtacagaatcttgggggtttaaataccctctagaggtttcccattggttacttggtgtatgccctatgtaaatgaagtagtgacccacagttggttggcttggttgtgggaagcgaacaatcagaggctgaattgaagttacagcattatattcccatacaaatgaagacttggcccacgaccagtctgattggttgcaggaggggactcatcagaggtactttcaatttttcatctgccactcagaaaaaagggggaggtattgcaaagggaatagcttctggttctttggtaacttgggcatggaaagttggggttttccttttgatttcgttctaggaagtcagcgtgaatcagccttaggttccctgcctccagaccctattctcctgcctcagtagggtggaaggaaacagctgtatggaagcagcagggttacaggtcaggcagtgtgacagccccaggactgctcccgtggggcagggctccccacaggcggcgcgctgagggcagggctcccgaagtcaatgcgtccagggcagggctccccacaggtgttgtgacagccccggggctgctcctgccaggcagggctcctcataggtggtgtgctgagagcagcagctcagggcaggtctgcagtcaggtttgtacccacttttaattacatgtagactaaggggcggtttatgcagaaatttctagggaaggggtagtaaccgttgtgtagtggggtcattgccatggaagtgggtggtaacgcctgggggttgccacggcaatggtaaacactggtgggtgtgtctcatggaaagccgcttccgccctggctgtgtttcagctagtcctcaattgggtctggggtccaagtcccacctctgatctcataagaagttgttacggattgtactgtgtcctccccaaaatctacgtgttaaagtcctaacccccagaacttcagaatgtgcccttatttggaaatagaatatttgcacgtgtgattagttaagatggggtcattagggtggccctaagccagtgactggtgtccttataagaaggagaaatctggacacagagacagacgggcatcaagggaagatgatgtgaggacacagggagaagacggccatgtacaagccaaggagaggctgacacagagcatccctcgcagcctcagagggagccagtcctgcccacacctgatctgggaccgtggcctccagaactgagttggccaatgtctgctattgaagctgccagccacagtgctttgttgtggccgcccccacaagctcacacaggggtggagaagcagacgttgccacaggtgtcacgtaaatcaagacagtgctatgccaatgtcatgccaaggcagttgatatttgggccaggcgcagtggtgcacacctgtagtcctagcactttgggaggccgaggtgggtggtggattgcttgagcccaggagtttgagattagcctaagcaacatggcaagaccccattgctacaaaaaaatacaaaaattggctgggcatggtggctcaggcctgtaatcccagcaatttggggagctgaggtggacagatcaattgaggtcaggagttcaagaccaccctggccaatatggtgaaaccgcctctactaaaaatacaaaaattagccaggtgtgcctgtagtcccagctactcaggaggctgaggtgggaggatcgcctgtgcccagggaggttgagactgcagtgagctgagattgcaccactgcactccagcctggacaacagagttagaccttgtcttgaaaaaaaaaaaaaagacagttgtcattttaggtcaaacggacaaattctttaaacaaaatataataaaacatcaaggggaaatagaaaatctgcatggtattattatcacagaagttacaaaaattaaaacagtagtgaaaaaaatatcccacaaaggaaaccgcagacttaggcggtttcatcagttgcatccacaaatgccttaggggatgattttagacaacagacaggaggaaatggtctcagcctcaccgtccgtggccacggcagcttcagtgagccagccctgcacaatcagctctgcaaggtgtgattggagggaacaggaaacagaaaacacaaagagaagccacccagagggttggtgaggatacaaagggttttcaacgccgctagtcagcagggaaatgcacatttaaaccgcgaaaaggctccactacacacccaccagagtggctaaaattagacagcagcattgtgtgctggagaaggggtgatgccatggggctcacatgccacagtgcacagccagagtggcacagaccccttagaacacgggtgacatgtactggagctcagcacacatgcaccacaacagccatgtcccccctgggtctgtgcccaaaggcaggcacagccacgtgcatgatttgatccgtaacggccccggttcgtgccatggaatgccacatggaagaaagtcacaatgagcaccgcttgcgacaacacagatggacgccagacagagcccagcccgagaacctgggtaccagagctcccagcatgactgttcatgtgtgggacttggacccggtgacccatgggctgggttcacccgcctggtttccactgagagggccccgtggggccggggtctgtctgtagcctgactcgggtgcttggtgctggagtctggaagcatgcagggcttgttgagctgcacggggtgcatctgccatgacactgtgtggaaattgtatagcaacaagttttttaaaagtgcctgccggcctggtgcagcaactcatgcctgtaatcccagcacgttgggaggccaaggcgggcagatcacgtgaggtcaggagttcaataccagcctggcaacatagtgaaaccccatctctactaaaaatacaaaaatgagccgggcgtggtggcagatgtctgtaaccccagctactcgagaggctgaggcaggagaatcgcttgaacccgggtggcagaggttgcagtgccagcctgggtgacagaacgagactccgtctcaaaaaaaaaaaaaaaaaaaaagtgtctgcctctcagtgcaccagctgagtccagccagcaggacagaagccactgggacatcacactgaaggttcaacaagggaaatggcaggatgagggggaggtgggcatgtccccccagacatcccggttcagccccagaaccgcggatgtgatcccacgtgccagagtggactcggtggccaccatcacacttggatcttggatgggagatgatgctgggtaatcgtctgggactgatgtaaccacagagggtcggggtgagaatcagggatatgaccacggaggcaaaggttagggtgaggcgaagaaggccacaagcccgggaatgcaaggagcacagctctcgaggtggagagcacgaggaacggagcacctggagcccctggagggcagggcctgcccacaccccagtggccgcgcagggaggtctggtctggactcctggcctccagaaccatcagagaacaaatatgctgcttgaagcatgaaagctgtggggatggtggccgtggctgggggacactcaggcggtggcttttctgggaccagtgtctctgcagaggctgctgctctgaaagcatggtgggccacagggcgccacacccccttgggtcctgtgcctgcttcaggaaaggggtaagcagtgtggtatacgccaagacctcctcctggtttccctcctccgcatggatcactcctcgaacacagcccctgcttcgtcagggttccgtagcctcacctatcgcaggctgcaccccatccttcctccagacccctggcctgtggggcttaccggggccgaggccaacagactgatctcgttgccaaagacccaacagccaccaaactggcttcaacagggctatgtgtggcaggtacttcacagggggacgtgttctcattttccttggctttctaataaactctggtctaaagaaaatctcttctataaaatgagtggccagtgccaaggggtagagacagaagctccttctggacactggctgctccagtagagcccatcctgcccaggtacggagcccaccagctgcgatcaggacactgcagttccaccgtgggggaccccgggccctcaccttgtcagtcctctgtttcagcttcgagggctttgacacagtctaaaatccacaggtacttagcgcatttctaagtcccacctcatgatcaatcataagacagggctttgatggtatgcagaaatatcaatgaaatccaagaggaaaacagaactaaactcatcgggatgaatgacagataaaccctttaagcatgtttcaagtcgcccaggtggagcctctgttggtcagtgaagcttcagggttcccatgagatggatctgcatgaggctcagtcctctcttgaaacactgcccatttgcaggagcgccggcccacagggcagaagtcagactgtcagagcaagtgccggtcctgaagctggaaggactttcaaggctcattccccaaactcatttttttttaaacctctgctcatcttaaagaaaggagaggcaggcgccccagaaacctctcaggagtaactgacacacgtgacttgcggtgtcttcaaaggaacactctgcaacttgctactgtttaaacagagcaagactaacagtcaccagcagctggttcacgcattaaggccctttgcaggtgaaagaaaacggcactggagggccgactgcagaagcagacgcagtgcacggtggacatctgccccttttgggggcctgccacggtgcttcctgaaaactcaggttaagctgactctgctgtcaggctgatgaggcgcctgcacccacctttgctcagcacgccagcccaaatggaagagcaggtgctatgacaaaaatcagttcttgtagcaactatttttgatactgaaactgaaaagcgagagcagcaaagctatccacggagctcctgacagttaggcccggctttggctgctgatgcaggggccgaatgacgctatttctgcagcagccggccgcaggcgctgacccaccgcgggatgtgccccaggaggcagaagaaacgctgggaaccgcacctaaccttgccagcatcccccaggcaaagccaggcactttgagttcatcggtggagttgggcgatactcctttcccgtttgtgtgcttagctgctgccaacaggatgtgctgggtgaagccttacagaacacagccagttgtgttttataaagcaaggctcccctcccccaaagacaaataaaaccacaggtgtggaagggacaccctccaccgcaccccgccctagtaatctgacaccgcaggcaaaggcacggcctgtgtgggtaagatccattgtaaacgtttaaaacttcctgttccctgatgcctcaacttccccgcagaccgacgcacctgtgatgggcagggccgtgtgacctcgtgaccttcacatacaccaatgaagaccctcacgccttctgctgtctttcccccactgctgacttgcctgcgggcccccactatctgccctgcctctggagccgcctcccgtgtgcccccccaggccccttaaggtgctgtgcgtcccatctggaaccggcagttcacccgctctcccagccacccttccagaccccacttacaacacacgactaagtaacacagtacaggccgtgtattcgtatttgttttcctgtgccaactcatctggtatcttcgtattttaatgaataaaactgaaagatgacaacttgaagccccagtgcattcctctcaaatcgagtgaagtttacaacaccttaattataaaagatttatctgatccagaagaaagcagagtgtacagaaacttcactgtcatctgcgaatggactgagagcatctggatttggttctcagcctgaatgagcatcctggaatggtgtgtctcgtccatttacgtggagacacatggaacaggcctgcaggcttggggctctgagcagggctcccaaagcccctcagctgcaaggaagccaggcccatgagaggggagcctgttccagaaacacaagaggatgaaagctgttattggcaaaggagacttagagcaaggaaatgaccaggaataaaaaaagattatatatttagaggccaggtgtggtggctcatgcctgtactcccagcacttcaaaaggccaaggcgggtgatcacttgagatcaggagttcgagaccagcctggacaacatggcaaaaccacatctctactaaaaatattaatacaaaaaagctggatgtggtggtgcacacttgtagtcgcagctactcaggaggctgaggcaagaagattgtgtgaacatgggaggtggaggctgctgtgagctgagaacaacaaaccccacaaaaactaacttcaacaaacattaaacttcactacacaccgagtccagcagttctccacccatctttctattccatttctctctctctgcctcaacctagggcaagataagcttgacctgatgggtcatggctctgtacaatagtttagtcttaaacaactaagatgctacgtatctaagaacactcatgactcaaaggcttgcaaagggctctttaaatttgctcttttaatgcataaataaatccatatcatgtattactttctggaaaagggttaaactcaaatatccgaaatactttcattataccaggtcaagaaaaatgccacagccagaaaaatttattttaaaatagaaacatacattaagctttaaaacaaccaactctcaaacaaaagaggaaagagcctttgatcccagagtccatgcggaatgaattccatacgtgtttgaaattcacataaggagcacttagaaaaccacctgaaatggaaatccaacagcccccttgcctgtgagggctcccacccctgcccgcgtgaggacatggccgaaccccggacactcgtgtgccgggagccaccacagctcaaggtgaccggcagcacccagctctgtgaccaagacagatgttcacacgtgggggcatcgtaagcgctaccagctccaaatctgacgtgatgggaacttgggagatgtctgagaaatgtccgaagggattttggcaacacagaaaacgcaatgtctaggaattcctccaaatgcttccaaaaatactcattgacaattcaagttgcacttggctggcggcagcccgggcggccttcagtccgtgtggggcgcccgcgtggccttctcctcgtaggactccccaaactcgttcactctgcgtttatccacaggataaagcctgcaatgacacaaatgagcacatcagcaaaccccactgcaggggctatttttctaagaggaggacttggcatcctcgatttattttccagtgagctgccacagtgagcaaattaactaaaaagtcgaatcctcagaagttttactgccgagggctgaggagggatttctgagttgtgttttgttctgcatggccaggccagcgtgctgctctcaaggaaagggaagcttggccgaagggacgacccggaggctgcacgggctgccatggctgaaggggggacccggaggctgcacgggctgccttctgggaacggtggcatcacggagactcgagtgatcagaacaggaaccgctgtgttcagggaccacgtctcatcagctgtgaacacttggcaggacactgccgccgcctttgatcctgagcgccccggaggccttgccaacatcacccctacgtatggatgaggaagctgtggcataaggggcagctctacacggccgcgaacagaagtacagggtttcagcttaaaaacagacctgctgcccaccaagggccctgtgatcctggccaaacaccgtttgcaggtctggagccattaacacagagcctgagtacagcacagtttcaggcacgccccgggccacctgcgaggacttgggagccacttgaagggctcacagttgagacaaccacaccaggcgcagcagcgtccggggctccgtggcttggacaacctcaggctctgcagggggcaggcggccagctgggggatctgagaatggtgaacgggggacaggaaccaagccccgtgtgagtgtgagtgttgcttatggtgaaatagcccttcccgctgacacaagctggagctcccgtgcaggggctgcacgagacagcaacagggtgggggggggcagccggcggccaggcctggagagctgggcaagggcggccatgagccactgtgtggatgtgccaactgcacccgaacaaagagttaaaccaggcagaaccatgccagcctcatgctggctgggacagaggctctgaacacctggcgaggttgagtgtggagacagggcggggacgggagcactgcgctccccaagggccggcaccccggacctaggcaggacccgccaggacggcctgggggaagaggatccctctcctgcactagtaggatggccccaggccctgagggaagggcacagggaagtgtcacagccgtggtgtgtgaggtggcctcctggtcgggggagggagatgggaaaagggaccttgagggccagagaaaggacacagccaggaggctctgagacacttggcagcaggtcttgggggtgacacacactccttcgacccctggacaaggccctggggccggcactggaggtgagggcagtgcttgcagtggtccagatgcaaccctcccgaggggagccccagatccctggctcctaccctggcacagcagagctggagggaggttttgctgactgagagggcagatgtcagccctggagggaagcgggaggcccagggtggggcctgagcctctgcatttggaaaattccgaggcccggggtggggcctgaacctctgcctttagaaaattccacaaagccttttcctcagccgtcagccttcaacctgaaggccaagctgagaggtccacgcaatgcagtcttcagaggccctggtggctggtttaaggggtgcggccaatgctgaggtgagtgagctccccagtgacattgtgccctgcaggtccacactgctggctggacacttcgaggctagcgccaagtatggattagccatgctacggtctctgttctgctcaatagcaatgactgtgaataggtaaataaatcgcttaaggagagacatttgctttcagtggctcatcaataattcacatagaaacgagcatttctaaagcacagtgaggagacagagctggaacagtttcttgccaggacatatcatgggcaactggaacccaagtttaggcaagacaggaaaaaccaccacctgcaaattatcttttccctcaaatggataaacaggcgcagggtgcggtgaaagccgtcattccgttcagcagcagccacgccgctgagacggagcaacggccgagcatacgcagccgcactcaccaccgctggtacaggtagaccagaaacaccacgtcgtcccggaagcaggccagccggtgagacgtgggcatggtgatgatgaaggcaaagacgtcatcaatgaaggtgttgaaagcctgcagggccagacgggaggagggtgaaccccagttgctggggctggaatcctactgtttttggtaacctaaccaagccaacggcttttggcagatgcttggaactaactggaactcctcacagcaacaacaaaaagagcagaaagccggcaagtggagatacggagctctgttccctcatgggctccccacagctgctggcacccgacacactgcgggtcttgcccaggctcccacatcggggaacccaaagagaatggcctgaaaccagagccaaggctggagccgcccgagacagagccaagggcagagccacctgagaccagagccaaggttagagccgcccgagaccacagccaaggggagagccaagggcagagccacctgagcattgccctttcctctgggagcttgtaattgacttcagaaaagtgcaaatcatgtcccattaactcagctccgcctgcaatgccgaggcacttctaatcccacacggagccaagggggcctccacagctctgccagcggggaatggccacggcccatgggggcttccattcctgcctgggaggcctgaatccgttgtccccaggcaccggttactgagccacagggggtctgtgtcttgacacatgtgctaagaagaaacataaacactaaaaaagaaaaagaaaaccaaatctgtgacaagtgagagaaccaccccagtctttcagggtctgaagttactctatagctcttcgcttttttttttaaaggagcctcctcaatattcttcaatgaaagggacaggtgcagatgcactctgggcagagatggccccagtaatgctgttggctggaagacgcccttgctcacgctctgctcagtgagaagggattcctcaccggctgtcacactcaccttgtaggtgaaggccttccagggcagatgtgccactgacttcaactgaaacaggagagacaggcccaggtcagctgtgggcagcacaggagacgggggccgggccgcgggcagcacacaccgccttaccttgtagttcacaaagagctggggcagcatgaagaggaaaccaaaggcatagacccctgcagaaagacagacagcactcacgaggtgcggagggccgggctgccacatctacgcacagacggcactcacgaggtgtggagggccgagctgccacgtctatgcatagtgggcagaaaacaaggtctgctatccagacaacttcagagtccatcatggtgtgaagcagctttctggctggtaagtttatcaagagtctacgacaactttggagagcaaagctcttctatttattaatcagtgtaactgctgcccacggggtcaagtcagtgagagcactggagcaccctggggctatgaggacacggcctcctgacccacaaggtcctgccccaggggtcaggtgtgggactcctaccagggaggacggcagtgcgggacccacctgctctgcaccagacaggccctcgcccttaatgctcacgggaccacttcccaaagagaccacaaagctgcgatcagggctggtctggtggggctgggggatggacacagcagcccatgcagctgaaggtgccacctggggcaggcgggcctcccaggacatgtggccccgagcacagggccctgcagggtccccacactgcatctgcaggagggggcgcgccgtgcaccagagcctggtgcttccatttgctctaaggaaccagacgtggatggtgttgttttaatgcttaggtttccttgtttccatgaagaaaacagacaacagtcatgcaccacacaatgctgttttcatcagcgatggacacatataccaggcggctgcaccacacagcctggcgtggaggggctgccacatctggtgtgagcactccctgtcgcttgtacaaggacctgtttctcagaaccagtcactgttgtgaagccacgcagggctttatctgctcacagcctggggggagccctgggcaccgcacatgtgccagaacagggtggggagggattagcctcaaatcacaggggggcttgccacactgggtttcaatgcaagaacaattaaatcgctgcctgctggaaacactagtactgaagcagggaagcacatggactcaccgttgacgaagctgttgattaaccaggagtaccagctgaaacggaaaaaaaaggagaagtcctatttctcgcatagcttaatatctgtattaaattgatgaaaaataattctttcattaaaaatcctctgaaaatacccagatgctctcacggcagctcggcagccaagagcagcagtgctgagcctggttctcaagctggagatagcgctgagcacccccggccgggcagcagaggccgggcactccaggaacccctcagctcagggccagcaccccacctgggcagcagaggccaggggctccaggacccctctcagctcagggccagcaccctgcccgggcagcagaggccgggcactccaggaacccctcagctcagggccagcaccccacctgggcagcagaggctggaggctccgggatccctcttggctcagggccagcagcccacctgggcaacagaggctgggggctccgggatccctcttggctcagggccagcaccccacccgggcagcagaggctggaggctccaggacccctctcggctcagggccaacaccccacccaggcagcagaggccgggggctccgggaaccctctcagctcagggccagcaccccacacgggcagcagaggccaggggctccaggacccctctcagctcaggcccaagcctcccatgaagcctggtggctggagtcagtcctctcagctacaccttaccagggggacactgaggcccagagcccaccgtgtgcagctgcaggggcaggacccctcccaggcgctggcccaagtgccccggccccgagtttgccctccagtcgcaccccgctctggctcagtcatccaaatggctttctcaggggaaagacgcagcaggggaagcgtgtgcggcctcctacctcttatatttgatattcaggagtgaatagacagcacccccgacacagagagggtacagcaggtatgacaagtacttcatggcctgcagggaacaaagatggcttccagttagaggcccaaacgccaagcctctgtaaacacactgcatggagctcaccccgacagggacagacagaacgagctacagcgctcagggtggcagggctgctctgcagggggcggcgtgaggggcacaggcagggcatcgcacactcgccacagccccaggaggcaccgcacacgccagagcccgggtgtaactacaggcgctagttaacaataatagatcaattctggttggtcaaaaccaaccaacccacaacagatcaataaacataatggaaagaaaccggaaaaaactgcctactaagtaatgtagttacatcgccagctgtatccattctgatttcagctttacaacgccatgcaagttggatataagctggtgaagattaaaccaaactaaaatcatctgtgcagaaatgttctattaactggaactgagaattccagctcccccaacagcaatggggaaaggtgacgccctgtgctgctggggccattcctccaggccactgtgcggccgcggccattatggccttggtgacggcaagtgcgttcccagcaaccacagcagacaccatggcctgcgtcagctgcctggcgaatgtgacttacaaacgatgaaaatatccctactatacggtgacacaggcaggctgtgcctgtaaggactgggaaatgagtctctgcgagcccccgcccttcccagtaagaaatttgggttttatgtgttcactcttgggatccctgctcaaggcgggctgacccgtactccaaggagacttccgcactgagcaatgacagtaaaagacccgtctttgagggtgttggatttgcctggcatgcacgtgccctgaatcacaagtgacttacctgagtatcgtactcctcggttttcctctcagattcgctgtaagtgccaaactgttgtgaaattcaacacagtgatattaatagactcagggaggatctgagcacagctgagctgtgactaaggggcgtcacatatggctgcagagctgacccaggcctcactacagagggcgctcaggtccctaccaggcgaggagaggacccagaggacggggatccgcatggatccttcccgcctcaccctggccctgggtcccacaagcctgatgggggcaacgcggcctcactgctgttccttgctcaggtggctcagttttcagtgccaccgcttcagtgagaacactgcagagcacacaggcacagccaacactgctccgaaccagccggccgggagccatggggcaaggtggaacacagtggctgcgctgcgtgtgctgggcttcctgacccccctgcccacagcatggaaaatctcagcctgtgactgctgagggccccgcctcaatcaaggtccatccgtgggcactgagattcaactccagctctgccccagccctcatctgcctaggggccttatgggcgcccaggcactggggcagccaccagctcagggcaggggctcactcacttcaggactgccaggactgtacggaccccagtcagcaccagcctcgggagcgcctctggacaaggtgccaaaacgaagcacacgtgctggccagccagcatccttcctcccagaggccggctcaggcccagaagtgctgccgtctgcactgaagacggtcagaactaggtatatgaccggggaatcctcaggcgaagagagtggctgcgatcagaaaccagtgctgcctccaccacggaggctcccctgacagaggcccgcagtgactcatctgcagatggccatcttactctttgcacagggggcaaaatcacactcttcagagaggcttggggttcagccattacaactaaatcctacctgaaattcgggcatcaggcctctccaaaaaatagtcatcttcaatgccttcttcactttccacagctgaggggagaaatcaggaaatagttcctttaatattcgaaggccaggagccacgaatgaacgacccagacagtaacgggtaggacctgctgcccatggccccgcgcagcccactgtcctcttcctgtccctcccacagctcaggccagagcgctggaggctggacctgggcctctaacccacacacggcaggcgtacctggtcaggtgggctgcacagccaccgcccagcggacaacaaaaacagcatgtgagggcagttctttacgccactctgcctgcacccaaataagcccccacctacttctgcctgccactcgggcagccctggagccagttttgggaccgctctgagagccgccacctcatggctaaatgcttggaaggcatcacccacggagctcaggacgggtgccagagccgggcacggcactgtgactcgtacctatggagggaccctgcctgtggagctccagcgtcatttcatccagctcctcctctacagggacattccatccagctcctcctctacagacacattccatgcagctcctcctctacagacacatttcatccagctcctcctctacagacacatttcatccagctcctcctcgacagacacatttcatttcatccagctcctcctccacagacacatttcatccagctcctcctctacagggacattccatccagctcctcctctaccgacacatttcatccagctcctcctctagacacattccatccagctcctcctctacagacacatttcatccagctcctcctctacagacacatttcatccagctcctcctctacagacacatttcatccagctcctcctctacggggacattccatccagctcctcctctacggggacattccatccagctcctcctctacggggacatttcatccagctcctcctctacagggacaatccatccagctcctcctctacagggacatttcatccagctcctcccctacagggacatttcatccagctcctcctcgacagacacatttcatccagctcctcctcgacagacacatttcatccagctcctcctctacggacacatttcatccagctcctcctctacggacacatttcatccagctcctcctctacggacacattccatccagctcctcctccacagggacatttcatccagctcctcctctacagggacattccacccagctcctcctctacagggacattccacccagctcctcctcgacagggacattccacccagctcctcctcgacagacacattccatccagctcctcctctacagacacatttcatccagctcctcctctacaggcacattccatccagctcctcctctacagacacattccatccagctcctcctctacagacacatttcatccagctcctcctcgacagacacattccatccagctcctcctctacagggacatttcatccagctcctcctctacagacacattccatccagctcctcctctacagacacattccatccagctcctcctctacaggcacattccatccagctcctcctctacaggcacattccatccagctcctcctctacagacatatttcatccagctcctcctctacagggacattccatccagctcctcctctacagggacatttcatccagctcctcctctacagggacattccatccagctcctcctctacagggacattccatccagctcctgctctacagggacattccatccagctcctcctctacagacacatttcatccagctcctcctctacagacacattccacccagctcctcctctacagacacatttcatccagctcctcctctacagacacatttcatcctgctcctcctctacagggacatttcatccagctcctcctctacagggacattccacccagctcctcctctacagggacattccacccagctcctcctctacagacacattccatccagctcctcctctacagacacattccatccagctcctcctctacagacacatttcatccagctcctcctctacagggacattccatccagctcctcctctacagacacattccatccagctcctcctcgacagacacattccatccagctcctcctctacagacacattccatccagctcctcctctacggacacattccatccagctcctcctctacagacacattccatccagctcctcctctacagacacattccatccagctcctcctctacagacacattccatccagctcctcctctacagacacattccatccagctcctcctctacagacacattccatccagctcctcctctacagacacattccatccagctcctcctctacagacacatttcatccagctcctcctctgcagacacattccatccagctcctcctctacaggatacattccatccagctcctcctctacaggatacatttcatccagctactcctctacggacacatttcatccagctcctcctctacggacacatttcatccagctcctcctctacagggacatttcttccagctcctcctctacagacacattccatccagctcctcctctacagacacatttcatccagctcctcctctgcagacacattccatccagctcctcctctacagacacatttcatccagctcctcctctgcagacacatttcatccagctcctcctctacggacacatttcatccagctcctcctctacagacagatttcatccagctcctcctctacagacacacttcatccagctcctcctctacagacagttttcttccagctccttgtctacagacacatttcatccagctcctcctctatagacacattccatccagctcctcctctacagacacatttcatccagctcctcctctacagggacactccatccagatcctcctctacagacacatttcatccagctcctgctctacagacacattgcatccagctcctcctctacagacacatttcatccagctcctcctctacagacacattccatccagctcctcctctacagacacatttcatccagctcctcctctacagggacattccatccagctcctcctctacagacacatttcatacagctcctcctctacagagacattgcatccagctcctcctctacaggcatgacggccgcagaggcccccacccacaggtgggccaccaactgcagagacctcagactccggctcctgccggtgtggcacagagctcagtgaggagctaggcttaagcctgggccctctcaccctgaaggccatcgcccccctcaagggtttgctcctgagctggtcttgccccacgaggctcccttttggccggcttttctgagctccggaacaaccaaggctggaccacagggcaccgagtgatttccgtcagtgcctacctggccgctgtgctcagatctccagccacagcacgagcctctcaaggccccacggtttacacctctgctttgcacatggtgggatttctcaagcacatgaaaacactctgtttggtggacagggcctcaggcctctgcttggtggacagggcctcaggactcaccactgcttggtggacagggcctcaggactctctgcttggtggacagagcctcaggactctctgcttggtggacagagcctcaggactctctgcttggtggacagggcctcaggactctctgcttggtggacagggcctcaggactctctgcctggtggacagggcctcaggactctctgcctggtggacagggcctcaggactctctgcctggtggacagggcctcaggactctctgcctggtggacagggcctcaggactctctgcttggtggacagagcctcaggactctctgcctggtggacagggcctcaggactctctgcctggtggacagagcctcaggactctctgcttggtggacagggcctcaggactctctgcttggtggacagggcctcaggactctctgcttggtggacagagcctcaggactctctgcttggtggacagagcctcaggactctctgcttggtggacagggcctcaggactctctgcttggtggacagggcctcaggactcaccactgcttggtggacagagcctcaggactatctgcttggtggacagggcctcaggactcaccactgcttggtggacagagcctcatgagctgataccacccgaacacgcatttccttgctctccacttgttcaaagcctttacacccactgcctgccatcctacagcttccagaacactgaggccatcgggaacaagcacacaggcttccacagaaggtctcaggtcccggggctgaaagcctttcctgagtgcgtggaggcacatggacctcagacagttcaggtcactgcccggaactcacctcaatggcggctccaacacccgccgggaccagcaccagcaggctcgtctgctcgtccagcaggaacagaaagatgaccacggtgctgaagcagcgccagagcactggggacaggatggtcgggctgggagggggtgcagggcagaccccacctgtgttccccaagtcacacacatacccccagtcccccatcccaacccaccacgcccaagaagcttcaggtactccccagtccacgtcaccccgtttaaaaagaacaatgaagcattcagcagctaggaaagtgtttggaaggctgctgaactgaacagctggccccacaccagctccacaaacacgtgtctgagccacacttgcaaatgagcatggaacgtgggcagaggtcggatgacggggccagcacagctttgcttccgggcgtgtcttatgccagttctgtgataagtgcgtggcttttcaagtctggatttgtttcctccccagctgtaccttaagaaccactccagcttattcaacacgtcttcctcctcctgcaagccctgagggcaagcaggggaagcagatccatggagccacaggacaaacagtggtcaaggccacagccactgtctgctggccactccaacccaggacgccaggaaaggcattagggttaggagacaacaacgcagaacacggcacggcccccaggcagccagggccacctgcttgggcgtcttttccatgcgagaaaaacaacttgttcgggtctgatactggagccaaatgctcctcactcctaaccaacaggacacacccactcctcgtcctcacacccatcaaccactcgcccctgtccagaccgccgctccccaccccactgtgacaacctccacccctgcaaggtcaggaggacccctcactcacttgggccccttaaaccacggcaggcacccttggccaggaacactggtcccagatcttggcaggcctcctgcctgggctgcctggcctcaggggtccctgactgccctacctaacagctgcacactgcctgtcactgctgggcacagcaccacgggcctattaactgccatcacctggcaggccccacacgcccaggcctgcgctggtcggggaaggaacaccggcgggaggacggggcgagggaaccaagacaggggatgtgaggccccagccctgctcagcgcccatgagcagccgggaaggctcgagaggtccatgcatctcactctccacagcccagacccagaccccatgcacgccagagtccgcagccaggccctgagctgtgcagccgctggaggccctgagccgtgcagcctctgatggtcccaccacaattgccgcaacggctccctgggctttacggtgcctggtctcagaggcttctcaagagccaagcacacacccggggctgtgtctgcagccagcagtgaggcaggcagccctctaccgcaggctccagtgagctccctggggcagagtatctgagcagggccacgctcggggggcctacctgccttggtggacatgccgatcatgctcttcttcttcttccagaaactgatgtcatttttaaaggccaggaaatcaaagagaagctagagagaacacacacgacagcaactcacatctcctgctgtttccatctcctgtgagacagagatagaggcttcgtgtgtgaccgtgagactgcaggtgtactcagcctcaggatgcatcaggataagtgtctacactcgggacccatgcctctacgcgcctggcctgccatgtcagaacctagagtgcccaggcgggctttgcaggggcacttctgaaataatacgtacctttgacttgaactgctggaaaggcccccaggcaattggaggctctggattcccctggatcatgggcctggctgggcacccctgctctcgggttgtgcccactcagggcagcctcatctctcaactgtgctaaaaacgcaagggctgggcatggtggcccatgcctgtgatcccagctactgcaaggccaaggccaaaggaccacctaagcccaggggtttgagaccagcctgggcaacgtactgagacctgtctccaaaaaaaacttaaaaaaacctaaccatgcatgatctcatccttagagacactgaccttttaataaaaatcgtttaattctctagtacggctttggaaagttttagcttgactggcagccaaactcccaggcactcagaaccagtgccggccgcactcctgggtgccgcctaaccacaggccagcctaacagccacaaggatggctctccctagcagctcagctccacgaacaacagtaagagcacagcagccttgctttacccatggtttactgtccatggtttcggttagcacggagcagtgcgatgagacagatattctgagagtgagaccacacccacgtaactttatcacactgtcctgtcctgtttctgttaccagttattgtgggtaatctcttactgtgcttaacttataaattgaactttatcaaaaccattacataatacatatattcattcattacgttatttgatacacatattcagacatccaatgagggtctttgaacacaccccctgaggatgagggactactgtataacaccagatgaggataaggggcggactactgtatatacactggatgagaacaaggggggactactgtatacacacgggatgaggataaggggggaatactgtagacacaccggataaggggggactactgtatacacaccggatgaggataaggggggactactgtatacacaccggatgaggataaggggggactactgtagacacaccggataaggggggactactgtatacacaccggatgaggataaggggggactactgtatacacaccggatgaggataaggggggactactgtagacacaccggataaggggggactactgtatacacaccagataaggggggactactgtatacacaccagatgaggataaggggagactactgtatacacacgggatgaggatgagggactattgtatacacaccacatgaggataaggggggactactgtatacacaccagatgaggataaggggggactactgtatacacacacgggatgaggataaggggggactactgtatacacattggatgaggataaggggggactattgtatacacaccagatgaggataaggggggactactgtatacacaccagatgaggataaggggggactactgtatacacaccggatgaggataaggggagactactgtatacacacgggatgaggatgagggactattgtatacacaccagatgaggataagggggactactgtatacacaccggctgaggataaggggggactactgtagacacaccgcaagaggataaggggggactactgtatacacaccggatgaggataaggggggactactgtatacacaccggatgaggataaggggggactactgtatacacatcgggtaagggggaactactgtatacacacaggactgactttttacatttaaaaatcagtgtgttagaatttatacaagagaactactttctgctagaggctgcccagctccgcccctggctctgcatggggtcggggcctgggcaggggagagtgagatgcctgcacctattcatctcattggtgtggacactgctcagagtaccctggatccttttcaaaaccgcccccaaagccagcacagcccaagtcgccaaggctgtggacggagctttatggtccctcccttttcagaactgggagaaaccgcccactgggacaatggtcgcctggtgtcaggcgtgctggctgctgagtgatgggagtggacacttgagggccacagggctggacggcgcccacaaaccccaggtccaggacccctgcaggaggcccggggcccagggagccccccaagtgcctgcggcaagccccccggtggatgactcacatggaacgctgcgacaaagaaggtcagcgccaggaagtataagttggtatctacaaaaattcctttcacctcatcagcatctttctctgaaaaccctgtcaaggaaaaaaaaacatacaatataaaacaggcaatgtcaatcttacctaacattacaaatacagttttcaatataaaaatttaggccgggcgcagtggctcatgcctgtaaacccagcactttgggaatctcaggcaggcagatcacttgaagtcaggagttcaagaccagcctggtaaacatggtgaaactccatctctactaaaaatacaaaaattagctgggcgtgggggcacgcgcctgtaatcctagctactggggggactgaggaacgagaatcgcttgaatccggggggcggaggctgcagtaaccagagattgtgccactgcactccagcctgggcaacagagcgagactctgtctcaaaacaaacaaacaaacaaacaaacaaacaataaaaataaatctcatcaacagttaaggttcatttgaccaaactcatggcatttcacatattatgttcttaataaagccaatttccagacaatcaaccaggtcatctccaaggagagaagtgaggcaggcgctgctagcctctgattcaacgtctgtgcaaggatctgcagcaggaggcgaggaggccccggcctgtgtcaggattcgcacttggatgcccgaggggctccagggcggcctcacccaggcgccggccgtgtgcggcacataccgaactgctgcagggagtacacggcgtcctgcatgtggatccagaagcgcagccgccccagtgagaccttgtcgtaggacacggtgaggggcagctcggtggtggagcggtttatgacctgatgaagaaagccacactgagggccctgccctcatacccttgcacccagctgcctggcagccctcgccaaccctgccatcccccttcccatccctgtgtggccccaggtcagccatgactgggcccagcaagcatcccccggtccctccttctgtcaccacaggcctcaggccccagcgtgctgcgttccagcacagggaggttcccacccccaccaggaccccgtgagctcagcagcgctagcagtgcctggtctgttttgctgcacgtgccccttcaattttaagccgcacaagtcctggtgccccggaggtggtgcttgccacccagctgctaggactccatggcgcagcatacggcgctgagcttggcctgcagagccctggcctgtcctaagagttggagaccacgggacaagcacagccctgcagcacatgggcaaagtgcctcagggcctaaagcacagagcagggggtggccgtggggacaacagaaagcttctggaacgcaaagctagcaggggcagtagggtggatgtgtaggtctcagaaccacgagcttagtcctgcagcctgccggggcctcacgcacacaggctgtgggccaggtcccagggtgccgaggtaggagggtgaggaacgggttcacaagcgcgagcccacaccctacagcacgtgggcagtgaccacacccggtgagcaccaccctccacgttctggaacatccagcgagggctgtgctctaatccacctgccctgcctcaagccctgacccgccaccctccacgtgctggaacatccagcgagggctgtgctctaatccacctgctctgctatggcgggcactgctggtgtcagggtgtggtggaatcagagccacaaccagcaggtgccaggttgccctgagagcggctgtcagggcgccccacgtagccaagtggcatcagtgcacatggatggtggcctccaggcacccctcgagctgccaagcgtgtccgaagggtgtcactgcctcctccatctggcagtgctgggcccagccacagaaggtgccgacttcctgcacctgctgcatcccagctgcctgcttcctctctcaagacagcacctctcgaatctggccccaagtgagacacagcaacagcgacacatgagagagactgtgatttgggggaaaagctgctgtcggcacacgtgtctccataaccactggaacgcaggccaccactggcacagctgcgccgcaaagcctgccccgggcctctaacaagacagatctgcagacagacacacagggcagccttctgcagctgcctgcccctgtccaccatctcctgaatgcctgcaaggagtcagcggcatgaggcttcacaagaggtgaccacgagctggtgccacagctcacacagctctgtatggggcattttagcagaacttgctgtcctgaggtttgtcagcagcacaccagcaaactccagcaaacagagaaagaggttggaattgcaggggccgacagagaaactactcagggataggctgcagcgccagacctgctcgccagccactgcctgtgcagcccccagcctgcaggttgtataggagcaatcagtgaccccagaagtgaaggaggcagtatgttagggactgcaaggggtctgagggaacagcagtacagggggactgttctgagagccgcagtcaggaggaggcagcagggcctgggcagagatcaaagcagccaggtgctcgcttctccgtttcccagctgcacccccccgctagcaagccctctgctctccccaggactgtttcctcttcaacaaaaaaggggaaagggcccgcctcagaggtcagaggtgccgagagccttaagtggaagtggccggctcctagtgggtgatccggagtgacagtgatggcttgcagggaggcagggcagggcagcgatcctgcagggccccagctgagcgttcacctctgaaccccaaataacctcatccgtgaaacaacgccacagctcccagcaccacaattacggaaagattcacaaagtgcttagcacagtgcctcatgtgcagtaagagacattaaatattaactgcttctgtgagtctcaatactggccaaaggttggccacatttcatctcctctcaaagcaactatgggagagcaggcgctgtcactgcccgtaatcacagggcgccagagcccagagagcagccacttctattcgcaaaggccttccgccttgccaggggccaccccgggggctccagctccgaggaagggctgtcacccgctcctctggccgcctgtgcaggaccagccctggtggggaaagcaggagtttgctcaggatactgtgaattaatctacatggcacaccaggagctcacacgcagacgctgtttcacacggtgctcatgtgcctgctctctaagcgctgccctctatgcgctgcataaacaatcttccaggggcctttcaccagtgacagtccagaggctcagcaggaccccgcagccagccaacggcaaacccagcacaccaccagggcccccctgtcacaaccctcgcacactcgaaggcagtgcttcccagcacgggtaaaagcaccgtgtcaaatgtcagcaatggcccggtccattagggtgcaggggctgcggggccagtcttggggtcagtgtctcgccagctgcacaaccagcgggcagaggtgtcactcaccatcaggtccttcacgcggttgctgagctggtcgatgaacaggatgggcaggtaatgcacggttttccccagctggatcctgggattaaagcacaactttccaaagtcagcaccaaggcttttacctttcaatgaatgaacacatccagacactgggtttaccccagagaagtgcccccagcaccaccctcaactgcttctcagtcacaatgaccccagggcacagcctccccaaacggaagcagagagcctgacaacatatgcacgcttgtgagacccgctccccaggaagtgatggccacaggtgggaaaatgcctccacggccactaggagcgggggaattacagggacacggccgtacagcagaacccagggccctctgcgcactgacacggagcaatcccaggatgcactgacacggagcaatcccaggatgtggccactacacttggaagggcgagtcacctgcagccacctgggaggcggagctgtggcgcaggagttgggggggggatccccaacacatgggagagacgaggtgagctcaggtggccaaacacgtgaattccaataattgtatttaactgatggcaagtgatactaaataatctaaagattaacttttaagtaaatctaaataagccaagttaaaaaatcacaaggggagcaaagcaacccgttcccatcagcgccttccgttttctctccatggaagccttcgccagccaggagcaaagggcaggatgctccagcttgaattcctttctccccccttccagccctgtggtagcagcacaacctcaatgaggaagtgggaaagagctgggaggcccgggcagcaagtgcctccccacagaggggactccacggtgcaggagtgacctgctggcgagttctggccagccagggtcccagcaccctccccccggcgctccagctctggggccccacttacatcttcatgtaccgatgcacatcggcaggcagggaggacccgtcaaagacaaagttgtccgccatcacgttcagcgccagccgcggtcgccagtgggacactggctcatccagggcactcgtcggcttcttctccgcctcgatctgctgttaagtgaggaaaacagaggccaatgctgggacagaaaaccatgcccaggacagcagggtcagccccctaacctgcgaacagaacggccacacagacaggcaaactgtgcccgggacagcagggtcagccccctaacctgtgaagagatggccacacagacgggcaaactgtgcccgggacagcagggtcagcaccctaacctgtgaagagaacggccacacagacgggcaaactgtgcccgggacagcagggtcagtgccctaacctgtgaacagatggccacacagactggcaaactgtgcccgggacagcagggtcagcgccctaacctgtgaacagatggccacgcacatggaaaaaccgcacccggacagcagggtcagcaccctaacctgtgaagagaacggccacacagacgggcaaactgtgcccgggacagcagggtcagcgccctaacctgtgaacagatggccacacagacgggcaaactgtgcccgggacagcagggtcagcgccctaacctgtgaacagatggccacgcacatggaaaaaccgcgcccggacagcagggtcggcaccctaacctgtgaagagaacggccacacagacgggcaaactgtgctcgggacagcagggtcagcgccctaacctgtgaacagatggccacacagatgggcaaactgtgcccgggacagcagagtcagcgccctaacctgtgaacagatggccacgcacatggaaaaaccgcgcccggacagcagggtcagcaccctaacctgtgaagagaacggccacacagacgggcaaactgtgctcgggacagcagggtcagcgccctaacctgtgaacagatggccacacagacgggcaaactgtgcccgggacagcagggtcagcgccctaacctgtgaacagatggccacgcacatggaaaaaccgcgcccggacagcagggtcggcaccctaacctgtgaacagatggccacgcacaggcacaaccgggggagagcccagcagagagggcacagcggccccacagctgccaattccgccagactctcgccaaacagccccccatccttatctccacggaagccagggggtgctgctttgcaacatgtggtcacggacttcaccaattccttttttgaggtcctcaaactaagtgggcccacttctcagaagttgagagggtgagagccccctgaccttggcagaagactgtgcaagtccagggctcacacagccctgtggctgtaaggagactgagagggagtcagcttctaaaaccaagcatgtggggctgcctaaaggagacatactgaggattacacacggctcttacatgaagttaaacaggacatggactgacagggaaatcttagctcggctcagatctgatacatcctaggaagcactatcgcctcacccatttcctacctaaagatgcagtttagcgataatttacattacaaacggttttccacacctcctcacatttgctacctgcaagggaggcttcctgcgctacatgctacatgcccatcaccccacccacttttcaggacactggagaggagggatgggggccccagatgaggggacacggagaggggcaggtgagggatgttcctaaggagacagatgcaaactggggagaggcaggagcaggtgttggtagggctttcaaaaccccatcaactccgagagacctctctttctctctgtctggaacaatttcacagcctcttcctggaaagctgccatctttcttgttttgtaaatttttctgaggcagagtctccctctgtcgcccaggctggaatgcagtggcatcatctcacctcacgggttcaaacgattctcctccctcagcctcctgagtagctgggattacaggcgcgtgccaccacatccagctaatttttgtatttttagtagagacggggtttcaccatgttggccaggctagtctcgaactcctgactgcaggtgatccacctgcctcagcctcccaaagtgctgggattacaggcatgagccactgcgcccggccttgttttgtaaattatctcaccttccactcaacacaaaggtgggtgtcagaattcccttgctttatccttggcctgtattcatgatcacttaagtgacttccagtttttatttgcgcatacacctttctacagatgtacaaactacaacttgtacctctaccttttccatgatctccaattacattcttagaataaacatctaggaatagcccagtcctacacagaatgcagaatgagcccagctctcatccccagagctcctggtgagaagctgcaggccagctctgcaggaccagcgctccatcccaccaggtgcagtaggtgcccccagagactgagagggcccattccccgagggactctgcatcgagaatgccagcgatcacttcaactgtccctaattttagataaaacttgtctgcaaactcatggatatgattcagctgaaagtcagaaatgcacgccagtaacattccaatggcttttggctttactcccaacaaagtcactggagccctagacatcgcccacctgtgtggagaaagacatgtccgttctgacggagaggcacagcctgttatcagtaacccatgaagaccctcacctgtgtatcagactccccggtgagcaggttgatttcttctggcttggggaccatgtaggtggtcagaggactgaccaggtgcacctgcttcccgtcgtgccacggcaggaccccagcgtgatggaggaagatgtaggcatacagcgtcccattgtttctcgttttctttggtacagaaacattaactgtcctgaaacagaacaatcatttccactacatacatattatattctgggtctctagaacctattcaaatacctttataaagctgcttcaataaactaattttagctcagaagtactaaaaatgaaacccacctgcccagggctttacgagtcgtgtgtgtgtgtgtgtgtgtgtgtgtgtgtgcacgcgcacgcgtgcgcgtcctgagaactcggcacaggtgtgggcgcctacagccgaaagcaaaacggcccgcacttcctgcctccatctcaggaggctgagaggtcaataacccacctgaggccacaaggctgaagcagccagatggaaagcagcttctgggcctggccagggtaccagctgaccacacacactgaatcacccaactccgaagcgacagaagggaagggcagcagccacgagggctccaggtgcctcggcccacactactggaacctcagaaatttaatgtgggtagtttttttgggtgttgtagttttaaggataaaaagatgatcagccacaaaggactgactttcaaggctgtcagtcctgactgtgtggcatccagcacgagctgaggaaaggcccggcgagctgctacaactgtaggcctcggaccaggtgcctggctcttcacccgcacaccagggcccgacgtccatactacagccccatggaaaaacctcgcattccacctgtttacggttacatgagttcttcttcctctttaaaagtctcttttttgagacaaggtctcgctgtcaccaggctgaaagtgtaggggtgcaatcacagctcactgcatcctcaacctcctgagctcaagtgacctcccgcctcagtctcccgagtagctgggactacaggtgggcgtgccaccatgcctggctaactttgaaaactgttgtagagatggggttttgtcacattgcctaggctggtcttgaactgctgggctcaagtgatcctcccgccttggcctcccaaactgctgggattataggcatgagtcactgtgcccggcctctccttaaaaatcttacggttggccgggtgcggcggctcacgcctgtaatcccagcaccttgggaggccgagccgggcagatcacgaggtcaggagtttcagaccagcccagccaacatggtgaaaccctgtctctactaaaaataaaaaaattagccaggcgtggtggcgggtgcctgtagtctcagctactttggaggctaaggcaggagaatcccttgaacccgggaaccggaggttgcagtgagcagagatcacgccactgcactccagcctggcaacagagcaagactctatctcaaaaaaaaaaaaaaatcttacggccaaagttgtaatgaaaaacacaaaggcatctatggctcctcactacccaggaaagagtatgtctccaccaggacatcttaagtgtctctcttcaggcagagggtgcagggctgtgtatttctggtggaatgtgcacttggagacacttgaggcggagctgtgttaagagtgctcattctccccacttcctccaggctgacactgtgccaggtcaaatggaaaacctcactagctgtccttggtaactggtacacggcccggcacggaagaggcagtattcatgtctgggaaattaaacccagtgcagatgaatcctgagcatttttgaaaggtgtgggcagctacaaccgagcgggcagcttggtcaccagcacttctggcctccccttcatggaggcagagagagggcaggaactggcttgaggtcacaggcctaaagaggccaggcagaaaccggcttgccagcgcctccagctacccccagggactggctgcctagagggcgtggagtccagcatgagtctcaaactttcattattctctcctcagatacctcccaggcaatgaagggaaatggttgacttgtgagaagcaaatcatatttaaaagtatacttattcaattaaaaaaaattaggccatgtcagatgtaatttttaagatactctacatatttggacattgatctattctaacgtcacatgtcacttatttatgattttctacaaaattgcttcctctatcctcatctcttttccctcacaagtaacttaagcgaactacttccaccaggaaaagacaagttggtccccggtagtgaaactgcagagttgtctgctctccactggttcaccaagcgtggcccctgctccttcggagcctccccgtccaggacatcccactgccatcacactctgctgagaactacttcttaaacgacattcatcacctgtcaccttccaggctgaatgtttacaggccacgcataggcctccagttaggatattctaccacttcaagacattcggtaagactagttctctaaaataaacatgttatgtacagaaagctaacttttaaacaatctgaacatgagtaatgttttccctttcactggcattttgtcctacgcccatacctttcaaatttggactccacatcaaagtcttccacattcaagaccaggtcgatgttgttctcagcacccaggtgggacctcgtcgtggtgtacacgctcagctggaaaggagggggcgtcgagagtcagctcggccaggccctggcaggacgcactcaaatcccacggccagctggagggcggaagacctggccgctggggaaccaggaaaggttccatctcgactcgcgcggccacagctccgaactgggacgggaaggcgacgtctggggcctggaggagaggcccccaccccaacccgcccggcccccggccccgcggacgctcacctgcagcttgggccgccgcgccaggtagggctggatgcagttggcgtcgccggagcacgggcgggtgtagacgatgccgtacatgacccagcaggtgtgcaccacgtagaccacgaacacgcccaccaccaagctggtgaaggagctgcggccgctccacatggcggccccgcgcccggcgcccccggcccgcccgcctctcagccgcgagccccgcccgcccggcgcccagcccgccgctccgggctccgccgctcactggagagccgccgcgcgccaccgccaccgccgcgggggaacgaatgcgccgcgcgccgcagaccgccggccgccccgcatgctccggccccgctcccacctggcgccgcgggattcgccggccccgcgcgccgcttccgggccccgccgcctgccggaagtggacgcgccgcgcggctccctgggaaacggagtctcggccgctgccgcgcacgcgcatccaggatgcggcgcgtgtgtgacgttacccgcccgcgccgccctcctggggtgcctctcagggacccaggcgctcagctgctggggccgcggagggcgttcgcggggcgccggggaaggctagggccgcggggtccccggggcgccgtccccaaccgccgacgcctgccagaccaccaagtgggggccatgtcgacgtgtgaggagcctggtggaaggtgatactggaattccggcactgggcctgcaacccttttagtccacagttgtcagaatctaaagggaagaaaaacgaagtgctcagaaccctcacagaagccgggcacctcatcccgtgtgtgggttttttccccagtacaaacccattgtggggacaggcgcttttgaagaaaaaattcagattcccgggctgaagtggctcctcacagccttgaacctgacccccagcttcccgctaaagccgagcgtgcccagcacggcgaggtcagcagaggcccctcggagggcggccgggggaggtcagtgggaggccctcgggcagccggtggatgggccggcgacctccccgactccatcagaagcgctcagtctgtgcccttcagttactggagcagctcccgtcctttggccagttctctggaaggtcgtttccgtttctgtgtctgccacacgtgcagcacaatcgaaaaaccttgttcgacatttctagaaagagtagatgctactgaagacccaatagattgctgagggttttcagagcagggacaattgcatcagggtgctgggctccgggaagaactcagaggacttggctgacgggcggatgggacagtgattggtgccgagaatgggccagagaagcaggtctgcctggagcaggcacccgaggccgcgtgcaggggcggtcctgggccaccaacccctggaagctgggccaggagcttcggagccatccgaggcttttttcgctcctctcactctcatgcacctagttaccctccccacagagtcttctcacggcgcggttttcggctaccattgcagtggtgtctgactacacccagtgcctctatcgttcccccctcagtctgttctcgcttcttggcaaacctgtccttggaaagccctctgctgactcctgttaccctgatcaaatgcatcctcctcaggaggctcgtgagccctttgcagggtggcccaagaggcatcacgccctgctctccagccactcttggggtcccctacagggccaggcgtcccacaccagcagcgggtgctggtcctgccacagtctgtgcgacacgtgaatgaaggctgtagacctgtggtctgcgggaggcaaattccaactccagctgtcaaacataaggggactggtaggctctccagggtggattgtctcacggcatccagaggcagggaccgaggcagggacctagccaggcctcgggaaggagctgcaaacccaggagctctggggtgtggcttcatctccctccctccctaccaccctacccccatctctccgtgttcttcagatgcctcattttcctgtcaggcacctggccagctctaggagctggaatcaggttggtgcctccttggtcagtgtccacccctagtgtcatccacagtgaccaacggaaggggtggccggtggtacaggacattggccatcttgggaaagatcacagagacaggaagggcctctccagctgagcagatgagaggttcttcagtggtctggacacatttgcccactcatggccgtccccctgccctgttgagagaaccgatgaggaaagggagccctgcatctgtcgccttccgactccgaccgcctgaggctgggctgccggcaagccgcggcagcatatgacagcacttgaaagaaagtccctggaaagaatgtggatcattgcagaactccctcaagaaagcgccagaggaggaccccattcagcaccccgcagaactcctcaaactcctgcgtccacactcccccagcgtggtggctgcttctatctccccaggcttagctggcctgggtcttgaaggcctctcctcctgctgtccccacgcagcatcctctctgccactgcacagctggatgcagtgaaggccaggcggctgtggctgcttgggcaacaggagctgcttgaccctgttctgctggccttgttggcctcactcccgtagagagccaaggttacccacgtctctgaagctaaggctttctttctaggcctgggggagtcacatcttgcattcgtactcactgagctccacgtctatggttagagaatttatcaacattttataatgcttttatcaattccatcatttgagggccctctggcaaggaacctgttagtacatggagtgcctggaatgttccagaaagctaacatgaacagctggcccttgctgtccttatacattgaggactccaaaggccactcaggatgacaggaaatgcaggctccaaaagattcggttcctttgtgaggccctcgggcagtggtcagacacgaaggaaataggaaatcacccccaaaccacacctctccctatgaggccacagctgccgatgcagccttgggagggggatggagggagagaggagggcatctgaatgtggacacaccctgctgtgagggctgtgggcggtcctgagactggagccagcccccaggctcagctcagctcggggacctcgtgcccgggctttcctggggaggaaggtgttggatctcctgcctctctgtattcattcacactttgggtttctctctcttccgcgcattctaacatctgcaggatttttttttttttttttttttttttgagacagagtctcgctctgttgcccaggctggagtggagtggtgcgatctcagctcgctgcaagctccgcctcctgggttcacgccattctcgtgcctcagcctccagagtaactgggaccacaggcgcccgccacgacgcccagctaattttttgtatttttagtagagacggggtttcaccgtgttagccaggatggtctcgatctcctgaccttgtgatccgcccgcctcggcctcccaaagtgctgggattacaggcgtgagccaccgggcctggcacatctgcaggatttatggatgaagggccacaaacccacatttggtctcattcaacagatgttgtgtcggcagggcgaggtgggactctgtctctgcccacgctacctgcccctggggttgtagttctaggaaagcaagtctcagactgagttttgatctggattaaattctgtcccaggtgcagatatttataggaaagcagggacaagagccctcaggcaaagctgggtatcctgcgatgggtgcctctgggagttcgcagataacaaggccttttcctggaggcaaagcagaagcgttctgtggcagaccagtcagctttctagcaggtcaggggcagggacatctaggcccaaggtgtcacaccgtgcctggctgctggctctcctcttgcaaggacacagctcttacaggacttcctagcccacccaagaacatctttccatcatcgaaaagttattgataagatttttccatcactgaaaagttattgataagtgccaccatgtgcatgagcagccctccaggtgctggcttgtgggccggtgtttgagaatcttgccagttgaatgtgggcatctgttgagggtttcccaaaggctaaccgttcgtgccagagggaggccgtggctcattcctagggccctgggtgagactggggctcatgcacacacaggtatcgatggcatgggctgtaacgagctgagtgcctgctgccctttcctagtggggggggtgggctcctggagaaagaggggaggctgctactctccagggccacagggagcccagaacgccacctcctggtgtcagcagcagcaaatccatataagtctgcagcaacttagcttttgcctcctcagaggaaataattcatccagggggcacaaggcagggtcagagactgaggtaagtttcagagcaagagtgaaagtttattaaaaaagtgttagagcaggaacgaaaggaaataaagtacacttggaggagggccaggtgggcatcttgagagagcaagtacacggttttgacctttgacttggggtttatatcttggcatgcttctgggggctgcgtcccttctcccctgattcttcccttggggtgggctgtccgcatgcgcagtggcctgccgacacttgggagggccgcgtgcacagtgtgcttactggagttgtgcggtgccctcttgaggcagtcttcccttaccagttcctaggggaaggtcacacgctggttaaactttgccactttgcctcgtagtgtgcatgcttgacctcactcaccaactcctgagattttttttaaattttttaatttaagttctagggtacatgtacacaacgtgcaggtttgttacataggtatgcatgtgccatgttggtttgctgcatccattaactcgtcatttacattaggtatttctcctaatgctatccttcccaccccacgacaggccctggtgtgtgatgttgcccgcactgtgtccaagtgttctcattgttcaattcccacctgtgccaactcctgggatcttatcgggaagtggctcatcatcagctttaggtgttttctatctattgggagcctgcctttctctggcaccagctgcaaccaataattattttagacagtttaacaacagcttgaccatcacctgatgatcacctgacatttctgttggggcgcgggcccatctcctgccccgctcatgtggggctcagtctgcccaacctttccccaagggcctgtctttacttcatttgtggtaagccccaagcagaatggactcttgttatgatcccaggaactgctcacggccacctctgtggatgaacacctagcggctgctctaaatacatgctgtgaaagttgtcagaatcaaaatggagtcactaatgttaagaaagccctgacaaagagttggtggggaaggccacgaagagaggacacttatgcttgcatgcctgataccagaaaggactacaaaaaccacagcccggcacagaggccatcacacccttacacaaaaaatatttctgcaaggacaactgcccagcaattgcctgtccaacctcagactggaatgacctttgttattgatgtttgtagccaaggagaattatctcaaaaccactgtgatcctgctcgcttttcctttaaagacctttgtcttccttgacctccctgaataggcatatggtttactatggcgtgtgtactctccttgtaatgttctgttctcaagctaacatctattcttctagagaacctctctcttaggctgacaacgctgtggatctgggctcccaggaactggggctgacatcagaggggtggaaagagaacagcagctctccctgaggggatgcttgccaaggataaggagacgatggttctctaaggattattttatattcatctaaaagggtatttttgttgttacccctagccttttttcccttttaattttctgatgaataatttatataaagaaaacccattctaagtgtgcaattctatgagttttgacaaatgtcttcacccatgcaaatcctgttacaacggagatatagaacatttccattacttaaaaacagccaggtgctgtagctcatgcctgtaatcccagcactttgggaggtcaaggcgagaggattacttgagcccaggagttcaagaccagcctgggcaacatagtgatgccccatctctacaaaaatagaagaaattagctgggcatggtggtacatgcctgtgatcccagctacttgggaggctgaggtaagaggattgcttgagcctgggaggtggaggctgcagtaagccacaatctcaccactgcactccagcctgggtgacagtcagaccctgactccaaacaaaaaccaaaacttccttcttgcccctttgcagccagactgttttctgtcattgtggatgcatctgttctagaaatttacatggatggaatcactcagtcttttgtatctggcttcttttacttggcacagtgtttgtgaaactcatcattgttccacataacagtagttccttttaattgccaagtggcattccttggtgtgaatgcaccacagctgggctagccacccagatgctgatgggcagtcgataggcggtaggatgtccccattttcagtcactatgaattggtctgctgtaagcactagagtagaaatttttttttagagacagggtctcgcaccgtcatccaggctggagggcagtggctcatagctcactgcagcctcaaactcctgagctcgggcaatcctcccacctcagcctcccaagtaactgaaactacaggcacacaccactacacttggctaatttttaaactttttgtagagacggggtcttgctgtgttgcccaggctggtcttgaactcctgggcacaagcaatcctcccacctctcaagtagctgtaataggtgtaagccactgcccagcaccatttattgaagaatctgtcctttccacagtgagtgtgcttagcacctttgttggaaatcagtcggctgtagaaagcggattaatttctgggccctctgttctgttccattggtctgtgtgtctgtttttatgccagttccatgctgttttggttactgtagctttgtagtgtattttgagtctgggagtgtgatgcctccagctttgttctttttgttcaggatggctttggctgtttggggtcttttgtggttccatacaaattttaatattatttctttttctaaaaagaatgccattggtattttgataggcattgccttaaatgtgtagattaccttgggtagcacagtcgttttaacaatattagttcttccaatccttgagcatgagatgtcttctcatttgttcatatcctcttcagtttcttgcgtcagttttttgtagttttccttgtagaggtctttcacctcttttgttaaatttatccctaggtattttatattatttgttgctattggaaataggattgccttcttgatctctttttcagctaggtttttgtttgtgtatagaaatgctactgatttttgtatattgccaggggcagcctgtgggactttttcttaggcccttattggaggcacagagccattgggcaggccaggggcgtacctgcaggtgggggtgccgtggggctgattttcaggccctgagctcctgctgtcagttgcttggaggctggtggggcctatgtggggagacctgcagctgtttggctcaagggtgggtttgctgcaggtgggaggagcagacagctggaagagctgcggtggggatgggtttccctgctgtgcaggaccagagtcacagctaagcctgggcccaagctctatgcagctggggttgtggtgttcagctgcccatgtgggcttggtggcatgaaagtggagccccagtgctggagaggtgcaggtgctactggccccagggcagagcccctccagatgcaatagcagcttggctcatggcatgggtgtggggtgggaggtgcacaccttttgctcctaatcgggggaacactgctgtgagaattccctgcagtgctcccaactgggctcagggcttgcaaggactgtgggttctcctgcagcaaggactgccagcatttgctgtagcagtggcagctggtggagatctgcttaccttttccttgccactggaagtccctctttgcttctaggtcaaactggtgggtgaggaagactgggcagcagacgccacctgggcttccgagcaccacaggggtgtctccggcctctgctgcagtccagcactctgccttcgacgctccagtcaaattgtcactgtcaacttgttgccttgatccttctttgtggggaggaggggatgagcgccagacagttctagtcagccctgttgccaacaccactctccttctggtgactttcaaagagaaaagttgaacatttttactacacccaacgtaatcatgtttcagtgataagagtttttgaattttgctgtatgtgttttgaatttctgttagtaggagcatactcatttggggtcattgtgtcctcttgatgtcttgaccctgttgtctttgtttatccctggtgatgttctttgttctgacatctgctttgtgtcgtgtcaacataactacttcagctttcttttgacagcgcttccctggcatgcccttttctctcctttaacttttaacctacctgtgttttaaatatacatatgtatctatctgtctatctacacatttttttttgagatggagttttgctcttgtcacccaggctggagtgcaatggcactatctaactctcactgcaacctgtgcctcttaggttcaagtgattctcctgcctcagcctcccgagtagctgggattataggcacctgccaccatgcccagctaattttgtagttttagtaaagtcaggttttcaccatgttggccagggtggtctcgaactcctgacctcaggtgatccacctgccttggcctcccaaagtgctgggattataggtgtgagccaccacgcccagcctattttcttttttttaagtaaaattttgagacagggttttgctctatcacccaggctggactggagtgcagtggtgccatcttggctcactgcagcgtcaccctcccaggctcaagcaatcctcccacctcagcctcccaagtagctgggaccacaggggttcaccaccacacccaggcaatatttgtatttcttatagagatggagttccaccctgtttcccaggctggtcttgaactcctggactcaagcaatcctcccacctcagcctcccaaagtgctgagattacaggcatgagccaccatgcttggccttaacctatctgtgtatttatatttaaaacgggtttcttgtaggcggcttagagttagttctgtatttcaatcaacaatctctgccatttaattagagtgttacatcatttaaatttaattatcaatgttttcttatttgtcctattcattctttgttctttttcctcttttttcttcctgcttttagaattataaatttatcttttttggattcaaatatgtcttcttattggcttttcagctagatctttttgctttacttgttcagtgtttttttctacaattgtcaatttgcagcttaaacttaagctagtctacttccagatattataacccttcatgtggattttaatgactttaaacagtaacgttccatttctgttctattcctttctttattgtcatacatttcactgtcacgtgaaatacagctgatatataccctttagcctcaataacttcctttagcattttgtgttatgcagatctactgaaaatggattctaccaattgtcatttgtatggaaatatccttaatttgccctcgttttttgaaagatattttcactaattctctaggttgatgggcactttttcttctgtcatgttaaatcacatcgttcagtccactgtcttctgacttgcattgcttctgatgacagattggcagctgttcttctgtgtttctttgtgttttgtgtgtatttgtctggcgcttttgccttgtctgtcctgcttggggcttactgagcttatcaatctgtgagattcaagtctgaaaatgttgcagtgaaggtttcttcaaatatttctctgcctgcctcttccttttgggactccagttatatgtatatgagctgcttgatgctgtttcaatgtcagtgaggttctgtttatgttgaaacttttctcccgtctccgtgcttaggtttgaatactttctaatgcttgtattcaagttcactggtatttttctgcagttttcaaactgctgctaagctcatattatgaatttttcatttcaaaggtatttttcatctcagaggttccatttagttatatatatacataatatattttgccaggatatatcctttggttaaaaaaaaagtctgtaggtattaaaagcagggttacgttgaatgaccttgaacatccattttctgatattttgtgactttaaggtagctgcttagaagtggaattgaaggatgcagttgtgaatgcacacaggtctttccgggcatctctgagcttcttcccgcaggggctgtgctgtttgcatcccagcagcaaagcatgaggttgtgtgtttccctgcagcctccccatcaaggtgtgctggtgaccttgtggagttttgccaacctgttaggagagatgttgtaactcagtgttttcatttctaattctcggatcatgaaaactaacaggcttggcttggtgtggtggctcatacctgtaatcccagcactttaggaggccaaggcgggtgtaacacctgaggtcaggagttcaagaccagcctggccagcatggtgaaaccccgtctctactaaaaatacaaaaaattagccgggcgtgatggtaggcgcctgtaatcccagctactcaggagactgaggcaggagaattgcctgaacctggaatgtggaagttgcagtgagccgagatcgtgccactgcactccagcctgggcaagagagagagagactccatctcaaaaaaaaaaagaaaaaagaaaacaacgggcttatttacagcaggagctgtggtgcccttgggggatgtgtgagctccagggatgacagcaaccaccatctgttcatctcagcacttgggagacaagccctgtaggattcattcagtaaaaataaaatcctacgaagtgaatttgtaggcttggggaagtgtggacaatgcagtttgggcatcacctacgtacaagcagaattcgcaggaacagctcctgggcattgggatgaaacatggcccctgccatctgcttcttggtgtcaggccggcctcctgcttcccgcctttgttgccagcgtggaggaagcagaggtcagggaggcagtcactgcttgtccctgacacgggactcagtctccagacaaggcggctgctctcagtgtcctgtctcatgactccaggaaggcccctccctgtagagtgtcagcaagtgcccacgacccacccacagagcagctgagcagtgcctagcgcgcagggcaggccacacggcgccaccatcagagcacgagaacagaagggctgggggccgtgggagcctgggcgggccagggtaacgaagagcccgccgtcggtctctgcatccgtcggctcacgtggccacggagcagcaccgtgttacagcaaagcggtcctgatccagaacccaagagagggttcttggatctcgcacaagatagaattcagggggagtccgcagtcccatgtggaagtgagtttattaagagagtaaagtggcgaaaggacggctgctccacagacagagcagggcattcccgaaactaagaggagaaaggcgcccacctggggtacaatcctggtgtatacggggagatgtgctctgctacgagcgttgatgataaagaattaattttcttaattagtatattttgcaagaatcaatatcatttatctttaaggcaaaattaggaatgcctttgttctctggatatcggggtatctggactcccaagtctgggtctgtttagtaaacattatttatttgttcctttatctctaaacgtctagaggctgggaatgcccgactctctgggagtggagcctagcaagtcctagcctcgttttccagccctcactcgaggtggagtcgctctggttccagcgcctctgacgaccgcaggctggggcttagacaggacatttattgctcaaagtacagtccatgaggctggcggtcccagatccaggtgtggcagggctacttcctcctgaggcctctccttggcttccagacaccgtcttctccctgagtcctcaccgggtcgcccctctctgcgtgtccgcctcctgatctcctgttcttacaaggacaccagtcacaacgggcccaccctcttgatctcatttaactgaaatcctttctgtaaagaccccatctccaaataccgtcacattctgaggtcctgggggtcaggacttgaacatttaaacttggaggcaggcacagtcccacccatcacagcccattgtcctccacagccctccaatctcacttcgagtcttccccaagggtcagcacaggctctgcccttgtgagcttcatgggaatgagggtcaagggggtaatgagggccggaagtgatcggagggaggcgggaagagggaggacttggactccgcccacggggtccccacctgggcatggctgtagggaggggcatcgtgctgctcttgtccttactgggggccagtgtctgaaggaggaagggatgggtggcaggcactgcggggaggagagggctctccagcactcatattttcatcctttgaccgtgctgcctgggcactgagcataggaggatgtcgctggctggcagggctgtcaccctcatcgatggcccagtttcccaggaggaaatggatgggatggcaggtttcagcagagagcacacaagccctgctattatcttacaaaaagccaggccagcctcccacagctctccatgtgacttagcatcaatattgggggttttgtagttacctcaggaaacctacttttgaacaaataatagattatgttctctggaacacagggggagctaaatgccttttgacagccagcagcttatggaaatagcccttttcctggcagaagaagtcggttccccacggccgcgcccgctgcttcatccagcttcccctcgctctgtgccgacgggtgcccggagttcagcccctggcctgactgttgctgtcactgcccccttgaccactgcaatggtgacagctgtgatgtacgattacaaagcgggacacagagccctcccagagcaaggcagctcccctgtgggccaagcggaccaggctactgggggaccctgggctcctgggacatggggtgcgggtgcagacagggcctgaagtgctcctaggtgcatgccagtggcaggaagccccatgtcacagagatgtggcccaagcgcttgacaagagacggcgtcagatgatgtaaaagaaaccaatggacctaagtgggtgccatcctgagccccgctggtggattaactcagttccatgcccacgactgcccgaggtggagcgatggtcatgcccactgcacagatgaggagccgaggccaggaagggttttgccagatgcctgggctggggccagggctcaggaccacccactgaactgcctgctcggcccaccctggcaagtgtgtgcaagggcccggtggtgccgacgaggagggccatggggaggagatgttgttgtcctgagactcccagccccacctgagggggaagagggtgggagagcaaggctgggagccacccttgggggctgtgcatgtgccccctgacattggaggacacaggccacgccacacctgtgccacccagggagtgggaaggaagcacgtggccgtggagaggccagcaggtggcaggaagggctgcaagcccccaaccacggggtcacacgtaggggacccagcaccccatgcaggagctgggctgtgccctgcatctgcacaggccggggcatgaactgggcatcagcaccgccctcccatggggacgaggacccatgcccgcgtggtgcaggagctgtgcactgagcagccccatgggcaactccagcccgcagtgccaggagcagggccaggccttgtggcaggggtgcaggctggctcctggcggtcgctcagctctcggggataggtgaggacgcgtggagaggggatagcatgggcatcaaggccgaggcactggcccctcccagaatggcctccaggaccaccctgcctgcccccgggtgccccgctgtgcctggggagggtaagggtcacttcgggttcacggctccagggtcgccgtcttgaaattcttaccaattttatcactaagcttatgtttttcaagcaagataggatgggcatggagcccctgcaagagcaagccacgggcgccctccgagcccatcgccgctcagaacctgaacagagcacataagcaaggctcacggcgaccacagggctggtcgcagccaggacggggtgaggagctgcagccccagcctcgccttcccttcaagccgagcagcttccagtgggaggaaacgccagcgaccgggcccccgtccttcctcctcaggcggtcctgtggaaggtgcggtgctgatggaacgtggtgactgggaggtggcataaaagcagccactgtgctggttttgtgggacctctcctcctttctgggggtccctgaggacaggtgcatgtgcgaactgtgaaacagaaactgagatttcaccttccggatgagttccatgctttcttttttgcatttaaaaccggacaatgtcacattggcaaaaatctagagttttctgccgtcttagctggaaatgagctgcaagtttttctcaagatgtagtgtgtaatccgtcagagcaaaacacggagagcccttagcagaagcccacttcaatgtattttcttcatatccctgaagttccttaaaaataggtgacgatgtattgggaagaggagaactgagaagttcccttgcaggttttgtatcagtgacatgtaaatgagcaattcacagatgagcgcgggcacagctctgtgtgctgcgtacatacgggccgggctatgatgtctcacactggatgatattccaccttcggaattttagtgtttgaatacagaaaatgggtttaataactcactgtggttttgatttatcttatattcatcatttcttaaaactcatttcttataaaatttaaagtaaaaaaaataaaactagacaatatatgaatggcagatccatgctaatgattctaattttcagaatttctttacttagaatgacataaaatagcaactaaaaaataagttgagaaactgtggaaaaagaaaaagctaggccgggcacggtggctcatgcctgtaatcccagcactttgagaggccaaggcgggcagatcacctgaggtcaggagttcaagaccagcctggccaacatggcgaaaccccgtttctactaaaaatactaaaattagctgggcttggtggctcatgcctgtagtcccagctacctgggaggctgaggcaggagaatcgcttgaacccaggagggagaagttgcggtgagctgagatggtgacattgcagtccagcctgggcgacagagcaagactctgataaaaaagaagaagttgtatattttactaccttgcacagcactttcccctgctttttaatgaggcactccacattttcactttgcacagtgccctgagtgggccagatggagggacccttcttctggggtctcaccctcatgtggctgctggtccacacgggccctgacagtggctctcggggtgatcacagcctgaggctcctcctttctcaggccctggtcactcaaaggattcccggcggggtccctccagtgaacacccagctcctgcttcagagcatgaggggtgtgagttttccaaaggtaccagctttggacagatggggtgagatggtggccaaggggtccctgcaggggcccctcatggtcctggggacaccctgactcttctggccgggtggagcacagccccagctacctttgcactctccaaggtcccaggggcacccaggctactgagtgtcccagacatccacacgcagggattgggggaccagaagctggggagccctaggggttttgagggcagagggcaggaggagggtgtggttaatggtgtccaatgcaggtgctaagccacaggaggagtgggctgtggcgtggacgatgcaggtagctgtcagcatcaatgagatgtgcagtgggctcattggggggtggggttttttaagaaaaaagcctcggggtgtggctgctggtgggtcgccctgtcagagaagagaaagctggagaggacacagggctggggtggagggcaggtctgcgggttccagggggcacactggaggggacactcaggggctggaggctgtcttgggttgggactgaggctggtgtggggtcttctgcaggctgggactcatggtggggctgggttcaggggtgggaagttggagggggatgctgggaatatctgctgtgacagtagaaaatgtacattcaggattagcctagtggctcacgcctgtaatcccagcactttgggaggccgagatgggtggatcacttgaggccaggagtttgagaccagcctggccaacatggtgataccccgtctctactaaaaaataataaaatcagctgagcatggtggcacgcgcctgtaatcccaactactccgaaagctgaagcaggagaattgcttgaacctgggcggcagaagttgtagtgagctgagatcgcaccattgcactccagcctgtgcaacagagcaacaccccgtctcaaaaaatatatatatatttattctattttgtccccagttcctgacacagtgcttcttggaatgtcctgcatggtaggaggtgagaattcataacaagcccctttcaacctctcctgagcttatgctatgaggggactcttggtaggcccctagatagcttgagggtggtgctggttgctaggggaaccagccgtgtgattgaaggctgaaactttcagccccatttctcaatctctggggccaggggttggagatggaggccaacgaaaaatcaccagtgatgtcaccaattgtgtccacatgacacctccatcagaaaccctcgcccatgggatttggagagcttcagagttggtgaacacgtccgcgtgtgggagggggcacaccccaagtccatggggacaatggaatgtcctgtgcccgggaccccaccagccctcgccccaggcacctccacatctggatgttcctgcgtattcttcacaatatcttctccaagaaagcagtaacggtaagcaaagtgtttccctgacttccacgagccatcccagcaaattactgaatttaaggaggggatggtgggcccatttgcagccacgttggacagacatgtgggtggcctggggacacgatgcttgtgactggctgagtgggggcagcctcacgggcaaagctttcaaccttccggatctgcgctagcgccgggcagtgttggagctgaattaaattgcaggacactcgggtggtgtctgcagagacccggagaattggctggtgtgcaaaacccacacgcttgttgtcagccgtgtcgtgaatcgaggaacagctttcctttatccacaaaaaaagcgagttttcattccctattcttaagtgcgtggctccagcttttgcaataattctagtccttatggtgaggagggtaatgtgagcgaggctgagaaagctccggaggagtccacacggccatcctctttctagttcagcaacaagagccaaaatcaggtttccacggaaagtgactggcgttggctctccatagggaaatgaaagtaaaagaagcagtttggccaggcgcagtggctcacacctgtaatcccagcacttcgggaggctgaggagggcgaatcacctgaagtcaggagtttgagaccagcctggtcaacacgggggaaaccctgtctctactaaaaatacaaaaattagccaggcatggtggcgggtgcctgtagtcccagctactcggaaggctgaggcaggagagtcactggaactgtggaggtggaggttgcaagtgagccgagatcacgccactgcactccagcctgggcgacagagcaagactctgtctcaaaagaaaaaaaaagcagcagtttgggccatgcagtgagcgctctgtggaccagtggactcatctgctggggtccaggcagcctgggctgctcctggttcttccctcctccctgcatgcacggccctgcactgccctccctggggctgctgtgggagttctcggggctctcggtggttgtcccgctggctgggaagggctgtggtgtgttgggtgctgcctaccctgaagtacacgaattagggttgtgctgtgttgggtgctgccctccccaggggctgctgtggtgtggtgctgggtgctggctgccctcaagtacatgaattaaaagttgcttctcctcttgccagatgcagggaggaggctgagcaaagccctagctcatttacgtagagacagacacacagaatttttgcaggaatttaatgcactctgatttttccggagatctaacaatagcgcacatcaaagtgggtggcactgggctctgttcagaacattccagcaactggtcactgcagcagcgcactcacatggcggtgggcgctcgggctgacctcaccttctggaaggtgcacctgtgtgcgcaggcatccgagtccatgtcacgtgctggcacggcaatcgccatcctttcacaacaggtcacgaagccgactgattttgaatggctgtgtgggtaatactgcccatgaccttcattccagagagcaacaggcatcaggaaacgtctgtcacaacaggcagggctggagcttgacacggttgagaatccctgatgctcaacccgtattttctggctaccagttgaaagaaagggtgagaggatgcttggctccgagcaagtggctgagaaacgcagccataacattcagctccttctcaccaaattctttaccccaaagaaccctgggaggaagacacatttgctaggagctgggtacctgaaatcaaggtaaggaaaggatgccaggttctctcctccaaatggcgctctgggcgtctcccctacaatcagagaggaacgaaacttacacgaaccttaaacttctgttagaggtaaaatagaataataataataaatcccagttcagtgaattcaggttaaccgcaaagctcatcggtttggagagacgagctctgacctttgtgaaattttgtgcacaaacaacctggaacactgctgtgagagagagggctgtgtcctggccagtgtcggccccacctgcaggaagtgcttatctgtttcatcggtgaggtttgctggctgggcttggggaataatgggttcccactttctcaattgtctccagggtttggaacacagggctctgcgcctctttcttcacttgctgttccacagagaaggtgacccgagctgcacagtccaaaccgaaggacacttagtccccaactggatgaaacagggaagaagcgaaagcatcttcatctatcggtctcagtatttgaaattttgctttctagataggcagaccaacggaatagaagataaagtctacaaatagaaccaaatatatgtagagattaggtatacaatgaggtggaaaaagacggacttttgacaataagtttttgggacagttggatagccacatagaaaatgatacaactggccaggcacagtggctcacacctgtaatcccagcactttgggaggccgaggagggcagatcacgaggtcaagaattcgagaccagcctggtcaatgtggcgaaacccatacatctactaaaaatacaaaagtggccaggcgtggtggctcatgcctgtaatcccagcactttgggagctgaggtgggcagatcacgaggccaggagtttgagaccatcctggccaacacagtgaaaccccatttctactaaaaatacaaaaattagccgggtgtggtggcgggtgcctgcaatcctagctacttgggaggctgaggcaggagaattgcttgaacctgggaagcggaggttgcagtgagctgagatcgtgccactgcactccagcctgagtgacagagcaagactatctcgaaaaaaaataaaagaaaactaaaagatacaacttaacctttagcttattttccacaccagaataatccccaaagaccagagatcttcatgcgagaaggaagtaaggtctggctgaaaacacagctgcaggatgtcacaggagaggaaaaggctttctaaccatatcttataatttagaagaacaaatacaagattgccaaaatttatcacttaaaataatgtatactcaggtctggcacggtggctcacacctataatcccagcattttgggaggccggggcaggaggatcacctgaggtcagcagattgagaccagcctggccaacattgtgaaaccgtatctctactaaaaatacaaaaattagccaggcctggtggcacacgcctgtaaccccagctactagcgaggctgaggcaggagaattgcatgaatccgggaggtggaggttgcagtaagccgagatcgcatcactgcactccaggagcatctcaaatatgtgtgtgtgtgtgtgcgcgcgtgtgtgtgtgtgtgtgtgtgtatactcaatcaaaatcctagcaagttattttgtggctattgagaaatgattctgaagtttacatagagagaggaaagacccagcacaaccaacacaaaattgaagaacaaagtcacgggactcatgctgcccggctcctaggctcctgatgaagctgcactaatcaaagcagggtggtgaatgaaagatggaggagggggcaggtagggggtggataagggggcgggtagggggcggataagggggcaggtggggcggataagcggggggtgggtagggggcgggtaagggggcagagtgcagaacccagaaacagacccatgtggatgcagtcagaggagcaaagccaattccatgggacaaagagtcttttcaacaaatggtgctgggacaacaggacacccatgtgcaggaaagcaaatccagacacagacttcccacccttcacaaaaacgaactcaaaatggatcagacctcaatgtaaaacacaaaactataacgctcctagaagataacatagaataaaacccagatggccttgggcatgagatgacgttttagattcaacattcaacatgcctgttggatctaaaaggcatgatccatgagagaaataatggataagttggacttcattaatgtgagaaacttctgctctgcaagagactctgtcaagagaagacaagccacagactgggagaaaatacttgcaaaagacttatctgatagaggacagtactgaaaatatgcaaaaaactcttaaaactcaacaataacaaatggacaatagattttaaaagtgagcaaaatactggaacgtaaatctcaccgaagaagttctgcacatggcaatacgtgtgtgagaatgttcagcgtcacgcatcatcggagaaatgcaaattacaccaacactgagacaccacacacacctactagcatggccaaaacccagggactggcaacatcaaatgctgacaaggatgcggaacagcaggaactcttgttcatcgctggtgggaatgcaaaatggggccgccgctgtggaagacagtttggcagtttctcacaaaactagacatgatctcaccgtacggtccagtaatcatattcctcggtatacacccaaagggactgaaaacttctgtccgcacaaaacctgtacatgggtgtttatagcagttttattcataagtttcaaaacctggaagcaaccgagatgcccttcagtaggtgaatgggtaagcaagctgtggcacatccagacagtggactcagcactaaaaggaaatgagctcccaaactatgaaaaggcagggaaagaacttaaatacttatcactgagtgaaagtggccaatgtgaaaaggctacaccctgtggggttccaactctaggacatttgggaaacggcaaaaccatggagatgttaaatagatcagtggttgtcagaggctggggggagggaaggttgaacaagaggagcatagaggatttttagggcagtgaaactcctctatgtgatgctataattgtagacacgtgccgttagatatttgtccaaacccttagagagtacagcaccgagactgagcctgaatgtaggcttcaggcgctgggtgatgacgacaggggtcagtgcaggagaatcagctgtaacacatgcacctctggtggggaagttcatcttggggaggctgtagttggggaggggggctataggaaatctctgtaccttcgtcttaattttactgtgaacctaaaactgctctaaaaaataaaacattaaaaatgaaatatacaggctgggcacagtggctcacgcctgttaataccagcactttgggaggccaaggagggtggatcacctgaggtgggagttcaagaccagcctggccaacatggtgaaaccccgtctctactaaaaatataaaaattagctgggcgtggtggcgcatgcctgtaatcccagctactcgggaggctgaggcaggagaattgtttgaacccaagaggcagaagttgtggtgagccaagatggtgccattgcactccagcctgggcaacaagagcgaaactccgtctcaaaaaaaaaaaccaaaaaacaaaaaaaacaaaacaaaacaaaaaaccaaaaaatacacatttttgcatgaacaaaagcccatgaagaaaatcaaaggtgtagcacatggggaaatgaaagccatagcacatgagaaaatgcttccaacttatcgcacaaaggatcaagctctgcaaactataacaagctcctaaaaatcaaggcaaaaacccaattatctgttggaaaaatgggcagaaaaagaaaggcaatgggcagaaaaagaaaggcaatgggctctcccctgtgaagacacactcagcctcacttacagtaaaagtgcaccacaccccaggtcctctgcacgccatttcccacccatcagatagtccgagaggcagacttccaacagcgcctctgcatccccaaggagatgggcggctcatggggcactgctgcctgggaggggaggtggcttttgttggagggcctcccacttacccagctacagcaacaaatgtgacatctgtccctgagatggccagacacgcagcaagtgctccatgctgttcactgtggcatcacatgtagtggcaaaggtttggaaacacccaaatatcaaagagcagaaggtttaataaataactgagacacacccacaaaatgcatctgtaaagaagaataaaaaaggtatctctgtgctgataggcagttatcttccagaggtggcgttctaagtgcaagggaggcgggagagcggtggggcttatttcttggtcacagccttgctgaccaaagccggacccggtccagatgaggtgaagtggagaagctggcaaagaccagcagatggtaagagggcaaaacctggctgcccttgttggtcactagcctaagacatgcctaccagtgccatgacagtttacaaatgccatgacagtttacggaaattaccactgctttttatggcaatgacctggaagttatcacccctttcctagaaagttctatataacctgccccttaatttgcattgacctgtcccttaagttgcacgtaattgaaagcgggtagaagtgggtataaatacagttgccaacaacccatatgctgtggactctgggcacactgcctatgagtcagccccactctgcaaggaacagtgctggtcaataaaagtttgctgcctaacactgccggattgcccttgaattctttcctgggcaaagccaagaaatctcctgggctaagccccagttttggcctgcatcacctggcagccatgaagatgaaacagtggggatggcagtggtgggtggaggggtggtgagacagcagagaggtgatggtccacagtgggtaggacaactgccatctctcaaaagaagtggtgaggtggcaattgacaagaaggctggacggtggagaggtagcaagacggcaaccagccgtctggtgactggctacacagtgactggtgagatgggaagaagcagcaatcagagatggcgattggctctgcagcgatcaaagaaaatcaaagtcatagagcggctagtgcagcggagctgtaacctcagccaaaggctctcttcagagccgtcatctttcctggtaggcagcagagccaagtggttgggggagtggccacagtgccaccaccacgcatgggacccctgccctggctggcaggtcaccggtccatgtgccagtctcctcacagcagctgagcctgcccaagctggggaaaccctgggaggagacctttacctagggattaaggtggagaatgatcagcaccattttggcccctgtggttgggtgagtgtcctcccctctgcccacccctgccaatatgagccaggaaattaggcctctggctagatggtcagtttgaagtccccgtagcacacctgaccagccacatccttgtcattccttctctcgatcctttctcctctaatgccatttcattgcctgttggccattttaatttctgctttgaaatgtacgtttcatctgcaatttttgctttggctccctgctaactctacttgggcagttacttaaggaaggacacttggttgtgggaggtcccctgttgtgctgaccctaggacagcagggtcatgttgttctgtggccccagcttggcctttggggttcactgtgggccaccagctagatgctctggggttttcgacattggtgtggggacccttgttggctgatactcgactgctctgggtttttggcatttgggattgtaggccacccccagacgctccagagtttttggcactggtattccctctaggattgtggcgttagaggccaccctaggggaatcttggccttgccttttctggtttcgtaccgaaagttattttctgaaacagcattttcttattgtcactttatctacacttttccttctacgctttgcttagtaaaaatagttgttttgtcatattttgtttgccagcacttttttaatgacttgctaccttgacttattccttctctgcaggacatgggaatctgaaaggggatgacagcaaaggtccagctgcttctgctcttgctagacttagaaaaatgcctgtgcccagtagaaatccttgctagacgtaaggatgatgatgagcatcccagaacacttgcagctggagtgtcttttatgctggccagtgtttgatgttcttcagggtactagttctagcccaagaacaattggcataaagctgtggttagacctgcagatttctgagcactggtcgtggtaagagcctggtggtgtagacattaaggttgcttggtttcgatgttctggaattgcatctgttttcaggcccggtgacggtacggctcatgaacgtaaaacgtcttctgacgaaatcagtatatgaattgatatttctatagggaagactactcaattgtcaacttcatcatagcttatagtaatcaatagctttcccggctttagatctgttgagtatggaagagtcaaaatttaagtcttcataatctgcatattcatagcatcagtatcatcagtgtcctgtagttttgatggtaagcgaagggttgttaatttcatctttgatgtatagaatatgtaatagatgaaattcggcttaaagaaattcattttctattgcaagaccatttgggtccaacacaaaaattagaaaatcaagagaactggtctaaacatggttctacacattataatgctatttcacaattggacttattccataaaaaagaagaaaagtgggaggaggtcccgtatgtacaggcttttatggccctttactggctcatgttacttccaggcaccaggaaggcacatctaagggaacccctcatagctgctccccctagaaggcctgtgccctccttggaccctcctcagttccccaattctgagggggtgttcagccagcgcaggattccacccagggccatcaggcccccaccccccttatccaactagccccagcctatacccccaactgccaagaaagcaggtccaacgggcacccccagaagcagggccccatatcagcccttcaagtcaaacttgtgtctgtggcaagaggcagctgacggaaatgggggcacttggagtgcaggtgccattttctatgtctgacttggcttcatgcaaggaggcacgtggccggttttcagataatccggggttttatggaagaatcaagtctaccatgctctttcacttgtcatgacttacaagcattgttgctcacttgctgtgacatgagggagaggcagagggagacgtgtgagattaagccagtcagttatgacaaggttagagaaataactcatggaaggatgaaaatcacactgtttcaaggtagtttggttgaggtactcgggaaacgtattgacgcaggccccaactccccagaagggcaaggtctcctgggtatacattttatccctgaacctgcctccgacatgaagaggaagctacagaaagcagaaacgggacctgaaacccctatgagccaactcctaaacatgccctttaaagtttgcaacaatacgaacagggcaagaggtagaattaaaagcaagataaatagccaaaatgtacaattgctaacagttgctgtcagtgccttgcccctcagccttacccatcctgagagtgtgttttaagattggcatctgacatgcccagacaagagctcttgacttgctggcccctgggtcagaatcagtgtgcctaccataagcaaaagggccattggcaatgagaatgtcctaaccctccctggtgagaaagataaaagctgcctgtcaatactagagttaaccttctgctagtcccaatgaggtgttttttgttttttgagattgtctcgctccatcacccaggctggagtgcagtggtgcaatcttggctcgctgcaacctccatctcctgggttcaagcgattctcctgcctcagccttccaagtagctgggactacaggtgtgcgccaccacgcctggctaatattttgtatttttagtagagacagggtttcaccatattagcaaggatggtctctatctcctgaccccaatgaggtgttctgctcaagtaagtgtactgggggcctcagacccttggcctggtggatggcttcctgcaggctgacaagtggccacttcaacatttttctttggtgtctccgctgctgggtgagctctctggtggctcagggactgcaaggtttcacactgagccatataattcaccaccacccccctttcctacagaatctttctccacttccccttgtctttcatacttatcagggcaaataaaatttggtcaggtaaacaggtcccaattttataaataatttgggtccagctgtcttgtacaggtcacttcatttgcatgatatgtgttgtgactagcatgctatcacattggcttataaataagagtgctcataaattaaacaaataagtctaaacatgttagtttgaagggaatgttgtgtcttctaaaatttaatttttacctaggtaaaccagatgttcataggttttggaatggttaaaatggctttaggtagtgagttttgtatggcttaaaaatcttgaaactgtagaatgcttctcatctacaggatgctaatgtctgttgggcagttgaagatttcttgcttcctacctgtatataaaatgtgctagggaagatacattattgggaaaagaataacttttgtccagaaagtattaaatgaggggctcaaaatatgagggaaccagtacgagtagaaaagagagaagtggggagttatacatacatacattttttttttcaggaggggtatgaagactgacttacaggtgcccgccaccatgcctggccaatttttgtatttttagtggagacggggtttcaccatgttggccaggctggtctcaaactcctgacctcaggtgatccacccaccccggcctcccaaagtgttgggattacaggcgtgagccactgcgcccggccaagaaagactgactttgtatgagaaaggatctctggtcccagaataaagagactggttgtgagggaggtgtaggacaggtcagagagtccaggcatgtcatgggtggtctgtgtgggttgtaatggggtttgtgaaggggaacttctgagagtagttttgtgtgcaattaagcctgctgtgattaaagaaatttttttttttttttttggagacagagtctcactctgtcacccaggctgcagtgcagtggcatgatctaggctcactgcaacctctgcctcccgggttcaagtgattctcctgcctcagcctcctgagtagctgggactacaggtgcccaccaccacgcctggctaatttttgtatttttagtggagacagggttgcaccatattggccaggctggtctcgaactcctggccttgtgatctgcccgccttggcctcctaaaagtgctaggattacaggcatgagccactgcaccctgctgattaaagaaaaatggtttgtggtagactttctagggaatgatctatgtattggaactgggttttcttaaggtattgatttattaagttatgagaatttttgcttttaatgctataaccagcttcttctaaaacttctttgggctgagtgcggtggctcacgcctgtaatcccagcactttgggaggccaaggcaggtggatcacttgagcctaggagttcacaaccagcctgggcaacatggcaaaaacccatctctacaaaaaaaaaacaacaaaaaaaaacaccacacaccactttttaaaaaaaatgtagtgtgggtgtggcgcacatctgcagtcccagccacttgggaggctgaggtgagaagatcacttgagaccaggaagtcaaggctgcagtgagctgtgatcacaccactgcactccagcctgggcgagagtgcgaaaccctgtcccacaaaacaaacaaaaactgcttgggttggtgtcccagaggttcagctgttgtgtcccactgctgttaaactgcaggaagtcactcgctgggtacacctgtccagggttaaacctgcgtcttctgcgtccctgcaggcatgaggggaggacagcatgcccttcacctgtgagcccctggaaggtgtaaagctgttgttttgcaaatacacaaatcacatggcgaggagcacgtgcagtcatcagccataccgattcttcttgcctggttttgacctttctcagggcctttaatgacatctcatcattactaacttttggctcccttattggattttgggtttcttcccacccaaccttcatttcccccatgatcagaccaacggtaaagcagcctcatcttcctttgatgcaggaaagatgagccctaaaattggggtttagccaggagggttcttggcttcacccaggaacgagttacagggcaagctggcggtgttagttgctccctgtggagccgggctgactcacaggcagtgtgcccagagtccacggcatatgggctgttggcaaccatgtgggctgtaggaaaggcaaaggacctttcccaggacctttaataactatcttatcattactaacttttggttcctagcaaagctcgagtcctgtagattgcaacaattccacacaaagacaacactggtacaggcttcaacccatcctatcctctgacctggagaatgaaagtgtctggcctctggggcccttagatcagagattttccctcctctaaccttacacaggacctatgcccatgaacacagcaggaagcaatcccagaagacagaccctgcccttctgcacccccttaagattaaggggaaggatctcatctctgaagagaggggatgaggtaggaaggtggcaggacttgttttctggtcactgacccactgaccaaaacaggatctgggctggatgggatgaagtggagacaggaatcaggggacggcgaggagggtgatccctggctgccatcattgctcattggcataagaccctcccaccagggccatgatggtttgcaaatgccatggcaatgacctggaagttaccaccttttttcattgcaacgacccagaagttgtcacttcttttctagacaattctaaataacccacccctcaatttgcattgatctctacttaatctgcatgtaaaagaagtgggtataaatacagttgccaacagcccatacactgtggactctgggcacactgcctgtgagtcaaccccgctccacaaggagcagctaacacctccagcttgccctcaaactctttcctgggtgaagtcaagaaccctcccggcaaaaccccaattttagggttcatctttcctgcattaaaggacaaaggggcagaaagggtgaggctgccttaccgttggtctgatcatgggggaaatgaaggttgggtgggaaggaacgtaaaatctaataaagcagttatctgtacagcagctagacctctccgaaggaatcctgctttgtggttttggcttccgagggtgtaagtcttccacgtctgcagaatgccgtcctcgccatcgaaaggaagaaggtgaactctaaagtggagacgtgagctggatggtgatgttggtctcctgtggctgccataacaaggtgctgcacaccacatggctcaggacaacaggcacctgccgtctcagaggccacagtgtgacagtgacgtgcaggcagagtgggctccttccacccccttctcccacacagtggctgctggtggccctgggtgtgccctggctgttggctgcatccctccagtctgctccctctcagcgtggggctcttcctctgtgctggtctgtctgatgtcttctcataagacatcagctacaggatttagggcccgccctaatccagaatgacttcactttaactcattgcatctgcaaagagcctgtttccaaacaagttcacattctgaagttgtgggcggacatgtgtttggagggcaccgtttgccacaggacaactacgtatcacagtgctggtgatgcaaccacacaagtgaaattatttcaagtaactgtctcaaaacaaaacaaaaaaagtgtctcgctctgttgcccaggctggagtgcagtgacgtggtctcggctcactgcaacctccacctcccgggttccagcaattctgtttcagcctcctgagtagctgggataccatgctggaataatttctgcatttttagtagagatggggtttcaccacgttggtcaggctggtcttcaactcctgacctcaggtgacccgcccgccttggcctcccgaagtgctgggattacaagcgtgagtcaccgcgcccagccttcaggtgacttgtgagcacagcactcacactgtttatacttagaaggatattctctgaggataaaagatcatcttgttttcatttttaatttctaaatagtcactttgaaatgatttcagtcatgaaaaaagttaccaaaaaacaaaaacagtgcaaagaattccagcatatcctgtaagtagtttccctagaagttatctcacatagaaccatggtgcaatgttcaaaattaggaaattaacaccaacgcaatactgtttttaaaaaattatttgaaatgcaatactattaactatctacaggccccatcccagtgtctttctggcccacagcccagtccaggaccacacattgcatttggttgtcattctccctggctgcctcccagtggggacagcttggagtgtggggccattgcccagcacacagctgcttctcatgactgcatttaggacatatcctctgggcaggacccacagaagctatgccaggctgacttcagggcatctcatcaggagacctatgatctgggcctgttactggcgtgttaagctcggttgctgggagcaggggtatctgccagggctctctgccacaaagttacattttccctttaattatcttgtcgggagatagtttgaggccaagaaatatcccatttctcatcctaattttgcctactaatcgcaacatccctcgatgattcctgtgttgtttgccaaatggccattctctgttaccatcgttccttctacattaactgcaattcggaggagacgagcatttcaccctcttcccccttatttatctatgcatttatgtatggacttgtgggcatttagccttatgagttatgaactattactataattttcttttttgagacagggtctcgctctgtcacccagactggagtgcagcagcatgatctcagctcactgcaacctctgcctcctgggttcagatgatcctcccacctcagcctgctgagtagctgggaccacaggcatgcaccaccatgcctagctgattttttatctcctgtagaggcaaggtttcacaccatgtgtccaggctggtctagaactccgggactcaagccatctacctgcctcagcctcccaaagtgctgggatcacaggcacgtgccactgcacctggcccattggtatacttatgtattgttttgctccgattatcccaggtttggctctggagccgtgtagattggtcctgcatccttttggccccccccgctgccctctgagcgctctgccttactgaccaccccccccccgctgccctttgggcgctctaccttactttgtgggaccactccatgttcacccgggaaccctccctgcctcagtgctatctgggggccccagtgcctgacctggagaaagatgttagtgcccgagatccggctctgggggtgcccatggctgtagggtgtccctgctccgaggtcccccagtggacacagctaaagcctcggtgagaacacacgggcccatgccaggctcacacccgtctacccagccgtctaccgacacttccgtgctgaaactacaagctcctactgccgcctcccctcccagcctcccatgctccttactggtagctcccttctccagcagcgaggctcgtcacccacagcgcggccgctggaaggtgacctgagcggccagacagggcctcctgctcagagtttaaagctctgctgtcgccatccttaagttcttaatgagttttgagcaagggccctgcaatctcatttcgcaccaggtcccgcaaattatgtatccggctctaattacccacaatatgcttgcttgctcaagtctggagcacacacaaagtaatctcataattgctaacccaggttgctgtgcaaaacagcttcacaaactagggcacagtgtctgcacgccattctttttgtcttacagcccatgatactgtcttccaaagcatgcggtaggttttcttttccccactcccttcgatgaggttcacctgttactgcttatattccatttcggcttcaagcccccatcccggttgattttaatcacttatttactttggggggtgtgtggtatatttctgtggtcctggaaggaacacaaaagctgcagcctccataataaggtaagcccaccagcgcctgtacctttgaggaccatgaagggaagggtctgtctccctgttccctccactaactccccccatctctctggcccccacttctccgcaaagtggaaactccctggggaggcagaagaccagggtggcaggactgcaggcagagagggattcctgccgccccatggctgggtaccgctggtaggccacgcacacactgggtaagtgtgcactaggcactgacgttaagggccaggcgttggtccctccgtctcacctgcgccaactcagcacagggagagagctgaggctctgacaaggatacagggcagagagcaggaaacagcagggctcaggctaggggtgggagcccagctgctcaggagttgaagtgcaggccccctgtgggcacctggcggacaggaaaggaaggagactaaggagtgtgttgtactttatctgtacctggctacgtgagatcagactgtcacccagcattaactaaaccttacagtggagacagtgggttgtatcactcccgagaagaaaccacagaacgctgtaggagtaatgcctgcaccggcttcccccgcaccacacggagcggctgcctacagctttgttaggggctgaatgcaaccccgatgcaggcaccagccctccagagcagcctccatggaggaatgcggttccccactctgcccacatacctctcctgcagcagcgctgaacaccgtctccggacgtctggagctatcaccatgggcaacactttcgtggtctttatttttagcttctcctttagaaactcactttttggggtggttttgtgtccagaagttggtgggttcttagtctcgtcttcaagaatgaagccgcagaccctcgcggtacatgttacggttacctaaaaacggtgtgtctggagtttgttccttctggtgcgttcgtggtctacactgatttcaggagtgaaactgcaaacctccgcggtgagtgtcacagctcctaaaaatgcagcacgtccagagttctttgcccctcccagtggatttacagtctcgctggcttcgggagtgaagctgtagacctttaccgtgagcgttgcagctcacaaacgcagcacagacacaaacactgagcagcagtaagatttactacgaaaaccaaacaaggaaagctcccagaggacaaaagcagaccctaacaggttgccaccgctggctcccgcagcctgcttttattcccttatctgaccccacccacatcctgctgactggtccattttacggagaactgattggtccattttacagagagctgattggtccattttacagagagctgattggtccatttggacagggtgctcattggtgcatttacaaacctttagctggctacagagtgctgattggtgtgtttacaatcctttaggtagacagaaaagttctccaagtcaccaccggagccagaagcccagccggcctcacctctcagtagcactccctttagaactttgcagcatctaccctgggcactccggcagcccagagggagctcatccccagatcaagcccagcaggcgtcggcgggccagccaagcctgcacccacccggaacccgcgccgcccgcgagcgctgcgctcagcccccgctcccgcccgcgcctctccgccagcagagggagctggctccagcctcggccagccccagagaggggccccacagcgcagcggcaggccgaagggctcctcaagcacggccagagccgacacggaggcccaggaagcgccgagagccagcgagggctgctggcacgttgtcacctttcagtttcacaatatcccaagatgcattagtagtttctgtagcctgagtgttagtggcccccaaaattgataggttggaacctaatacccaatgtgatggtgttaacaggtgggacctttgggaggtgagtagatcacgagggctgtgctctcacgaatggcatgagtggcccgggtagaagaggctccagggagctccctcgctctgtccaccctgagccttgtcaagaggcgccgtcttggaagcagagggctgcctcatgggacgccacatccgccagcaccctggtcttggacttgcagcctccaggactgtgagcaataaatgtctgttgttgacaagtggctcaggcttaggcgtgttgtcacagcagcccgaatggactccagcagcagagcagccagggctgcacgtggtgcgtgaacacacccgccgccctcctcctcctggggcatatgacggctgcgcagctctgtgtatccagtttctgggctgttccatgcccctattcccctgattgctatgttctccagctgaaagagcacactgattttgccttcagcccactctcattttcccccagatttcactaagtgattttgctaagtgatggctgtgcttcccctgtgcgcttctctctggctggtttctactccggatccactagtggggccagaaagagcagtaggaaggaacctgaggttcctgtccaccatcaggcatgtgcacacctacacccacacacccctgcactgtgcacacccacacacacttgcacacccacctgtgcacacgtacatccatacacacatgctcacccctacatggacacccacacacgtgcacgtgcccatacacatgcacacccatgcacacacctgtctgtgcacaccccacacccatgcatgccggcacacacccatgcacatccacacacaggcacatacccacacacacgtgcacacctcacacccatgcatacctgcacacgcttgcacactcacctgtgcacacacatcatacctgcatgcacacccccacacgcacacccacacacctgtgcacacacccatacacatgcatgcccacacccacatgtgcacacaccgtacatacctgcacacacccacgtatgcacatggtcaacactcacccactcaggcactcttacatgcacacacgcacacagacatggccactttcatggacacacacccacacacgcccacatatacacgcatgcccacctaaaccactcacttgaccaaatagcttttcaggagagcgcctggtcctgaagctttctaggcatttcggtagccaaaccctcttgatcgtgtggttccagaaaaagccagtgcggcacccacttacccgtactgcagccaccgtggtcccgggaagagggcttgcttccgcagagctaatgagggaagcggtctctgagagtagaagggaggcctcggagggaggtggtctccaagagtagccgggaggcctgggaggatggtggtctccgagagtagcagggaggcctgggagggtggtggtctccgacagtagcagggaggcctgggagggtggtggtctccgacagtagcagggaggcctgggagggtggtggtctccgacagtagcagggaggcctcagagggaggtggcagagcaggctctgctctcaggaagtgccccgtgggtggcgacgggccgagaggtgctctgggggacgagttatggctctgagctgcccgtgcaaggcagggaggctgcgttttcatgcgttcacacctcagtccctgggtaaggacctcccaggatgtacctccagcaccccggtcatgcagaggcctgggaaagcctgttgaggctggaggggtgggggcacattggtgtgttgggggacctgtgtcgtgggcacggctccccgtttagggaaggcactgcggtgatcacgcactaccagtaagtatttcagagcccaaaacgcgaaaacaaatggaagaagttccatgtcatagtgagtcttggctgggcgcagtggctcacctgtaatcccagcactttgggaggctgaggcgggcagatcacttgaggtcaggagtttgagaccagccgggccaacatggtgaaaccctctctctattaaaaatacaaagctactcgggaggctgaggcagaagaattgcttgaacctgggaggtggaggttgcggtgagccaagatggcgccactgcactccagcttgggcaacagagtgagactccatctcaaaaacaaaaaagaaaaacaaataaaaatacaaaaattagctgggtgtggtggcgggcgcctgtaatcccagctactcgggaggctgaggcaggagaatcgcttgatcccaggaggttgtagtgagccaagatggcgacattgcactgtagcctgggcgatggggtgagactctgtctcaaaacaaacaaataaacacacacagaaaacagtgaatgtttttattacaaataataaatgtttgcttttacataccacacctcatttccccaggggttcctgagcacccccccccaaccccctgctcaccctccttcattctctccacagtgtttgaggcatattttgtgccaggcccctcccagggctgctaacaagaaaatacaaaataaaaatggcagagaagcgagtgcctcccgcgtatggcaatgccagccatgagccaaacggctgaacagagtccacatctccctccgggtttccggggtcccctgctcaggcctcaccagtgcagcgcggacgtcagaccgctaccgtctcactcaaggctcccgtgggccagggtccacctgggttctgctccttgggatattggcaggatcccgggccctgctgctgtggactcgggacttcacttccttgctggtcagaggctgcgtgagcttccagaggccacctgctgttcgtgtcacctggggctccccggcccagccatgtgcttcctcaaagccacaggggagggccaggttccaaagaaatgggcattgcaatcctgtgtaacacaatcacattcctccactccactttgctgtgttctgtgggttagaatatggttatcgccgggacaccaaacaagggtgtcaccaacaggaggtggggggccttgggagtatggctaccacacaggaggaaacgacagctccactccatcactgaacctgagctcagcagtggctttgagtgtcctggcagccacgcaggaaagggactctgctcagaacacacatgcagaagcaggacacactcctgctctcaagcccagcgctgagcccatcagcgctgagcccatcagcgctgagcccatcaggggcaggtgtcctgggcctccggtctcaagggcatcatttctgtgcaggcaacagtcactgggctgtcctttcacgccctttggacacacagacaacacaagtggttcccagaagccctctgtgtcctgtcaggtggtgacaggtgggggtggctgtcctgtgtcctctcaagtggtgacaggtgggggtggctgtcccctctgtcctccgtggtggtgacaggtgggggtgactgtcctgtatcctgtcaggtggtggcagctgcaggtggctgtcctctgcatcatgaccccacctgtgtcatggcagggccaggctcatctccagaaacctcaggtcagtgatccctacggatgcctttttacatcatgtcagaatctggaaagtctgttttatttcattatctgaaatacttttaagagtggtaacggtagcattagactgatagttacatggatctgggacatctgtggcactggtgcataaacatctgagccatttccatcatttaacttgtccaatcctttcaacagccttctgtgctgggcgcgatgactgctctctgcacagatgagtaaactgaggcacaggtgcctggacccttgccaaaaagtgagggagccagagattctaacccagggcccagccctgagtctcacccctgccctctcccatctgcctgtccctccacaaccccgatgcccgaccctgggtggagaaagtacagggagggtctgcagctgcccagctgcagtgtccagtgtcctggcgacaggacccggagcaaaccaggcctcggcatgggcacgggatgaactcagggcaccggtggggtgttggccgtttctgggtaacagacctacaggtgatgtcttttcctcttctttctaagctttaaaaattaccattgtgattaatagaactttttagaattttagatacagtttctctgtgtgaacaaggcttaaaggatatgagaatgtttctgtagcttacaggtcataatactatctttgaatacacaaattgaaaagccacttaaatattaacctttgattctgaaccaggatgagcttaacgatggggacctgcagggcgtggccggctggagcccatggccagcagccatggctgagggcatacttcaaaacggtcatttctgtcccactaatactgtgtaaatatgtatgccatccaatttttaaccaaataaaatgttctgtctgacgggtcccttcatctatatgacaaacagttttaagtaaagaggatctcagcttgcagaataagtaaaattgtttaaattctagataaatgtgttcagtgcttgatgggctaatgctggctggatttgtgaaggtcagtcatatacatgaaaattcttctgcattcaccttagaagagcgatatttttaggcgaacagagcttgcgatattttctttccgtgccacctgcccgcttggtgtctggccctgcgtcctcagaggaccatggtgggctggtgttcttggagaggaggcctctccagggccaccaagagggggctgtggcccaggctgagctcttctagtggcatgagggacacgctttattcatgtgagcgttgtgaggtgggaagggaggccagccaggggaagggtagaagaatgcctgttgcttgggtcggaggctcgatggactttcactgctggacatagtgtgaacatgggagacgcaggttcagcgatcacgtttcactttcccattccccggacctcaaactcaggcctgtgtaactaaaggttctgtgctgccaggctgggcgtttcccccagggaaggcacgagagagcgaatgcagggccggccgcggggcctggggactgtgccgctcagcaccacccacagggcgttccttggggcgtggcctgggccggggcggtaggatcccttggacagggccgtgtctggctcacaggaagctgccctcatggaacggggtgcagtggggtgttcgtggtcctgctgccgttggctgggcgcagccatccctgccggctcccccgcgcctgtgccccgggccctactgcacctgcacgggcggggctctggggccggcgcggggagtgaggccgccggtctcctctgctgtggtcggtgtgggggccggcggggcgcagagctgctcacccggctgactcccccttctgccctgctggcctctgagccggttctctggggcctctgtgagcgccttgtgccccccacacccgccgtgagtaccgtgtctctctgctcagccaggtgggcgcctctgctgctgctgttgccaccgagtgactgactgacgcagcgacgcagccaccgacacccaaacactggagcgttttacccaaatgtaaaagtctaaacttccggcttctcgaacaaaagaaaacaaaggaaaacgtcagacgccccgttgacgccgggcgcgcgtggccgcggccgagccgctcttcaaagccggcagcgtctggtctggcctggctccgccagctgcccgcaggtcctgtcctcgtggctttctgccctgtcctccctggccggctgcactggcttctgtgacgctcgctccttgctgcgcaagtgcaggtgcagcaccttaaaggctgagtgcgtcccacacagcggggctggagctcggcaccttcagtccaccccaaacaccgccctcccgctgtgcacggccactgctgcccctcctgcaccccgacacatggggagctggtctgagcctgccccacctgctctcctgtgatctcccctctgccacccccatctcgcgctccataccccttcctcagacctccttgcctggggctgctctccccaactcagcagagcagggctggccagtactaaggacaggggcgaccactggtccccagaaatcctgaatccaggaaacccctcagtccggggcagaccgggatggccgaccaccctgactaggggagggccgtcagaagtctttgaccctgatctaagttctctctgctttgctgagtctctttctgtctctctcctcctctccctgtctgaatgtctgcctgcctctctcccacacacatctgagacacaggtctcacagaaccgcccttgcccttctgtctgtctgtctgcctgcctctctctcacacacacacctgagacacaggtctcacagaaccgcccttggccctctctgccaggagggtggttctcggaggttcccatcctctgtggggatgctgacagctgtgggtcatggctggtgccgcggttctcactcttggctgcagagagcagctgggggctgcaggtcaggccacacctagctccccatggccctggttcacttaagtgacaggctggggtgaggggaggaggaggccagggtgtgaccgtcggttccatttctgctttcccccagctccccaagtcaggcaccggcacagtggcagttggtttgtgggacacccatctgtcagtagggcatgagtatccttggctctgtcctgctgaatcatgggtcctggctgtggcgccagggccagctgtgtttgtgatctggtgcatcctcaggagaaggagagcaggcgaggcgccagggaatgtaggatttgctgatgaattgtgtttcaaaaaaggtggctttttttctgttgtcagacttcaggaaacaggtagaggctgtgacctttgggccagtcaaatgaatctacagagattaaaggaggattgtggagtgaaaaccaaaaaaagccaaacatagaggaatccgtttaccctttcagccacgtatttcgctgttgccgaacctttattaaaatgcttctctcaaaggaaaaattgtatagttttttcaggcttcaaaaacccaggccatgtgtctaaaggtcacttgacgaacagcacaaatcccatggtaaagggcagggtgcggaggggtgagcctcccatccagcaccaaagcgggctctgagggcttatccctcccagggagtccagcaccaaagcgggctctgcgggctcatccctcccagggagcttgggttctgcacaaacagtgtcagaagaaaagacttcttcagacattttgaaattaacatttcatttgtattaaacagcccaaccctccttggagagcaagtactaaatacagcatccacacagtcagccctgtaggggaatttgagattggaccagaatggcagacttgtacatgtcatccaggaccttccacccatccatctgcacatctatctatccctccacctacccatccactcatccattcctccatccatctatccatccacccatgcttccatgcatccatccatccacacatccatctgtccatccacccaagcatccatccatccatgcatccatctatccatccattcatccgttcatccatctatctatccatccctccaaccgtccataacctacccacacatccattcacccatctacccatccatccatccatccattcatccatccacaaatccacttgtccatccatccatccatccataacctactcactccccacatcaacccatccacccacacaccacctattcattcatccatccatcacatacccacccacccgtctatccatccatccatcatcaacccatccactcacctacccattcatctatccacccatccatgcatctattcatccagccatcatctgcctgcccatccatctaactatccatatatccatccacccaaccgtaatcaaccatccatccacccagccatctacacaccccctttccctctcaccctccctccctatctccctttcttctttcttttgtccatcaatacatccattcatttactcatccatctatccaactatccactcatccatttattcatccaaccatccaccactcatccattaacccacctgtgcattcatcaatctatttatccctttctctctctctcacccttttctctctctcctttcatccatccctcccttcttctgtccatccatccagccttccatctatccattcatcccttatctatccacccatttatccatccacccgccccttcacccacccattcattcatccatctgtaaacatattaaatgccactgccttccatgtaccaggcctgttcaaaacactggcagaagcgagtgagactaactcagttcctgctgggacaagagacaatgggccatctgccttcaacagaacacctgctagggaagacctggggagaatgggggtttcaaggaacactggccaagggccagaacccagaagctggagtgggaacagactttcagtggtgatgtctatgggaagctgacatggcataggagctagtgggttaaggttggaaagttcatggtgagaacagaaagaaggtagttccagatgagggaacaagaggaagtacaggatgggaaggagagtgaagcctgggaaggcatcccaagggccgggaatgtctcaggtacccaaaggcccaggcaggatggacctgctttctctaatgccctggggacgcccaccacaaacggggcacttgaaacaacagaagtttatggtctcaccgctctggaggccagaagtttgaagtcacagtgttgccagggttggttcctgctggaggctctggggcagcatctgttccatgtgtgtgtctccagcccctggtgatggctggcagccctctgtgagtcttggtttgcaggtgcatctcttgatgtctgcctccgtagtcacacagaggtcctgtcctggtctctgtttcactgtgtctcttttccccttcccaggacaccagtggtgttggattcagggtctcagtgtctctcctctcctcttatagagtccctgtcatattggatttagggcccaccctactcaggtatgacctcgtcttatctaatcgcatctgcaacaaccttattttcacataaggagtcaggacttcagccttctcaggggatacagttcgacctttcccaccgccccaccagccagtgccgcagctcacgggcccttcctgccgtgagcgtccactcagtgctggcctttctgctgctcccagtctcggcttttatggccactgcacacgcctctccatgtgtctctgtcaggcacacagagagaactgtggggcttgtgattgtctcccttaggctggaatcctgcagactcttgagtggagattgttgtgctcctgaatcactgggactcacaggagaaaaccaggagagagcgggaaacaggccaagggaagggagaggccggagcagacatggtttcaggtggggtccagtggtcctaacggggactctggacatatatcagactgcaggtcaggatgggggctttgacctcaaggtggtcagggtctggccctgggccaccctggcacaggcttcaatcccaggggcccctggacgagggcccagctgcccaaggcaggccctgggggctccaggtgtgagctgttagggcggcaactgcagcgggggtggtggccacccccaagggtaaggggcatcctatgtgacaatggggagggtaggagcagcagtaaggctgcaggcaggttcagcccttgtcgttttcctgccatggagttcactcacccacctgaaagctgcgagtgtccttcctgtccacacgcagccacacaggggctcagactttccaaagcccagctgctgtgatgggtgtgctgccacattacgtggtggctgactgcatttccatgacaactggtgaggttgggcctcttttcataggtttgttggccacttgggcactttcccttgctggcagtcattcaaggtctctgtccacttttctgatggggtgtctgcctcttccctatggacttgtggagttccttctacattcagggtgtaagtgcccctccctcacctgctccctcaggcactccctgagtaaatcactcggcacgaggaccccgtgtgcacctgcagtatgctgttccggttgcctgcagagcctgcaagctcgccagcggccagcgggcatggggctggtaccagatgccatccacagctcacacgccagagaggcgggatcccacatgtggctcctcactcctccctgctcgaggactgccgtgcctttgttttggggctggggaatgtcctcagctggttcttcccccaatgccctgatcctgggctcacatgtgaccggtccctgcagaccccacacctctgagaaaggcttcagtggcatgggcctaggctggaccacgacgcactcagcaaagcccagctgtctctgtgaccaacggccatctgggccggcccctgctgccgagctgctgggaccacaggcagaggagacccacctggcagaggagacccacccagccccggagcccacctctccgattgctggggcttaatacagtcgcccatgaggctgtcggagtccttcaagtctcagcccacatagtgacttatggccacccacacagtatctgcctggaccccagacctgagtggtcagcagggatcacacctgggggctccaggtgtgagctgttagggcggcacctgtagcaggcggggtgtagccacccctgagggtaagggacatcctatgtgacgatggggacggtgggaggcactagttaggctgcaggcaggttcagcccttgtaggcagcccagcacttttcctgccacagagttcactgacccagctgaaaggtgcgagcgtccttcctgtccacacgcagccacgtgctacgatgcagcagtagcagtgtccccatgtgctgccaggcacaaagccctcggtgtggggtgtgccctcaggcagttcggggcagggctgcaggtggggctggcgggagtgacgcttgtcttatcacaggtcactgcctggaaggggtgctgccatcctatcacaggtcatgctgagggcattggcatttactccaagcaccaggataaccctggggtttccttgggctgtggcctgatctgttggtatctagaagaatcctttaggccacagagcgacgaaagatgcttggactgaggaccaggacccaccaagatctgggatggggcttggaagaggctcttgggttgggcagtggcagtggtcaggcagtgtggttggagtctggacatgtggggagcccacaagaatgctgaaaggcaggtgaggggctgggggagagcggtgcaggatggcacggaggtccagcctgagcaagtgggcacgacataaggtgccacaacctgccacaggggaccctgcaggagtgcagacgtgggtgagggtcaccactgcacccaacactgtgtgggagcccagggaaggggaaacagatcacacccagcacacctgcgtgggagcctggggagggggacaggtcacacccagcacacctgcgtgggagcctggggaggggggacaggtcacacccagcacacctgcgtgggagcctggggagggcgacaggtcacacccagcacacctgcatgggagcgtagtggatgtgttatctctaacgtctaggctttaggggaggccggaaggagagatgggccaagaggcatcaggtgaggaagctttgctgtgggtgggctggggtggggcagaagctgacctcggggtgtgagcaggtgagctgggcctgtgtaaatggatggggcccagcggaaacgagacaaggaggctgaggcagtttttccattgtggatgtcttttattgtgcttagggacccacacgatgctgaatagagattggtcaacagacacggacaacacctgggttgctacacggagcccctccccagaaggcgatggggaagccgctctctgtgctgactgcagcagccagaaggcgatggggaagctgtcctctgtgtccctccccagaaggcaatggggaagccgctctctgtgctgactgcagcggccagaaggcgatggggaagccgtcctctgtgtccctccccagaaggcaatggggaagccatcctctgtgctaactgcagctgcctggcagttcacaggctgacggctcccaccgagcattacactccagtccctggagcccacgtccacttgcaggggacagccatgactggccctgcacggcccctgccctcgggcagcacgtaggcaggcccaccgacttgaggcatgaattcaaactatggcatccactttcctgtcagtggcagagggcagtgtgccatggaaagcagcttttggccaacatccttcactcagtattgctaattattcttgtaaacaaaaactgcaatatcagcaagcaggctcgcggatactgcattcttgaatttgttttgattcacaggtaaatatgattttaaaaagccattcgcaaacataaaaactgttgttattgatgtggcacgcacctgagagaaataaaattccagtggggtcccttcctggaggtcacggctcaaggccaggcagagtgtggtctgcaggctgcctgcatgggggccctgcatgcgtcctggggtagtacacgctcctgtgggggccctgcatgcatcctggggtagtacacgctcctgtgggggccctgcatgcgtcctggggtagtacacgctcctgtgggggccctgcatgcgtccggtccggggataggacacgctcctgtgggggccctgcatgcgtcctggggtagtacacgctccagtgggggccctgcatgcgtcctggggtagtacacgctcctgtgggggccctgcatgcgttctggggtagtacacgctcctgtgggggccctgcatgcgtccagggataggacatgctcctgtgggggccctgcatgcgtccggggataggacacgctcctgtgggggccctgcatgcgtccggggataggacacgctcctctcaggccgttccctacaccgcaaggacccacaggctgcctgagtggcagtagcctgagctggtttcaaggagttttatttctctctgctgcaagaacataaacagaatttcctggtgagacataccaggacccccatcctccaatgcctctgaacagactgtgtgtgcaatgtgtcaactgaacgctcaatttacggcctctgctgggagccacgcatcgggaaaggactttgcatgaatttgtggtttcctccatttcaccttctgcacagctaaaccaagcaggacacttggctttttaatatgggcaaagtaaatggtctaggaagctactgtgagcacggggattctcagcaggtgcgtctacaaggatcgtgatccccgcctgagaacacagtgcccctggggcagcctcagagccgggagcagggagcagggagggagggagcctcacacagacagcatgaagttagacgtttttctgccctgcagggacaacaacggggtggacctcgctgcacagatctacgtcgttattacagcaacacaagacacggcgaggtgcgctcccggcacggaaaggtgtaaacagtcagaagagaggagtcttctgctttgttgtttgtgttttcagtagaggttgaagagtagaagttgccctcctttctctcgaaacttagatttccttggtttgttcgtgtctctcccattgcaggatgacttcctggggtcttcgtctctgctccctctacaccttgagccagtggcggagctggaaagaaaacaggtttagtcagaaaccctggggcgatgccccatttaagagcagctgaaggtggctaagagcagctgaaggcagtgagcagacagtttgcagcctcctggccatgtgccctgggagaaggggaggctcactgggggcctggaccaacacacccttgacaggtgccggggatccgatgcctcactcaaactcctacaaatcaatcattacttgattttttaaaaagccgcgagactgtggttggcgtggtggccatggtggcctctggggcgtattgatcagatgctgagtctgagagatgccgggcaccctcagtccggggctggttccagattgtaccccagcgacttgggtgtgacgtggtgatgtcacctctcccccacgtccctccaccttcctgggggcctcagatgctccatctgagtgacacgctacctctgcagctccatcttgctctctgcgacccgcgcgggcgcaagtgcagcgtgtactgactgctcagggactgatgagatagacaagagcagctatttacaccaccagcaaatgtcatcgcctgctattatgcagctgttagttcagcctgtttgtgagcgtcaaagcccctcgatagcactaaatggcaagaagtgtgcttcataaacatttccaaacaggggcatgcggggcttgcttctgacaaaaccacgccggccatggaaagccactgaagctaccactggccacaccacaggctcctctggccactccaccaagagcctgcagggacctcctcagccagggtgggatcacactgcaggccaggggtctaccgggggcctggagcagccacagctgatgccggcacaggcagcatccaggtgcagctcggggctcagtgaaggaagctggggtcagggagcccggccaggacccacagccacgggtggcccagcggccagagccctagagcagaggctctgggtggcagccccacttcaggccagcgatgaagaagccatgcttgggacttcagctgtgagtcctgttccccccagggtgaagcagttctctgtcctgcccgctgacagaagcagaaggtcccctctggagggaggtgtgcttgggggctcccagattcccacagattaagatcaagccataaatgtaaaataaagactagaaacagaagacagcagcccactctaagtgagtccccagaaaccagacctcaatttgaccccaaaggaccagagctgttgcaacagattacaggatggccacataggggactctcagaaaagtgacacacgcacatcacagtgatgagaatttcacaagaaacagcagagcacacgcagcttccagagatgaagtagaaaagagaatggctgggctgcagaccaggggcagggcagtggaatgcggagcgtccatagctgagctgtggtttgttcacgtgacggacggtcagggaccagtcacggtaggtgaaacgtagctatggatacaaacatttctgacctagatggaataatagggatcgaatttaccctactgtccgaaacaagccccaaataaacaaaacacacgtgccgatgaccttccagaccatggacaccagaccgtaagggtcagtggtccctgagggaagccaacaaggaggcccccctgccaccctgctcgctgcctcgagggagtttctagattgcagcacagggagggtgcagcctggtggactccctgagctgagacgctggaaaggagagcgagggaaacggaggtggctgcagtttgcaggccagagcgccagagaggaggtggccacacagagagaagacccgggacaatggcccaggacccctgctgagggttcagacgagcacatcccgctgtgaatgaggaggctggggagagatccgccgacaggaccagagggaaccacctctgatgcccacacagggctgggcacggtgcctgttcccccagccaggagaatgtcatgattctcggggatctgcatggggtgtgcataaggtcttgcttcagtaggggttggttagggctacaccaaatgctgaagccaaataatacactttttaggaagctagaaatatctagagatgcaacaaatgaaattgtaagaagggcaagggaaagatcaagctagacgcagatacagaatgggtgaggggcaggcagtttccacagtgcctagttttcctgctgtgggctcacaagggtctgtttgttacggtgacaaattaataatttaaaaatggccacatgtggattaatgataagatgcgttactcaactgtgcacctaagattcaaatgaaaacaaaatctaagtgaggtaagagacctggaggacctggggcaaagtggacacaccagagcctcataatggaaaacacagaaaggcagaaggcaatctttcagtgtgtggctgcaaaattcacagaatgacaagatttgtgaatccacagatctggccacaaaacatatccagagaggaaaaataaaaagaaaccaaattaacggtggtgggtgcctgcagtcccagctactcgggaggctgaggcaggagaatggcgcgaacccgggaggcggagcttgcagtgagccgagattgcgccactgcactccagcctgggctacagagcgagactccgtctcaaaacaaacaaacaaacaaacaaacaaacaaaaaagaaacccaggtcccgacactccgcagtgaacccgagacaccgaaagcagaagaaactcggccgagtgcaggggtgaggacagagcccaccacagagcaggctcacaggccacacatctcagcacagccacggaacactgctgcctccaaaacgcagagatagtccctgtcagcctagagccctgagcccacaacagtgctttcgtgaatgagagcagcataaagatgttcccacacaccaatagtgagggaggttagcaagagacctcctcaaaggaatgtctgaaggatgctttttgaaaagcaagaaaatggtcctagaagaaaagaagtctataacaaaagtatgggaaccaacaaacaccaagcatgcacattagtccaaggaacatgatgagtccgtacaaaataataacaggaacaagaacaatgacagtgtctaatttgtgggttaaaaagagagaaatagatttgaacaatcatagcatgttcatcagtaggaggaagatttaagtccttataatgggatggggttaaaatatggtttctctgtagagtttaagtgaaatatgtatggtacaacctcaagagtagctattaaaggaatagaataagtccgggcacagtggcttacggctgtaatcccagcactctgggaggcagaggtgggcagatcacttgagatcaggagttcgagaccagcctggccaacatggtcaaaccctgtctctactaaaaacacaaaaattatccaggtgtggtgctgggcacctgtaatcccagccacttgagaggctgaggcaggacaattgcttgaacctgggaggtggaggttgcagtgagcagagatcatgccactgcactccagcctgggtgacagagcaagactccacctcaaaaaaaaaaaaaaaaagaataaagtatgtaagttctaagcaagtagagagaaataaacggaataataaaaatgagtgaaccaagaaaaatcaatctataaaaaggcaataaaggagaaaaacactaaaaatgggacaaatggaatagaaagatagtagaatcaagttcaaatatatcaataatcacaacaaatgtaaatggattaacctgtccaactgtcagcgtaaaaaattctaggtgtatgctgggtttgcaaaagacacttaaatcttaaggttactgcaaagttgaaagttaacagattgcaaaagatatttcaggcaaactcaaaagacaccttggtgcaactatatttgtcccagacaagtcagactttaagaccaaaagcatcattagggagtgaagatgggttcatgtcaccaggaagagatgatcattctggacatggatcaacctcagaaaacagcctcagactacagagagcaaaacctgatagaactgaggggaacagcaaacaaatatactatcgaggaagaatatgccacaggcatctctcaattactcatggtcaagcacccctaaagttgacaaagtgtagacaatgtgggtaacataatatataaccctactcaaatggacagaagcagaactgtgtattgacaattagagaatagcacattcatctaaaagatgtgcaacgtttatcaaagttgatcaggtataaggccctaaagaagtctcaatacattttaaataataggtatcatatgtgtttgagaacaatgaaattaatttggaaattaaaaacaaaaagatgagtaaaacatccccatatatttggcaatttaaaagcagacttttaagcaagttatgagttgacaaagtcacaatggaaattaaaatatacttagaactaaaagataaagaaaaatactatgaaaaaaatacggaatgtagcacatggagtattttgagggaaattcatagccttgagtgcttacattagaaaagaacgaaggatgcaaattaataacctaaagtccaaattaaaaagttagcaaaactacaacagagtagatccaagaaaaagagaggaagaaatgataaatacaggagctgagattaatgtgacaggaagcacagcaagaatctcacatgaagtcaaagttggatatttgagaagactagcaagatcctgtacaaagtgtaagacccccaggtataggactctcagaggagagaggctctggtggagcaggaccggaggcccagtggggaggggctggcatcctctggatggatgggatgggatgcctggacacgcctcctgtgtttggagcaggcaagtcactgggaggacagaggaaagggtgccaacggagggaggcagagcccacaccagaacctgacttgcaggtaggaggaacaagctgtgcagatgtgtgctcagaggcggcttctccccactgcctcccggcctgttccctgctcagcgtcttgttctgcctgacgtcaccacccacctcccaggcacgcagagccccctccctctgcctgtatccttgctcctgccccatgtgcaggtccatgctgggctcctcaggcatgagctcatgagacccctgctgggaggactgtttacgagaaccagccacagagaggtggccagagggaagaggcaggtccgaggtggggccgcggtgccctccacactatggccttctctcccttttgctgtgcattggactctgctgtcggcattaggctgacattagtgtatggaatcaggtcggggctgaggccactctggggtggaggaggacagtgccacaggcccagggcacagcccaggctcctctcctgctggccagctctgtgatcttgggcacattccggggcctctctgggtctcagggaccctggtgtagagagagtgctgggctgcctcttagggttgtgggagtaaaaacacgtcaagtgtttactgtgtgcccccgggaatgcacctgctccacggcatgtcatgaacatgttcgcacacagctggatggctcccagggtgcaggaaacacctggcgtgaccatcctctcactcgaagccagccccaggctcccaggcctctcccagcccctccagggctccctgtggcttcccatggccaggcccaggcctcccctcctggcactgtcccttgtgcccccgtgaggcggggggtggtggtcccttgctggtccttaacctggctcagctggccccacagggcactgctcccgtggtctgggacatgcccaggtctttgcagggaccatgctttcctcaggcgtctgtgcagccgcacctcccagggcccctgcgtcatgtgccccccgtcccgggcacacgtctccactgtcacctgccaccactggctgcctgtcctcatagagcacatgctggctgagtaaatgagcaccatctacaccagcctcggagccccctgggggctaagaacactgagcttgggatcattctgaattcccccgagagaggcccagcagggacctcgacctcaccgtgaactggcgcacctcccctctgtccaccagctcacggtccttctcgggtgcaatggcgtaggccagtttctgaaagagaaagagagtgcaggggtcagtgcagaccagtacccactgccagcacccaccactgactcacactgccaggaccctcacctaccagcaccctcaccaccagccctcctcacctgccaatgcccacccgctggcatccatatcaccagcgcccaccactgacttacactgccagtgcccatgccgaccagacagccagtcaggcccgaagccaacatcctgacggtccccttaaagtctagcgcagagcagaacatcagcatttgagcactcgcttgaaaataaaacgtggtcctggagatggctcttgagtctaagctaccacgtgtgctgggctctgagtaagaaagtgcccacacccaggtgggctgcctcggggccacctgcagctgacatattccgggagcacttgctttttgtcacctgcagctcttgggagagtgaacgctgaacgtgccttccttccactgcccgctgcggcagctcctggggcctggacagcaccaagtcctcccagagcaggcagcatcttcaggagctcaccctctcgggcctggatgcctgtatctggatctctgaggccaggtgacctctcacagggacccctgtgcgtggccccaccccacgtcatcaccacaattccacttgtacctggctcagctgctgaggttttacttggcttacgttcagagtttggccatgcgaggggtaagggcgctggctgttcaggtccgccgggctgtaggcatcctccagctctgcgctgacctgggccccgcctcccacgtccatccagcctcccagaggagctctctccctgcagagactgcccggctgcctgctcttcccctgtgctgggtcctggcaggtcagcccgtgggggtctgagagggactgtccacagcagggtgccatcagctttcctggggccatggcagccccagagctcctcccactccacagaagcccccagccacacactgtgctttgcctgcactgggccttggcctggccaggctgctggagagactggctcagtgccagcctgagagcatctgccacatgtctgagcccaggggacaccgtgtggccctaaggtggaatccttgaacacagctggtgctgctctggggttttgatgcccagggccaagcgacttctttctgagaaaggctgggatttgaggaagcagcttccgggagttcccagtggtgggatctcaggtgagggcgaccctcttccaaggagggagtgtccttgtctcttcctggaaccacagagccagggtgttaaacagggccctgtgactggtccacctgcctttcttctacacaaaggcggcaaaaccaagcagaagaaaggagatgcatatcagtgactccgcaaagcttcctccactgcctggggctccagctgcccctgttcagccaaagttgggctcggccctggggggacctagatcacccctgattctaccgccctcagatggcttctgtgtccaccacaccacataccatagtgtgtaggcatgcatgcactcagtacacacaaacacttggacacgaatgtgagcacagacccaggcccacacacacgtgcacacggagacagcacatacagacacagagatacagtggatgtacacacaggcacaagcacacacagacacatgcacacacagacacacacagacatgtggacatacttaaagacacacacatgaatacacatatggacgcacacccatgggcccgggcgtgcaggtggacacacacacgcacacacacacacagtgttgtggtggccacctccagtctcctcctcttggtcacagatgacccaggcaggtgaggactggggccatggacacccacggagcctttctggtggcctcacactcgggtgaaggcgccccgtccaaatacccacctctcgaaaggacccaggcaggctgcagaacttgtaggccgcatagatgggcaccatggccatggaggatgtggcgatgacccagcccagcgcgttggcccagtcggggaagatgtaggctccgtagtgggggggtctgaaggtcacaatgctgaccacgaccacgaactgcaaccagcagatacggaggtcaggcagctctcagccggcggtgccaggacaaagcagggagcgggcaggcacttcatggcagagcgtctctcagcccccacagctgtgcaggtgcagaccccggccaggcctgcagacatggcagctgggcatcccgggcaagccatgtcctcctgaagcatgatctagcctgtcctgtcctatcctgttctctcccctcccctcccctcccatcccatcctgttccatcacctctcctcccctcccctcccatcctgttctgtcccctcccctcccctcccatcctgttccatcccctctcctcccttcccctcccatcccatcctgttctatcccctctccacccctcccctcccatcccatcccatcatgttctattccatcccatcctgttctattacatcacatccggctctatcccatcccatctcatcccatcccatcccttcctgctgtatcccaccttgttctattccttgcttgaaatcacaacaaaaacaaaactgaccaccacccagtacactgaatctatgatcatccatggcctgaagacacaactgtgacccatgcgggaccaccctagggccatctgagctccgtatctctactcattctagaagcaaatctgataaagtcacagacctgtctcagaaccaccaccacaaaggtaacagagggtggggtggatgccacttggaggggaagcttcccactctccagaggcttctcctgaggcagctcatgcatttgttcatccactccacaaacatgcaccaccatctaaaaagatcctccatgtgcctacatatgcacctcacacctgaatgtgttctgtgcacccacatgtgtactgcacgcctacatatgtgttctgtgtacctgggtccttcccaaggacacaagtgaagctctgatagatatggtttatgatgtagcttaaaatgcaattttttcccaattgtccacttttcctcaatttcagaaaagttaaaacatccaagccttccacctgaaaagcatgttggacgcgtgtctcatgccagtctcagcatcttcttcatgcgctacttcgggccacttgtaggtttggagccggctagcggaggactgggatgggggctggggttgtgtctgtcacgtgaccagagccagggtgagcagcactttggcgagcaaagccagtggcctgcagcatctgggatcaggggcggctgccttggctgcacagagagccctgagcatgctttaaagagccacgcgtgcatccctggacatgtcgggcttgcctatctgacatggaggtattgctgtaatgtgctctctgttattgttcaacttcttgctagataccacctttcttagaaagaactatgcctccccaggccacagtctgcccatgtaagttgcctttgctccggcataatttccctttagcagtgctgtggcatcatctgccacagagtcatctgaacgagactggggcgtgtacaccccttacgaccagaggttagcacacgccagagggcaggggactatgccgcagcctgaagaaacagatgataacacagacgttatgtttctgctgaaatcagaacttcataggaattgtgctgtcatattaaatacaaaatgacatagagcaatgaaattgtcatgtttctctcaacttggcaaaatgaagtcctgtttcgtatagtttgtagttttatctaaaagtctacgaagtgtgtttctgtatgaagttcgaagatttagtctaagatcagtagaacattttatctcctaagatcgaggtcagccctctgggtgttaagaggagacaccccctcaaggggagccaacgtcctcatgcaaaacagctcagtttcaactaatggcgggggagagcgggaggtgggcaggaggctgactttctgggagggcttcctcactttcccctctgaacagccatgtagtctgtgtgctattaaaagcccgccccatgaatccaggaaccttcaacgggagccttcacaagcgcagttaactggacttggaacacttacgtgcagccacaccacgcggcctggagctcagacacggtggaatttctacaagatgccgggcaggttctctcacactatgttgagctcctccgggatagggcctgtctctcatctctttcctgtaccccggaacccctctgctggcaactacggggctgtcccacctgctgtgtgcaggtgacgaggggtctccacgcacgcgggccctgctgactccgggaccagcccttggtccatgagagggtgcggcctgagtcccctgccctgttccagtccccacctcgtgcctgctggactcgggaggtgcttctctacgtgagcagctcacgggacactctgcttagacttggacagctcttaggttgcctgcccagatcaggcagcacagctttgctttgtgagcctccaggcctctgtggttcagtgggggaagcactgcaggcaatccctaatgaaagtgtgcggccaccttccaacaaaactctatttacaaacaccagcgtccgacggccttgcccggtgggccctgactcggtggcggatgacagaagcaagtgcctactggacacagtgatgctgctgagccagagggaggcttgcgtgagcaacgggacagggccgaggcccctgctgctctcatccagcactcggcatcctttgtccccactgccccggaagctctaacttcgaccttgatcctcacaggcagaggtgagtggacagcccgactcacctccagcttcccctcccaacacagaggcgcggcccaagtgcaggactcacaacggactgtgacaggggtccaagaccatgtgaggttttttcggtgggtctagaggggagggtggaagccaccttaaggagactatagatgagttcagggatcagcctgtccttctgggccgagtcttgaggcccctgactccagccacagtgacaacccacatgcgggcgctggacctcggggcaggtgccagagtgggggcagtgggcagacgggggaagggcaggtggcccgaggtcccttaccaggagaaagcaggggctgaccagcttccagcacagccgccagtacaggctgggccgctgcccggtcatctgctggatgtcgtcgctgaactgcccaacacctgagggagaagaggtggcatcagtgtccatcagggcagcgcattcccccgatgctggacacgtgtgggggtcctcgctgactcccaagggccccacctaccggccccaggcttcggctgcacccagcctcctgcagaggaggccaggccacaccccagcccactgggtgcctcgctgacgggtgggagcccacgtgcctgctccaccctggagatgcaccaggaagagccgtgccccggtgggtgctccccgcgccccggcaacagcccctcgggtcctcctccccatcccatggctctccctgtctccctctgctgctggtacttcttgttcacttaaaaagattatggtaaaatacacataacataaaacttaccattttagccatttttacgtgtagagttcataatgtatcaatacattcacatactgtgtaaccgtcaccaccgtccatcttcagaactccatcctccaaaatgaaattctgccctcatgaaacgccaactcctcccgacccccaacccccgccaggccccagcaccatctgctttttgtctcaatgaatgtgactcctgtgggaacctcccgtcagtggagtcacgtggcacggggcctctgcaactggtttactccctgtagagtgtccctgaggccacctaagctgcggcccacatcagcattcccttttgttttaaggccgaatcatattccgtcatgagtataccgtcactgtgtcttcagaaaagctatttctagaaacataataaacattttttctccccaaaagcaagtaccagcagtccactttagaaatgcatatttttctttcactggtattatctaacttcttctaggaactgaccctaagaccttaagagtggtttctccccccacatctggcctggtctgtgactcgtgtgaccgcggctgatctgctgagatttggggcaagtcttgcggttgtcccgtgtcccatcagaaagaagcctccacacagaatcccctcttcacacccacccggcatccgagctctgaacatgagagatggacgtgggaggcagcggggatgcagagaggtgaggccggaagaagccaagaggggcgtccccaaggcccctcggcccccagaccctgcctcccaccagcaggtcccagggcccctgccggagcaggagtcatcagctccaagacctacaggaccgcagcgtgctggaagcccggcgtattccggcacatcttattagagcacaggttttgtttcatttagatgagaggggcgtggatttctctttggaaaaataacacagatctcggtcttctgaggtgcaggtcgccagggccggcttctccccatctcccgtggttccggaggccatggcaattcaaggcactttctcccttcctctgtgaaggtctatgttttgttcagggaagatgacaatgaaagtgatagacaaagtattgattccagacacccgtgaaagtacgaggttgatatttccgttttctgtagagcaacactgaaaacaatgttctcagaataggagctatttgtcctatttgtctcaaagtcagggcgtttatctggctataaattccatggctctgaggcagagaaaatgacccccttgtttgcaggtgagctgggcgtcctccaaaaccggggctcacgcagcaccacagacattcagctttcagccggatccacacatttttttttttcttttttgagacggagtctcactctgttgcccaggctggagtgcagtggtgcaatctcgggtgactgcaacctccgccttccgggttcaagctgaatcatattccatcgtaagtataccatcagtgtcttcagaaaatctacagcctcctgactagctgggattatagccttgtgccaccacacccggctaattttttttttttttttttttagtaaagatggggttttaccatgttggccaggatggtcttgatctcttgatctcgtgatctgcccacctcggcctcccaaagtgctgggattacaggcgtgagtcaccgcgcccggaccacacattcatggcgagatgccctggctcggcctcgccacttccctggaagtgagctggccaggtaagcggagctggcactcctgcccattttacagaagagagaactaaggggagtcgaggtgacttggccaggtcagcatgtggggaaagggcagccctggctccaggctgggttttctgttgctgtttaaaccactggtggaagtcccaggacacagctggggcagcacaatgggccaggagctgcaccccgttcgagcgccgcccgctgatcatcaggtcccgactgctctggagctctggtcagcagggaaggcgatgacggcacctttgaaaggcaccatgtcacatccctgctgaaaccccagcatggatctgattgtgttcccctgggtgggatgggctcaccggtgcctctagcgtggaggaggcagaagacgcccagccgctgtgaacacctctgaccacagtgtgcctcttctggctgccatccaagctaagcacctcactgatcccatcaaagggaccagtgccaagcctctcccctctgcggagctgtgatgaccacaacccaggcttcctgcagctgaaaggtgtttcctcacggagcctttttcagaaggggagtggcacagccaccaaacaagagggtgccggcttggctgccttcccccggactcaccatagaaccaggccactccgatggcttcgatgagcactccaaagaggatggacgtgccggctgcaaaatggtccaggagcgtgaagacgtagatgccaccctggaagagaggggagcctgtggacctacagaagggctttccccagaggtaaacccagcctggggattcactgtgcacacaaattgtttcactcactgcaaaactctggtttgagtccctaaaattcaacccacatgcagcttcttagggccacgtgtattacaaaaaataaaaataaaaatgacctctcctttcccttcctagggcttcatgtgcaattagaaatggaagcaaccgaatgcggaacttggcagtgcctcccttggagagggagtggcctgggctccccctcccacatagataggctctgccaccacacccccagcgtcccccagtccccaaaccctagaaggaggcagcttcagcctggggtgcttctgggggagctgtccaggtgctgaagtgactctgggaccaagctctggatgtgcagttaccctgtgcactgctacgagtcattttctggggtgggtgggttcgcagctccctggaagtgctgcggttctgtctggcctgacagtcccagccagggcgccccgtgccacgtgctaaggagacatgaccaggagaaggcgaagccggcgatggtacgtacgttggtgacgcagaacagggacaggaggaaggtcgccaggacgatgaagagcgtgaagagctcacggtgtctgtgcagcagctggaactcatcgatgagcccggtgatcactgactccataccacccatctgcacacagagcacagggtcgggctgcaggctggcggcgctcctgaccgcagcctgggccaagacctacttgtcacccagtggacgcacacccggggatggacacggaacaggggccacatggccgtccagggtgcaggatgcctggtagtgaggacagctgttggcgagggcttctatggcggcacgaccgcaggagagaggacccagagggaccactgtatccacggtagggggtttgcagcctcttcgatgtctctgggtgggtaagatgcggcatggctaaaacaatgccaccctgtccactgctgctgcacgggctcagcttggccagattttcctgagcaagatgcgtcccccacacaaagctttcagctgcacctggccttgtcattggctacgccttcccgacggagcctcctgactcctggcatcctcctgtgggacaccgtccaggcacctgactcctgcttagtgaccttcttcaccttgctcggtccagacccctcttcatgctctcatgtcaggaacatgaggcctgtggtcaacccaggagccgtggacgaggtggataggatatgagtggggggccgggtgcatgaccagcgtttataaggcaagggattctgggttctttggtcccaagcagctgacagggaagaaggagcctgagaaagtccagcaggcccagggccgatcaagggctggacccgccacccccagcagctggaggtgcaggggccacacacaaagcattcagggccaggcctgggattggtaggtgccaggaacttctgttgccctcctcattcctcatggggtagctgaggcttccagggaaagccacgagatatgaaggacctgctgtgtgccctccaaggccccaccactctggtgtccggagccaagtctttggtggctggggccctgctacacgtgcatgcatgtctgtgtgtatgtgtgtgtgcatgcgtgtgtgcatgtgtgtgttcgtgtgtgttcatgtatgtgtgcgtgtgtgcatgtgtgtatgtgtgtgcatgcatgtgcatggtggtgtgaatgtgtgtgttcgtgtatgtgtgtatgtgtgtgcgtgtatgtgtgtgtgtgcatgtgtgtgcacatttccaggatctgtaggtggctcagggcagggcacacaggtaccccagagtagggggataaagggaaaggtaaactccagtcaccactcactccagccccgagaaaggtctcccaaataatcacggggctcgcccaagtcaaggacaggaggtctgggggccgtacgtgagcccagggatcttgcctagccctgggagggcagggccccctcgggtggaaggaacccaactgccgaggacagggccgggcggtgcgggttactcacggcgctgtcgatacccagggtgagcagcatgatgaagaagaccacggcccaggctgaggacagagggagcgtggcgatggcttccgggtagatgatgaagatcagccctggccctgaaacagaacccgccctgcttgccacagagcccacgctgtgctctcccgcccatcctgccccaccccgccccgagaagcatggcctgccacaggcctgtagagactagggctggtgcggctctgctgaaaagcccccttctatatgtccagcagaccaggcctgggactcaggaaagcggaaacccagaactgatcagagggctttggttcatgcccaccacccagcaatggggctcctccaaattacctgggtccttttgggctatgggggtgcttggggaaggaaggggcagaaacaaggaggagcaggaggggcttccaggctggtcctgcccttcatcccagggacatctgctaatgtccttcgagttagtttttcctgcacataccatgcaacatacacactcagacacacataccatgcaacatacacactcagacacacataccatgcaacatacacactcagacacacataccatgcaacatacacactcagacacacataccatgcaacatacacactcagacacacataccatgcaatatacacacacagacacatgcgcgcacatgcacgaacactcatttgtgcattcaaactcatacatgcaagaacacatccacatgcacgcacacaaacagaaacatgcacgtgcatgcacaaacatacacaaacacacacaaatcacacacgtccacactatttagtagcctaatttctaaaattagcaaaacatttagggatacattcctgacctccgccttccaggcacatgaggaagctgaggtcctcccaccctcccgtgaggttagacgtggctgcacttggccaatgtgctgtgaacccacagtgagcgggtccctgcgagtgaagcttggtagaggagtgtgtggctccttcaccacctcccctgccacggggtcccgcaggccatattgagacaggctcctgtcagctggggccctgagggctcagaggagcagcacatcctgatggatacaggggcccagatggtggcttcggagataagagacaaacctgctaccttgctatccctgcaagcgaggggcacgccaggctgagggcggcatggcaaaggcggaggaggtgttcccccacagctctctcgggaaacgacacgtgcttcctgctaccagcaggcagactcggatggaggtggaggggacgagagtgtggcaggcaggcgaccaaaagtcaacgcagtttggtctctgatgaccccaaagtaaaaaccagtcccataggcaattgacctttgaccagttcagaagccttgcatcctccactggctggcaccatctcccaagcccaatctgcctgcaacagagtcccctgtcctcagcaggcacgtgtctggccctagcctgcaggaataggcctgcgcctggccttttaaataatcaaaggccagagccaggggccgttcctaaaggtctttcaggggcctcactatcagccaagcagcggggtcagacctgggctctcgcagctcccagataccctggtggatggggacagcctccgcgtggggtgggggaggcaggctcgcctgagatgttccggggcttagatggtaccagccaggagcctgggaagatattcatcagctcagggaatgtaatgtcaacacccgtgccttcgtttctgtacctaagttaacatttcaccctattttcttgagaattaaggctgtaaatgacatttactttgcttgtcatgaaatgcacagaagaacattcaactttattttcaggggttaatagaggtttttgcaaactatgctcgttatcataataaagggcaaaaagaatgaagaaaaggcataaatgcggcaccgagtaaatttcactgctctgcatttatttgcagatgttccctttctcccgctttgactgaattttttttcctagtggaaagaacatgggcttaggaggcccacctctgagctgtggctccagccccgaccctggctttctgttgcaagcatggagtgggccctttcaggtctctgagcgtgttcctacctgcggtgcgagggtcctagtgccccactctgtgagactcaggtgcggtcaaggctgcccccgcttggtgcttgtcgctccccgccttcactgagcctcaacacccaggcagagatcagtcagcaacaacacccacacgggacaggggcttttctgagcacaatcattcctcacaaatgtagtgcccccaccccacccccggagaagacagtccagaggaagcaaggatggggacgcagcatgggagcccacccaacccaggcccgtgtctggagcgaaagggcctcctccacatccatgtggcagagcaggccgggaggcaggcgggggctctgctggctgacagagaccgagaagccttgggactccctggagctctcggttggcccaagttagggctgccaacgccacagctttccactgcaagcaccggagccaggcacgtggccccaagtgcctgggcccactctcagcccctgcatctgtgcccgtcctgccggctccatcctgcctggcacacttcttgctgtggccaaggcctccccccaccactcacctgtgcctgcccgagacctggacccctccccaccctctcggagacaactccaggcgtgcagggacactcagcgtcatgcgtcggcctggggagcgggcagggacaccctgtgctggaggccccagggatctcccggctgctctgtggccaagctgatgtttgcggcatcccagcctgcctccaggtgttgctggaacagctcattcctccccttccctgagctgcatgttcccacaccgcgtggcaagcaaacatctagagggccctcagtgacagcatctacgccagaccctggtggggcagcctcatgtggcatctgcagggagcagtcgagggactgaggcaccaactatgctgctcagctgcatttgggcttgaggttccccagcccagggtggaggccccgagaggcacaaggcaggaaaggcactgggtgggggggcctggaggggcagggcggagaaggcactgggtggggggccgggaggggcagggcggggaaggcgctgggtggggggcctggagggtcagggcggggaaggcgctgggtgggggcaggtctgtactgggatgtgtaccaccatccttcaagagtgaggcagcaactctcactgaagtcgctcagggcccatgcgtctccttcctctttcacaaggaaaaaggctttgctgagagctcggcgctggtgctacacggagcaggcccaggtgcagcaggaggggctcaccgtccttggccacgtccccgatgggcacactgtgcttctgtgccatgtaccccaggaaggagaagacgacgaagccggaggagaagctcgtcagggagttgatggaggtggtgacaatcgcgtccctgtaagaacaagacacgccgtctcaggaaccagctgagctgcagcagctgcaattttccagtcattattcttaatttactgtgtctggggaagggggcgggaggtcttcggaggggctacttttcccagtgagcacggccagctccttagcagcctggcagagctgtccttgggccaccttggtcccctagctgagctgctcaaggctcagccatcaaggctcagggtgggcagaacagacggccacccctgtgccccgcgagtcttgctgtggacacctcacccagggcagatcttccagaagctctaggagcacacacctcccggggacctcgctaagatgcagatcctgactgggcgggtctcatggggtctcgggggctgtgtttcccatgaactcccaagggcgctggtgctgcgggcctgggggccccactctgagggctgagaaaccagagagctggaggcaagccgaggacgaagagggagggtcctggggtcggtcagggcaccagcgtcctcctcctgggttcctgtgtggcctgtcactggggtggaaggaccacacacttcatctgcagagcacctggacccaggtggacgcaggtgggctgcgggcaggcgctgcctcaggacctgccttccagcgcccactctcagtgaagcctgcttgtggtctgcctggccattctgctgcacacactgtccttgtatttttaccgtgtttctagcctcatgatgtgaaagaaagtaagcacaagtccataaaggtcaaagcaagcagatcccggataaaattctgtttctgtttttagaaggatttggggtccggcacggggcttcaggtgcctgccagtaagtaacacggggtgcatatgggatggggggacacactcagggggttgtgaagctggaccctgagcgagggaggacagagaccccccggggctggtgtgagggaagagggtgtgtccttgtggagggaggggctggccttgccacttggggccctcagtggggaatctggccgcaggtcaaacctaggcaggtccctcccaggaggcgacacacccactggcttcagtgccttctttaaagttgaaatcaaaatcgtgttgggagctgcctcgctgtcgcttctgtctgaaaggccacgcagctcacaggtttgcgggttcagtggatccgccctgttctcatccagggacaccctatttctggaagtcagcgacctctccctagtattgatgaggcccctgcctggccctgctaggggctcacctgtagcagttgttggtgaacttgttgtagctggagaaggcgatcagcaccccgaaccccacgcccagggagaagcacacctgggtggccgcgtcaatccaaacctgcagagccagagggcggtgagaggctgtcccaggagaacgcaggccacaggcaaaggacctgagcacccttcctgtcccagccccacactgaggcctctaaactcaacaccatcctcaagaagtaaatggcagcagacgactggtggaagatgcagcctcaggaagtgaaccctcccgggccagcggcctggaagagcctgagaagcagggggctgtctgtgttcattgatctgggattccccatggagtatcaaggcagcgattccacagaaccagaggtgccctgctccatagccctcagaacagcatggactcatccacacacggcatgaccacagcatcctaataaccctcggaacagcacggcctcatctacacaaaacatgaccacagtgtcctaataacccttagaacagcacggactcacccacacacagcatgacctcagcgtcctaatagccctcagaacagcacggactcatccacacacggcatgaccgcagcgtcctaatagccctcggaacagcacggactcatccacacacaacatgaccgcagcgtcctaatagccctcagaagagcacggactcatccacacacaacatgaccgcagcgtcctaatagccctcggaacagcacggcctcatccacacatgacatgaccgcagcgtcctaacaaccctcggaacagcacagattcatccacatgctgcataatggtggcaccctgataaccctcagaaagtcatgaactcatccacacacaacatgaccgcggcgccctgataaccctcagaacagcatggacttatccacactcacaccagccctagcggggtctccatcctcccttgatcagggtccagcttcacaaccccctgagtgtgtccccccatcccatgtgcgccctgctgcatgatggcagtgccctgaaaccctcagaacagcacagactcaaccacaggctacatgatggcggcttcctgataaccctcagaacatcatggatcatccacaagctgcatgatggcggcaccctgataaccctcagaacaacataaactcatcctgaggctacatgatggtggcgccctgacaaccctcagaacagcatggatcatccacatgctgcatgatggcagcaccctgataaccctcagaaaggcatggacccatccacatgctacatgatgatggtgtcctgattggacgcacaccagcaatgcctgctttgtatccttcccaaaacatgagcagtacaggtgcacagtggcacctgggcatggccaggagcccacctgcagcttagacactggtgctgtgcctgcccaactcggtggatggcacccacaactctggacgtgaaaggaaaggtgggtgggtccacgattggaccattgtggtattttaaaaagtcagccacaacctgctaaggtgagggcactgcggagaccattcagtgactcagatgcccaccttgcccttccctgggccttggatgggggagaaggagcagccaacccccccttggaggcatttgagggactggtcagttagggctgttcctgcaagtccctggagcccatggtacaggcctgtgggccaggatgctgaggcagagacaccagatgctcctgctggggaggccaggtgggtctcagggcatcccaggtcccaggtcaagcttgcctgagcttgactgagcaagggaactgggtgctgcagagggggcggggccctctgtcctttgggcagcatgtcagagcctcaaggcaaccccagttcctgggacagtgtgtttggaggcagatggccaggccccgcagtcaggactctgcaggtggccagtgtttcagcatgaggggaggagccaagccgagagtggctgggtgggcagtgggtggagaggctcataccctgatgtggctcccatgctggtcatcagccacctgcctggccaggtagctgcaggctggatcaggtgtcacaggggagcgcctgcctcctcctcaccctgatctgtgtgtgcagctcaggcctctgcccagtgccacaccagagccagtgcccatggctgcctccctgggcctcacctcatgcactttcactccaggacctgcaattgtcctttccttccatttctttcctgatttctcttcactatacatcaatccatcatctattatctatcaatcaatcatctatctatctatcatctatcactgtcatctatcaatccatcaccaatctatcatctatctgtcatctctataatctatcaactgtctatcatctatgaatatatgaagtacatgtgtgtgtatgtatctatctccatccatcctattgtatatccatcctatctacctacctatcatccatccatccacccatccatcatccatccatccacccatccatcatcgatccatccatccatgctatccagctacctatcattcatccatccacccatctgtccatcatccaccaatccacccatcatccatccatccatccatccacccacccatccatcatccatccatccatccatgctgtccagctacctacctatcattcatccatccacccaaacatccatcatccagcaatccacccatcatccatccacccacccgtcatccatccattatccatccatccatccatcctatctagctacctacctatcattcatccacccattcacccatccatcatccattcatcatccatccatccatccatccatgctatccagctacctacctatcattcatccatccacccagccatccatcatccagcaatccacccatcatccatccacccatccatcccatccatcatccatccattatccatctgtccatcctatctagctacctatcattcatccacccattcacccatgcatcatccattcatccatccatccatccatccatcatccatccatccatactatccagctacctacctatcatccattcctccatccacccatccattatccatacatccagccagccagccttccatccacccatcctatctatccattctatctacccacctatcatccatccatcctatccacctacttacctagcattcatccatccatccatccatccatccatccatccatccatccatccaatctattcactgtctccacctaatttctgacatctcatctttgtaatttgttctccagtgagcatcttttcacatgcactggtgtttgttttgcctcctcctggggctgcctattcttcctagctggagcactaggctctacgttgtccttgtggtatttttctttgattctgtaacatgccatggtcctgcaaacacaccccttggcttcattttggcctttctggggtccagacgccctgctgacttcatggtttgtctggctccagctctgcacttgtgggcccccggccacagctgattttgctgccctggttgtcctcctctctcttccttggctcccgtgggccacacgcctgctgcacccatggctcctgtctccacagggcttccccttcagtgctgcaggcacacttccacccccacccccacccaactgctttgcagcctcataggtgccatgtccaggtgccctctctcactgcacctgtcagctgctgcctccctggggcttcctgggaggcttgcagccatgcccgcccatcccttctccccttccttcccacttctgccttgggtttacacttgcctcatgacccccagacctggctccaccggcccacccacacagggcagcacctctcaacggcctcctgcctcataccccagctgagatggcccttcccctcttgctgcattgtcctcccacagcaacatggatgcccctgggcggctttggggcctgccacctcccccacttagtccacctgatttccaacgatgccccaagggagctgccatttttgtcatgccagccttccgccaacagctgcttctgtttctgggcctgtttgccaggaagggtgcagccctttctcctccctttctggacaagggtcccatcccccatggcccagctgagtctcctctctccgggaggctttctccaaatattttaaccctgaattatgtcttcttttgctgaccaaagctgcagcctgagtctagatggagtttctgatgtaatttgatttagatgactcgtgctgtatcacaggtacttgggatttggcttcccgaggaaagaaccctggtgtctgcaactctgacacctctcagccctggcaggcaatgcatatggaaacctgggaatgccagagcccctgtggactgtgaagcagtgagcagactgtactcacagacgcctcgcagagccggtagaagtcaacgctcaggtatgctctgatgccgtctatggctccagggagggtgaccccacgcaggagcagggcagtgaggaccacgtatggcatggtggctgtgatccataccacctgcaggagaggacagtgtcaccaggctgcacaggcagggcccttggtgggagcagacactggcacaagggcaccgggttgccctgacggcttgtcctctgattgggaggcccaattcccaaacatttcctgggacgccagctcagcaaatgctccttggagtgagaaggtcagacactttctgtcctgcacaccctccccagggatggaaagggaagaacaacttggcagggaaagcacattccttttctgcattcggtccgccagagtagctgggagtggctgatgggggttttctgatgcgtgggacgtgagtggattcacactggtgaacggcactgtggcacgatgaagggctgctctgggctgtgtccctgcgggaggctcccgtggctggtgccatactacacgcacttttccaagggaggagtcgtatcttggtcaaaactggttttggcccatataacaaccaaagttttttttttttttttgagatggagttttgctctttttgcccaggctggagtgcagtggcatgatcttggctcactgcaacctccgcctcctgggttcaagcaattctcctgcctcagcctcccacgtagctgggattataggcacgtgccaccgtgcctggctaatttcgtatttttagtagagatggggtttcatcatgttggccaggctgttcttgaacccctgacctcaggtgatctgcccgtctcagcctcccaaagtatttttatcaacaaaaaaatcatcttgtgtgtgccttcctgccagaaacactgctgtgagtgcttccctcctgaggcccttccccaaacacagccacacgtggacccaaaacccaactgtgccgtgtgtccgcccagcccagccacggccacgtgtccccccacccacccatggccgcgcgtctacccaagccaacccggcacagccacaggtgtaccctcaatcatggccacgtgtacactcccaacctgattatggccatatgagtctcccaaactcaaccatggccatgtgtccaccccaacctggccatggccacattggtagcacaaaacccaactgaggccacacgtgcacctcctgtccagccacggccacatgtccacttggtggccccatgtctacaggcccaattggtgacccccgagcctcaccttccctgaggtcttcacgcccttccagaggctgaagtagagcagcacgatgaccagcaccaggcaggctgtgagctgccaccgcggaggccccaggtcgtcgatgccatggctctggtggaggtgcagcacgccacgtctgcagaggggagtcagcgggggactctgtgggtggctgtcaacccacctggaactggacggctgagcttgtccggggacctgccctccccatctcagcagggcagaaagcatccagtctgatggacaagagatgccttccagggaggtctggcccacaggccctggcagaggtgtcagggccgcccttgaaacccgtggagcacgtgggaggcccaggaagccctgcctcaccatctgcccttggcacccctgctcactggagctgcggaggcggagggcaccttgggtctttgaagaaaaggagccacagaaaccaaaaggaagttgctgagcaatgctgggtgcccaccactgcccagtgctgcccaaggtgctgggtgcccaccgctgcccacggtgctgcccacgctgctgggtacccaccgctgcccacagtgctgcccacgctgctgggtgcccaccgctgcccacagtgctgcccacgctgctgggtgcccaccgctgcccacagtgctgcccacgctgctgggtgcccaccgctgcccacagtgctgcccacgctgctgggtacccaccgctgcccagtgctgcccatggtgctgggtgcccaccgctgcccagtgctgcccatggtgctgggtacccaccgctgcccagtgctgcccatggtgctgggtgcccaccactgcccacagtgctgcccacggtgctgggtgcccaccgctgcccagtgctgcccaaggtgctgggtgcccaccgctgcccacagtgctgcccacggtgctgggtacccaccgctgcccacagtgctgcccacgctgctgggtgcccactgctgcccagtgctgcccatggtgctgggtgcccaccgctgcccacagtgctgcccatggtgctgggtacccaccgctgcccacagtgctgcccacgctgctgggtgcccaccgctgcccacagtgctgcccacgctgctgggtacccactgctgcccagtgctgcccaaggtgctgggtacccaccactgcccacagtactgcccaaggtgctgggtacccaccactgcccacagtgctgcccacgctgctgggtacccactgctgcccagtgctgcccacggtgctaggtgcccaccgctgcccacagtgctgcccacgctgctgggtacccactgctgcccagtgctgcccatggtgctgggtacccaccgctgcccacggtgctgcccatggtgctgggtacccactgctgcccacagtgctgcccacggtgctgggtacccaccgctgcccagtgctgcccatggtgctgggtacccaccgctgcccacagtgctgcccacgctgctgggtacccactgctgcccacaagacagccccagggtccctctccagggatgaggccgagcaaactctagcaataatgccaggcacacaggcaccagccagcactgttttctgaggggatggctcacgacacctgctcacagcccctgctgctatgaggattccacaaggaacagattcagtggatgactctgctccagcaggaatcactcgcagttccactgttttttgaaggatccctcagaaagggcagaacagagggtgagggatggagctgaccatcggctgtgctccttgccccaggcacaggtcctgggccccctgtctgtgggaactcattccatcctggtgacactctctgtgggagaacatgcaccaggctacatgcagtcagctgcgtcaccactgcagactgtgacttggggccaagacaactgtaacagccgtgccgctgccaccgtagagtcaggagctgtgacagacagaagccgcgttatgaaaggagctcccactgcccaggtttgcttctgttgtgggtccactgaggtccccaggagataaaacctgacacccatggtggagcccggccgaatgcagccttggggagctttccaccagctgcaaggcattccagggccatgtcccaggaattcggctcttcaaactccagacaccctggctgttgccaacagggaaggccccacgagccctgaaagcgctggacagtgcctccgtcctatcctggagggcgcccaggcagaggccccaggttggcacaggccccaggttggccgggtgggcagatggctgcaccactgaaaccatcgccaccattctctgctcttctccaagggcaagccgctcagtcaagaggctatccgtcacccgcactgtgcagggaaagttgtctgagctggtggaggtcacgcctgcccttcctggggcagtggggactcccagggccttcttctcactcctcctgccacacaccaggctctgcccctgctggccagtccccgtcgttctgctcccccagcaaggtctcatcactgagagcttctgaggcctcccagcaactttgtgcagcccggaccactcctccctcactgcccatggtgtcactgaccccagcatgccacccgcccacgccttggcagcctcagtgcctcacacacacacgtgcatgaattatacacccagaccagacagcacaccctctccccttgcccaggtgcctggagaaaaggcaaactcagtcaatgcccatacatggcacaggccctcctggaccagagggggaggcccagggaaagtgcgctgccccactcagggccttctcacaaggttgcgtccacagcacacacagctggctcagcattaaacatgaaagcaacttcagacgcagcaccttgacacactgggtcgagccccttctcgagattagaaagtcactgtgctttgaggttatttgaaaacccacagacaatttagagaaccggacactcctgagtgctgtcaacctgagaagaggggtccccagagctgaccacagcatgggaaccagccatgccaggagcaggggaaagctgtgcgtctcccagcatgtccatgcttctgagggcagggcaggcgagaccatcaaaggacatccgggaaggggcataagtgcaggcctacgtgcaggtgtttcataagccataatagttgggaggaccagaaaagtgttggcccaggatgttcaccaggcgtggaaaagacacagcacctcccaatttctactgggctctttttcctggtgaattcctggccatgattcaattgggtgacagtggagccactggtggtgatccaggcatgctcaggcctcccatgcatcattgtgatgacctcacagcagccctctcaccccgttttaccaagagggagccaaggcagtgaggtggtgaaacaagcttccaaaggataaaattgtgccaaagcacgttaacttcctattagaacaaatctaacactctctaaacaaagacgacaagagctaaatacccattagtataaaactcaccaagtctagcatctaataaaaaattactagacatgacaagaagcaagaaatgtgatctacaaccagaggaaaatcagtgaacagctacttcacatgatatagaaaaattaacccaaaatggataaaagagccaaatataagggctaaaaccataaaactcttggaagagaacatagggtaaattttcacaatcttggacttggcaatggtttcttagatatgacacaaaagatgaattagacttcatcaaaatttaaaacttttgtgcactgaaggatgctatcaacagagtaaaaaggaaaactacagaatgagagaaaatattcgcaaatcacctattaatctgaaaagagaataatatctaaaatatgtaaagaactcctaaaactcaacaacaaaaaaactgacagcccaattcaaaaatgggcaaaggacttgaatagacattgctccaaagatggtatacaaatggccaataaacacatgaagagatgcccaacagtcactaatcattaaggaaatacaaatcaaaaatacaatgacagaccacttcacacccattaggatggctatgatcaaaacaaacaagcagaaagtaagtgttgtcaaggatgtggggaaatcagaacccttgttccgatttgcatccactgtgggaaacagtgtggtggttcctcaaaaaattaaacacagaattatcacatagctccagcaattcaacttctgggtatctactcaaaagtaggtactcaatattgaggttcatagcagcatgtttttacagtagccaaaatgtggaagcaacccaaatgtccatcaaaagataaatagatctggccaggtgtggtggcttacatttgtaattgcagtgctctgggaagctgaggcgggaggattacttgagcccaggagtttaagacaggcctgggcaacatggcaagaccccactttacaaaaaattttaaaaaattagctggatgtggtgtcacatacctgtagtcccagctacttgggagtctgaggcaggaggattgcttgagcccagaaggtcaagcctgcagtgagccgtgatcataccactgcacactccagcctggacaacagagtgagatcctgtctctaaaaaggaaaaagataaatggataaacaaaacatgatatatccatacaatggaattgaaatattatttggccttaaaaaggaatgaaattctggttctgaaattcttcaataaaccttgaagacatatgctaaggaaaataaggcagtcacaaaaaaataagtgattctactcagaggagggacctagagtagacaaattcacagagacagaaagtagaatggtagctgccaggggctgcagtgagggaggatggggacttagtatttaatggggacaaaatttcagctttggaggaggagaaagttccaatggatgatggtgatggttgcacaacatgtgattgtacctaatgccactgaactgtgcactgaaaaatggctaaggtggtaaaagcaatgctatgtatagcttaccacaatagccccccaagaaggcaaaatacttttgtgtaaagcccaagctgaatcactgccagcaagtctacgctatgagaaatgttaaaggaaattctttagatggaaggaagatggtcttaaatggaaaccccaatctacatacaggaccggacagcactagagatggtgcctatgcaggtaaatataaaacatttctttgttagatttatttaaaatgtaaatgcttaaagcagaaataacagcaaggtatcatggagttgatacatattgtagaaaaaatctatgacaacaattgcacaaaggatgtgaggaaaacagaagtctactgttgtaggcccttctgctctgtgaaacggtatcatgtcacttgaaggtcagtgttgtaaaccctataaaatccacgaagaaatgaaaacagaaagctatagtttattagctaatagtgttgataaaatgaaagcctaaaaaattaattcaaaacagggaataaatagaggaaacaagaaacaaagaagagagggggcaaaaaattagcacaatgaaagacttaaagccagccatgctgacaattattgaatggaaactctaattaaaaaacagagattgccatattggataaaaagcacaaccaactcgatggtgtctacaataaatccattttatatataatgacacatatagtttaaaaataaaaggctggaaaagataataccacacaaacagtaatgatacgaaagttggggtggccatattactatcagacaaagtagacttcagaatgagaaatctaacccagaaggatagttcaaatggtaaagcggtcaattcaaagactaataataataaacatgtatgcacttcatagcagagtttcaaaatatgtgaatgaagcaaatactgatagaagtgaaaagaaatagacaaatctacagttacagttgagatttcagtacccctttctcagtaactgatagagcaagtggacagaaaaccagtaaggatgtagaaaacctgaaaaacacagtgacctaacataacctctctgacatttatgcaactttccaaccaaagacagcagaatacatattctttctaggtgcatgtggaacattcaccagtgtaaaccatctagtaggtcataaacaagtctcaatatattttaaaatattaaagatgtataagcatatcatcttgccaaaacagaattaaattagaaatcaataacaaaaatacctagcaaacttccaaatatttcaaattaaacaacaaacttctaaaaatgccctgttagtcaaatagaaatcaaaagaaagattagggaatactttgaactgaatgaaatgaaaacacagcatatcaaactttgtgggatgcagctaatgcagtccatttgaggcaaattaatagctttaatttataactttataatttatattaggcagtgtcgataagtatttatggaagtggggaaactgtaaactggtcacagttatgtggtcctgtgttggggttttgaaatgaaaacacagcatatcaaatgtgtgggatgcagccaatcaataatctccaggttcctacaaggaagggttccttttatttctctccaggaaccatttctgccacctccctctggtgggtgggctgctgcccaagaaggaagtggcaatgcaggaaggctggctgggacccctgggggccaggggtgccagcaactcccacaggatacgtttcatttgccaaccctgaggaacactgggtgtaggagccagcactgcacacggcattcatggcacatggaggaagcaccattagtggggcagctcagcagtcttttctgacttagggcccagaagacatggccagcgtctcacccaggcctcacggttacttcctgaggacctgtagtaaaaacgcacgataaagccctttcagatctactcttgctaacatcttagttgtcaaatttatgacacgtcatgtttcgtacattgtgtaggaaactcaatcccttttgtgtttactgacaaagtcaggcatgcccattatctaggacaaagagctgcactgtcacacatcacagccactctggcctttatggcctttcacagtgagacatccactccctgattacatgcgctggcaggaacccgacatcctttgtccagtgatctgctttataaagcaggtttgaaatccagaacacccctggcagacctgagtacgttatgactgtcctccctgctccccaagagtcactggggcctgtttcaggggaacctcagtcccactggtgggaaaatcaccttcagaaggtgtgggaggagtgccaagcacctcagtgccttgggtcattaatcatttgtgaattgatcttaaaccttctccaactccccacgtctgctcttgagctgccgccagctaaagtgtaccacgtgccagtgcggacagggcttctgagtgaagggctgggcacctttctgtttccttccttccttccagactgagaacttccaaatagacttcgctagtctgatcgtttaaaataataacacgtgttctttccttctagcattcttaacaacgaatcactggactaatcactacactaagttttcctctccaattctgttatctgaacatcacttcaatgagtcaacatttcccacactgcccaaacaaaaggaaggcagtccccacatggcagcacttggcagctttaacagggcgttgggtaccgggaactctcaggctcttgccctggtggcacctctgcggccactgcccctggctctgtgtgccccccactttctcctgctgtcccctctctttgatcacatgctaaccctgagagctgggcacacagcaagagcacatgcagcccaggcctggggtgttcatagggtgtttaattagggaaatgtaggtgtgaacaggccatccacacgcaatgggagttttctccctcgagaatgttaaaatctacttggacaaatggtgtgactgtgcattatttggacaattttatcatttcccgcttgcctgtcacccacgagagtcctgcccagactctcccacgaccttgcctgcagctgcgcttcctatggcgatgctcgtccaagtgtcttcaggcaaacaaccaggactcatcccgggtgcatcttctctcccgcctgccccaccttctgaaaccagccctcctcccatgagcgtcttcctggcgccccagcctggactctctctcctcctgctgggtacttggtgggccactctctcccatcgtggcaccgtcctcctcagagttcgtcactcttgtccactcccaaaccacaagaaaggcccacacaacgctcaccccacgcgacccatggtcttcctcatgctctaagaaaaacgccgggtggccccccaaaacctgcctccgccccggctcctcacctccaaggccagtcccggcaatatgcttccatgcccgaggaggaattctagaaccaagcacttgggttctgaatatctgagtgcagagcagtttgagagaccgaacgcctgcccaccatgagaactggtccctttccggcagcatcagccctactttctccaagccacaccctccggcctgcccgaaggctccatctcagccatctctgacaagacccccaggagagggcgaaggtcccttccttgcaggaaaagacctcagtgatgtctgttgatacggttttcagatatagaaagcatctctctgggaggctgtgcgccccgaaggtgcccttgcagaccaagaccaaagttctcggggatgatcctcccccagcagaggcgaggtgggggcttctctgctcaccgtgacttgtacaaacttaacatgagatgcttcatttgctttcaagctccctgttttctgactcaaaacaaattttgcaaaggcacctactggctgttaaaactggaattagtcttactggtcaaagttccttttcctaccttgaagtggactcattttggcatataaacgtgaataagatgcgtctaaccaggcaagcgagccccccaaacaggagcgccctggctgacgggaaacactggatctggggagaatcgcgcaaacgtcctctcctcctctccaaaatgacggctgagtcgaagagtccgcctgtcagaacggcgacccggcacgaggccccaggcccggctggcgagggcaggaagatgtggatggcacccgccagatccagattccgggaggggtccaggggaaacagggaacccctgggtgtcagtaacgacccttccaggctgggccaggccattggtgacgagggagggccagcaggagctggcacggggcccaaaaggacttcatcggagacatcggggtccaggaaagcaggtgctgcaggtggatggtgaggggcgggcctggcctgggtggacagtgagggacgggcctggatggggtggactgtgcgggttggggctggtcggggtggactgtatgggttgggcctggctgggatggacagtgaggatcaggcctggccggggtggatagtgagggatgggcctgaccagggtagacagtgtgggttgggtctggccggggtggacggtgaggggtgggcctggccggggtggaccatgaggggcgggcctggccggggtggacggtgtggggtgggcctggccgaggtggacagtgagcgaggggcctggctggggtggactttgtggtttgtgcctggctggggtggacagtgagggttgggcctggctggggtgggcagtgaggaactggcctggctggggaggactgtgcaggtcaggcctggccagggcaaagggagaaggagacagagacccaggaggctgctggacatgggatacgctggcatgggaggtggaaagaggcccagagacagaaataaagccatgcaggtaaacaacaaaacaaagtcttgtgaaaagcggacggctgggtcaccctggagacaggggctgcctactgaagccagagacttcttgcgcagtgacacccagaaagttatcttttctggagagggcaagggccggagtgaggcagttcggccccatgctatgcacagaggaagggtagggaggaagggagcccagacatggggctgactcactccagaggcgccactgcagctcccagagactggagcttgcccaccttctgcacagggtctgctgtgttccccatcagccccctcccaccctgagctgccgctgggagcagccaagggagctgctcagtgccccatccttgctgagggctcgccctacaggagctttgctgagctcctggagaccaccacagtgattcagaaatgtgcccctccagagcactaaagggatggagtgcaggaacagattccccctccccgcaccctgggagtcagcaagggctggtgtccaaccaaggggctaccatgggggacctggctgcgccatctctcccgttcccgaggacccgactcccacttactcaaagtactcggcagcaggtgtggtcccaaaagtgtcgttgaggcccgagctgtctccactggagtcaccaggatgggcatccgagcagttggggctgttccaggagttgttgcagtggatccaggggagctccgtggtgaaggaggagaagagatagtgcagcgcccaggcgatgatgacgttgtagaagaagccgacatacagtgagatgaggatgaccgtgaagcccacacctagcgggaagggggaggccatggagcccacgcaggtggagcacagagccaccatcagcaacgtgtcccttccacctcccctcacgggagacattaccctgagcccacaactcctggacagccctgatgccagacagcatcttcaaacatgcagggaaggcccgtgaaaggcctgagaccagatttcacactgtgaccacagagaagaagctgcccactccccaaaaccatgcactcctacacaaggactttcagggtgtctctgagagacatccagagccagcaaggtcacccagagctatggcgagtatccagggccacccttatgcacccagagacccccagggacaccaaggtcacccccagccacatatggtcatccatccccaccacagccactcaggatccataagagtcatccaggatcattgaggccatctagggggtactctgggtgacccagggcaacccaactcatggttaaccagggctatcaggcccatgtgaaaccatcagaatcatccacggccacccagaagcacccagggctacctagggagcccatgcaaatagggccatcaaaggtcgtctagggttacccaggaacacccagtgtctcccagagccatcaagactgccaatagtcactaagggacacctggggtccttgacagccatccacagccaccaggctcattcaggactatggagggcacccaggaccacatatggccacccaggctaatccagggccactttgagtcgcaaagggccatccatggccatccacagccatctccattcatccacaactatccacggtcagctatagccatcctcaaccattcatggccatccacggccatccacatccatccgcagccatccacaaccatccatccatccacagccatccacagtcatccatggccatccacatccacccctggccatccacaacaatccactgtcattcacaaccatccacagccatctacagacatccacaactatccatggtcatccatagccatccacaaccattcatggccacccacatccatccacggccatccacaaccatccatccatccacaaccatccacaaccatccacacccaaccacaaccatccacatccatccacagccatccatagtcatccatggctgtccacaacctcccacatccacccggccatccacaacaatccattgccattcaaaaccatccacagccatctacagacatccacagccatccacagccatctgcatttgcccataactatctacggccacccatggccatccacagccacccatggccatccagagccatccatagccatctatggccatctgtggccatctatagctgctcatgcctctcctgcaagactactcagaagagtcatctgagctcatttacagccatgtggagccatccaaggtcacactacccatgcatgaccatccagggtttcctagaatcacccagggccacgtaagaccaccagggtccacccagggcaggtaagatcatccagggcttccttgggccacccagagccttgcatgactatgaaggactactcagagccatgtttgaccttccgaggccttccagggccatgcaagaccatcaaggaccagccagggccatccatggccatgcaatgccatccacggtcttttagagtgtcctttccaggatcacagacctggaatttgggatccatcatttatggggtctcctggtaaactggaacagaactcagtggtcagcagtctttggagtggtgtcagcctgactggtgggcagatgggcctggtggtcagatgacttcctccttgttcactgctggatgggcaccatcacgttcttgctactggggttcactttactaagctccaggaagaatttttttcatttccacatttcttctcagtttactgacatgattagtttgggtacaactttgatttactttattaaaacccagtaggcaccatgccgtcttgcctgttcatggccggggtaggagcataccttcaaggtgacttttgcagggggtggcctgggttgctttttatagtctcaatccttaggagaagctgttctcatgctcagggcacctcagtaaagttctcttcctaccttgaaagcaaaaagaagactcaaaactttgttttaaacaaatctaagatcaggagtgcgggtaatcccccttggaaagcatgctcagtagtggtgacgatgaaagccaagctgctgcctttggaaggtcttctcctatgtggatctggtccatattaaataattctgtattgcagtctgtcatggattgcctttgaaaagccttctcccatatggatctggtccatattaaataattctgtattgcagtctgtcacggattgcctttggaaggccttctcccacgtggatctggtccatattaaataattctatattaccgtctgtccggagtcccttaggagagaggctgggttgtgatccacagtgaactcacccaggcagccaggttgggagccacactgtcggtgagccacattgtttccagagagcactgtggagggttttccatggtggtaactgacaatggatgcccaatttgaaaatttttcaaggttaaaactttctgtatcaagtccttagcttgaaaccctgggaagtccttgatcaaacaacaccagagtggatcccaatccagagtccgaaggaagggctgagtgtaccctgggtccttccagagccactgcttttgtttcctgccctgcaccacagattggcagggccaccatgggagcatgacatcaccatggccactgtgcatgacatcaccactggccacactggctgatgcggctcccagagcaaatgggcctgctctgatccatgggacagtgcagcccccacaccagagtcccccttaccaagaggacccctcctggttgaggatgcttccctgggcacctgggtgaggaaatggctcccttgaacagtggtggccagtcccctcctggatgcttccctgggcacctgggtgaggaaatggctcccttgaacggtggtggccagtcccctcctggatgcttccctgggcacctgggtgaggaaatggctcccttgaacggtggtggccagtcccctcctggatgcttccctgggcacctgggtgaggaaacagccttgaacggtggtggccagtcccctcctggatgcttccctgggcacctcggtgaggaaatggctcccttgaacagtggtggccagtcccctcctggatgcttccctgggcacctgggtgaggaaatggctcccttgaacggtggtggccagtcccctcctggatgcttccctgggcacctgggtgaggaaatggctcccttgaacggtagtggccactgtcagcactggccaggcattcggttgctccaagccctgcccttcaggaaggcagagcccgtgacctgggccactgacgtgtagcccctcaccaaccagccaacttcaccacgagctcctcaagctcaccgaaggcccagacgcaggaaacccactaccctgttggctacagaaccttctagtgatctctgctcatgacatcactgtgggctccagatgaagcttcacctgacacatttcttaaacaccatttagtgacacgggagactgtctccccaaggaggtggcaggcaaagtccctgctcgcgtttccagggaaaacattcatgagaacatggcgcttcccttccctcctgtctgactcccaggaagctcggcgctaagtgtgaatttcaagcatagcttttaaggcccatttccttaaatttatttttgcttagtatctttttctttatattaaccttgtgtgtagtttgttttgtgtttgagaaatcacactaaaatatgactgactataaaatattttatattcaatacaatcgaatggtggggtttccagggcgtctgtaaaatggggttgcccttggcaatgaaaccgaaacctgacaaacagaagctggctgagggtcgtgcctgcaaatggtgcttcgcccaccagagcacggccaccctgccccacccctgaacaccagtacaccccagggcacccacgagagctggcttcaggagagggtgttgtcaacgcatgcacggatcccgctggaggaagcctctggtggagcagcccctccatacctcacaggaggcatcaagcaggtctttggaagaaagacgggagagggccgggcacggtggctcactcctgtaatcccagcactttgggaggccaaggcaggcggatcatgaggtcaggcaatcgagaccatcctggctaacacgatgaaaccccgtctctactaaaaatacaaaaaattagcggggcgtggtggcacgcacctgtagccccagctactcgggaggctggggcaggagaatcgcttgaacccgggacgcgcaggtgacagtgagccaagatcgcgcctttgcactccagcctggtgacagagcgagattccgtctcacaaaaaaaaaaaaaaagaaaagaaaagaaaagaaagaggggagaggacgcctctctgtgtcagtctgggtgtctttagtatgtgcttgctgtgtgtgcatgtacccgagagacagagaggagaagagacagaacaggagagagacagagaggagaagagacagaacaggagagacacagagaggaaaggagggagagagacagaaagagacacagggagagggaaagagagcgaggggaagggaggagacggagagttttaaagacccacctctcaggaccctggggtgcactgcggggctgaaggccccagaggcccctctggctgtggtgcccatcccaagctgctctggtgagactacgggaaatgcacgtgagtcatggctgctgagcagctgggcctcaccatgaactccgccctctggctttcaagggtggatcttggctcccagttaggagcagggagcagctcgcaggtctgcaggaggggaagggccaggaacaaccaggcttcctggagagctggcttcattcccccatggaattgccacctgctcagggctggatggccttttagctccacaatgattgttgtgggcactttagctgatgtcttaatttaccacgtggcacttctattcaatgagacatgaaaacgtatgcatttttattaaccagatttttaaaaaggacaaaggcacatgtatcagggtgccgggggtgcatggtgtacatctgatttcataagcaatgtcagtctcctctaaactggcatcctgcgcttgacaggtaggcagaacaaacgggacgctggcaccggaacctgcagcgttactgagattcaacaactcaaggtgtctcgtctctaaaaagaagtccagctttcttgcactgattcatctatcccttcgtgggtcgaacagttcactttctgtgatttgcaattttccttcctcaccaggactaggtgagtgtgaacaagctcttccaaagatccgcccgcagggccacgagccttccttcctgcggccacactttgcagccctcctctgggactagtcttcagaacgaagctggcggcatggaatgcgggattgtgggcacagggtctgtgacacagaggagacgactgcccagcttaccccggctgcgcattgtggagaacacgatgatatgcccacatccgccggctgcatttcttcagaacgaggtgccactgctcacgctgatggaagtcagaggttctctactagcccaagttgagttggtgctctgaaattcttagaaattgcttcttgacattttatggactacaccatgaagacccacaacggcagctttgttggtgaacatcctttctgtgtgacagacggaaggaaatgagctcttatctgcggacctacaggtccctttttctcctgccctcctcacctgaatcctgcacgtgaagaggtgatttaacttgctccaacaccaggctcactggcgggagtggggcacagggctgtgccttgtaagacacttgtgacagactccagggtggctcttgaaaatccagcaaagcacagttggatgtcgctgcagcctgtgccagagcacaatgtcatctttcctcagtttcccctaccccagtgggccggcttcatctctgactcagcatcccaaggctctgagccctcagatactctccaacacggaccacggtgcaagctcagcatcgcttacgactgcaggctcagaggcagcactcctctctgaaacacgtccgtgccgctggccagggccaggcaggacacaggtaacgcgtgtgtcttgaactgttagcctgggttcactgcaacgccggatttgacacgcgcccctgtctgtctgtaacaacaggcctgggtcttccgttggctgctgcattcttccattggggacctgatatcagggacctgacgacggggacggaggcatgttctgagcaattcttgtacggtttgacttctacctgcagatcccgccaaggggtcgtctggcaccagtgcatgcccaaagcatctgtctctcactctgaagatgtgtgtggggtgggggtgggggtgggggtggggagggaggcctctttttgtagctccatttcagtgcagtccagtgaatgtggagcccaggaagaaattactgaagtcataagattcactaagcagcccattaagaacgaatgagtctcgtgcctgggaagaaccatcacacctgtccagtctcagcatttccacggctgcgagactgacttctcctcctggagcctgacctcagcagctgtgaaatgccatctgttccctctcctaagggaggtcagcggctcgcctctgaaggcccacccctgacccattggtaatgcgtcctggggccgtgctgggctcggctgtcttctaaacacacagcttttccagaacttctatgagtatccagaggctgaccttctagtagtcattttcttaatgccaattttctgagtggtgcctctgagtaaatgcctgaatcctccctgaaccaccagcccctgagaggtggctttggcccactgggcaagtgtgacctgcgtgcagggattggggagtcagaaacgtctgggatcaaatcccactctgctctgatggggacaccagggggaacgaccccggccctctggtcctcagcatctgcatctccaacacagagacctagtgtgtgcccttcccagggtctcatgaagatggagtcagaaatgctgggagagggcttggtgctgggcaggccacctgcactcaacacgaggtgtgtccatttcatggtgccagagcaggacatcagcagccggaaaggttcagtgacaactagatggatcaatagctagtagataggtggataggtcgataggtggatggatggattaatccatacatagatgaatggatggatggatggaacaatggataacagatggatgaatggatagatgaatagatagaagatggatggatggatagatccatagatagatgaatggatgaatgaatggatggatggatagatagatggatcgataggtagatggatggatcaatagataatggatggatggatggatggatggatgatagatccatagatagatgaatggatggatgactggatgggtggatgaatggattgataggtggatggatcaaaagaaaatagatggatgaatgggtagatgacagacgggtgaatagatgattgacagataatagatagatagaattgtgggatgtgtccctcccccaagttcatctgttgaagttctaaccccgcaaacctcagaatgtgactttatttagagacaaggtctttacagaggtgatcaagttaaagtgaggtcattagggtgtccctaatccaacaggacttctggcctcataaaaacggggaatttggttacagagatatgcatagaagcaagaagtataaagaggcacagggagaagacaatctctgagacaaggagagaggctagaacagatgcttcctcacagcctcagaagaaatcaaccctacagacaccttgatcttggacttccagcctccagagctgtgagacaataaacgtgtgccttttaagcctcccagtttgtgtcatttgttacagcagccacaggaaacagatacagatagatgatgatgatagatggatggattaggtagataggtagatagatgggtgaatgattgatataaagaccaataataaagatagataatggatagatagataacagatgattgacggatggaccgatggatgataggtagatggcagatccatagatagacatgtgttttatcttaactacattttagcatcttgacaaagtaacaccacaaatgaattgcaggaccctcacctagagcaagggaatgaactatccagtccttgtcactgcagatctttaaggtgggacccaagttcaagtggcgtgtgccatgcctgtgtcttagcaaagcagggctggatgcccatgatgcggctggcttgctttgaaagctccagcgtcaccaccatgatccgcgctgcgccagggtgaagggaggcacccgcatattacctttcagtatggggcagatcttccagacaccagcggccccttccctgttgaactggccgagggccagctccatgtagaaaagtggcatcccagcaatgaccatgaagagcaggtaggggaccaggaaggcacctgtggggcagaaaagcacctttagtttggggcctcggaagggcccatctctcctcgtgggagcttgggcggctaccccagtcaaccatccatgtcctggcccgaccgtttcaccccactctccaacgggaagtcccaggctagggtagatgagtcccgaagcccgccctccactgctgtcagtgcctgacacgtccctcctggatccccttgtcattgcgcacctcgagccatgaagtcaaacccaagagcagcacagggaggccctgcccatgctcagcggtgctctaggtactatccttccccgtggtggttcggggtgcccttccctggcctgcgtccccccactttgccctgatcctctttgcctcttgagttgtgaatctgtgaccccccaactctgcccctgagctgagtgggccctgccgtcctgtccccagcttctggggccctcagaggctgcccctattgactgtggctgagggtctttgctgccccaggtgctgggagcccctccagggtaaggcctagttcctctgaaacctgcacctcccccacccccagcacaaagcccagggaaccctggtctcaacctcctaaattccctctgctcccctggtctgactggagtgagggagagctttgccactctctctcctggggaaggggagggggaggcatggggcccctcagctctgtctttgggcaacttcctgtacctgctgaggcctctgtcttcccctcttcaagatgggcataaaaatgccagcctcctcgcaggcatcctgtgaggcttccccccagattcttccccaggcctcccttcccagacctagcatagaggccaatgagggaggccaggcagggaggctgggaggcccaaagaggctcctgggaagggatcaccaatgttcttggacgtccccggtggccctgcctcgatcttcgctttctggctctgtggctgggttctggagggtgcaggggacacagtcactttccaggggcctcctctgccactccaggctccagggcctccacatgctttgcaaaggctgccaaagtcagccgcaggctgttctttggacctttgagaattttctttccaaagcgaagatagcctctggaaacaggaggcagagccaagctgccccgtctgccgccccccaccccccagcttcttcgcgggcctccctttcctaaactctgaaatgcaggcgtgggacaaggcagctccgagtcctgctcaatggttttgtgacatcctctgggaggatctgcaccggccgtgagctctcacagggagctccgtcttcacgcatgggaacagcttcatctcgtttccgtacgtgccttggccccggctgcccctacgacccccgcccggccagcatgctcagggaggctgagatgggacttaccgccaccatttttgtagcacaggtaggggaaccgccagacgttggccaggtccacagcaaagccaatgacggacaggagaaagtcgatcttcttgccccaggtctcccgatcctgggcctccacggggctctgccgcgggttggtgagggtggagctggtgagctgcactccgttctgctccttgacaaggatgagctccacctccttcgggcccacggcattgggctccttagccggggccaccacggaagacatgagtcccacggagcatttgctcttactcatgggcacactgggagttgaggaattctgtgcttcttccctcttggtcttcagccaatatgaaaaataaacacacaacaggaacgcaacaattcagcagccagggctagggacatacctggtgcggagggcagagccccgaggcattcacgggcattcctctgggaagggatgggtgcgttctcagcgcctgagatggtacagctggggcacgggagctgggaagtgccagccctcaccacagggctggggcactggcccatggaggcctcaagacagacactctggtttgctcctccttcccctgcacccctccccactcctatcaaagtgcaaggctgtgtgagctctagcgtggctttctactaacaatgcatggatatcacttccaccttcccccacacttccagagagcaattttacaatcttctaagagcattcaataatgtcttcatgactgttcatctgtattcttttttcttttttttttcttttttaacatgaggtctcgctctgtcacccaggatggaatacagttgtgattacaactcactgcctcaacctcctaggttcaagtgatccttgcacctcagcctcctgagcagctgagactacaggtgtgcaccacagcaccggctaagttatttttgtgtgtgtttttttgttgttgttgttgtttttttgtagagacagggtctgactattgttgccaaggctggttttgaactcctggtctcaagcgatcctcctgcctcagcctcccaaagtgctgagatgacaggcatgagccaccaagcctgggcttgtattgtttatactcagtaataaagagcaaaatttatataagcaaaatcagccactgtaaagagagtttagacataaccatcacaactgcttcctcaaaaagtgtctgtacaagccacatccacctagggcaggtggcaccaccccgcgtgagagagctgggcggaggatggacagggcttcatcgcgggaactagctcctgggccagcagggtaacccgggaaatcctgtccaaacggctacataaacccccctgcaacgctgagcaggagagcaggagcgtctacaccgccccagccagggcgacttctgtcatttggatccagcagcagcgtgtttcagtcgagcgcacgcacacacagacacacagacacacacagactcacaaacacacatacacactcacacacacacaggcgagcacatacacttacacacatacacactcacactcacacaggtgcgcacacacacacaacgactgaagcaggtcgcaggtggaggctctaacaggcaactttccctgcatccaagtttgcacaaaacctcacccatgagctatcacgagctatgccacgcacccggtgacaacctcaaccggcacgacccctccggtgggtaaaacacccgacgaaggggttccgcggcgcgagcaggaggccgcgttgggagagggcgtcgggtgcggagctcgcgagtctccggcaagccgccgccgccctggggccacctgaccccgctcctgaacgcggggcctcagaggcgagttttgggtctacacagcaaaatgtggcccgttagaaagcgtccttcctcaccgcgcccagaacaagaagcggcgctgcctggcgagtcctcgcccggcctccccgccctgcccagagcaccagcgcgcagcccgcgcccgccaccacccccaacacagacaaagcccccgcgagatggagcggcggtgtacaaaacccactccgatcggatctgggatgcgccgcacccctagtagggttaagttccacgagaagaaaccaggacccccgagtcacagccatagacacccacgcgtcccctaagccggtgactgcaccaggtgcccacccggttctgagcctggagcggggccaggggcagccctgcaccctcctcgcgcctcggacggcctcagggtcccatgtggcgctatcggggggcgctcggcacccgcgactgttggcgactttggagacggcgcggtctgcgggacgcggggaccccagactgtgcccggtccccggcccccgcccctgcgccccacttcggggtctgcggaaggagcggggcgccccgatgccgcgagcgacgctggcctcacctggccgcccgggcctgggctttgcacgcgagtcctggcgccgagagcgttccgcgaagcctcccctcccgctccgcagcgctgggcggtctcagcctcggcctcgggctcttatccagtagacagggtccgccccgccccgccctcccgccgacggggtcgccctcgcgtcctggctcctcctcctcacctccccgggccgcgcccccgcccccgcctcccggacgcagatggctgggagcaagcggcggccacggctccactgtcctgcgcgtcccggagcctcggacacggggtgcgggggcctgggggtccgagacgtcgcggatctagacgggcgccttccacgcgggaactgtctgggccgccctgcgccctcgcccgcacccctgggggctgccccgggcacccgctatcttcggacgcccagggctggcggctcccagctggacggggagggagccgagcggctgcgcgcggggaactgctgggaccccgtcgggccgacccttccctccctccccatcaggggcccctacggtggccccgcgctctggggaccacgtccctcccaccccgccgctccacgctctgaccagcgggctcgggaccccgccgaggactctgcacgcttcccggcgtgcgggctgcggagacgcgggtccttccgtggccttggggtctccgcgacctcgaggcgaccgaggtcccctaggcctagtccccgccttgccggcatctagtgcgcctgcatcggaaccgaggggcccgcctcgcactcgcctaagaaaaccatttcccgggacgggcctggaaagccctggcggttggtatccggctccagggccgaatggactccgaggctttccttccgagatgcccgggcgggatttcttccattcagcgcgcggaggaatggagcccccaggccgccaaggcccaggatgtccaggtccttagaaggcaccgaggtgagtggccgcgcccagctctgtgcgtcgcccctagggtcgcccgaaccccggcgttcctccccgtgtaccctcgatgctcccgtgagcaccctgccctgggcgtccaggcgagccctggggctctccagccattccagcctgcagcccgcgctcagcagtgagggaggcagggacccttggtgcctgactccgcagtacccgcccgcagcgcggtgccccaccccctgcgcccgccccgcactccccgcacaccaccactcccctccccgccccccgctcccccgctccccggctccggggctggcacccaggcagaaggcactcagggctgtgacttcacgctgagttggaatcaagtgggagcctagtgaccccctggggctgccatgacaaaaaactccacagcctgggggctcacacagaagccttcagttctaacggttccggtgtcccgaggccaaggcctggccgactctgtgtctggtgagggccgcttcggggttgcagacagttctctccctgtatctccccacgctaggggctggggagagctctccggggtctgttttatgagggcactggtcccattcctcagggtggagccctcctggcctgaccacctgaaggcctcctgacaccacctcccatcaggggtgggacttcatcgccagggaggtgaggggacttcaggagagggggatgtgccacctcgaagctgtagtgtgtcagtggctgccacagaagggatctctctacccctattatctccatggcacaggtttcaataaaaaggagtgagccagggcttggccccagagcagcagggttgaggaaacagtctcccaaggaaaagactttgcaggtgactaagggaggctgttaaataaaacacacagagagaatggatgttcaggtatattaaaaatacacaggccaggagtgagggctcacgcttgtaatcccagcactctgggaggccgaggaaggaggatctcttgaggccaggagtttgagaccagcctgggccacatggcaaaaccctgactttgaaaaaaaaaattagccaggcatggtggcccacacctgtagtcccagctactggggaggcagaggtgggagaatagcttgaagctgggaggtggaggttgtagtgagctgagattgcgccatgggccacaggatgagaccctgtctcgaaacaaaacaacacacaggcaaacagcgtttgggaagggattgcaagcgctgcaggagcgcagaattgtgacccaaagctaacagggtacaggcaggaaggtgcagggactagaggtcacacagatccaggcagccacatccttactgaccagtagcacacagagaatgcacctgccacactcaggaggggccactgagtgatcacacgcatgtaacctgcactctgccaagacaaagagcattcctccacccctgcctcaaccccgacttccaaccacatgaattaggataaaggagctttgggtgctctaagaacaaactaactgcgggagttgggctcatggatgacacagagaaagcgcagctgccagacccatgggtctccatcctcccacaacaggaatgctccgtgaactgtgcaggtagaagcagtgtggttatgagctgatcagacctgatcaaacggccgacctttgcatgcttggaactttgccctaaatgccaagtcggctttattatcattgtggttgctgctatggctgctgtgggctcagggcgttgggtgcaggcaggtggagcccccctgcctctgcacttcctgcccatgctcggcacctctccccactggctgggaccgcagctgcagagcccacaaggagcccctggccactgactgctgtctccaaagcaaccttgccccacgggtccccccctccttgggtgctgcctcctgggaggccctcagtgctggatgttgcgccgtgtctcgcctgatttggaagacagcgtggatgctccccccatgagtgctgtatctgagaggggacaaggacagtggcagtgagatgacagtcatttacctgcctgcgtatttactatattgagggtgtcctgtgtgctttcagaggaagtcccagggagcccagctcagactttaaagaccctgacctctcactggtgctgggaaggccctgggggtagggggcttggggagggcaagggtgaggggaccagtaggcaggagtggcttttgtccaggggtgtgggaggtcagatcccagaccttgtctgtccaggttctgagaatgatggaaggcgctggctccaggcaggaggccctgctcactgctgcacccctgtgccctccccgggggccacctcaggaggaagatagtttcctctcgcatggcagggacagcagtacacttccaatgtcttctctcagagctgtatcattttccggctcactgctggttttagtcacagacatcttgatggtcttgccactgcacagcatgtcacagtggtctgccacactgatgacatcaggctgaatcaacctggggagcaaaaaatgaaaaatgccaagatgccttggtaagacacgtgcttgctaaaaattgaaagagaaaataacacattttcaaaacctgccaccttggtgaactttctgtgggtccattggtctgattggtcaaaatatatatccaaaatgaaagaaaacacgctgtctctggcaagcaagactaccactaagaaagaagtatcatacctggtgaggctcttccggtttgcaaggtgaggtgcaagacactcggggatgcttctctggcctgttttgtgttcttcataaagcgtttggttttgagtagggcccagagtaagaaagattctgctgctggtctggatgggcaaactgctgccccaggaggccacacaatctggtgttgttctccgactttagctggcatcacagtcacctacataaacctttacatgcataggaggcacagatcacctcgctccatccttagagacgcaggttcagtaggtctgaggtgggacctgaggacctgcgtttctagaaagttctcaggtgacactgacactgctggcccaggaaccacactttgagaacactgatctggcagaaccagtgggaattggagggtctgtggcagacggagatgttgtaggcagcgcagaaaataacagttgaataagagctgaacgctctcctgaaaatacgtcctctccttttgagtaagagcagccctgagccatagcagaaactcagggagctctgttaccatagaagctgagcggcccagcagaagctatttgttatcggacccacagaggttgtgtagacagcatcctgccgtcctcaggtggaatgtttacgacacaaggcttgagcaggtctggaaggcgcggtaagttccatgaacaagcacccactcttgctgcatggagaccgcttccttaacccagacctctggctacatgagagtttcttatgacattgaacggagaaaatgcatgcgttggggcctggattacatgtgtctatacacagcacactgcccccaccagaaagtgaaaggctgtgtcatcatggccttctgtgaaagaccagtgaaagaaagcttcccagtgggcacatattcaaaccatgcatcaggttcatcatctagccaaggagggatggccagaggatggccctaaatgtagtcataggcagtggcgaatggtctggctacagagcaggaggcttcaaaaaaattgaaatattgatgacaagatttttcttggtagatttcatgtttgtagaaaatcttgaggaatttacaaaaaaaactactagaatttataaatgaatttagcaaggccatgggatctgatgaaaaccaattgtatttctatgtactggcagcaaacaattctaaaataaaacctaaaataatttcacttacaatagcatccgaaacaaaatgcttggggataaaccgtatccgagacttgtgcaccgaaaacctagaatccattgctgtgagaaacttttgaaagtctagacaagcagaggagtgtgtatgtttatgactgaaaggctcaatattaatgagatgtcagttcttcataaattgacatacagattcaatgcaatctcaatcaaaattctaacttgcttttttgaagaaattaaccagctggttctaaaatgtgtttggagatgtgggtgacctagagtagccaaaagaatcttgaaaaagaacaacattggaggacctaggagatgcctttcatgattccaccatcctaacaaaactaattggactttttttctgtaattatctcggcatttgtgtctttttaaaaacagccttgctgaggtgtaatggatatgcaatattccaagtgtacgatttggtgagtgactgatggggtcatggcaacgcgatcacacagaagatgatggcggtgctcctccctcgctgacttccctctgtctgtgtctctttacctcttcccatccccaggcaaccactgatttgctctttttttttttctttttgtgagacggagtctcactctgtcacctaggctggagtgcagtggcacgatctcagctcactgcagcctttgcctcctcagttcaagcaattctcctgcctcagcctcccaagtagctgggattacagatgcccgccaccacacccagctaatttttgtatttttagtagaaacagggtttcactatgttggccaggctggtcttgaactcccaacctcaagtgataagctcgccttggcctcccaaaatgctggaatgatttgttttctatgattacagaacaatgtgtttttaatagaattttatataaatagaattatacagtaagtactgttttctgggtctgttcttttgcattcagcatatttgagaagaatccacatcattgcatcggcagttaattcccttttagttatttgtagtatccattgtgtggatatgggatatgatgtacttggcttttccatctgcctattgatggacatctggctgttacaaataaagctactatgaacatttgtgtgcaagtctttgaacataggctctcattactttggggtaggtaagaagtacttagttgtggaatggctgggtcatttggtagatatatgtttaactatttttttaaaaactgttaaattcttttgcaaagtagttgtatcattttatatttccatgaacagtgtatgataattttagttacttcatattctaatgaatacttagtatggtcagtcttttaaattttagacatcctaatatgtttacagtaatatctcctatactgctctaatgagcgtgatctttgaacaatttctcatgtgttagctgcatcagtgccacttcgtgttaagtgtctgttcaaatcttttgcacctttgggttgtttaattgagctttgagagttctttactattctggataccagtcccttatcagatgggtactttgcaaatatttttcccagtcttggactgtcttttcattctcttaatagattctttcagagaagagttattaattttgatgaagtctaatttatccatttttctttcacaaatcctgcttttagtgttgtatttaagaaatctttaccaaattcatgatcatcaagattttctcctaagttttcctctaaactttttaaaattatagattgcatatttacacatagaatgcatttttaaactagcttggtgtaagacaccaattgaagttcaccactttacctccgaagagccagttgttcagtgttctgctgagttgcttttgcacctttgttgaaaagtcagttgtatgtatgtgtgtgggtttatttctgggctccctgttttatgatctgttaatccattttgatgctaataccacacttgctgtggactgaattgtttacccccaaattcataggttaaagccataacccccgcaaggtgactttatctggagatagggactttaggaggtgattaggttaaattaaattgtaagggtggggtcataacccaggaggactgtggccttataagaagaggaagctctctccattttactttctgccatgtgaagatagagtaagaaggcggccatctgcaagccaggaagagaggcctcaccaggaactgaatcagctgccttgatgttggatttccagtctccagaactgtgagaaacaaatgtctgctgttaagcccccaaatctggtgtattttgttgtggcagcctgaggagattaaaatggcactatcttcattatggtccttttgtaatacatttttaaatcaggtaatgtaagtcagacaactttgttcattttcaaagttgttttgcatattctcggtcctgtgctttttcatatgaattttggaatcaacttttcaatttcttccaaagtcccgcatgggatttcttcgggtggaggctgccatcaatttttcatcattattcctttctgaggcaggcttcatgtctagattttcttctaagttctgatttagccaaattccaaaaggtttgatatgttgttttgttttcattcagttcaaaatattttctgatttcccttttgatgtcctctttgacccattgttatttagatatgtgattagtttccaaatattagaagattgtctggatatgcttctttattttttgccaaatgtatttttcattttggtatcaaatttatttgcctctttgttggagaacttgctttacatgacctgaatccctaaagggaattttatggtctggactgtggtcgatcttggtaagcttttcacatgcatttgaaaagaatgtgcactctgctattattggatgaattattgtataaatatcaaataggtcatgttagttcgtactgtttttgaagtcttctgtatccttaaaagtttctgtgtatctgttctatcaactattgggaaagaggtattgaaatctccaagtataattgtgtgaactgctctatgtctccttgcagttctatcagtttttgccgaattcattatgaagctatgttattaggtacataaacatttgtccttttggtgaactgatgctatcataaaatgacattgtttagacatgatctgttttgaggattcctatggcttgcatattaggtggcttgaagtcccacagttctctgatgcttggttcatttttttgtcttttttctttatttcattttgaacagtttgcactgccatctatgtcttcaagttcatgtatccttttcttctgagatgttgaatccacccctcctctcaaccagtgcattttgcatctgagacattgtggttttcatctctagaatttggattcgtgtcttttaaaaaatatctgtcatgtctctattaaacatattcagtctcgcctttggctagctcttggaatgaggtaacagttatgattgtcaatgcctctctaattctatcatttgagtcatctctgggtctgtttctattgactgtaggtcacattttcccacctttttgcattgtgctaatttccagtggatgcccagcattgtgaatttaaccctgttggtttgtggataacactgtactaatgtgaatattctggaagtttgtctttggccactggtaaggtccctgttgtagtttggtcattctttcaggttttgcttttaagctctgcccaggaggaccagagcagtgctttgctcagggctgatgcttcttccctgctggggcaaagcccatcttagtttcctgcctaatgaccatgtttcccaacggagggtaaatccagtctctactcttccattttgactggaagcaaaaattgtggacccattcacttttctttttcttttcttttctttgagatggagtctcactctgttgcccaggctggagtgcagtggtgcgatctcagctctctgcaacttctgcttcctgggttcaggcaattctcttgcctcagcctccagagtacctgggattacaggcgtgcaccaccacacccagctaattttcatattcttagtagagacagggtttcactgtgatggccacactggtctggaactcctaaccttaggtgatctacatgcctcagcctcccaaagtgttgggattacaggcatgagccactgcgtctggcccccattcacttttcttacatttaagggagttaatctgcacctagccagcccttgacagtgtcacatgacagacagtggcctcctcatcagtattgctgtttctgttgtccttcctcagcaaatgttttccagttactcttgtagcttctcggccaagtagctcagttctgccctggggaatatgggcagaattcatgacatttagcttcatttagatgagagaacatactctgtgtgatttaaacatcttgaaatttatctgtggtcaagcacatggtctattttgtcaactgcttcatgtgcacttgaaatggcacatcatttggtgaatagagctgtttttctatgtaactacatttttccctgatagttatatcagttactaagagatctatgttaaaatctccaactatgtttgcagatttatttctcctttaaatcctgtcattgatgcttaatatatttaagctatgtgattagatacatgcacttttaaacattgtgtcttccaattgaattgaacttttatcatcatgaaatgtctgtctttaactctgctaattctcctagtctctaaagtacacttagtatgacaatcttgccaaataaactttcttaagcttctgtttgcatgttataaatatatttcctattcttttactttcttacctttttgttttgtttttttatgagacagagtcttgctgtgtcacccaggctggagtgcagtggcgcaatctcagctcactgcaacctctgcctccagggttcacgtgattctcctgccttagcctcccaagtagctgggactacaggcatgtgccaccacgcccagctaatttttttgcatttttactagagatggggtttcaccatgttggacaggctggtcttgacctcctgacctcaaatgatctacctgcctcggcctcccaaagtgctgggattataggcaggagccactgtgcccagtctattcttctactttcaatctatatttaaacagcatatagttaggtattttattttttatttagtccaacaaatgtcatttaggagtttttagtctatgtttaatgtaactactaatatgattggatttaaatttaccgttttgctatttgttatctatctctattctctgtcctttgttctttccctcccaccttccttcttagtttgggtaaaatttacatttatattagttatacttctttggttctgttttctatgggagggggtcttactatgtgtattttttttttagatgaagttttgctcggtcacctcaggctggaatgcaatggcaccatatcagctcactgcaacctccacctccagggttcaagagattctcccacgtcagcctctcaagtagctgggattacaggtacccaccatcatgcccagctaattttcatacttttagtagagatggggttttaccatgttggccacaaggtctcctgacctcagatgatccacccacttcaggctcccaaagtgctgggattacaggcgtgagccaccatgcctggccactatgtgtatttttaacttaccacaatctaccttcaaatgccaaatttctgcatggtattctcttatttctgcctgtagaactctgaacattgttggtcattatgaactactggcagtgaattatctcagcttttgctgatttcaaaatgtttctattatgccttgatttttaaaagatgtttattttagcatagaattccagggtggccgcattcgtctttagtctctttgaagatgtcatcagtcatctggattgcatagtttctgcagagaagtcggctgtcgttcttatctctgttccttgacgtctgggttgcatagtttctgcagagaagtcggctgtcgttcttatctctgttccttgacgtctgggttgcatagtttctgcagagaagtcggctgtcgttcttatctctgttccttgacgtctgggttgcatagtttctgcagagaagtcggctgtcgttcttatctctgttccttgacgtctgggttgcatagtttctgcagagaagtcggctgtcgttcttatctctgttccttgacgtctgggttgcatagtttctgcagagaagtcggctgtcgttcttatctctgttccttgacgtctgggttgcatagtttctgaagagaagttggctttcattcttatctctgttccttgatagtatgttgtttttctccagctgcttttaagatttttctccttttcagtggctttcagcagtttgtgatgtgtgtcttgctgtgtttttattgtattttttctgctgggtgcttattgagcttcttgggtatcttagtttatagatttggaaaaaaattctggccattatttcatcaaataatttttctgtcctgtcctcctccttttctagggctccaatcacatgtgatgggccttgtggcattattccatgcccacttgtgcttcatttctgtctgtttctccctgtacatcactgggatcacacatagctgtattttcaggttcactaatattttctcttgtcgggtctaatttactatagtctctgccagggtatgacacaggcagtggatgtcagcctgtctctttcccagccaacccagtgtttacctagtagaaccattcacaaactggcactggaaatagggatggagatgagtgtggatcaataacaaagagctgacccatcaacaatagcctaggctgatgcagttatcactggactcccaacatgtcggaagcagggtcccatacttagccccaggtaccattcctgggaaagggactagccagccccctggaggcaggttgatcacactggactcttccatcattgaggggagtatctcttggtttcagtttcccttattccagatatggatatgtctttgctgtcagtgtttctgacagcaccatctgtgggcttgatgaatggcttattcactattgcccacctcctatggcattgttaaggaatttactttacagcaaaggcagcatgggaacaggtacacacttggaatccctacgtttcccatcacccagaggtggcgagtctgattaagcatgaggatgacttcttgaagctttaggtgtgctgcccatggggaggcagtgccctagaacattggactgccatcctgatggatgacatagtgcctcgaactgtggaccaagacatggcaccatttcatagccagaataatgtctgtaatgtcaatgggtggaattaggactggccctgctcaccaccacatccaacacttactgcagggtggctggtgctgctgttcagaagatccttcacctcctttgaggggctcccttgcaggactgtacgtctctggctgttgaattcagctgtgaccacatggcttactgtggccaatgaaccaggagtgggcctgctgtattctgcgtctgtgcagcagatatagcatctaggcaggcttgcttgtcacctcttcccaaagccatctgactggctgagtcccaggaggggctgctgtgctgggctggatgcagatggtgacatggagcagagctgcagctgacctgcctgggagagacgggcgaggaccacatctgtttagctgaccctgacattttattggtttacatttgttaccgcagcatcaatgatcccaggtgggctgataaacccattcacatatttttgcttttctcgtctgtgacattgggctcttttgctttcaaggtcctagttcccaaggctggaatgctcttacctgggacctcagtgatccatctgctgcactgaaagttcagacggtcatcagctatttggggctgctcatgccacttagctaatagttcaagaaaagcatgctgtcttggtggggatggctaaccccaatgaaagggctatcttggagctcagtggattctctaggtacttctcagggctttcacgactaatattgtgactaataaaggcaagacgacaactgcatcagacacactggaatgaaaatttgagtcctctcactgggtaaagaaccctgaccatccaaagtactgcttagggtcaagaaaacgtggaatgggtagtggaagaaggaaggtgtagctgctgatcacagcctcacagctagccacagcatgaggacagtaccgggcatgcaggctgtcctctttgcttgctgtgtgtgtgtgagcatgtgtgggagcatataagtgtgtgtgcattagtgtgagcatgtgcatgcatgtgaatgtgtctatgaagacgtgcatgtgaatgtgctcatgagcatgtgtgtacatgtgtaagcctcagcatgagtgcatgtgagcatgtgtgtacatgtaagcctcagcatgagtgcatgtgagcatgtgtacatgtgtaagcctcagcatgagtgcatgtgagcatgtgtgtacatatgtaagcctcagcatgagtgcatgtgagcatgtgtacatgtgtaagcctcagcatgagtgcatgtgagcctcagcgtgagtgcatgtgagcgtgtgtacatgtgtaagcctcagcatgagtgcatgtgagcatgtgtgtacatgtgtaagcctcagcatgagtgcatgtgagcatgtgtgtacatgtaagcctcagcatgagtgcatgtgagcatgtgtacatgtgtaagcctcagcatgagtgcatgtgagcatgtgtgtacatatgtaagcctcagcatgagtgcatgtgagcatgtgtacatgtgtaagcctcagcatgagtgcatgtgagcctcagcatgagtgcatgtgagcgtgtgtacatgtgtaagcctcagcatgagtgcatgtgagcatgtgtgtacatgtgtaagcctcagcatgagtgcatgtgagcacgtgtgtacgtgtaagcctcagcatgagtgcatgtgagcctcagcatgagtgcatgttagcatgtgtgtacatgcataagcctcagcatgagtgcatgttagcatgtgtgtacatgtgtaagcctcagcatgagtgcatgtgagcatgtgtacatgtgtaagcctcagcatgagtgcatgtgagcatgtgtgtacatgtgtaagcctcagcatgagtgtatgtgagcatgtgtacatgtgtaagcctcagcatgagtgcatgtgagcatgtgtgtacgtgtaagcctcagcatgagtgcatgtgagcatgtgtacatgtgtaagcctcagcatgagtgcatgtgagcatgtgtgtacatgtgtaagcctcagcatgagtgcatgtgagcatgtgtgtatcattgtgtgagcatgtgtggatgtgaacgtgttgtgcatatgtgtgtgtgcacgtgagcctgggagcatgcaagtcatttgttaattgattactccttttcccacttctcagcatccccactattttacacagggaatattagtggtggtagatggtgaactttgaaacttaagtttttcatgtcagagaatgtccagatgggtttgtggctgtaccagagaaaccattagtggcgaggaagactcatgggtctctactgtttctggggaaggtaaaagcacctgtgttgggtggtgtgagtacagcactgtgttggaagatgtctgagtgtgtgacttacttgtggacacagagcagctatgggtgggcagtgctggtccttcactaccccgtttgccctcagccccacttctcacattcccgagctctgcttcatggcccagggggcctctcctcccagccagcttctggttaggatcagcatacggcagcagcaggtgcagctgtgtcaactcttgcgtgaacaccgggggtgggtttgctttttcctgagggtcccaccttcatttctcctggctccaatgctgagtggagaactgaacacacagaagcagaggcatgttcgatgacaaacacgtgtgctgcaaaccacacgagttaccctgctgtgcacctccctgggtgcacccgccgggcacggccaccctgcccacagccggcacctggagccagacgctcctccttctcggtctcacaccagccgcatcttagcagcagctgccccatccagtgctgatgtgagatgtgcagatctagtttttgttttaacccttgctttacttgaaaacttggacatattcaatcaatttcaacggcagtattcatgtattaaaaatggcagatgcatacaagcatgaagaagtaaaaattgctgttgcttgaaataatgtcattttggtgcacattctctctcactgagtgaggtcagactgcacagctccctggtagcccatgtctgagacttaccttcctgaacaacgtaacgattcatttttcaccctcccagtatcccccataattgtacacagggaaatgagtggtggtggatggtgaactttgaaacttcgtttttcaaagtattaaaaaggagaaacacctgcagattggccatttgcaggcttttctccttaagtttttcttccatccttgacgctggtggagacacaatcttgattcttcacgtgcagttttggagagtgtaacaggtgagctcggtcttttacctctagttccaaaccttttactgactcagtaggactaaatgccagatccatttttctctctgcttgacaaaacacgcttaaggtgcatgtgcttaaaattgagaaattacgtaagtcaacccaccaggcaggcagccagccatccatgtttcaactctgtaagatgtctgctactgtccgcaaaataccagaacactccggagcgtccctttgccaccaagacagctttttctacagaatttcaattcttgtgttttccaaacatgctagaaccacaccaagaaaatctcatctggattggctctcctctcacaatgcctgttgttgtctttctaataaagaaccacacaaaacaacatcattcccaagtatagactttatgttttacttattccattgaaaatcccaagttccttcatggcaaccctccctctgccctcacaccccagcaggtccccgcaggccagggcccatgccccacctgcccacgggtctggcccccaggccaccaggggcccgctgcgtcctgtctcacacacaaggggtttaactcaacgctatgtacattcacagttccgaatatccgccaactctaagtcgccacgaagagaaaagagaatcaggaggaacaaacatccattcaaagtctgtttatcagagatttttttttctgaaaatgcacagtggcatttcattcaaaaaacccttcatctgcagacctgcggaagggaggtggcctgggtcccttccctcggaatatcttagtttatttacctccttcattggccatttgcaggctcttctccttgaaaaatgaatctttacgcattctccaattataaaatcagtgactgttagctaccaaaggctgcactaggatttcttctgtgtccaacacgccaagagccctgaaattgacttcggtttactccatccctgtctgtcctgctggcagtgtccatgctaaaaaacaagtcgaggtgattgattgaaacggagctgaaggttgtttttaaatgtctgtcagtggagaaacgcgtatcacgaatctctgaggaagttagtaaacattttttcccgtacttactggccttggtggtcactttctatagagatgccccaaatataaaaatcaattcatttcagaaatcaaaaaaattttccaaacaaacccggagcctttgctttaggaagcaaactcaagtctgtgatgattattgtgcttgaaggatgggtttctaattctgtcataaaaaatgatgaaacaaggtgatgtcagagggacttcaggtgacaccaaaataccccatccagggcctggaagcccctctgcctttgactctaagatggggcaggacccccgctctcagtgggatgtgcccgcgaccctggcgctgggaggcagtgctgcagagaggagtggtgtccgcacggctgtcaccagacatcctcatcaccctccgcaccgcacagcccagcgtctccttccacaagcaaaagactgcggctcctctctctgcaggaaggtcagccatactcgggtggcagcgtgacgccagtcaccatggaaaccagggctgacaggaagatgcagccgcttccagcagggacctggcctgagggcaagggaggagcggcacggcggcggctgccttgcgcttccagctcgggccctgctcatttgaacaatatccaacctcggactggagtttaggctacagtgaaatggattacgttatgccgaaacagaaacatcaaactgccaacgtttataaaaaaagaggggactggaaaaggattaagaaaagcagaaaaagtgttttggggctaggcccttgagttcacacctccccaggggacccagcggaaggagagggattacctgctggagagaggcctgatggaaatttaagtcaaagcccaaacagcctcagattctatattaaaaaaaaaaaggcctataaataaatatcctttgactctaaaaggaaaaatgaagtagaaaaacaattttcattttttaaaaaatgctgcatgtttcctgaagtacctaaatggagtttataaaatactcatgagctccagcacacaggtagaaacactgaaaaatcaggtgacggctcggctctgccaggctgtggatccacagaggcagacaggtttcgtgccagagtgacacgcaagcgcacgctgtgtggcaggcgtgtgctgagtgtgcacggaggcccgcccggtgcacgctgccgccggaagaggctgtgacagagactcgaaaccagggccccgtgggagaacacggggcggcaccggtgccccccgcccggcagggaacctgaccccacggggtccaggccaggcgggggatccgcgtgcgcgccctccgattctcgcacagtaagcgtcggttctgcaacacacttcacaaaggaacaagatacaaacagcccccgaggcccctggaactcgggctgaagacagtgcctgtacagcaggagtgaggagcggagcccagaggtcactcgcaaagaggctcatggcggtgatgtccacgcgggaggggccgcgtctctccgcaaccctgggtcctaatccagcttcttgcgaacaggcttccgcccagcgtctgagttttccgggctgaaatcggcacagaagccgtttgggattggcgcaggtgaggtctctgcacagctttcgaaatgtgtctgatccgggtacaggtattcctctgcgaaggcagggtacatttctgcaaacctgtggaagtcagctggaaagacaaaggcacctgtggttatggaatggggacgagcaaatgtctaggatttggaggctcttttcccacggccctggtgggtgtccacctcacgccctccagggagggttagaatcgtctgtgaaacggatgctttcagggtggagagaagaacttgcgcttactttctttgcagcgcacgaagtcaccctgacaaccggggcaggctgggtgtcctcctagctactcctgcttttgggaaaccctacagaaagcttctggttttgaattcgacagatatatcccggggacttagcacttctgaggcactgtaaccagagaagctggctcagggacaagaccaagtgtctggacagtgatctggcgctctgagggggtgccctcgtccaccctgggagacccggtaggcagggggccgtcctcacagagcctcggactgcacttcctggccgctgtgcctggctctcatgtgagcgccctcggctcctactgatggcgccatggaggcttctggtggggctggaaagatggtgcagggggctggtcatgctaggccgctccagaaacactcagaacctgggccacccggcgagaaatccagggatggttgttactggaaattgcagctggtcggtgcccactacaaacatcaaagagcccgaacacaccaaggagcattgcacactttcaaaatggaaacaagcgcacgcatgcacacatgcaaacacacacacacggtaaccaaacacacatgtgcgcacacacacaaaacaagcacacacacggtaaccacacatgcgtgcacacacacacacacaaaagcacgcacacacacggtaaacaaacacatgcgtgcacacacacaaagagcacacacggtaaacacacatgcacgcagacacacacacacaaaacaagcacacacacggtaaccaaacacacgtgcgcgcacacacacaaaacaagcacacacacggtaatcacacatgcacgtacacacaagtgtgtgcacacacagtaaacacatgcgtgtacacacaaacaagtgcacacacacactaactaaacacacatgcgtgcacacacaaaacaagcacatgcacacacatggtaactacacacatgcacgcacacatacaaaacaagtgcaggcacacaataacatgcacacacacaaaacaagcgcaggcacactaaacacacatgcgcacgcacaagtgcacgcacacacacggtaacacacatgcgcatgcacacatggtaaccacacacatgcacacacacacaaaacaagcgcaggcacacgcggtaactaaacatgcatgcacacacacaaaacaagcgcacgcgcacacagtaactaaacacacatgcgtgcacacacacaaaacaagcacacgcacacagcaactaaacacacatgcccatgcacacacacacacgcacggtactaaacacatgtgcgcgcacacacaaaacaagcgcaggcacacagtaactaaacacacatgcgcacgcacaagtgcacgaacacacacggtaacacacatgcgcatgcacacatggtaactacacacatgcacacacacacaaaacaagcgcaggtacacacagtaactaaacatgcatgcacacacaaaacaagcgcatgcgcacacagtaaacacgtgtgcacacacacaaaacaagcacacgcacacagcaactaaacacacatgcccatgcacacacacaagtgcacgcacacacacgcacggtactaaacacgtgcgcacacacacaaaacaagcacaagagcacagacacggtaactaaacacgtgcgctttcaaaatggaaacaagcatgcacatgcacgcacacacacaacacacatgcacgctttcaatactgaaacaagcatgcacatgtgtacacaaacgtgtgcacacacggtaaacatgcacactttcaatactaaaagaagcacgcacatgcgtgcacacacgtatgcacacgcacggtaacacacgtgcactttcagtactgaaacaaacgcacacaggcgcacacgcacacacacgtgtgcacacacacggtaactaagcacacatacatgctttcaatactgaaaccagtgcgcacatccgcactcccgcctccggctcccgcacagacgctgtgcacgcagctctctcgcgtgtcttctcgtttgctctaagaccacccttccaggtggggattgcggcatccactgggatgagccaggccagggcctgcagccctgggagcctcgggtggatgcagcgtagctcattcacactgcaggggagggaggcgccccgggctcgggtttgcggattttgcacagcaatttaaaaatatcaaccgccattaccgacagctcgtgctctggaatcagccgtgttaccagtcacctcatcagatgtccacagacaggtgacttttctcgggaggaagtcgccctgggcggcacgggggtctgcagcgcccgcctcctcgtctctcctgacagacccctcccaggagaggggggatgtgagccacaccccggctggcagtgacaaagcccacagccagtggcctcgagggctgggtgcgggtatcccccacctccaggcacctgaatcccagcctgaggccatggaaggctcagagcttcggaactgggcttggagtttggttgtataaatcgccaaaacaaatcagattggaaagctccctacgaacgctgccaggtctccagaaccttccttctcaggggtttctttgttacacagggcagcctggtgaatggaggcatggcggggactccactcctgtcctgggctcccacggagactcgccccacactctgtccagcccccgcccaagggtcgcgaggacgaccccactcctgcgggctcaccgaatgtgatcttccccttctcctcttggtcaatggctcggaataggtcggtcacggtgagctctgccacccccagggccgtcttgaggatgcaggacaggtcaccttcgccgacgctgccgtcctcttgcgctccgtacatctgcaaggcaaactgggtgtcaccttgcagcctcctcccctgcccaccaggccccgctgtccagggacaccccagcggccctgccccgctttgtcagagctgctgggcacctgcagggccggcggctggacacgggggactcgaacttccatgtggagccatgcagcagggccctgtgccttggatctggccccctccaggtgcctcgtggacacacagcagatgcttctcgggcaagggctcagacgctgcccaccccacccgtgatagctcagcaaaccaaccagcacctgtggctgccactgggaggccgcggaagggtcctgcctgggccacgggcgagcgcggcgcaagcttgggctgcagagttcccccacggccctctgcctccgtgagtgcaggctacagcagacgtgttcacaagcatcagtaataacatgaaaaagccctgatggttagaaatcgacagggcagatcaggagcagaaatgagaaggacctgggagggccgggagggctccagcggggagcgggaaccaggacccgggctctctcctgagcctcacaggtgacgcctcctctcttcctcctgctccaggtgtggaagcggcggctccggccctctctgtgcagggcccagtgccgaggcgcctgccgcccgctccccacaactccacggcgctcccctccctggctccacagactctgtgccctggagatcccccagaccccttgatcagggcccgcccatccctttgtccctctacctccttggaaacacttcggctccctgtggggtgcagggtcctgccccagccctttaggctcgagcctggcccacgggctctgtggcagcgctttcctctgaggtcccgacgccgcccgtgtccttccgctccaacccctgggctccctcagatgggccagctcttctacctggttcttccccgagtcctctgcctggggctgcaccatcccccgccgccctctctctgcctggggtccacggttcctggggctccgacggggccgtcgccctgactccgagtccccttctcctgctgcagcgcgctgtgatcttgtgagcgtttgctccaccctccgagaagtgggctgcctgggaggggcgtcgcctctcttcagggaggggctgcgccttcacctccaccggctcctccttgcggtgctcagggaagaaacgctgacgcctccagccttccggcctggcaagctgtccccgcgttcacaccctcttcccggggctctgcctctaccccatggaccgggtctgctccaggctctggagaccccatcctcaccctccactctccctcccctgggcctcatcccacagcgctgccgcacgcggccaccctgtcttctgtggtctacgtttctctcctgcttttagatttgctgaatcaaacatctgcagacgaaatccacgttggtgctgctggagcctcagaacgggccgggtcaaagcttcagggccacggcctccagctcctcggccgacctggatcccactcagcccccagagagcagtgcctcctgccccctagtggttgcaaggctgcttcagaggggacaacctgacatctgtgtccatttggacatgagaactgcagatgcagactttcatcctcggagcctcgtgtagatgctgcacgggaaacgcagaggtaggactgaggctaccacaggcaacagacttcaggttctcactgtccactctctgagccgctggaagttttaccagggccacatcttaaggttttacacagtaatgataacgaaaaagaacaaaaacaaggggccccaaggcgtatgaagcacatcggtgcgctgcgcatccggagtcctgagcaccggcgtgtgcctctggggtcctaggtgctaggagttctgtgtccacagtcagcctggggggcagcttcctggatctctgtgtcccaagagcagcacgctggtgcacttgccacgctctctcacacgagactgctccgtggaagcccttcctgctacatgcagtccccccaaaatgtgccaggctcccagagcagcagcaaagcacaatggccatgacttcaggaaatgatgacaccatgaaaggtataggctgcagaaagggacatggggaagcagcctgcaagaaggtgggaggaaggcagggcatatgccgtcagggcggagctgctgagggtggaggtgaggcgggaccctggtgtcgccgtggggacgcagccatatcagcggcctggaagcctgtgagaagtgatcacccatcctgcccccaggtggtcactgtagggcagcctaggcctcacccatcgcctggttcacctgagaaagacctcctagtggcacataccccaccccaagcagggacccggtgcacactgtcctccccagtgggcgtcctcaaccctaccacagccagttaccctgcccacctgcaggtggggcctgtgcaggaggaaggtcgggctccacagcccattgttcatgttgagaaacaaggcaactgccacccccaccgctgctgtcattcgttccaggttatacttggtggctctacagcaggggctggtggggccagcagaacggggggcggtggctcatgcctgtaatcccagcactttgggatgctgaggtgggcggatcacttgaggccaggagtttgagaccagcctggccaacatggtgaaaccctgtttcttctaaaattacaaaaattagctgggtttcatggcacgagcctgtaatcccagctacttgggaggctgaggcacgagaatcacttgaacctgggaggcagaggttggttgcagtgagccaagattgcaccactgcactccagcctggacaacagagcgagactccatctcacaaaaattaaaaaaaaaaaaacaacagggcacagtgaaagcagggccagcaggggccgcagggccacagggcctggctcagatcaggggccctaggaggcaggctgcccaggcctggaggaggcagggaggtcaggctttcttacacagagctgccaaatgttgaggtcatcgagttcatcctgcctggggagacccccacaagccaccttttcatgtggggacattccccagtgttgctatggagagggtgtgctggcatcagaagcaagagaagggagttcttggagcggtgagagacatgcagcttcttgactcacacatcaggcccttgagagtctggctaatgtgagtcacaccctgtgagaagccttggtaaagttgtgcctctgtacaggcaaagccggcccagctctatcccagatgaaatccacattggtgctgctggagcctcagaataggccaggccaaaccctcagggttacctggcctcccgccctccaggcctgacccagaccccactcagcccccagggagcagcgactccctgtggtgaggcaagtcctgagggcttcccaggagtctgcagctgggctgggaccccagactcccccagtggtgatgactgggctctgacatgttgtagcccctggggttagctgaggtgggcagctcctaagccgtgctttcttcaccagtccagtctgagggggagagaggacaaggtgtgtggcccctgtgttaggtgccctgtagtgtgtgatggtgtgtgacaccaccagccccacccaggtgccccaggacccaggaagaacctcagccatgctttctgatgagtgtggattgctaatccaggaagaaataaatctgggagttttctgggcattttacaaacaacagctagcataaaaagcttacgtaaaaatagcagttgggcaaatgatcaattaactgatattataaggaactagagaaatgaacaatggtgctgacgtgactgcaccgccctgtcccccagtcaaacccatgtgaactctgcactcaccttgaaagccagctggatggtgtccagggtccgggccggccggcagacgacagacagggcaaccacacactctcgcaggtccacctcgccgctgccgctctgtggggagagacgctctcagccacagctcggccgccttcggcactggagaaacgccagggtttcccatgggtgctggtgttaattaaaggccagtcccactctcactcgggaacatctgcccatgtgaaaagggaatcttggaaggacattccaccaagtgggttctccaaggtcactgtgctgtgtacacttcttggggccattacagtgacttcccaaagttcacaaggctgccacaggaagcacctcagcaaagcacacgcgatgcaatgcctgccacctgccaagacctgcatttgagaataccccctaagcaccaaggctgattctgggctctaggaatttggtctgtttatggcaacccattcatcctgcagaaagaaggtgaacccggagctgtgacgtggagctaaacagcagcccgggccagctgtggaagccagtcagccccgcgctgaccattctcagtaactcctcacacccagagtctccagggacatgctgatgtgagtgagcagggtggtgagaggcacaggtgggggctgacagggtctgcacttctgatgacgatggggacagtctgcacgtggctgcagggtgtgcccaggccacaggaaagaacctcccaggtcagggcgggaggaggtgaggagcaccctggacagggggaagactctctggtcagacagcaggagatgaagggcaaccaggagtggagggaggactccctggtcagagagcaggaggaggttccaggaagagagcagcaggagcatccaggggtagagggagggctcccaggttggagagaacaagagaaggtggggagcacacaggataggggggaggactctggccagacagcaagtgagaagcactcaggacagggggaggactctggccagagagcaggaggtgaggagcacccagaggcaaaggaaggactccctggctggagagagcaagagaagatgaggagcacccaggagaggggaagaattttggccagagagcaggaggtgaggagcacccaggacagggggaggactctggccaggcagcaggaggtgaggagcactcaggagcagagggaggacgctctggtcagagagcaggaggaagtgaggtgaggagcacccggggcagaaggactccctgatcagagagcaggagataatgagcacccaggatgggggaggactctggccagagagcaggtgaggagcacccaagggcagagggagaactctggtcagagcaggaggaggcgaggtgaggagcacccaggggcggagggaggactccctgttcagagagctgggggtgaggagcacccaggggcagagggaggactttgaccagtagttgcccctctacgcttgtgtcaaccacaggctgtaggtgctaggggtgagacgcacacaggtccctgaaggtcttctgagggaactgatagctttctttgtgctacgccacaccccttgcaaggcatggggtactgggactggggcttgggtgtctggcaggtggaaatgactacaagtccctggctctgggaaggtctcgggctgggggtctgaggtcctgggtgtaaatggagaatactcctatctcataagatcatgtgcatctgaaattgcagaaaataaaaaattaactgcacagtgtcataagttacctcggaacgctgcctgtgaaaccaggaatgctgcctgtgaaaccagtattcaccatttcacgtttcaattccaacactgtggtaaagcgcctgagtgcaaacgcagctgcagatgtggcacggacaccaggagggactcgcatccgagacgctgtttgcagctgcctcgtagtagagggcccgcggctgggggccccttacggacactaggcaggactcgcatccgagacactgtttccagctgccctgtagcagagggcccgtggctgggggcccctctggtgcgtgtgggagaatgccctgagccacgcactgtgatccttggttcacactctgcatacttgcggggcagcctccttcccctgtcctgggtgctcctcatcttctcttgctctctccaaccagggagtccttcctttgcctctgggtgctcctctctcctctctcctgctcctcctgctctctggccagagtcctccccctgtcctgagtgctcctcacctgctctctggccagagaggcctcctgccagcccgggttccacccaggactgcactgcagctgctccgtgttggtgcaaaacatatatggtgctgtcacacgggacatctcatgctgtcacgggacagtcatggctgaaggagccttctacacacaccctggcgtatcaccagctcctccaggaccacagtgtaagtgtccatgctgtgacactaacacccagaaaagaccaggattggctgacctgtggctcgccgtggcagcctgtcccgcccctgcgtgccacctcgcaccttcctaaaaccccgaggctgcccgccatgttcagggccacctggtgtgaccgtggatgcctcatcaattacgacgaggacctggggatgacgtgcggagggcagaggggagtgggcggctgcaccaggaggaagccctcccacacccacaggctggcggggagcctctaagccgatcatagaggacgggcgtgttattagaaactctgacgctgcaaagagcagctctggtgagcaggggaccctgcagggggtcccttggagaccccaggctgggtgtcacagtttgatattgttagactccgtaatcacactgaaaaaccccaaaccacaggcacattcatcagtgaagggtaatttaaacgtcaaatgtgtacgcgtatttaaaagcatggaaggaaatacctcaaaatgatagggtggggtggtgatgggtggttttccttctctgctttcaaattcatgtgaaaatatatttatattagaatgagaacaatttatgcttcaaaaagcaggaggttttgccgacaccccgctccgcaggcgacaggccctacctcgtcgaacagtgaaaacatgtcttccagcaagtcagaaacggggacttccagggaggcggcaaactccgcaatacctatcttctctcctcccttcatcctggctctttctgagtatctgtccagatctttttcaagcttttctggttttagcctagacaaaaaaagagggaaagctttctttttcaataaaatggtggcatgctaacaccagatttacttctaagactcatcaaaatattaaaattgacagaagtagcagcaagctagttttcaagaaaaggcattctcctaattggaaggtgcgagatgtgttctgtccaagtgtcaagccaggtgcctgtgcccctgagacccgttcccttcccagggcagatgtggcaggacacagggtggccctcctggggcgccgtccacagacagctgatgccagcattaagggattgaaagacactatgtgggcttaagttgatatttgaaaaaagccagaataattaataattatctcctcagttcccctccctgcaaccccaggcagcccccgccagacctcggcatcacgggttctagctaatgctcaaggaagaagaaccaggtactcacccgaggccccgcacgagcctggcaaattctaaaaggcaagtgtcagcggggagacggagctgtccttccgccagggccagctggcagtcctcgaacgtgtagtcagtcacggagacacccaaggccctacaaggagggcagcacccccgtcagcccagcctcgtggcagccagttcccaccgccccaccagaataaccgccgatggctcaggggagaggagggctgaggagaggcagtgagacaggggaagggccagttggtggggcagtcacacattcatcgatcagcttcacctctcgtgggtgctctgaaacgatgacggcagccacaccaaaggtcactgacacagactgccatcacgtgagaatcgtgaaaaagtttgaaacagcgtgagaattaccaacatgtgacgtggagacacgaagtgagctcatggtgcgagaattaccaacatgtgacccagagacgccaaagtgagctcacagtgctggaaaacggtgctaataggcttgctcaacacagggttgccacaaacgtgaagcaactgtctgtgaagcacaggatgaagcagagtgaggcgggctgtgccctcactcccagagctgctctgaccgtgccgtgtgtgctcaggtgatgagaaatcggtgcctctctagtcctagttgagagccgctgaagttccccttggcaaacttaggcatcatcatttggaacaggagagtctcaggctatccacaggaaaggacaaggcccaactgagcggctgcctgtgaagctcacagcagtcctggccccgccacgtgtgactccaggtgagccagggaccaggagccctggctgggagaaccacccacctccgccagccacacctggggccatccccggcctctcctgatggcccgtcctgggtggcacctctctccaggccagcactgcacacgtgccattcaactcagaaccccaaatgactcagaggcgagccccatcccagatgaaaaccaaggtcgaggaaggatgtgtgcgctctgccaccctggggtgccaccatccttgcagcgggcaggggaggagaacgtgcttccaacactgcctcctaggacccactacgggagtgggaaaccagccgggaggaggcgctgccagggtccctctgctcatgatcaactccactccccgagtggggctgcacggccccgcccaggcccaggagtccgaggctgcttctccctcccatctccactccgagtggggctgcacggccccgcccaggcccaggagtccgaggctgcttctccctcccatctccactccgagtggggctgcacaggccccgcccaggcccaggagtccgaggctgcttctccctcccatgttccttggtggggggttttgctgacggtggcagaccctccaacggccctgatgtgatgaaacatggttcccggtaatttggagtggggttttaagagaattctgaattactacaatggaaatggcagcactctcgtaccctaaaaaaagtacaagttcctcggagtctgtttgaaatggagggttctaaaataatcagtgaggcccacaggcgatctctccttgagcgctgtaaaatctgttctcttcctcctaagtccccaaaagtagccagtattggcagcagtccggagcacaggtgctgggcttggaggtgggaatgcattcccctggctcggcgcctactcagctttttgtattgttcagagctgcctgtggtgggggttgagtggagctttgcgggacagagactgccgggcccatttctaccctcggacacatgctttgggagggagaggatgggaacgtgagggaacgggccgcccagctgaatcttcagggtgtcagcggagtgtgaacaaagtggctcggaggtcagagccaggcacactctgatgggcccggccgtggctgtgttcccctctgggctctctggagaggcgagttcccagccatgccccgacctgctgcagcaggagcctgggggtggggccggtggaccgaggctgatgtggcttccaagtggctcctgcagctcaggtctggcagccgcccggtggaggggaggctctagctccactggacagagctttgcaggaggaagcactagggaggcattcacgtgggggtaaatccaggtgggaggacctggttgcaaggacaagtgtggggggaatgggacctgagggatggaaggtgctgagggagggagatggggagggcgtgtgagctgacgacaccgagggagggagagggggagggcgtgtgagccgacagcactgccggccatgcaagcaggggacctgggaccatgggaagcgaggacattccctcttcctaactgcatgcacgacaactgtgtaagcagcacaagtcaccttacaagattaaatgtgcaaccaccactccactctgccagatagagtaagggcaccggtaagtatcacacaggcagatgagcaaaggtaggcgtggaggccgccgcacagatgtgaagttggcccaggccagcgccccgctggctcatttagggcacaggaaacataggacctggggtgaccaacggctgcaggcaggtctggaagaaccagtcatgcatcttcccaacgtcaaagagggtgaaatgccatcagagggaaacaaggggcgcacagcacaaggccaagcacgcttcaccaacatgcatgaagctggttcccgcacttctgcaagaatgccttttcctaacgctgttgcctattttaaatatacagagagcagcactctgggagacacccctgactcggcgatgggggaaggagctgctcacttactcggccatgactcgccgcacgttgctggcatacagcgcggggttcctcttctcctcctcagaagggctgtacacaggaaggaactgagcacacagagaagctgcgttagtatcacaagacgctgacctcagcaacaccactgtaacgacaactgggaggctgacaaaattaagatctgtcaaagtaacgcccattagaaccgtcttcaaagttgtcatgtgcacgtgtgtgtacgcacacacacacccccatgatgcatgaactgcacacgtgtgcacctgaacactcaccctcattggtgctccctgggagcacctgctgcctacatcagaacatctggctctctgatctacactcacccccgtcagtgctcctgggagcgcctgctgcctacatcagaacatctggctctctgatctacactcacccccgtcagtgctcctgggagcgcctgctgcctacatcagaacatctggctctctgatctacactcacccccgtcagtgctcctgggagcgcctgctgcctacatcagaacatctggctctctgatctacactcacccccgtcagtgctcctaggagctcctgctgcctacatcagaacatccagctccctgatctcacagaggggcttacgcccatttctgacagcatcagttttgtaatggggataatgcagcacgttattcctacatttggaacagattattggcagcgttttgccacctcttgcattttgaaactaaaagaaattgtccagtgaatttgtccttgcctgagtgacacttacacagcattttaaatgaatatgacccacattgcacaccgtgcaggaaagggcacaagactaccctggatgtccttttctctaatacggaatatttcagaatacaggaacatcctgaaaccccaattctcgctgttatttctaaaacttccgtaaactgccatggttccttgcatcaacatttgcatcttgtatgaacaacgtcagtaaggacagagcagttttgcacagccattcactttcttttcaactcttaaaattagaaaccaggcctgtaacacagccattcaacaccagggagccctcaggagctcaagctcagtccctgtgttctgtgctcgaggcctggcctgcaccctcccggccactgcccatccagccacataatcctgctgggtgggggaactgccgccctcaggcccgggccaaggcccttcagcccagtcctctgtaatgagagcagcttatcctctatggaagtgaacagccgggctgaaagccgggcttatcagtatatttcgtagtgctctccaatatttactggaaggcagcaaattgttttattaaatatttaagttgcatgtgactcatcttttatttccagatgcaagaagtgcatattaattccccttttaaacaaaaaaagaactactcctcatcgacttagaaaaaattgtgcaggctacacagaggtgactatttagcccaaggtcttgactctggagacctgggtcaggctgagaagggggatctttgactatgtattaaacagagcatggtgctatgacaaactcagcggtattaaatatgtaaagtaaaactaagagccaaacaaataattatttgcatgtgggccagcttcatatgtccttaagctacaatacccataataatgaatggtaggtttaaaaaatcctttccagctgggtgtggtagttcacgcctgtgatccccgcactttaggaggccaaggcgggaggactgcttgagcccaggagtttgagaccagcctaggcaacaaagtgagacccatctctacaaaaattagctaggcatggcgtgtgcctgtggtccaattacgtgggaggctgaggcagggagatcacttgggcccaggaggtcgaggctgcagtgagcccatcgctacactccagcctgggcaacagagcaagacaaaatgctgtctcagaaaaacaaaaaacaaaaacccaaatcttatccaagtggttcagactcataagagagacataaagtcattgaggtaaagcaaccagaggctcctcgaaggagccctgagaaacggaaagatgggaaagcaagacaggtgatatgccacattgtacaaggcttatctgggcatcctggtgtaagctcgtgtctgcatcaaccactgtgaagagcgctgtgtaactctgtatccatacgaacctcgatttccacttggttgtgaaactgacacagcgtgagccacaggatttccagccttaaaaacagattgcacaatgttgagcagatttacaactcgggttaacaagtaaattactcccaattctggggtgaaacctccagacgcgccctccagagggcgctactgggcagtcaacgctcccacggagggggtgccgtgcccacccagcccgagccccagtgtgacgtacagccgtgggtggaaatgctgcttttccgattcaacacctgcttatgtccttggttcgtctagcaagacaccatctcctgcttgttctccagctcaggagacctcgtacagcccacacctgtccctccacacctgtccctcctcgctgtccccaggcagcacaggccctgagtcatgccacccctccagcctggatccagagttcccaacctcagaaccaggatcaccatgcctttgccgcaggagttgacatgggagacatttggagctgctcccagggccacactcacgcctccgacagcccagtgccccaaccctctttctgccctgtcatgccagccccagccctaggcccccgggaccccagaacccctataccccccagagccccctcccagctctgctcccagctgggagcctccacacgccagcctgggaagcgctgacctcaacaccttcacctgaaagcaccagcggacccacatcctcccaaacgcctggaatggagctgctctctcttggactttcccgctccctctgccctcccgacgtcagctcagacgtcagcacccaggaggccctgaccagcctgtggccccgggcttctcctctggggctcgttcccgtttccgtggggttgttcattgttgcataactgaccttcggtgtctctgctggggggacagggacactgactcacagcaggggctggcctcccacccagcactactcagtctctgtgcctgtgcagacggggtccccccagacacccctaaatctccactttccttaccaggggccaccaggtggacgatcgtaattactgagctggcaaactcgagagccgaatgcaagactcacagttcccttcctgctggtactgaagacgtgcaacttaagaccccagaatccaggagtgtcaggcctgggccacatctgtacgtttagttttcacaatgggctgaacctaacggctgtcccacacctgctttcacaactgcaaaagtaactagcgtgcacagcagaccccaagcagcccctacgtgttcatggaacaacaggacaaagaggacgacacggcgttcaacttacgctccaggtccttgccacgtccatgtgatggtgtcctggggaggaagaggcagggtcagtcagcatggggcctgcacccagggcctgcaccaagcacctgcgtgtgccggcctctccctgcacctcccaccccacagaggcgctgcagccagggggaagggaggagggtgctagagctccagcttcccagagtccctgaggatccaggactcggaccagccccccagcctgaggtccccagagcctctaaggacccagagaaccaccaccttacaggtccccagagttcccccaagatccccagagtccctgaggactcagagccccctcttataggttcccagagcccctagaacctgggaaaagggtcctccctccctccatgctctccgctttcctcacagacctggggacaccaattccagtgtgcacccgggaccccggccctctcctgcccaccgctctctccaactgctctgagtgttcccccaaccacggtgcatgtgcagggcccccccaccccgggtgctctgaagtcccatcttcagcctcagtgccgccgggcagggaccacacagcaggggccgtgacggcagaggcccagacaccgtcaccctggggtggaccttctgctcccctgacggctaagctagtgcttcttggtgcatccagtaatagtgtgttgctttaaacacactgatgaaacaatcttaaaagtggaaaatctagatgactgcacagatcggagtctgttgtcctcgggttgacttttttgcccacaggagttgcactggggcatggtttccgcagcggaggcaggaaggggcccttggccaggagcctggctgacaaacgcacgcctcagtaagaaggaagaggtctttcagtcgtgtgactaccggccctgactccattttattacctgaccgtcagctctccgggcaaagtgaagcccggcaggcatcggacgggacaggccctgaggcggatggcccagaaccctggcacccagaggtcacctgtatttctgccccactatgtcctgacccacgggggttttctttcagtgctttcctttgccatcacaaaaaggaactgctatagacgggaagctcgctgtgatgggcaggccctcctcggtgctctcccaggtgggtgctccaccaggaacctgcagtgagcccagcaccagtgggctgtggggaccttgagtccacctgggccgtctttatgctccaggagcccatgaaggggcaaggacgtgcgggagtcattgctgtggaagggggaacacagctggcaccaaatgaatgccctgagctcggcggcaggcctgtgaggcaaggtcgcctaagtggggagggccgatggaaccggaagtattctgagcatccaagagcacctggaggaaggtgcagcagccaccttacaggtgtggaaactgaggcaggacatgggcaggggcggggccagggggtggggctgcggccaggggcagggatggagcagggcctgggtcagggtggggtggggcggggcagagccagggcaggctggggtggggtggggtgggctggggtggggtggggtggggcagagccagggcagggtggggtggggtagggtggggcagagccagggcagggtggggtggggtagggtggggcagagccagggcaggctggggtgggctggggtggggcagagccggggtgggctgggatggggggagtagggtggggcaggggggtggggtgtgatgggccagggcaggctggggtggggtggggtggggtgtggtgtggtgggctggggcagagccagggcaggctaagcctcctccagaggtggcaaggccccatggggaactgctctgaggcctcgtccagactttctcccaaaggtggggtctaccaccttcgccccaggagctctggaccccaaacaccttcttaattctcttaaaccaaacaaaacctgggcactaaatgataaaaccacagaaatgacaaagtcacctcctttctctccagctgcctccgccttccagaacttaaatgttcttaatttttaaacttcctggagcatgaactgggtactgaaagctgctgggggccctggccggtgatgtggggtggaggggttccctctccaaaatgtcaggtttggcacacagaggaaagggcagctttgtcaggagctatggaagcagggctctggaacactggggaccactgtggaatcatggggttgatcagggacacgtgcatagaacaggccgcactcaggaacgtgccgagcccagggcccgggcctgtcctgccctctccagcgctgaggccggatggactcagcatggccaagtgtgggctgtggcgcagataaagggtgtggagagacagagacacaggtgcacagaggcagctcgcagttcccgtttcccggagaattcccctggaagctgaccccaaaaaaacacaactcaccagtttatttggatatcgtaaaaccacaggctggacgggcgctccagggatgaatgcacctgccgagaaaggaacagcggtgttgcccatggcagcccacgcaggacagcgtctgctgcgtttctcatgctgcccttgggataaaagtgagcttttctgtctaggccttcctggtcagcaagggcactccatgcctggactatctcaacatctgtcctgaccgtaaacacccagcgccacagcacggatgggcaggaacatcggccaggggcgctccccggccaaggaacactttctgttttaacatcgcgcacgagctggaaggcgtctgtggagggacacttgctattataacattgcgcacgagctggaagacatccgtggagggacactttctgctgtaacatcgcgcaccagctggaaggcgtctgtggagggacacttgctattataacattgcgcacgagctggaagacatccgtggagggacactttctgctgtaacatcgcgcaccagctggaaggcgttggtgggaagtgggggtcctcatcaccggccaatgctcacccacctgacggggttagggtctgccctcaccaagcacatacacctgcctcggggccccacccgccgccctcacctgcccctggggctcagctccaaggacgggcggatttggtcacagctgcagcaggacttgcccgggatctgcctctctctctgagtggctgccacctgatgggctcccaaggaatcaggaggagccccagcgaggcccagctagagccacaggccgcagaggctcaggaacttccccgttcccactggaattgtggctgtacccagcagccgctccgccttctcactggccggagttgtgcccttgcgggctggagcaaggcaccatggactctgtggatgattgtttctattttaaattctatttaaatctctattgaaagagtttttttttaactggaaaactcaggcatcagaagtactcaagttttagaacaggacaggcggtctgagaagggctcccctgcctggcgacctgaggccaggcgagtgaatccccaggcagcgcgttcctcccaggctgccagcgagtgccactatgtggtcaggaatttgcggttggtcctggctgcgggattctgcacaattctgggagaatggagcccatgcttgcccctagggctgccaggagcatctgtccctagggccaattctcccttcacggtggcctgaggccagaggcccgtgtcagggctgaaacctcgagctgaccgcccaacccactcagctttcgtctcatgcaggacatccacctgcagccttggtgtccagctggcaccccaagtccctgggcccgtgtgccgcagcttcccctgtgtccagggctgggttgcccccttcctgctgtgggctcagcagcacctgggagcaagctcatgcagttcacccagcacttctgtgtgcccaggtggacgggtggctgtgtcaggggaggcggaggacggcagggctggcacagacccaggctcaagccccagctccaacgcccggcagccgagtcccagcccctgagacacgcagtgtggaggaaatagtcctcattgcctgggaccgagcccaggagctcgcgtggagtgccgagacctgctgtgggtgataatggctctgctgctacagctcagccgaccatcccgaccatgtgtcaaggggacatcactgaaactcagtaagaatcaaaggaaacctcaacaccctggggacctctcaggaaagtccccaagagcacagaaaccccccatgagcctgaaaggtcagtgacttcatggaaaccccggtgggaaagtctgcaggggtctctggcctccctagtctcccaggggcccctgagggtcagcagcacccagaccccgccagcactgtgcacccaaggcaggctgcacaagggccaggctgggagaaaccaagatgaggcagtcagagcccagggcttctgaatgcaggaacaaggcagaggggaggaggtacgggcttctaaactgcacagaaacctccatattcacacatagcttgtgctgggcatgggtctaacgtcactagttgcaacaatgaggctggaagatggctcaacagggacccctgaggcccccagaaggccatcaccccttattcaccctgaccatcccccacagcccaccccacaggaagaaacagtccaccatggcccagggagagccgtgtccacatctgctcacagcctgtgcagggaggccgctgcggcccagggacagccacatccatgtttgctcacagcccgtgcagggaggccggggagagccacatccatgtctgctcacagcccgtgcagggaggccagggacagccacatccatgtctgttcacagcccgtgcagggaggccactgtggcccagtgagagctgcgtccacttccaacagccacatttggtaggcccgcctctgcatcctcgggctgtcagtgccatcgctgggcccacgcagcatcctagggccttctcactgtcttcgttctgctctgagagcttggagctggcaggcaggtggggtgtgggcatgtggagggcgtgggcccaggaccttccaggaggggtctccaggtacccagtcagccgatcctgctctagtgccctctgacaaggagggtgagtgtgggcactggagaggaaggccccacatgggcctgtttcaggagcaactcaatccctgcagagccctgagggtcccagccctgctggaacccaccacatccagcagaccccacagggctctgctgagctctatcacccccacctcagaggcggcacccctggcctgggtcagaacagggggactcaggtggacagtgcagcacagacaagagacactcaggagaaagaaggcacctgggcccaggaggaccctgctggaggctccaggagcctgaggctggaggctgggcccaatgcgggaaggtctccggggatgaaggcggggagtcagcagtggtgcccaaacgggtgaccccagcaacgtgaccccaacatcaggcgatgtgatgaaaggtggagggagggtctgagtgggtgggccgccatggagctggggcagctggagcccaaggcagcaggcaggttgtgggtgcctctgttgtcaggctcaaggcgggagacgctcacggctctggagggctccaccacgaggcgtcaggatcacgcaccacacaccaatttcacctccaaacaggaggctccaaacctcactaagaagtacccagctaagacgcatgagggcctgagagattccgaacacgtgactcgaggcagccggaaacccgggcctgcaggtgacacgcagaactcatggctggacccactggaaatggagacggggaaaggatcaagccaccgtgtcatctccccagaccccgtggaacccacctggggcacagcgagcgcccttcctggactgcacacagtgagctcctactgggcaccgccatgagtcccctcagtttcgtgatatgcgttgagaggtgctcacacgccctcctcacgtctacacaagggccgccagctccaaagggcaaggcgatggccctccccaggggcctccctgcaggcacgaggctccactcccgaggaacacctgcccttgaagacagggcccagcagtgaccccagccccacaccttgtcaaggaacaagacctgacgtacttgccagctgtcttctgcctggggccttggaaacacggcccacttttggccttcaccaaggttgcccacaggccacgcccaggcacggacccagtgtggtgggggcacacggattcgtggtcaccgcgtgccgcctccaccctctgcatctctggttctgatcagaatgactcagaaccacgcgtggtgaagacaatggaacgggaagacccagtaccttccagtgagctgcaccttgaagcctctggaagcaaaacctgccatctgcctgctgcaccaaggagggtggagggtgaaagcacgcgcatctgagctggaggaggatggccacaggctcggaaaccccggcggcacacgtaggtgagtgactgcacactttctcgacccagctcccataaatgtccagacactcaggttgtgcggccccagaggcacggcagccggtgccagtgtgagcgaggaactggcttttccacgtgactctgatccaaccacagagaacaggtctgcatgccccagtatggacacagttgatgaaagccactactttctaaagtagcccaagggagacgacaggatccaggtggaccctctgacacgcccaagggtgtgcagaattccaaacagactcagcgcaaaaagaacagtggactctgctgcctgctcactgccacacttcccacgtcaggggtgcagacacaatactagacccaggtaaccgccgcgggagagacccaagttcacacgcatttgagtcccccctccccagggagaaagaacactgtctaactggggacctggggacctgggcatctgaggatggctcgcagggatttgcacctgggattcccaggcgggtgtggggcgtgagctgcaggccaagaagggtaccctgagccattcaaaggcagtgagaccccttctgcacccaggaccctccagcctggacctcaaaacagaggcaaacatctatctccacctcactggaaacgccctgaaaccgtaacttctccatcttcctgcaaacatgataatccttgactcacattctcaaatttattttgtgacttttcttaggaatacctctaagaaccccagcacacaggataaaaacagaaactgttcttatgtgcacagcacattattaaaaaacatctctttaatattttccttttctctaggagaaaaataaacatttaagaaaactacataccaggtttgaaggtaattaggcaggtcctgtttgtacaagttccttctggaaaaatcattatctggaagtgggagaaagaaaaggatcagcattaaactgtacataattataattcagaacttacagaagccaatcttcacaatatattttcttttctgctgacataatttggacactgggatcatattcagtacgtaaattattaaaatgcattatggaagcacacactgatttccgctctgctggttttattaaagtgtggcagcagtatcaacacataaacagcatctgtcttcttcaactacagctcctggaaagcagggtagtcgcggtgcccacctctcagacactgtgagaaccaggtgcaactaaatataacccacggttggcacaacataagcacctcctgggggcagcactcattctcatcaggaagaaggtggggaggagagcaaagacccacaaatggcagtgcctggctgatggagccgaaggagggctgggaccccagcaacgggctggatgagcaggagagccgagccccagtcaggcagcaccctccacaccggggtttcaattcctctgactcacactggcctgcagtagcaaaaactgccctcaaactcccagccccgcaggctgctttcagttctgggccccgtgaggaagacagacctcaggggggccggcgtgggctcaccctggcaagcccagggccaggcaggccaaatgggtgggggaggagactgcctgtgcttgtgggggcagagctggggcaccctggaagtcacagccctctaaggggctgcccccagccagaccctgcaaagcttcgtgagcaatcagatcccagacagttctgtaaaatttccagcttctaatgatggctcagctggaaagaagaaaaacagcgtccaagctaatgtcagccaggctgcccctacagctgggttcagggcttctgaatgcctgggaaccagcatacagaccaggaagtttactccaggacttttagaaccacccaacacacagcaggctgacacatcaactccaaaatcacactgcaccaggagggcgggtgagcaatgctctatctgagtcagaacttagttcagaaaagatgattcaggaagctgagatgcatttgagaattgtttttctttactttaactttaaatttcgggcaccgaaaccaaatgcacctgacagcaccctcctgagttcacgcactcactaggagaagagaatcagaatccagccccggaggaagggccccagtttctttctccccactcgcgaagttcgcatctttcggaataaaaccactgaaacacaatcagggctacgtgcattacctgtggccactttccgttggactgcgcccgtctcttgatttcttctactgttttcctgcgagaatcctggtctgaccgggacacgaacacaggccgtatatactggatcagagctggaagagaggaggggagacggatcacgtggaatgcacggctcccgccaggcagggctgaggaacgagggggctgctgcatggcgcccagtggggcgggagactccagatgccccaggggctgtgccatgggtgggctgtgagtccctgactgagggaacgggaacagcacccgggcttcccatgtctgtgacagcaggaagccctgtagcctcagtgccacaagtaacaaacagcaagtccatggcacccactgaggacacggagcacagaacggaggtcaatgaaagacaaccgccaggaagcagaaagtctttagacccagtcttcacagaggacagggttgaaggtgccaggtccctagattggaccccgccttccccctgtgggttgaactgcatcccctaaaagacaagctgaagtcctcactcctggtaccgggaatgggacctcatttcgaaacaggtggtagcagatgctcaaggtcccggatgagggtccctatgggaagaggagacaaagcagatgctcaagctaaaatcccagatgagggtccctatgggaagaggagacgaagagggggagagaaggccaatgatgaaggaggccataggaggatgtggccacagcccccaccacgagccgggagggccgagggagctttcagaagagcccaatgcggccagcactttgttctggcacttctgcttttagacggcgggagaatatgctcctgttgttctaagccaccagctttgtggtgctttgtagcacagccccagtttaaccatggcaccccacagcctcctggaagttgcggggttggggtacaagggacaccccgccatgaaggctggtgctcagccaaggcaacgtggccacggcacctgtctttatgcaagaggattgaacgtggaaacatacttcttagaagagaaaactctgaagactttaaaatgaaactatccctgagtggagcttcctttttgaggacggtgagcaggatccacactcggcgccccccaggccaactgaaggtgtctctgaccccaccacgggctcctaggctgctgcaggtcagccccacctccccaccccagttcagtttccagtttcttttcttagcgtttttattacttgtaaggagtagcatttcatgacttttattctttcttggaggttctctcctccaagaacgagaaaattgaagccaaaaaaagaaaattccttctatcctgtggttgtctgttgggtctctgtggtctttcctatgtcttgaatcattttgctttttcacgattccagtactatttcatccttgctcattggttattcccaactctgctaaaaaggacgatatcggaagggaagaatggcggagtcgtctcgcaggatggcgcacgcgtcaaagaaatggccctgattgcgtggtctttcggtgcccactgttttggccaaagcgtcccatggtcgtcccggggcacacggggtctggagatgccaccctgccaccttcgcaatcctgtcagctggtttgttttgtgaacagagtagagatttcagaagttgtctataacggcaaagaacagaacagtgacgtgggggcgtttcacactcagatgggtccgggggtaaatgtggggggtgcagggggaggactctgagtgaagtcttgggccgtgcgtcttcatgtgggttgcccggtgtgcctttcacgctgagaactgggcagtgaggacagcaaggagatgcagctcctccccgggagccctgaggagggaagggtgtcaccacgccgtgtgcacagccacactgggactggcgcattttgccagcaagaaggtgacagcgttctgccgtcttttgggcatttgaggctcaaactttccttgacaaggtttgtaaatggatggtgcggatatgaaggtcactgaaaggaaactaagaatgtgaaaaggaaaaaatctacctggttgatcactttcattggtattcatacatctaaaaatgaaaatattttgaacattttcaacattcatctcttcaacaaagttgtgagctgtcaaagggtccacgaaaggaaccagaagtcaccacaagctgggaccggggagatgtctctgagctggaaacctgggagacgtctctgagctgggaccaggggagatgtctctgagctggaaaccagggagatgtctctgagctgggaccatgggagatgtctctgagctggaaaccagggagatgtctctgagctgggaccaggggagattatctctgagctgggaccacgggagatgtctctgagctggaaacctgggagatgtctctgagctgggatgggggaagattatctctgagctgggacaaggggagatgtctctgagctggagcctggggagatgtctctgagctggggcctggggagatgtctctgagctggggcctggggagacgtctctgagctggggcctggggagacgtctctgagctgggacctggggagatggttttgagctggagcctggggagatggttctgagctcccgaatcagagggtgctccaggtgcgcagtagaggagtcatcagccacgccccaggacaatggccctttcaggccaggacagcagggagcaaaatgtggttcctgctaagtctgaattctaacgaaaatttctgataagcttgtcactatcctctccctcgcaggtccgtggattatctctgaaatgacaagaaacacgcagggctccacgagggccctgcacacagcagcatggctgtggccattcttcctgctgcgccgagatgaaggtcagtgctgggtcacctgggttgatctggaagaggtggaatcccgggcctctagaaagataggactacaacgtccagcatcaccgcaccaaacactccttcgtgttggtgatctccacagggaggcaccaagggcacagggagagcccggccaacgctcagattcactctgggtgggccagcccccctgtccaaggacccctgcagctgcgagggtgcctgggcacacagctccacaatccccctccctgagcagatgacgaacgaccatgggcctttgccacagtgggggggcaaatgtggtttcctgggtatgccagaaccacaggatgcaaagggtgctgagtggagctggggctgccccatgagcctgtggagcttgcagagcggggaaacctgcgtgtgagagccaaggccatcactcggatggtgcctgcagtccagtctcgcctccagtgccggcctgtccagcctcctccaggcttccccagccagccgagtgcagtgagccctccagtcccaccaaggaccatccccgatttccaagcctgacccaggcagactcaggccacccacaactgcccagctgcaggcacagtctggacaccttccggctgctgcaggcccaatcccccatgggccatgccagggttcctcaccccacttccagccttcctatcccagctgtggccaccaccctcagtggtcagcaccacagaatgtgtccccacccagtccatactgaatgtgtccccccagatattttctagaaaactgtcatatgaatgctaacgttaggtgctgcatggaaacagagccggcaggagtggccaggtgagggccccggccataggcgacgcccctcgtcccagcagctccttccctcagcaccgcccactgccaacccaggacccgctgtgccccagaacgtgctgtgctgtgctgatggcggctgtgaaggaagcacgaggacagaacgggcccagatgccctgaagagcaccgaggacagagctctggtgggtcagggctgcgcacaggtgggtgaggggccacgcagggctccctacagtcctcccagagcagtgggtccatgggatgcggcccccaaagatgggtccacatgctgaccctggaacctgtgaatgaggccttatttggaaaaaggtttttacagaggtaactcgttaaggatctcaagatgagagcatcctggatcacccaatgactggtccttataagagactcatagaggagacacttggagggagccaggccagtcgaccactgaggcagagtcagagggacgcagcccccagaggaggaagaggcaggaaggggccgcacagggccttcggcccagagacgccttgattttgaacatctggtccccagatgacgagagggtgaatctctgctggagtgagcccccggctttggggaggggtcatggcagcaccaggtgctcaggtccctggttcacggcacagccgcggtgcatgggcagccctgccgacctgctcccaaagacttccagaactgccatctgataaaggtgagccacccttgccaagcagacctggagcaagcactgtagcctgttatgatagtgagggcactcaggtagcaataaaaataacaattacaacaacgaaaattacacaggaaaccccaacacagaaaatggggcacaaggccaatgtgcccggcggtgggggctcgatcactcccagcagcgtggtggggggggggtccctcactcccggtagggagggtctcactcatttccagcaagtgggggtccctcattcctagcagggagggatctctcattcccagaagggggggtccctaaaaaccagcagggagtccctcattgccagcaacagagggggtctcactcattcccaacagaggggggaccctcactcccagcaggaggggaatctcttattctcagcagcagggggatctctcactcccagcagggcagggtccctcactcccagcaggggtgtctcactcacttccccagatcgggatgtctctgctctctgccttcatcacgatggaggacatcgtcatggtcacagggatggcgtcgaagtaggacgagtgaggcgcgagcgtgaggatggccgcctcggtgggcagcgcctgccgccccttcacggccacccggtggaagccgccggcgaaccacatggtgcgcatgatggccttcagcaggaagtccacaaccctgcaaaagagggcgctgcgtcacgcgggcacacgtccgcagtctcggagtctgtgtgaggcacaggggcggtcccacgggagagccctccagggcgcagtccaggccacgggcttcctgtggtcgccgccgctgggacatctgcttgagggaagaaaagacgccgcgccttcctggcctccgcctgtgtcctcgctggctgcgctctcacctacgaaggaggccgcggggccctgtgtggtggtcacgggccacgcggcaggcagtgctaagacaacatgagactccggaacacagacagcacaagtcccaggccagaagacctccggaccttcatgttagcaacttatttatcagcatcctatcattttgaaatgggaaaaacattaaaacacctaaaacgcatatcgcaatatgggggggggggcgctcagagctgagggcggaagctgagacctgcgtgcgaacttccccatctcatctccacaggaagcccggggttcccacaaccaggcgttgtgttattacagtgagctgtttctggaggaaattagatggccaataaatttggaaaataaagtaaaaatcagcctaggttaactgaatttgtgtactctttatctctttttaaaataggtacaaatttagccttttaaatgtgtgtattctctttcatatgaggctacagattaggctgttattccacacctcagggaattgacacgcagcagacagccacggtgtgtgagaagccacaggtcaggcacacagagaacatgattcagcctcatgagaagccacggctcactcggccactgggttaagtgggcgcatcacactgggtaaaactactgcacagagaaacaagagcctgaagccttactggattcacattgccctgggacagatgatcctcccagaagacttccaggacaggggtccccatggatctgagccaaggcggttgctaggagggcacaaagcctcctctcgtggggccaccttggaatggcctttgtgtatgcgatgataaacaatggtgtagttacacatggagatgctggggaccaacccagggtgctgcttgtctagaggaggggggattacacaggtgtgcacttcttcaccacctgttcagctgcatatttaaaaatgtacttttctggccaggcatggtggctcacgcctgtaatcacagcactttgggaggccaaggcaggtggatcacttgaggtcaggagctcaagaccagcctggccaacatggtgaaaccctgtctctactaaaaatacaaaaattagctgggcatggtggcaggcgtctgtaatcccagccactcaggaggctgaggcaggagaactgcttgaacccaaggggcggaggttgcagtgagccgagattgcgccactgcactccagcctgggcaacagagtgagactccatctcaagaaaactaaactaaaaaataaagatgtactcttctgtgcacatgttattttccacaaaaaaaaaaaaaaagtacaaaaacaaaagccaaaagacagcaattgatgcctaagatggaagaaacaaggcagcggcggagggagaagcgaggctgcagaggaggctgcggcgggcacaggagccagtctcgggcaggttcaggggcaggagagaggacagcaggaaaaaagggattaaaacccgggcggcccttagaggaacacacctgccagccccgaaatcgaattgcagggacagacggtgctcaggggaactgtactgggtgtcggggcagggaaaaagcagcctcagctcttctctaaagatcttggaggaattttatacccaaactatccacatgtgaaaacccggactgctggtgtggatgacagcctcagagaaaagctccgcttgctgtggggttggggacccagcacccactcaccccaaggcaaagggagccagcacaagcccagggtgctcaggtgcctgggcagcccattcccaggggaatgtgaatcaggagacagatctatctgcatacacaggagcggggatctccatcagcacccccaggaaatccactccgtcctaccgccctcacacggacgacacccaagaatcctgagacgtgtgagcagagcagctgcatggcagagaagcagaggactgaccccaaggagacagaaagggtgggcatcagaagaggggtgcaaaacatccaccacgctcgccaagagagtctggaagaacagggcgctgaggagcatcaaagaacaagaaaacactctcagaaaccaagactgtcactgtggagacaagcaactcacaggaaagactgggaaatgaggaggaggaaatctcccagaacatgcagggggaaaaggaggaaggccgctgaggcctgcatctcctcaagccagctggcatgtggaggtgggtgggggaagcccacacgtctaagcgtgaggggcaggcgcaggccccggctctgcaggttccaaatgttctgctgtgagcagaggcaagaggcagagtggagccggccctggagaggacctatacacatgtacacagcctgagtgctggctacggtgccaggccggtggggtagggcaaaggaaggggccctggggccacgtctggcggggcttcaaggaggcaagtctggtgatccacgtggactggcttagctgggtcagggacggcccctgggtggctggtgggagggctgtgttgggctggcaggtgtggaggggcagccccaggctgagacgcaggtggcaggagctgctggcttctgggagaccagataaagtggtttgatgctcaagttcaagctccaacctccaggccaacatacggggagtacctagagtcatcatggggatggcatccccaaacttgggaacaggtgttcccacacagagcattcaggaaagggcaccatccatctgaaccatccggtcagccaaggggcagcccctggaggacctggcccagctcaggacttggcccgtggtcaaggattcagcgccagatggtcgggttttgcaaaacgatgctacggtttttacaacactgtgtgtgttgaaagtaacctaaaatagtgccttttgcgaaactgtgtcattctttcactgaaagcctaagatttttattttaggaaggtttgaatgacactaacttacattcagcaaaaatgctttaactcaacatcaatctattcaaaactgcagagcagtgcataacgtgagccaacttaagtcccaaaccgactacgtaattattattcagcggtacactctgaatctgctacttcaaattcagtttcctcctgatcccttacgtccctaactacggtgcttttgtgactaggtttccccttggggagttcagaattcagggtttgttctctcttctgtttcccttcaagcaccaagccaagcccaggactgtgcaccaagggagctttcatcagtgctgctgaaggagacgccaggggctccaggggtgggattcaatgccaggcagcacgggcagcttgagtgcccacaagcatccgtgcatactcccacacatgcacacattcacacacatacacagacatcgctctcatatatactcacatgcacacacacactcatacatatgtacacacatatactctcatacacatatgtacatacatcatacatatgtggacacctgtacacacgtacacttgcacatatacacacatacatgtacacagagacacaacactcatacacatgtacatactgtcatacacgtacatgtatgtgcacacgatacagacatccatccacacatatatacatgcactcacatgtacacacatgcaacacacacaggtacacagcacacacatatacacatgtacacactcatatacatacttgcactggtggctcacatacacacagtaatgcaaccacatgcctatgcacgtgtacctactcccgtgcatgcacatacatatgtatgcacactcacatgcgcatatgtacacacatacacaccctctctgcacacatacacagaatgatacacagtgagtctccacagacccagatgcccaaagatctcatgaagcagaggtgacagaaaccccaacgtgtcactgtcacgggacacttcacacgtgcatgagatgtacatatgaaagcgtctctgaagtacagctgctagaataagagtccgttgtggtccatttatcgcctgttgaagccaagtcaattattcacagacaagaaaagccaccagggttagcatggggaaacggaaaattgccccaagccatctattttgaaaagtgagagcctgtgctcgcacggggctcctgcctcctgagacagaacagaggaggcggggagctcggcccaacaaggacatggggagtaaagagaaagggtgccaggaaagcaccacgaagccattgctgcccacagcaccacattcagccaggtcacctgcagcctgcttcacactacagaacccccactttctagctgtggcttctcaactcagcaaaactgaaagcctgactttgatttggccttgatttactttgcttggttatatatttgatacccaattcctattttatggttaaaacgtttcaaaaacaaaccaagataaagcataagagaattttaaaaatctagaaaggctaaatgaagaactaaagcccgtgatgatcctgttgcccctaaagaacccaggctgccccaggtcaccactacccagcaagctgctcaacgtccacccactcagcagggcccagagtgagggacccccaacgcccaggctgccctctcctatgactgcaccatgtctgcacgcaacggtgcggcaaacccgggaccctaagtggccgtcttccccctgcacctaactgcatgtctgagtgtggccctgggtatcaaggagaaccgaagctcaggcagatgtcagctcccatcagcctcttggccgtgaaaactttgctgtacatacagcccacagatcagacaccttccagagaaaggaggggcctgggcgtgcccattacacctttcggaggccaggcagtgaatgctttaggctctacagccacacggcctctgtcccaaccacttggcccggttccccaccaatgcagccctgaacatcacgcaggccagcgggcgtgcccgtgctccagtaaaactttacttatagtcaggcagcagcctccaattaactactaatattcctgtttctgaataaaagattcagggagcccctccagaatgatgagagatgttaacaacacatggaagggttgtcagagctgtggttataggacacgcgtggtaacttttaagatactgaaaatatttatcaaagtatttatgatatctcaacattttcatttgctaatggcagatactttcagtgaaagcacagctttaggaatttaaatgaaacaagaaaatattgtgatttagtttaattttttgtggtctgttttggatctttagtttagatgtattcctcatcgggtaggtgtgctttatttatattccgtgcttaaaagccaggaagagtttaggtccacaaagcaagggaaagacggccctggaggggtggtgccagcaggcagcaggccaggccaacgttggctgacatgcagtctcggagggcggggaaggacccgagcctcctgtccgaaggcagggctgggcctggctccagccagttgctggccctggggagcaggcccactggccggagcttctgcttttccaagactggccagaaatggggattttgatgtcaagtcactgggcctttatgatgcggcaacctgttcccacttagcccaattttaataagaccaggttggcaggagatgccatggtggccacctgccccccgagcagccgttcccctcattgctgctgctctcacacctggaagatggcccctcccgggcccacggcacccgcaccaggtccagctgcctctgcatgggggccactgaggcagggacctccctaggcaggaggcctgtgtcactgtggtgccctgtcactgcagcccaagaggccgtccactcacagcctgagggtctgggggaggccaccatgggcagggccagagctgagggtctgaggtgggaggggtaagggggctggcagtcacaaagccaggagagggtgtccagaagcctgactttcgtcagcaacagggagccccagcaggttctggagcagagaaggctacggtgtaggggggtctctgtgctcaagggtgtgaaacaatggccacacatcctgcagacccctggaaaatgaagcctcagagcaggactgtggcggaggcgtggaagacagaggcaacggggaggtggccgagtccccactggcagggggcagccctggtggacgctgggctcagaagggaaggacagatgggctagggtggagacacacaaccccactgtgagaatgggttcccactgtttggccgcgtgacccccccccaggaggagagcacggcacacgcgcaacttacttcctccacagggccgggggctgctcgggttccttctccgcagagcccagggatgcgacaagtgcgaggggccaggccagcagcatcatggcagcggcaaccaggagccggaccgggaagagcgtcagtgtcatgagggccacctgcagacagaggggggcattatccagagaatccatgtaggacaccaggcagctccccacccggcagaggctagcccagggggacagcgcgccccacagagtggagagctggggaggggctgcagctggcacacagctgaccaaggctgaccagcactgaccgaggctgaccaaggctgaccgaggctgaccagtgctgaccggcgctgaccaaggctaaccaacactgaccagcactgaccaaggctgaccggcactgaccaaggctgaccaacattgaccggcactgaccaaggctgaccggtgctgaccaaggctgaccaacactgaccgacactgaccaaggctgaccggcactgaccaaggctgaccaacactgactggcactgaccaaggctgaccaacattgaccggcactgaccaaggctgaccggtgctgatgccgaccagccctcaccatggctgtggacactggccagtgctaactgacatgctccacacccactgcccgcttcctgcctccttactggagctgccatgcgagtggcttgggaaagacacagtggagcttcctcctgcaaattcaagggccactctgacgcacagcacacaaccccaggcagctccaccccgcaggacgcacccccaggcagctccgccccccaggacacacccccagccccgcaggcctcactcagcagtttcccctgaaatcgtttatgtggacatgagaaagcatacgccaccctaggcagtgttggtgacagggggaggtagctggcctgcagtttgcaaggttggacctcagaccacagagaagacggcagaatgaaggtgtgactttccagtcaatggaagacccggcgcaggagagtgaactggcgggagggcaggggcgcaggtcaatatgggggctcaggaaggccccccaagccagggcaggcccccacgccagggaaggccgaggctgcgggatcccaggcagcccagggtctgaggatggaggtccctccgggaggcgggcaggactccgtgtagaggggcgggaggctttgggtgaggggaaacaccagtcctgaggtgcaggtttagtgcagagacagaaaagacccaggagacacagatcgcagttggctccccaagcagcagccatccctaatttctgatgcctgtcatgagagcgctcccactctgtttccttggtttgtccctagattcttttgagttctcaacggagaccagcatagcccaagagaaacatgcctgccacatgtgtcattttaaattttcgagtggccacatttaaagggaatgacaatttaatatacagctcatttcgcccggtatatctgaaataccatttcgacacgtactcaacgtagaagttgctactggctagtttgcattttctcccgctgtcttggattgtgcagtgtgcatttcactctcacagctttatgccacctgtgtctccaccctgtcagtgcccagtggtcaggtcagcctgttgggtacgcagatctagatgatgtttggtttgcgagagatgacgacctctaatttgcctccccttccccgcttctgggggctgctcctcaccccagctgggtccccgcttcagcatcacactcgctgcaccaagcaaccacaaatctgttatcccgccattggtgacgatcgtcactgttccacatcgtgttactggcattcagcaaacgttctttactgctgagaaaccttcccaggcctcatcctccacgagttttgttttgctttaaatcatgaacgggtgctgagctttatcagaggtgcttcctgcatctgtggggagagccacgtgctgcaccccacttgaagccactaacatgatgccttacggccacggtctctctaatactgaaagctagcttcagcgctgtgcagctccctgttaggattgttttttaaaaggaattgctaggtctgttggccactgtcaccagctgttaagtaagacgcgcctgtccgctcctgtcacttcctcccagcggcttcctcgtgcagtcgccctacgaaaccagcgtgcacccatgcctgccggcagctcctgaccgttgcttcactgtttccaaccctactgtttatagagtccctttttttttgggttgacttcactgtttttgtcaatggggaagtctcttcctggaagcttttggcatatttttccctcccgctaccaaggtctcctcccagctgcctttaagataacagtgaagttgcccgccacactctccttgggttcacaccaagcctgctctctggtttcatttctaatgttatctgtgtctgccaaaacttctaaacctacgttgccagatatttttctattttatcagtcttcttacagaaccatctcagatttgttcattctctctgttgtgacctaatcatattaattttggctcttattagttcctctttcttctgctttgaatttagtcaaatattcttttcaactttttgagggaaaagcccactttgttattttcagaaataccttatttcctgatagtcacatttagaacacagctccccctaaggaccaccttgatttaatcccacaagtctaaggggcagccttttcctttatttgtaaaatttagttttaaatatttcatccattcccatctactgttactgtttcatattttgttgcagtggaggagagcgcagcggaggagaccgtagcctcggccttcagcccacctggtcccggggctccagggccactggctgggaagaggccactctgcagtgcacaagcctggctctgctgttaaagccgacagctaggcagggcgtggtggctcacatctgtaatcccagcactttgggaggccaaggtgggaggatcgcctgcggtcgggagttcgagaccagcctgaccaacatggagaacccatctctactaaaaatacaaaagtagccgggcgtggtggcgcatgcctgtaatcacagctactctggaggctgaggcaggagaatcacttggacccgggagtcagaggttgtggggagctgagatcgcgccattgcgctctagcctgggcaacaagagcgaatctccgtcccaaaaaaaaaaaaaaaaaagccgacaataagttactgggtaagtttcttcttcccataaattgttcttagaacttgttgcctccctgggcagacgccatgaagtgtgataaccagcactaagagaaagtctcaggaagttccccggtctgaccctggggccacggcagcctctgcccacagcctgcttcagtccacctctggtgctgcagccaactctgctacaacactgcttccaccagagtgtggaataaaatcacagtaacccacgctgttcctgaaacagcaccgactgcaacactgcttccaccacagggtgtggaataaaatcacagtaacccacgctgttcctgaaacagcaccgactgcaacactgcttccaccacagggtgtggaataaaatcacagtaacccacgctgttcctgaaacagcaccgactgcaacactgcttccaccagagtgcggaataaaatcacagtaacccacggtgttcctgaaacagcaccgactacaactgcttccaccagagtgcggaataaaatcacagtaacccacgctgttcctgaaacagcaccgactgcaacactgcttccaccacagagtgcggaataaaatcacagtaacccacggtgttcctgaaacagcaccgactgcaacactgcttccaccacagagtgcggaataaaatcacagtaacccatggtgttcctgaaacagcaccgactgccaggccattccatatgggaccacccactgggaacagctccggggctgggggagcagcgccccatccccaaacaacacggggcatgaatgagccctgggtggggaggccccgcctgcatgccgggtagggtagtcaggctgctggttccttagcacatggggttcgctgggccagccagtggggggatgcactgtgagaccacgtgaccctcggctcgcgcacccccacgcctgctcctgcgcacatagggaagactgggcgaccacctgagcctcgacttacacattcctgcacctactccttcgtgcatgggttagactgcatgaccacctgaccctcacctcacacgtccatgcatccatgcacctgctgcttcgtgcacggggaggactgtgtgaacaagcgaccctctgcaccagctccttcctctatgggaaagaatgcctgaccacgtgacgctccccaccagtcagtgcacctgctcctgttggtgtcccagaagactgcgtgaccatgtgaccctcagcccatgcacccttgtgcctgctcgtggtgcatcagggaatgcggcccacacttctgagcttagcagcagcaaggagaggggcctgtgggccacagttgcaatcgctgatcctcggcaaacagggctgatgaggggagggtggagagggaacatggccagggaggagctccccaggcctccagcagatggggagggtgtggctctgctctgcagtgctgtgaaaactgcttggagagggggcctctgggagccccaggcaggaggaggagcagtgctcctggtgaagagcccgggcatctccaccgatctgccttcacctcacccagctctgggcctgtgcgctctttctctgccaggctcggcccgcccacaggagccgggagccctcaccaaggagaacaggacttgggtgactgctgtgactccttccagaagcttctatgaccccgcaccttctgcccacctgtctccacaggccccacccctgcgtccagcctcaggcacacccattgggatccctccattcctcatgcccccacgccctggtgcagatgggtctggatggactctagccccccaggagatgccagtatggcagagctgcagagagagaagctggctggggcagctgcggcagccaagcccctcagcactggcaggggtggccaagggaggtgctgggtaggcaaagaaattcttagaaaatcctggctagaccagaaactcccagaacaatggccttctaaggaaagggggctggcagtgcggtctcccaggcagcgtcgctggagactcaggtcgctaaagtggaggcaccgggcacagccattcctccccacggcagctttgcagtgtccactgagagcaagcgtgcgtttacagacctgcagccacagggacacctgtcagagagggcaggtgtcttttctcctcacccaagcaaggcccagcaccctggctcagaggcttccacagaagcccccttggcaccttcaccagaggcagcaggggcagcatgcggaaccaagacatcactgcaagtcgtggacagggataagcgggctgttgcgtggacaaggtgggcagatgtggggagcgggagcagcctccaggcagtgtggccagaggctgaatgcggagagggggagggactcagagaggagaacacggaccacgctcctggtcaccataggtcaggcctccacgcggcagccaactggggaggacactgtggacgcctgcagacgctgagacctgcaacctggccctcccaagacaagctgctgatggctgtgggccggctgtcaccacaggaccctgatcaccctgagaaatgccagttggaggaagcaatatggtcaccctgtgtgagccataagtgagggtttatagcataatcctaatgaggaactttgtctgaagtctgaggctgagttacttcaactatttgaaagttggctacgataaaccaaggcgtccaggcagtgacctgcccacatgcagggagagctttggtgcggactgcggccgctaaataccacttcccagcagaaggcccagggctcctcagccgggcttgctccaggtcacaggtgggaaatgcagagatgaagatgggacagcacggcgcagcccacatcagggaagctggcaggggccgggcgcgagcgctcacgcctgcagtcccaggagcacggcgcggcccaaatcaaggaagctggcagaggctcatgtcctgagggctggctcgaggcctacccagctcacactcacgtgacagggatccagcaacagccaatgccgagcctggtgcaggagcagcacagcagaccagtgggaactgaccctctctgggtctagatgcaaccaccagcttccagaaagccccaatgacaacaaggagatgcaatcaatcagaaaatgcagagaactccacaaaacacaaaactccacaaaacacagcccaaatccattgcagcaaacaaggaggagctggaggaccctcctgatccacacgcaccagaggcccgagggagctcgttcacccctccaccttgcgaggacacagaaggtgccatctattgacccggagggggtccttggcagacaccaaatctgccggcatcttggacttcagcctccagaatggtgagaagtccgtgtctgctgttcgggaggcacctcgtctgtggtcctttgtgacagcagccgcgccaagcagggcgggcacagtgggagtgcacgtgaccccggcctggccacacaccgtcattctccgggctgggggacaggtcccaggcccactgtattatcactgctttggtgttctaggttgaattttgcgcccccttccccaattcctgttgatgtcctgacccccaggactccagagggtgactgtatttggagaaggggctgttagagaggtgacgaaggtaaaatgagatcatctgggggccctgagccaacctgcctggtgtccttataagaagaggagatgaggacacagacacccagggagggacggccctgtgaggacacagggtgaaggcaccatctgcaagcccaggagaggcctcaggaggaaccagccctgctgcgcctcggtctcggactccagcctccaggatggtgagacaacaatgtctgtggtttaagccccatctgaggcactttattatggaagccggagctgagaaagacacttgacatatgtttgaaatttcccatgataaaaagtataaaagtgatgactcttggcttaaatgtaaacaaggccatccaaatgttggcctggaactctctggaagtctctgagtgaaggcgccggtggtcacaatgcccaggcccaggctctctgatttcaggcagctggcaaacccctccctcgtctctgccaaggccaccagggccgtaagcagccccaggccccagcatcacccggcgtttcacaatcagtgctgaactcaaagacacgaggttgccagagaacatgtggtccctctcgtgtgcactcacgccatcgcggtgcccgacaggggtgctgcggtgaggcccaccaggcgcagggatgctgcggcccctccgagcccacttgttggagatgtcacactgcagtgccccagccagtgaaggggctccaatgcacctgcaacgaggcaggggtggaggggaggctgcccaaaggatggcgcccggagactcgaaggccgcgaggccgcgtcatcccgcacaggcggccggggcagatgctcagtgccagccaccccaagctctacggttagcgcggtggaggcagctcacaggaatgctccctcctgaaggtgcatcccaacattgctgctttaattatgtggaaaaaattatcccaactccacaggatatgcgacagtgcgcttgcaaatccacgagagaacgtgcactcctcccacggtgagagggcgagcggcagcacacacaggcgcctgctgtccaaggtctaattttcttttgctttttccttttagagaaagaacaacttttgaaagcacagcagttctctcaggcatcccatcggacggagccggcacttccatcagcaaacaccaccagcccgccctcggcatctgagagctggagggagcttcgggaatcacccactcccgtccagcacccacagacacaaaccgagcaaggcagcagcaggaaacgccctccttttctgtctccccaatcaccaaaacacctttattcatataacatgacttctggtccaaaccaaacgctgccttcaaatatggaatcagcgatcacgcccacaatgaattctcatagttaacgatactaaaagcagacaggaaatgtgaaaaagtggcaccgtgaaccattgccgtgggctctctctgcacacacaaccactcttacctttggagcccgaggcacggcccgcaccccaggcgctacagatcccaggtgaggcctgcaggaggagggatgggctgccaggagcgcaggccacatcacggggacggtttcattgggaggaacattttatgtttactgggtattcccatttctgtttgtgtaattttctcatttatataaatgtatatacatcttgtgccaaatttatggtgggtttctttgtctttttctcacccctgtgaacatc

SEQ ID NO: 2 - DNA, 599 bp, Homo sapiens
acttcaactgaaacaggagagacaggcccaggtcagctgtgggcagcacaggagacgggggccgggccgcgggcagcacacaccgccttaccttgtagttcacaaagagctggggcagcatgaagaggaaaccaaaggcatagacccctgcagaaagacagacagcactcacgaggtgcggagggccgggctgccacatctacgcacagacggcactcacgaggtgtggagggccgagctgccacgtctatgcatagtgggcagaaaacaaggtctgctatccagacaacttcagagtcyatcatggtgtgaagcagctttctggctggtaagtttatcaagagtctacgacaactttggagagcaaagctcttctatttattaatcagtgtaactgctgcccacggggtcaagtcagtgagagcactggagcaccctggggctatgaggacacggcctcctgacccacaaggtcctgccccaggggtcaggtgtgggactcctaccagggaggacggcagtgcgggacccacctgctctgcaccagacaggccctcgcccttaatgctcacgggaccacttcccaaagagaccacaaa

SEQ ID NO: 3 - DNA, 599 bp, Homo sapiens
tcaccatgttggccaggctggtctcaaactcctgacctcaagtgatctgcccgccttggcctcccacagtgctgggattacaggtgcaagccaccgtgcccggcataccttgatcttttaaaatgaagtctgaaacattgctacccttgtcctgagcaataagacccttagtgtattttagctctggccaccccccagcctgtgtgctgttttccctgctgacttagttctatctcaggcatcttgacacccccacaagctaagcattattaatattgttttccgtgttgagtgtttctktagctttgcccccgccctgcttttcctcctttgttccccgtctgtcttctgtctcaggcccgccgtctggggtccccttccttgtcctttgcgtggttcttctgtcttgttattgctggtaaaccccagctttacctgtgctggcctccatggcatctagcgacgtccggggacctctgcttatgatgcacagatgaagatgtggagactcacgaggagggcggtcatcttggcccgtgagtgtctggagcaccacgtggccagcgttccttagccagtgagtgacagcaacgtccgct

SEQ ID NO: 4 - DNA, 599 bp, Homo sapiens
agcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcrggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgccccta

(19) United States
(12) Patent Application Publication
(10) Pub. No.: US 2011/0212855 A1
(43) Pub. Date: Sep. 1, 2011

(54) Genetic Variants Predictive of Cancer Risk

(75) Inventors: Thorunn Rafnar, Reykjavik (IS); Patrick Sulem, Reykjavik (IS); Simon Stacey, Kopavogur (IS)

(73) Assignee: deCODE Genetics ehf., Reykjavik (IS)

(21) Appl. No.: 13/058,784

(22) PCT Filed: Aug. 17, 2009
(86) PCT No.: PCT/IS09/00011

§ 371 Date: May 11, 2011

(30) Foreign Application Priority Data
Aug. 15, 2008 (IS) 8756
Jan. 16, 2009 (IS) 8783

(51) Int. Cl. C40B 30/04; C12Q 1/68

(52) U.S. Cl. 506/9; 435/6.11

(57) ABSTRACT

The invention discloses genetic variants that have been determined to be susceptibility variants of cancer. Methods of disease management, including determining increased susceptibility to cancer, methods of predicting response to therapy and methods of predicting prognosis of cancer using such variants are described. The invention further relates to kits useful in the methods of the invention.

### INTRODUCTION

Cancer, the uncontrolled growth of malignant cells, is a major health problem of the modern medical era and is one of the leading causes of death in developed countries. In the United States, one in four deaths is caused by cancer (Jemal, A. et al., CA Cancer J. Clin. 52:23-47 (2002)). Cancer initiation results from the complex interplay of genetic and environmental factors. The estimated contribution of genetic factors varies widely between cancer sites, with prostate cancer generally considered to have the largest genetic component {Lichtenstein, 2000 #18}. However, genetic factors also play a role in cancer types with strong environmental factors such as lung cancer (Jonsson, S., et al. JAMA 292:2977-83 (2004); Hemminki, K., et al. Genet Epidemiol 20:107-116 (2001)).

All cancers are subject to the accumulation of genetic changes that lead to aberrant cell growth and survival. Thus, it could be expected that genetic polymorphisms that affect certain basic cellular processes, such as DNA repair, cell cycle regulation and apoptosis could increase an individual's life-long risk of developing cancer—the actual site of cancer could be determined by other factors, environmental or genetic (Hanahan, D. and Weinber, R. A., Cell 100:57-70 (2000). Indeed, studies on cancer risk in relatives of cancer patients lends strong evidence for shared genetic factors that increase the risk of more than one cancer type (Cannon-Albright, L. A., et al. Cancer Res 54:2378-85 1994); Amundadottir, L. T., et al. PLoS Med 1:e65 (2004)). Furthermore, mutations in strongly cancer-predisposing genes are associated with an increased risk of more than one type of cancer, as exemplified by the spectrum of cancer types in Li-Fraumeni syndrome that are caused by mutations in TP53 (Malkin, D., et al. Science 250:1233-38 (1990). However, highly penetrant mutations explain only a small fraction of total cancer cases and the majority of genetic cancer risk is thought to be due to the presence of multiple common genetic variants of low penetrance.

Basal Cell Carcinoma. Cutaneous basal cell carcinoma (BCC) is the most common cancer amongst whites and incidence rates show an increasing trend. The average lifetime risk for Caucasians to develop BCC is approximately 30% [Roewert-Huber, et al., (2007), Br Dermatol, 157 Suppl 2, 47-51]. Although it is rarely invasive, BCC can cause considerable morbidity and 40-50% of patients will develop new primary lesions within 5 years[Lear, et al., (2005), Clin Exp Dermatol, 30, 49-55]. Indices of exposure to ultraviolet (UV) light are strongly associated with risk of BCC [Xu and Koo, (2006), Int J Dermatol, 45, 1275-83]. In particular, chronic sun exposure (rather than intense episodic sun exposures as in melanoma) appears to be the major risk factor [Roewert-Huber, et al., (2007), Br J Dermatol, 157 Suppl 2, 47-51]. Squamous cell carcinoma of the skin (SCC) shares these risk factors, as well as several genetic risk factors with BCC [Xu and Koo, (2006), Int J Dermatol, 45, 1275-83; Bastiaens, et al., (2001), Am 3 Hum Genet, 68, 884-94; Han, et al., (2006), Int J Epidemiol, 35, 1514-21]. Photochemotherapy for skin conditions such as psoriasis with psoralen and UV irradiation (PUVA) have been associated with increased risk of SCC and BCC. Immunosuppressive treatments increase the incidence of both SCC and BCC, with the incidence rate of BCC in transplant recipients being up to 100 times the population risk [Hartevelt, et al., (1990), Transplantation, 49, 506-9; Lindelof, et al., (2000), Br 3 Dermatol, 143, 513-9]. BCC's may be particularly aggressive in immunosuppressed individuals.

There is an unmet clinical need to identify individuals who are at increased risk of BCC and/or SCC. Such individuals might be offered regular skin examinations to identify incipient tumours, and they might be counseled to avoid excessive UV exposure. Chemoprevention either using sunscreens or pharmaceutical agents [Bowden, (2004), Nat Rev Cancer, 4, 23-35.] might, be employed. For individuals who have been diagnosed with BCC or SCC, knowledge of the underlying genetic predisposition may be useful in determining appropriate treatments and evaluating risks of recurrence and new primary tumours. Screening for susceptibility to BCC or SCC might be important in planning the clinical management of transplant recipients and other immunosuppressed individuals.

Melanoma. Cutaneous Melanoma (CM) was once a rare cancer but has over the past 40 years shown rapidly increasing incidence rates. In the U.S.A. and Canada, CM incidence has increased at a faster rate than any other cancer except bronchogenic carcinoma in women. Until recently incidence rates increased at 5-7% a year, doubling the population risk every 10-15 years.

The current worldwide incidence is in excess of 130,000 new cases diagnosed each year [Parkin, et al., (2001), Int Cancer, 94, 153-6]. The incidence is highest in developed countries, particularly where fair-skinned people live in sunny areas. The highest incidence rates occur in Australia and New Zealand with approximately 36 cases per 100,000 per year. The U.S.A. has the second highest worldwide incidence rates with about 11 cases per 100,000. In Northern Europe rates of approximately 9-12 per 100,000 are typically observed, with the highest rates in the Nordic countries. Currently in the U.S.A., CM is the sixth most commonly diagnosed cancer (excluding non-melanoma skin cancers). In the year 2008 it is estimated that 62,480 new cases of invasive CM will have been diagnosed in the U.S.A. and 8,420 people will have died from metastatic melanoma. A further 54,020 cases of in-situ CM are expected to be diagnosed during the year.

Deaths from CM have also been on the increase although at lower rates than incidence. However, the death rate from CM continues to rise faster than for most cancers, except non-Hodgkin's lymphoma, testicular cancer and lung cancer in women [Lens and Dawes, (2004), Br 3 Dermatol, 150, 179-85.]. When identified early, CM is highly treatable by surgical excision, with 5 year survival rates over 90%. However, malignant melanoma has an exceptional ability to metastasize to almost every organ system in the body. Once it has done so, the prognosis is very poor. Median survival for disseminated (stage 1V) disease is 7 ½ months, with no improvements in this figure for the past 22 years. Clearly, early detection is of paramount importance in melanoma control.

CM shows environmental and endogenous host risk factors, the latter including genetic factors. These factors interact with each other in complex ways. The major environmental risk factor is UV irradiation. Intense episodic exposures rather than total dose represent the major risk [Markovic, et al., (2007), Mayo Clin Proc, 82, 364-80].

It has long been recognized that pigmentation characteristics such as light or red hair, blue eyes, fair skin and a tendency to freckle predispose for CM, with relative risks typically 1.5-2.5. Numbers of nevi represent strong risk factors for CM. Relative risks as high as 46-fold have been reported for individuals with >50 nevi. Dysplastic or clinically atypical nevi are also important risk factors with odds ratios that can exceed 30-fold [Xu and Koo, (2006), Int J Dermatol, 45, 1275-83].

Lung Cancer. Lung cancer causes more deaths from cancer worldwide than any other form of cancer (Goodman, G. E., Thorax 57:994-999 (2002)). In the United States, lung cancer is the primary cause of cancer death among both men and women. In 2007, the death rate from lung cancer was an estimated 160,390 deaths, exceeding the combined total for breast, prostate and colon cancer (America Cancer Society, www.cancer.org). Lung cancer is also the leading cause of cancer death in all European countries and is rapidly increasing in developing countries. While environmental factors, such as lifestyle factors (e.g., smoking) and dietary factors, play an important role in lung cancer, genetic factors also contribute to the disease. For example, a family of enzymes responsible for carcinogen activation, degradation and subsequent DNA repair has been implicated in susceptibility to lung cancer. In addition, an increased risk to familial members outside of the nuclear family has been shown by deCODE geneticists by analysing all lung cancer cases diagnosed in Iceland over 48 years. This increased risk could not be entirely accounted for by smoking indicating that genetic variants may predispose certain individuals to lung cancer (Jonsson et al., JAMA 292(24):2977-83 (2004); Amundadottir PLoS Med. 1(3):e65 (2004)).

The five-year survival rate among all lung cancer patients, regardless of the stage of disease at diagnosis, is only 13%. This contrasts with a five-year survival rate of 46% among cases detected while the disease is still localized. However, only 16% of lung cancers are discovered before the disease has spread. Early detection is difficult as clinical symptoms are often not observed until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy.

In spite of considerable research into therapies for this and other cancers, lung cancer remains difficult to diagnose and treat effectively. Accordingly, there is a great need in the art for improved methods for detecting and treating such cancers.

Smoking of tobacco products, and in particular cigarettes, is the largest known risk factor lung cancer with a global attributable proportion estimated to be approximately 90% in men and 80% in women. Although the risk of lung cancer associated with tobacco smoking is strongly related to duration of smoking, and declines with increasing time from cessation, the estimated lifetime risk of lung cancer among former smokers remains high, ranging from approximately 6% in smokers who give up at the age of 50, to 10% for smokers who give up at age 60, compared to 15% for lifelong smokers and less than 1% in never-smokers (Peto et al. 2000 BMJ, 321, 323-32, Brennan, et al. 2006 μm 3 Epidemiol 164, 1233-1241). In populations where the large majority of smokers have quit smoking, such as men in the US and UK, the majority of lung cancer cases now occurs among ex-smokers (Doll et al. 1994 BMJ 309, 901-911, Zhu et al. 2001 Cancer Res, 61, 7825-7829). This emphasizes the importance of developing alternative prevention measures for lung cancer including the identification of high risk subgroups.

Notably, only about 15% of lifelong smokers will develop lung cancer by the age of 75, and approximately 5 to 10% of lifetime smokers will develop another tobacco-related cancer (Kjaerheim et al. 1998 Cancer Causes Control 9, 99-108). A possible explanation for these large differences in risk for individuals with similar level of tobacco exposures could be that genetic factors play a determining role in lung cancer susceptibility (Spitz et al. 2005 J Clin Oncol 23, 267-275). Identifying genes, which influence the risk of lung cancer could be important for several aspects of management of the disease.

Segregation analyses predict that the majority of genetic risk for lung cancer is most likely to be polygenic in nature, with multiple risk alleles that confer low to moderate risk and which may interact with each other and with environmental risk factors. Many studies have investigated lung cancer susceptibility based on the presence of low-penetrance, high-frequency single nucleotide polymorphisms in candidate genes belonging to specific metabolic pathways. Genetic polymorphisms of xenobiotic metabolism, DNA repair, cell-cycle control, immunity, addiction and nutritional status have been described as promising candidates but have in many cases proven difficult to confirm (Hung et al. 2005 3 Natl Cancer Inst 97, 567-576, Hung et al. 2006 Cancer Res 66, 8280-8286, Landi et al. 2006 Carcinogenesis, in press, Brennan et al. 2005 Lancet 366, 1558-60, Hung et al. 2007 Carcinogenesis 28, 1334-40, Campa et al. 2007 Cancer Causes Control 18, 449-455, Gemignani et al. 2007 Carcinogenesis 28(6), 1287-93, Hall et al. 2007 Carcinogenesis 28, 665-671, Campa et al. 2005 Cancer Epidemiol Biomarkers Prev 14, 2457-2458, Campa et al. 2005 Cancer Epidemiol Biomarkers Prev 14, 538-539, Hashibe et al. 2006 Cancer Epidemiol Biomarkers Prev 15, 696-703).

For cancers that show a familial risk of around two-fold such as lung cancer (Jonsson et al. 2004 JAMA 292, 2977-2983, Li and Hemminki 2005 Lung Cancer 47, 301-307, Goldgar et al. 1994 J Natl Cancer Inst 86, 1600-1608), the majority of cases will arise from approximately 10%-15% of the population at greatest risk (Pharoah et al. 2002 Nat Genet 31, 33-36). The identification of common genetic variants that affect the risk of lung cancer may enable the identification of individuals who are at a very high risk because of their increased genetic susceptibility or, in the case of genes related to nicotine metabolism, because of their inability to quit smoking. Such findings could potentially lead to chemoprevention programs for high risk individuals, and are especially of importance given the high residual risk that remains among ex-smokers, among whom the majority of lung cancers in the US and Europe now occur.

Bladder Cancer. Urinary bladder cancer (UBC) is the 6th most common type of cancer in the United States with approximately 67,000 new cases and 14,000 deaths from the disease in 2007. UBC tends to occur most commonly in individuals over 60 years of age. Exposure to certain industrially used chemicals (derivatives of compounds called arylamines) is strong risk factor for the development of bladder cancers. Tobacco use (specifically cigarette smoking) is thought to cause 50% of bladder cancers discovered in male patients and 30% of those found in female patients. Thirty percent of bladder tumors probably result from occupational exposure in the workplace to carcinogens such as benzidine. Occupations at risk are metal industry workers, rubber industry workers, workers in the textile industry and people who work in printing. Certain drugs such as cyclophosphamide and phenacetin are known to predispose to bladder cancer. Chronic bladder irritation (infection, bladder stones, catheters, and bilharzia) predisposes to squamous cell carcinoma of the bladder.

Familial clustering of UBC cases suggests that there is a genetic component to the risk of the disease (Aben, K. K. et al. Int J Cancer 98, 274-8 (2002); Amundadottir, L. T. et al. PLoS Med 1, e65 Epub 2004 Dec. 28 (2004); Murta-Nascimento, C. et al. Cancer Epidemiol Biomarkers Prev 16, 1595-600 (2007)). Genetic segregation analyses have suggested that this component is multifactorial with many genes conferring small risks (Aben, K. K. et al. Eur J Cancer 42, 1428-33 (2006)). Many epidemiological studies have evaluated potential associations between sequence variants in candidate genes and bladder cancer, but the most consistent risk association to the disease is found for variations in the NAT2 gene. (Sanderson, S. et al., Am J Epidemiol 166, 741-51 (2007)).

Majority (>90%) of bladder cancers are transitional cell carcinomas (TCC) and arise from the urothelium. Other bladder cancer types include squamous cell carcinoma, adenocarcinoma, sarcoma, small cell carcinoma and secondary deposits from cancers elsewhere in the body. TCCs are often multifocal, with 30-40% of patients having a more than one tumor at diagnosis. The pattern of growth of TCCs can be papillary, sessile (flat) or carcinoma-in-situ (CIS). Superficial tumors are defined as tumors that either do not invade, or those that invade but stay superficial to the deep muscle wall of the bladder. At initial diagnosis, 70% of patients with bladder cancers have superficial disease. Tumors that are clinically superficial are composed of three distinctive pathologic types. The majority of superficial urothelial carcinomas present as noninvasive, papillary tumors (pathologic stage pTa). About 70% of these superficial papillary tumors will recur over a prolonged clinical course, causing significant morbidity. In addition, 5-10% of these papillary lesions will eventually progress to invasive carcinomas. These tumors are pathologically graded as either low malignant potential, low grade or high grade. High grade tumors have a higher risk of progression. Flat urothelial carcinoma in situ (CIS) are highly aggressive lesions and progress more rapidly than the papillary tumors. A minority of tumors invade only superficially into the lamina propria. These tumors recur 80% of the time, and eventually invade the detrusor muscle in 30% of cases. Approximately 30% of urothelial carcinomas invade the detrusor muscle at presentation. These cancers are highly aggressive. Those invasive tumors may spread by way of the lymph and blood systems to invade bone, liver, and lungs and have high morbidity (Kaufman, D. S. Ann Oncol 17, v 106-112 (2006)).

The treatment of transitional cell or urothelial carcinoma is different for superficial tumors and muscle invasive tumors. Superficial bladder cancers can be managed without cystectomy (removing the bladder). The standard initial treatment of superficial tumors includes cystoscopy with trans-urethral resection of the tumor (TUR). The cystoscope allows visualization and entire removal of a bladder tumor. Adjuvant intravesical drug therapy after TUR is commonly prescribed for patients with tumors that are large, multiple, high grade or superficially invasive. Intravesical therapy consists of drugs placed directly into the bladder through a urethral catheter, in an attempt to minimize the risk of tumor recurrence and progression. About 50-70% of patients with superficial bladder cancer have a very good response to intravesical therapy. The current standard of care consists of urethro-cystoscopy and urine cytology every 3-4 months for the first two years and at a longer interval in subsequent years.

Cystectomy is indicated when bladder cancer is invasive into the muscle wall of the bladder or when patients with superficial tumors have frequent recurrences that are not responsive to intravesical therapy. The benefits of surgically removing the bladder are disease control, eradication of symptoms associated with bladder cancer, and long-term survival. For advanced bladder cancer that has extended beyond the bladder wall, radiation and chemotherapy are treatment options. Local lymph nodes are frequently radiated as part of the therapy to treat the microscopic cancer cells which may have spread to the nodes. Current treatment of advanced bladder cancer can involve a combination of radiation and chemotherapy.

Early detection can improve prognosis, treatment options as well as quality of life of the patient. If screening methods could detect bladder cancers destined to become muscle invading while they are still superficial it is likely that a significant reduction in morbidity and mortality would result. Cystoscopic examination is costly and causes substantial discomfort for the patient. Urine cytology has poor sensitivity in detecting low-grade disease and its accuracy can vary between pathology labs. Many urine-based tumor markers have been developed for detection and surveillance of the disease and some of these are used in routine patient care (Lokeshwar, V. B. et al. Urology 66, 35-63 (2005); Friedrich, M. G. et al. BJU Int 92, 389-92 (2003); Ramakumar, S. et al. J Urol 161, 388-94 (1999); Sozen, S. et al. Eur Urol 36, 225-9 (1999); Heicappell, R. et al. Urol Int 65, 181-4 (2000)). However, no biomarker reported to date has shown sufficient sensitivity and specificity for detecting all types of bladder cancers in the clinic. It should be remembered that efficiency of screening increases with the disease's prevalence in the screened population. Therefore, the efficiency of the test could be increased by limiting the screening program to people at high risk. For bladder cancer, this may mean restricting participation to people with occupational exposure to known bladder carcinogens or individuals with known cancer predisposing variants.

The genetic polymorphisms in a number of metabolic enzymes and other genes have been found as the modulators of bladder cancer risk. The most studied polymorphisms in connection with bladder cancer risk are polymorphisms in genes for some important enzymes, especially N-acetyltransferases (NATs), glutathione S-transferases (GSTs), DNA repair enzymes, and many others. An improved understanding of the molecular biology of urothelial malignancies is helping to define more clearly the role of new prognostic indices and multidisciplinary treatment for this disease.

Despite intensive efforts, the genes that account for a substantial fraction of bladder cancer risk have not been identified. Although studies have implied that genetic factors are likely to be prominent in bladder cancer, only few genes have been identified as being associated with an increased risk for the disease. Thus, it is clear that the majority of genetic risk factors for bladder cancer remain to be found. It is likely that these genetic risk factors will include a relatively high number of low-to-medium risk genetic variants. These low-to-medium risk genetic variants may, however, be responsible for a substantial fraction of bladder cancer, and their identification, therefore, a great benefit for public health.

There is clearly a need for improved diagnostic procedures that would facilitate early-stage bladder cancer detection and prognosis, as well as aid in preventive and curative treatments of the disease. In addition, there is a need to develop tools to better identify those patients who are more likely to have aggressive forms of bladder cancer from those patients that are diagnosed with the superficial disease. This would help to avoid invasive and costly procedures for patients not at significant risk.

Prostate Cancer. The incidence of prostate cancer has dramatically increased over the last decades and prostate cancer is now a leading cause of death in the United States and Western Europe (Peschel, R. E. and J. W. Colberg, Lancet 4:233-41 (2003); Nelson, W. G. et al., N. Engl. J. Med. 349(4):366-81 (2003)). Prostate cancer is the most frequently diagnosed noncutaneous malignancy among men in industrialized countries, and in the United States, 1 in 8 men will develop prostate cancer during his life (Simard, J. et al., Endocrinology 143(6):2029-40 (2002)). Although environmental factors, such as dietary factors and lifestyle-related factors, contribute to the risk of prostate cancer, genetic factors have also been shown to play an important role. Indeed, a positive family history is among the strongest epidemiological risk factors for prostate cancer, and twin studies comparing the concordant occurrence of prostate cancer in monozygotic twins have consistently revealed a stronger hereditary component in the risk of prostate cancer than in any other type of cancer (Nelson, W. G. et al., N. Engl. J. Med. 349(4):366-81 (2003); Lichtenstein P. et. al., N. Engl. J. Med. 343(2):78-85 (2000)). In addition, an increased risk of prostate cancer is seen in 1^{st} to 5^{th} degree relatives of prostate cancer cases in a nation wide study on the familiality of all cancer cases diagnosed in Iceland from 1955-2003 (Amundadottir PLoS Medicine 1(3):e65 (2004)). The genetic basis for this disease, emphasized by the increased risk among relatives, is further supported by studies of prostate cancer among particular populations: for example, African Americans have among the highest incidence of prostate cancer and mortality rate attributable to this disease: they are 1.6 times as likely to develop prostate cancer and 2.4 times as likely to die from this disease than European Americans (Ries, L. A. G. et al., NIH Pub. No. 99-4649 (1999)).

An average 40% reduction in life expectancy affects males with prostate cancer. If detected early, prior to metastasis and local spread beyond the capsule, prostate cancer can be cured (e.g., using surgery). However, if diagnosed after spread and metastasis from the prostate, prostate cancer is typically a fatal disease with low cure rates. While prostate-specific antigen (PSA)-based screening has aided early diagnosis of prostate cancer, it is neither highly sensitive nor specific (Punglia et. al, N Engl J. Med. 349(4):335-42 (2003)). This means that a high percentage of false negative and false positive diagnoses are associated with the test. The consequences are both many instances of missed cancers and unnecessary follow-up biopsies for those without cancer. As many as 65 to 85% of individuals (depending on age) with prostate cancer have a PSA value less than or equal to 4.0 ng/mL, which has traditionally been used as the upper limit for a normal PSA level (Punglia et. al., N Engl J. Med. 349(4):335-42 (2003); Cookston, M. S., Cancer Control 8(2):133-40 (2001); Thompson, I. M. et. al., N Engl J. Med. 350:2239-46 (2004)). A significant fraction of those cancers with low PSA levels are scored as Gleason grade 7 or higher, which is a measure of an aggressive prostate cancer.

In addition to the sensitivity problem outlined above, PSA testing also has difficulty with specificity and predicting prognosis. PSA levels can be abnormal in those without prostate cancer. For example, benign prostatic hyperplasia (BPH) is one common cause of a false-positive PSA test. In addition, a variety of non-cancer conditions may elevate serum PSA levels, including urinary retention, prostatitis, vigorous prostate massage and ejaculation. Subsequent confirmation of prostate cancer using needle biopsy in patients with positive PSA levels is difficult if the tumor is too small to see by ultrasound. Multiple random samples are typically taken but diagnosis of prostate cancer may be missed because of the sampling of only small amounts of tissue. Digital rectal examination (DRE) also misses many cancers because only the posterior lobe of the prostate is examined. As early cancers are nonpalpable, cancers detected by DRE may already have spread outside the prostate (Mistry K. J., Am. Board Fam. Pract.16(2):95-101 (2003)).

Thus, there is clearly a great need for improved diagnostic procedures that would facilitate early-stage prostate cancer detection and prognosis, as well as aid in preventive and curative treatments of the disease. In addition, there is a need to develop tools to better identify those patients who are more likely to have aggressive forms of prostate cancer from those patients that are more likely to have more benign forms of prostate cancer that remain localized within the prostate and do not contribute significantly to morbidity or mortality. This would help to avoid invasive and costly procedures for patients not at significant risk.

Although genetic factors are among the strongest epidemiological risk factors for prostate cancer, the search for genetic determinants involved in the disease has been challenging. Studies have revealed that linking candidate genetic markers to prostate cancer has been more difficult than identifying susceptibility genes for other cancers, such as breast, ovary and colon cancer. Several reasons have been proposed for this increased difficulty including: the fact that prostate cancer is often diagnosed at a late age thereby often making it difficult to obtain DNA samples from living affected individuals for more than one generation; the presence within high-risk pedigrees of phenocopies that are associated with a lack of distinguishing features between hereditary and sporadic forms; and the genetic heterogeneity of prostate cancer and the accompanying difficulty of developing appropriate statistical transmission models for this complex disease (Simard, J. et al., Endocrinology 143(6):2029-40 (2002)).

Various genome scans for prostate cancer-susceptibility genes have been conducted and several prostate cancer susceptibility loci have been reported. For example, HPC1 (1q24-q25), PCAP (1q42-q43), HCPX (Xq27-q28), CAPB (1p36), HPC20 (20q13), HPC2/ELAC2 (17 μl) and 16q23 have been proposed as prostate cancer susceptibility loci (Simard, J. et al., Endocrinology 143(6):2029-40 (2002); Nwosu, V. et al., Hum. Mol. Genet. 10(20):2313-18 (2001)). In a genome scan conducted by Smith et al., the strongest evidence for linkage was at HPC1, although two-point analysis also revealed a LOD score of a 1.5 at D4S430 and LOD scores a 1.0 at several loci, including markers at Xq27-28 (Ostrander E. A. and J. L. Stanford, Am. J. Hum. Genet. 67:1367-75 (2000)). In other genome scans, two-point LOD scores of a 1.5 for chromosomes 10q, 12q and 14q using an autosomal dominant model of inheritance, and chromosomes 1q, 8q, 10q and 16p using a recessive model of inheritance, have been reported, as well as nominal evidence for linkage to chr 2q, 12p, 15q, 16q and 16p. A genome scan for prostate cancer predisposition loci using a small set of Utah high risk prostate cancer pedigrees and a set of 300 polymorphic markers provided evidence for linkage to a locus on chromosome 17p (Simard, J. et al., Endocrinology 143(6):2029-40 (2002)). Eight new linkage analyses were published in late 2003, which depicted remarkable heterogeneity. Eleven peaks with LOD scores higher than 2.0 were reported, none of which overlapped (see Actane consortium, Schleutker et. al, Wiklund et. al., Witte et. al., Janer et. al., Xu et. al., Lange et. al., Cunningham et al.; all of which appear in Prostate, vol. 57 (2003)).

As described above, identification of particular genes involved in prostate cancer has been challenging. One gene that has been implicated is RNASEL, which encodes a widely expressed latent endoribonuclease that participates in an interferon-inducible RNA-decay pathway believed to degrade viral and cellular RNA, and has been linked to the HPC locus (Carpten, J. et al., Nat. Genet. 30:181-84 (2002); Casey, G. et al., Nat. Genet. 32(4):581-83 (2002)). Mutations in RNASEL have been associated with increased susceptibility to prostate cancer. For example, in one family, four brothers with prostate cancer carried a disabling mutation in RNASEL, while in another family, four of six brothers with prostate cancer carried a base substitution affecting the initiator methionine codon of RNASEL. Other studies have revealed mutant RNASEL alleles associated with an increased risk of prostate cancer in Finnish men with familial prostate cancer and an Ashkenazi Jewish population (Rokman, A. et al., Am J. Hum. Genet. 70:1299-1304 (2002); Rennert, H. et al., Am J. Hum. Genet. 71:981-84 (2002)). In addition, the Ser217Leu genotype has been proposed to account for approximately 9% of all sporadic cases in Caucasian Americans younger than 65 years (Stanford, J. L., Cancer Epidemiol. Biomarkers Prev. 12(9):876-81 (2003)). In contrast to these positive reports, however, some studies have failed to detect any association between RNASEL alleles with inactivating mutations and prostate cancer (Wang, L. et al., Am. J. Hum. Genet. 71:116-23 (2002); Wiklund, F. et al., Clin. Cancer Res. 10(21):7150-56 (2004); Maier, C. et. al., Br. J. Cancer 92(6):1159-64 (2005)).

The macrophage-scavenger receptor 1 (MSR1) gene, which is located at 8p22, has also been identified as a candidate prostate cancer-susceptibility gene (Xu, J. et al., Nat. Genet. 32:321-25 (2002)). A mutant MSR1 allele was detected in approximately 3% of men with nonhereditary prostate cancer but only 0.4% of unaffected men. However, not all subsequent reports have confirmed these initial findings (see, e.g., Lindmark, F. et al., Prostate 59(2):132-40 (2004); Seppala, E. H. et al., Clin. Cancer Res. 9(14):5252-56 (2003); Wang, L. et al., Nat. Genet. 35(2):128-29 (2003); Miller, D. C. et al., Cancer Res. 63(13):3486-89 (2003)). MSR1 encodes subunits of a macrophage-scavenger receptor that is capable of binding a variety of ligands, including bacterial lipopolysaccharide and lipoteicholic acid, and oxidized high-density lipoprotein and low-density lipoprotein in serum (Nelson, W. G. et al., N. Engl. J. Med. 349(4):366-81 (2003)).

The ELAC2 gene on Chr17p was the first prostate cancer susceptibility gene to be cloned in high risk prostate cancer families from Utah (Tavtigian, S. V., et al., Nat. Genet. 27(2):172-80 (2001)). A frameshift mutation (1641InsG) was found in one pedigree. Three additional missense changes: Ser217Leu; Ala541Thr; and Arg781H is, were also found to associate with an increased risk of prostate cancer. The relative risk of prostate cancer in men carrying both Ser217Leu and Ala541Thr was found to be 2.37 in a cohort not selected on the basis of family history of prostate cancer (Rebbeck, T. R., et al., Am. J. Hum. Genet. 67(4):1014-19 (2000)). Another study described a new termination mutation (GIu216X) in one high incidence prostate cancer family (Wang, L., et al., Cancer Res. 61(17):6494-99 (2001)). Other reports have not demonstrated strong association with the three missense mutations, and a recent metaanalysis suggests that the familial risk associated with these mutations is more moderate than was indicated in initial reports (Vesprini, D., et al., Am. J. Hum. Genet. 68(4):912-17 (2001); Shea, P. R., et al., Hum. Genet. 111(4-5):398-400 (2002); Suarez, B. K., et al., Cancer Res. 61(13):4982-84 (2001); Severi, G., et al., J. Natl. Cancer Inst. 95(11):818-24 (2003); Fujiwara, H., et al., J. Hum. Genet. 47(12):641-48 (2002); Camp, N. J., et al., Am. J. Hum. Genet. 71(6):1475-78 (2002)).

Polymorphic variants of genes involved in androgen action (e.g., the androgen receptor (AR) gene, the cytochrome P-450c17 (CYP17) gene, and the steroid-5-α-reductase type II (SRD5A2) gene), have also been implicated in increased risk of prostate cancer (Nelson, W. G. et al., N. Engl. J. Med. 349(4):366-81 (2003)). With respect to AR, which encodes the androgen receptor, several genetic epidemiological studies have shown a correlation between an increased risk of prostate cancer and the presence of short androgen-receptor polyglutamine repeats, while other studies have failed to detect such a correlation. Linkage data has also implicated an allelic form of CYP17, an enzyme that catalyzes key reactions in sex-steroid biosynthesis, with prostate cancer (Chang, B. et al., Int. J. Cancer 95:354-59 (2001)). Allelic variants of SRD5A2, which encodes the predominant isozyme of 5-α-reductase in the prostate and functions to convert testosterone to the more potent dihydrotestosterone, have been associated with an increased risk of prostate cancer and with a poor prognosis for men with prostate cancer (Makridakis, N. M. et al., Lancet 354:975-78 (1999); Nam, R. K. et al., Urology 57:199-204 (2001)).

It is likely that a relatively high number of low-to-medium risk genetic variants contribute to risk of prostate cancer. These low-to-medium risk genetic variants may, however, be responsible for a substantial fraction of prostate cancer, and their identification, therefore, a great benefit for public health. Several such variants have been identified in the last two years, mainly through large-scale genome-wide association studies (Gudmundsson, J., et al. Nat Genet 40:281-283 (2008); Thomas, G., et al. Nat Genet 40:310-315 (2008); Gudmundsson, J., et al. Nat Genet 39:977-983 (2007); Yeager, M., et al Nat Genet 39:645-649 (2007); Gudmundsson, J., et al. Nat Genet 39:631-637 (2007); Amundadottir, L. T., et al. Nat Genet 38:652-658 (2007); Haiman, C. A., et al. Nat Genet 39:638-644 (2007)).

Colorectal Cancer (CRC) is one of the most commonly diagnosed cancers and one of the leading causes of cancer mortality (Parkin D M, et. al. CA Cancer J Clin, 55:74-108 (2005)). Cancers of the colon and rectum accounted for about 1 million new cases in 2002 (9.4% of cancer cases world-wide) and it affects men and women almost equally. The average lifetime risk for an individual in the US to develop CRC is 6% (Jemal A, et. al. CA Cancer J. Clin., 56:106-30 (2006)). The prognosis is strongly associated with the stage of the disease at diagnosis; therefore, CRC screening presents an opportunity for early cancer detection and cancer prevention.

Colorectal cancer is a consequence of environmental exposures acting upon a background of genetically determined susceptibility. Studies indicate that 30-35% of colorectal cancer risk could be explained by genetic factors (Lichtenstein P, et. al. N Engl J Med, 343:78-85 (2000);) Peto 3 and Mack T M. Nat Genet, 26:411-4 (2000); Risch N. Cancer Epidemiol Biomarkers Prev, 10:733-41 (2001)). The analysis of cancer occurrence in relatives of cancer patients also lends strong evidence for genetic factors that increase the risk of cancer.

At present only a small percentage of the heritable risk of CRC is identified, usually through the investigation of rare cancer syndromes. High-penetrance mutations in several genes have been identified in rare hereditary colorectal cancer syndromes. The most common of these are the familial adenomatous polyposis (FAP) syndrome and hereditary non-polyposis colorectal cancer (HNPCC) or Lynch syndrome (LS). FAP, caused by mutations in the APC gene, is an autosomal dominant syndrome, characterized by early onset of multiple adenomatous polyps in the colon that eventually progress to cancer. LS is caused by mutations in DNA mismatch repair (MMR) genes and is considered to be the most common hereditary CRC syndrome, comprising approximately 3-5% of all CRCs (de la Chapelle, A. Fam Cancer, 4:233-7 (2005)).

The search for additional highly-penetrant CRC genes has not been fruitful and accumulating evidence supports the notion that no single susceptibility gene is likely to explain a large proportion of highly familial or early onset CRC. This has led to the currently favored hypothesis that most of the inherited CRC risk is due to multiple, low genetic risk variants. Each such variant would be expected to carry a small increase in risk; however, if the variant is common, it may contribute significantly to the population attributable risk (PAR).

Cervical Cancer. Cervical cancer (CC) is the second most common cancer and the third most frequent cause of cancer death in women, accounting for over 490,000 cases and nearly 300,000 deaths annually (Parkin, D. M., et al., CA Cancer J Clin, 55: 74-108 (2005)). CC used to be a major cause of death in women in child-bearing age in the US and Europe but with the introduction of the Papanicolaou (PAP) smear in the 1950s, the incidence of invasive cervical cancer declined dramatically. Currently, about 70% of cervical cancer deaths occur in low-to medium income countries where population-based screening has not been implemented and access to healthcare is poor. In 2008, an estimated 11,070 women in the United States will be diagnosed with CC, and an estimated 3,870 will die of the disease (SEER Cancer Statistics Review, 1975-2005. Bethesda: National Cancer Institute, (2007)). In certain populations and geographic areas of the United States, cervical cancer death rates are still high, in large part due to limited access to health care and cervical cancer screening.

CC is almost invariably associated with infection by an oncogenic subtype of Human Papillomavirus (HPV) (Munoz, N. J Clin Virol, 19: 1-5 (2000); Walboomers, J. M., et al., J Pathol, 189: 12-9 (1999)). The majority of cases, or close to 70% of cervical cancer worldwide, is caused by HPV16 and 18, while HPV45, 31, 33 and other less common variants are found in the remaining cases. Infection by HPV causes dysplastic lesions in the cervical epithelium that, in the great majority of cases, are self-limiting, demonstrating effective host immune response to the virally infected cells (zur Hausen, H., J Natl Cancer Inst, 92: 690-8 (2000)). However, in some cases, the immune system fails to clear the infection which may become chronic and eventually lead to growth of malignant cells and the development of invasive cancer. Several cofactors have been identified that slightly increase the risk of cervical cancer in HPV-infected individuals, e.g. previous chlamydia infection (Anttila, T., et al., JAMA, 285: 47-51 (2001)), multiple sexual partners and cigarette smoking (Murthy, N. S, and Mathew, A., Eur J Cancer Prey, 9: 5-14 (2000)).

Genetic factors have been shown to play a role in the development of CC. Studies based on the nationwide Swedish Family-Cancer Database suggested that close to 22% of CC risk could be attributed to genetic factors while shared environmental effects did not contribute to the disease (Czene, K., et al., Int J Cancer, 99: 260-6 (2002)). In a subsequent study, full and half-siblings were identified from the Family-Cancer Database and it was shown that the familial risk for full siblings was 1.84, compared with 1.40 for maternal and 1.27 for paternal half-siblings. These data were used to apportion familial risk for cervical tumors in full siblings into a heritable component, accounting for 64%, and an environmental component, accounting for 36% of the total risk (Hemminki, K., and Chen, B., Cancer Epidemiol Biomarkers Prev, 15: 1413-4 (2006)). Finally, a study of 18,199 women with invasive and/or in situ cervix cancers and 72,796 women free of cervical tumors suggested a heritable component of 71% and an environmental component of 29% in young familial cervical tumors (Couto, E., and Hemminki, K., Int J Cancer, 119: 2699-701 (2006)). Taken together, these studies show that genetic factors play a substantial role in cervical cancer development, possibly by affecting immunological mechanisms that help clear HPV infection.

While cytological examination of PAP smears is highly effective in detecting dysplastic lesions and early stage CC which can be effectively treated by cone operation, a fraction of cases present with a persistent infection or re-infection which may progress to invasive cancer (Schiffman, M., et al., Lancet, 370: 890-907 (2007)). These cases often need to be followed for years and subjected to repeated biopsies. There is an unmet clinical need to identify women with persistent or recurring infection that have the greatest risk of progressing to invasive CC. Such individuals might be subjected to a more rigorous follow-up protocols or advised on how to reduce the risk by lifestyle changes. Knowledge of the underlying genetic predisposition might be useful in evaluating risks of progression. Screening for susceptibility to CC might also be important in planning the clinical management of transplant recipients and other immunosuppressed individuals.

Recently, vaccines against the major oncogenic subtypes of HPV have been developed that hold a great promise in the battle against the disease (Cutts, F. T., et al., Bull World Health Organ, 85: 719-26 (2007)). However, the vaccines are only effective in women with no prior infection with HPV and therefore it will take decades before the majority of women are protected. Furthermore, until all oncogenic subtypes are included in the vaccine, some form of organized screening will be necessary in order to catch those cases.

Clearly, identification of markers and genes that are responsible for susceptibility to cancer is one of the major challenges facing oncology today. Some of the pathways underlying cancer are shared in different forms of cancer. As a consequence, genetic risk factors identified for one particular form of cancer may also represent a risk factor for other cancer types. Diagnostic and therapeutic methods utilizing these risk factors may therefore have a common utility. Accordingly, therapeutic measures developed to target such risk factors may have implications for cancer in general, and not necessarily only the cancer for which the risk factor is originally identified. There is a need to identify means for the early detection of individuals that have a genetic susceptibility to cancer so that more aggressive screening and intervention regimens may be instituted for the early detection and treatment of cancer. Cancer genes may also reveal key molecular pathways that may be manipulated (e.g., using small or large molecule weight drugs) and may lead to more effective treatments regardless of the cancer stage when a particular cancer is first diagnosed.

Recently, genome-wide association studies of several cancers have identified common genetic variants that associate with increased cancer risk (Gudmundsson, J., et al. Nat Genet 39:631-637 (2007); Stacey, S. N., et al., Nat. Genet. 39:865-69 (2007); Yeager, M. et al. Nat Genet. 39:645-649 (2007); Gudmundsson, J., et al. Nat Genet 39:977-983 (2007); Haiman, C. A., et al. Nat Genet 39:638-644 (2007); Eason, D. F., et al. Nature 447:1087-1093 (2007); Tomlinson, I., et al. Nat Genet 39:984-988 (2007); Gudbjartsson, D. F., et al. Nat Genet. 40:886-891 (2008); Stacey, S. N., et al. Nat Genet 40:703-706 (2008); Thorgeirsson, T. E., et al. Nature 452:638-642 (2008); Gudmundsson, J., et al. Nat Genet 40:281-283 (2008); Eeles, R. A., et al. Nat Genet 40:316-321 (2008); Hung, R. J., et al. Nature 452:633-637 (2008); Amos, C. I., et al. Nat Genet 40:616-622 (2008); Thomas, G., et al. Nat Genet. 40:310-315 (2008)). Notably, in most cases the reported variants seem to be specific to the particular cancer type under study. This tissue specificity even holds true in the region on chromosome 8q24, which has been found to associate with several different types of cancer.

The present inventors have now surprisingly found that variants on chromosome 5p13.3 associate with risk of several cancer types.

### SUMMARY OF THE INVENTION

The present invention relates to methods of risk management of cancer, based on the discovery that certain genetic variants are correlated with risk of cancer. Thus, the invention includes methods of determining an increased susceptibility or increased risk of cancer, as well as methods of determining a decreased susceptibility of cancer, through evaluation of certain markers that have been found to be correlated with susceptibility of cancer in humans. Other aspects of the invention relate to methods of assessing prognosis of individuals diagnosed with cancer, methods of assessing the probability of response to a therapeutic agents or therapy for cancer, as well as methods of monitoring progress of treatment of individuals diagnosed with cancer.

In one aspect, the present invention relates to a method of diagnosing a susceptibility to cancer in a human individual, the method comprising determining the presence or absence of at least one allele of at least one polymorphic marker on selected from rs401681 (SEQ ID NO:2), rs2736100 (SEQ ID NO:3) and rs2736098 (SEQ ID NO:4), and markers in linkage disequilibrium therewith, in a nucleic acid sample obtained from the individual, wherein the presence of the at least one allele is indicative of a susceptibility to cancer. The invention also relates to a method of determining a susceptibility to cancer, by determining the presence or absence of at least one allele of at least one polymorphic marker selected from rs401681 (SEQ ID NO:2), rs2736100 (SEQ ID NO:3) and rs2736098 (SEQ ID NO:4), and markers in linkage disequilibrium therewith, wherein the determination of the presence of the at least one allele is indicative of a susceptibility to cancer.

In another aspect the invention further relates to a method for determining a susceptibility to cancer in a human individual, comprising determining whether at least one allele of at least one polymorphic marker is present in a nucleic acid sample obtained from the individual, or in a genotype dataset derived from the individual, wherein the at least one polymorphic marker is selected from rs401681, rs2736100 and rs2736098, and markers in linkage disequilibrium therewith, and wherein the presence of the at least one allele is indicative of a susceptibility to cancer for the individual.

In another aspect, the invention relates to a method of determining a susceptibility to cancer in a human individual, comprising determining whether at least one at-risk allele in at least one polymorphic marker is present in a genotype dataset derived from the individual, wherein the at least one polymorphic marker is selected from markers rs401681, rs2736100 and rs2736098, and markers in linkage disequilibrium therewith, and wherein determination of the presence of the at least one at-risk allele is indicative of increased susceptibility to cancer in the individual.

The genotype dataset comprises in one embodiment information about marker identity and the allelic status of the individual for at least one allele of a marker, i.e. information about the identity of at least one allele of the marker in the individual. The genotype dataset may comprise allelic information (information about allelic status) about one or more marker, including two or more markers, three or more markers, five or more markers, ten or more markers, one hundred or more markers, and so on. In some embodiments, the genotype dataset comprises genotype information from a whole-genome assessment of the individual, that may include hundreds of thousands of markers, or even one million or more markers spanning the entire genome of the individual.

Another aspect of the invention relates to a method of determining a susceptibility to cancer in a human individual, the method comprising:

obtaining nucleic acid sequence data about a human individual identifying at least one allele of at least one polymorphic marker selected from rs401681, rs2736100 and rs2736098, and markers in linkage disequilibrium therewith, wherein different alleles of the at least one polymorphic marker are associated with different susceptibilities to cancer in humans, and determining a susceptibility to cancer from the nucleic acid sequence data.

The invention also relates to a method of determining a susceptibility to cancer in a human individual, the method comprising obtaining nucleic acid sequence data about a human individual identifying at least one allele of at least one polymorphic marker associated with the human telomerase reverse transcriptase (TERT) gene and/or the human cisplatin resistance related protein CRR9p (CLPTM1L) gene, and markers in linkage disequilibrium therewith, wherein different alleles of the at least one polymorphic marker are associated with different susceptibilities to cancer in humans, and determining a susceptibility to cancer from the nucleic acid sequence data. Markers that are useful for determining susceptibility to cancer are correlated with risk of cancer in humans. In one embodiment, the at least one marker is a marker associated with the human TERT gene. In certain embodiments, the at least one marker is a marker within the genomic segment with sequence as set forth in SEQ ID NO:1. In certain embodiments, the at least one marker is selected from the group consisting of the markers set forth in Table 8 and Table 9. In certain embodiments, the at least one marker is selected from the group consisting of the markers listed in Table 9 herein.

In general, polymorphic genetic markers lead to alternate sequences at the nucleic acid level. If the nucleic acid marker changes the codon of a polypeptide encoded by the nucleic acid, then the marker will also result in alternate sequence at the amino acid level of the encoded polypeptide (polypeptide markers). Determination of the identity of particular alleles at polymorphic markers in a nucleic acid or particular alleles at polypeptide markers comprises whether particular alleles are present at a certain position in the sequence. Sequence data identifying a particular allele at a marker comprises sufficient sequence to detect the particular allele. For single nucleotide polymorphisms (SNPs) or amino acid polymorphisms described herein, sequence data can comprise sequence at a single position, i.e. the identity of a nucleotide or amino acid at a single position within a sequence. The sequence data can optionally include information about sequence flanking the polymorphic site, which in the case of SNPs spans a single nucleotide.

In certain embodiments, it may be useful to determine the nucleic acid sequence for at least two polymorphic markers. In other embodiments, the nucleic acid sequence for at least three, at least four or at least five or more polymorphic markers is determined. Haplotype information can be derived from an analysis of two or more polymorphic markers. Thus, in certain embodiments, a further step is performed, whereby haplotype information is derived based on sequence data for at least two polymorphic markers.

The invention also provides a method of determining a susceptibility to cancer in a human individual, the method comprising obtaining nucleic acid sequence data about a human individual identifying both alleles of at least two polymorphic markers selected from rs401681, rs2736100 and rs2736098, and markers in linkage disequilibrium therewith, determine the identity of at least one haplotype based on the sequence data, and determine a susceptibility to cancer from the haplotype data.

In certain embodiments, determination of a susceptibility comprises comparing the nucleic acid sequence data to a database containing correlation data between the at least one polymorphic marker and susceptibility to cancer. In some embodiments, the database comprises at least one risk measure of susceptibility to cancer for the at least one marker. The sequence database can for example be provided as a look-up table that contains data that indicates the susceptibility of cancer for any one, or a plurality of, particular polymorphisms. The database may also contain data that indicates the susceptibility for a particular haplotype that comprises at least two polymorphic markers.

Obtaining nucleic acid sequence data can in certain embodiments comprise obtaining a biological sample from the human individual and analyzing sequence of the at least one polymorphic marker in nucleic acid in the sample. Analyzing sequence can comprise determining the presence or absence of at least one allele of the at least one polymorphic marker. Determination of the presence of a particular susceptibility allele (e.g., an at-risk allele) is indicative of susceptibility to cancer in the human individual. Determination of the absence of a particular susceptibility allele is indicative that the particular susceptibility due to the at least one polymorphism is not present in the individual.

In some embodiments, obtaining nucleic acid sequence data comprises obtaining nucleic acid sequence information from a preexisting record. The preexisting record can for example be a computer file or database containing sequence data, such as genotype data, for the human individual, for at least one polymorphic marker.

Susceptibility determined by the diagnostic methods of the invention can be reported to a particular entity. In some embodiments, the at least one entity is selected from the group consisting of the individual, a guardian of the individual, a genetic service provider, a physician, a medical organization, and a medical insurer.

In certain embodiments, the at least one polymorphic marker is associated with the TERT gene. In certain other embodiments, the at least one polymorphic marker is associated with the CLPTM1L gene.

In certain embodiments of the invention, determination of a susceptibility comprises comparing the nucleic acid sequence data to a database containing correlation data between the at least one polymorphic marker and susceptibility to cancer. In one such embodiment, the database comprises at least one risk measure of susceptibility to cancer for the at least one polymorphic marker. In another embodiment, the database comprises a look-up table containing at least one risk measure of the at least one condition for the at least one polymorphic marker.

In certain embodiments, obtaining nucleic acid sequence data comprises obtaining a biological sample from the human individual and analyzing sequence of the at least one polymorphic marker in nucleic acid in the sample. Analyzing sequence of the at least one polymorphic marker can comprise determining the presence or absence of at least one allele of the at least one polymorphic marker. Obtaining nucleic acid sequence data can also comprise obtaining nucleic acid sequence information from a preexisting record.

Certain embodiments of the invention relate to obtaining nucleic acid sequence data about at least two polymorphic markers selected from rs401681, rs2736100 and rs2736098, and markers in linkage disequilibrium therewith.

In certain embodiments of the invention, the at least one polymorphic marker is selected from the markers set forth in Table 5, Table 6 and Table 7. In one embodiment, the at least one polymorphic marker is selected from the markers as set forth in Table 5. In another embodiment, the at least one polymorphic marker is selected from the markers as set forth in Table 6. In another embodiment, the at least one polymorphic marker is selected from the markers as set forth in Table 7.

Another aspect of the invention relates to a method of determining a susceptibility to cancer in a human individual, the method comprising determining whether at least one allele of at least one polymorphic marker is present in a nucleic acid sample obtained from the individual, or in a genotype dataset derived from the individual, wherein the at least one polymorphic marker is associated with the TERT gene, and wherein the presence of the at least one allele is indicative of a susceptibility to cancer for the individual. In certain embodiments, the markers associated with the TERT gene are in linkage disequilibrium with the TERT gene. In certain embodiments, the at least one polymorphic marker is selected from the markers set forth in Table 8.

In certain embodiments of the invention, a further step of assessing the frequency of at least one haplotype in the individual is performed. In such embodiments, two or more markers, including three, four, five, six, seven, eight, nine or ten or more markers can be included in the haplotype. In certain embodiments, the at least one haplotype comprises markers selected from rs401681, rs2736100 and rs2736098, and markers in linkage disequilibrium therewith. In certain such embodiments, the at least one haplotype is representative of the genomic structure of a particular genomic region (such as an LD block), to which any one of the above-mentioned markers reside.

The markers conferring risk of cancer, as described herein, can be combined with other genetic markers for cancer. Such markers are typically not in linkage disequilibrium with any one of the markers described herein, in particular markers rs401681, rs2736100 and rs2736098. Any of the methods described herein can be practiced by combining the genetic risk factors described herein with additional genetic risk factors for cancer. Such additional risk factors are in certain embodiments risk factors for a particular type of cancer, i.e. cancer at a particular site. In certain other embodiments, such additional risk factors are susceptibility variants for multiple forms of cancer.

Thus, in certain embodiments, a further step is included, comprising determining whether at least one at-risk allele of at least one at-risk variant for cancer not in linkage disequilibrium with any one of the markers rs401681, rs2736100 and rs2736098 are present in a sample comprising genomic DNA from a human individual or a genotype dataset derived from a human individual. In other words, genetic markers in other locations in the genome can be useful in combination with the markers of the present invention, so as to determine overall risk of cancer based on multiple genetic variants. In one embodiment, the at least one at-risk variant for cancer is not in linkage disequilibrium with marker rs2736098. Selection of markers that are not in linkage disequilibrium (not in LD) can be based on a suitable measure for linkage disequilibrium, as described further herein. In certain embodiments, markers that are not in linkage disequilibrium have values for the LD measure r² correlating the markers of less than 0.2. In certain other embodiments, markers that are not in LD have values for r² correlating the markers of less than 0.15, including less than 0.10, less than 0.05, less than 0.02 and less than 0.01. Other suitable numerical values for establishing that markers are not in LD are contemplated, including values bridging any of the above-mentioned values.

In certain embodiments, multiple markers as described herein are determined to determine overall risk of cancer. Thus, in certain embodiments, an additional step is included, the step comprising determining whether at least one allele in each of at least two polymorphic markers is present in a sample comprising genomic DNA from a human individual or a genotype dataset derived from a human individual, wherein the presence of the at least one allele in the at least two polymorphic markers is indicative of an increased susceptibility to cancer. In one embodiment, the markers are selected from rs401681, rs2736100 and rs2736098, and markers in linkage disequilibrium therewith.

The genetic markers of the invention can also be combined with non-genetic information to establish overall risk for an individual. Thus, in certain embodiments, a further step is included, comprising analyzing non-genetic information to make risk assessment, diagnosis, or prognosis of the individual. The non-genetic information can be any information pertaining to the disease status of the individual or other information that can influence the estimate of overall risk of cancer for the individual. In one embodiment, the non-genetic information is selected from age, gender, ethnicity, socioeconomic status, previous disease diagnosis, medical history of subject, family history of cancer, biochemical measurements, and clinical measurements.

The invention also provides computer-implemented aspects. In one such aspect, the invention provides a computer-readable medium having computer executable instructions for determining susceptibility to cancer in an individual, the computer readable medium comprising: data representing at least one polymorphic marker; and a routine stored on the computer readable medium and adapted to be executed by a processor to determine susceptibility to cancer in an individual based on the allelic status of at least one allele of said at least one polymorphic marker in the individual.

In one embodiment, said data representing at least one polymorphic marker comprises at least one parameter indicative of the susceptibility to cancer linked to said at least one polymorphic marker. In another embodiment, said data representing at least one polymorphic marker comprises data indicative of the allelic status of at least one allele of said at least one allelic marker in said individual. In another embodiment, said routine is adapted to receive input data indicative of the allelic status for at least one allele of said at least one allelic marker in said individual. In a preferred embodiment, the at least one marker is selected from rs401681, rs2736100 and rs2736098, and markers in linkage disequilibrium therewith. In another preferred embodiment, the at least one polymorphic marker is selected from the markers set forth in Table 5, Table 6 and Table 7.

The invention further provides an apparatus for determining a genetic indicator for cancer in a human individual, comprising:

a processor,

a computer readable memory having computer executable instructions adapted to be executed on the processor to analyze marker and/or haplotype information for at least one human individual with respect to cancer, and generate an output based on the marker or haplotype information, wherein the output comprises a risk measure of the at least one marker or haplotype as a genetic indicator of cancer for the human individual. In one embodiment, the computer readable memory comprises data indicative of the frequency of at least one allele of at least one polymorphic marker or at least one haplotype in a plurality of individuals diagnosed with cancer, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with cancer. In one embodiment, the computer readable memory further comprises data indicative of a risk of developing cancer associated with at least one allele of at least one polymorphic marker or at least one haplotype, and wherein a risk measure for the human individual is based on a comparison of the at least one marker and/or haplotype status for the human individual to the risk associated with the at least one allele of the at least one polymorphic marker or the at least one haplotype. In another embodiment, the computer readable memory further comprises data indicative of the frequency of at least one allele of at least one polymorphic marker or at least one haplotype in a plurality of individuals diagnosed with cancer, and data indicative of the frequency of at the least one allele of at least one polymorphic marker or at least one haplotype in a plurality of reference individuals, and wherein risk of developing cancer is based on a comparison of the frequency of the at least one allele or haplotype in individuals diagnosed with cancer, and reference individuals. In a preferred embodiment, the at least one marker is selected from rs401681, rs2736100 and rs2736098, and markers in linkage disequilibrium therewith. In another preferred embodiment, the at least one polymorphic marker is selected from the markers set forth in Table 5, Table 6 and Table 7.

In another aspect, the invention relates to a method of identification of a marker for use in assessing susceptibility to cancer, the method comprising: identifying at least one polymorphic marker in linkage disequilibrium with at least one of rs401681, rs2736100 and rs2736098; determining the genotype status of a sample of individuals diagnosed with, or having a susceptibility to, cancer; and determining the genotype status of a sample of control individuals; wherein a significant difference in frequency of at least one allele in at least one polymorphism in individuals diagnosed with, or having a susceptibility to, cancer, as compared with the frequency of the at least one allele in the control sample is indicative of the at least one polymorphism being useful for assessing susceptibility to cancer. Significant difference can be estimated on statistical analysis of allelic counts at certain polymorphic markers in cancer patients and controls. In one embodiment, a significant difference is based on a calculated P-value between cancer patients and controls of less than 0.05. In other embodiments, a significant difference is based on a lower value of the calculated P-value, such as less than 0.005, 0.0005, or less than 0.00005. In one embodiment, an increase in frequency of the at least one allele in the at least one polymorphism in individuals diagnosed with, or having a susceptibility to, cancer, as compared with the frequency of the at least one allele in the control sample is indicative of the at least one polymorphism being useful for assessing increased susceptibility to cancer. In another embodiment, a decrease in frequency of the at least one allele in the at least one polymorphism in individuals diagnosed with, or having a susceptibility to, cancer, as compared with the frequency of the at least one allele in the control sample is indicative of the at least one polymorphism being useful for assessing decreased susceptibility to, or protection against, cancer.

The invention also relates to a method of genotyping a nucleic acid sample obtained from a human individual comprising determining whether at least one allele of at least one polymorphic marker is present in a nucleic acid sample from the individual sample, wherein the at least one marker is selected from rs401681, rs2736100 and rs2736098, and markers in linkage disequilibrium therewith, and wherein determination of the presence of the at least one allele in the sample is indicative of a susceptibility to cancer in the individual. In one embodiment, determination of the presence of allele C of rs401681, allele G of rs2736100 and/or allele A of rs2736098 is indicative of increased susceptibility of cancer in the individual. Alternatively, marker alleles in linkage disequilibrium with any one of allele C of rs401681, allele G of rs2736100 and/or allele A of rs2736098 are indicative of increased susceptibility of the cancer. In another embodiment, determination of the presence of allele C of rs401681, allele G of rs2736100 and/or allele A of rs2736098, or marker alleles in linkage disequilibrium therewith is indicative of a decreased susceptibility of melanoma cancer or colorectal cancer in an individual. In one embodiment, genotyping comprises amplifying a segment of a nucleic acid that comprises the at least one polymorphic marker by Polymerase Chain Reaction (PCR), using a nucleotide primer pair flanking the at least one polymorphic marker. In another embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, single-stranded conformation analysis and microarray technology. In one embodiment, the microarray technology is Molecular Inversion Probe array technology or BeadArray Technologies. In one embodiment, the process comprises allele-specific probe hybridization. In another embodiment, the process comprises microrray technology. One preferred embodiment comprises the steps of (1) contacting copies of the nucleic acid with a detection oligonucleotide probe and an enhancer oligonucleotide probe under conditions for specific hybridization of the oligonucleotide probe with the nucleic acid; wherein (a) the detection oligonucleotide probe is from 5-100 nucleotides in length and specifically hybridizes to a first segment of a nucleic acid whose nucleotide sequence is set forth in SEQ ID NO:1; (b) the detection oligonucleotide probe comprises a detectable label at its 3′ terminus and a quenching moiety at its 5′ terminus; (c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and (d) a single base gap exists between the first segment and the second segment, such that when the oligonucleotide probe and the enhancer oligonucleotide probe are both hybridized to the nucleic acid, a single base gap exists between the oligonucleotides; (2) treating the nucleic acid with an endonuclease that will cleave the detectable label from the 3′ terminus of the detection probe to release free detectable label when the detection probe is hybridized to the nucleic acid; and (3) measuring free detectable label, wherein the presence of the free detectable label indicates that the detection probe specifically hybridizes to the first segment of the nucleic acid, and indicates the sequence of the polymorphic site as the complement of the detection probe.

A further aspect of the invention pertains to a method of assessing an individual for probability of response to a cancer therapeutic agent, comprising: determining whether at least one allele of at least one polymorphic marker is present in a nucleic acid sample obtained from the individual, or in a genotype dataset derived from the individual, wherein the at least one polymorphic marker is selected from rs401681, rs2736100 and rs2736098, and markers in linkage disequilibrium therewith, wherein the presence of the at least one allele of the at least one marker is indicative of a probability of a positive response to the therapeutic agent

The invention in another aspect relates to a method of predicting prognosis of an individual diagnosed with cancer, the method comprising determining whether at least one allele of at least one polymorphic marker is present in a nucleic acid sample obtained from the individual, or in a genotype dataset derived from the individual, wherein the at least one polymorphic marker is selected from rs401681, rs2736100 and rs2736098, and markers in linkage disequilibrium therewith, wherein the presence of the at least one allele is indicative of a worse prognosis of the cancer in the individual.

Yet another aspect of the invention relates to a method of monitoring progress of treatment of an individual undergoing treatment for cancer, the method comprising determining whether at least one allele of at least one polymorphic marker is present in a nucleic acid sample obtained from the individual, or in a genotype dataset derived from the individual, wherein the at least one polymorphic marker is selected rs401681, rs2736100 and rs2736098, and markers in linkage disequilibrium therewith, wherein the presence of the at least one allele is indicative of the treatment outcome of the individual. In one embodiment, the treatment is treatment by surgery, treatment by radiation therapy, or treatment by drug administration.

The invention also relates to the use of an oligonucleotide probe in the manufacture of a reagent for diagnosing and/or assessing susceptibility to cancer in a human individual, wherein the probe hybridizes to a segment of a nucleic acid with nucleotide sequence as set forth in SEQ ID NO:1, wherein the probe is 15-500 nucleotides in length. In certain embodiments, the probe is about 16 to about 100 nucleotides in length. In certain other embodiments, the probe is about 20 to about 50 nucleotides in length. In certain other embodiments, the probe is about 20 to about 30 nucleotides in length.

The present invention, in its broadest sense relates to cancer in general, as the variants disclosed have been shown to be associated with risk of a variety of cancer types. In certain embodiments, the invention relates to certain cancer types, i.e. cancer at certain sites. Thus in certain embodiments of the invention, including any of the methods, kits, apparatus, uses and procedures as described herein, the cancer is selected from Basal Cell Carcinoma, Lung Cancer, Bladder Cancer, Prostate Cancer, Cervical Cancer, Thyroid Cancer, Melanoma Cancer (e.g., Cutaneous Melanoma), Colorectal Cancer and Endometrial Cancer. Any combinations of these cancer types are also contemplated, and within scope of the invention. Also, the present invention is contemplated to relate to any particular sub-phenotype of these cancer types.

In some embodiments of the methods of the invention, the susceptibility determined in the method is increased susceptibility. In one such embodiment, the increased susceptibility is characterized by a relative risk (RR) of at least 1.08. In another embodiment, the increased susceptibility is characterized by a relative risk of at least 1.10. In another embodiment, the increased susceptibility is characterized by a relative risk of at least 1.11. In another embodiment, the increased susceptibility is characterized by a relative risk of at least 1.12. In yet another embodiment, the increased susceptibility is characterized by a relative risk of at least 1.13. In a further embodiment, the increased susceptibility is characterized by a relative risk of at least 1.14. In a further embodiment, the increased susceptibility is characterized by a relative risk of at least 1.15. In yet another embodiment, the increased susceptibility is characterized by a relative risk of at least 1.20. Certain other embodiments are characterized by relative risk of the at-risk variant of at least 1.16, 1.17, 1.18, 1.19, 1.21, 1.22, 1.23, 1.24, 1.25, 1.26, 1.27, 1.28, 1.29, 1.30, and so on. Other numeric values of odds ratios, including those bridging any of these above-mentioned values are also possible, and these are also within scope of the invention.

In some embodiments of the methods of the invention, the susceptibility determined in the method is decreased susceptibility. In one such embodiment, the decreased susceptibility is characterized by a relative risk (RR) of less than 0.9. In another embodiment, the decreased susceptibility is characterized by a relative risk (RR) of less than 0.85. In another embodiment, the decreased susceptibility is characterized by a relative risk (RR) of less than 0.8. In yet another embodiment, the decreased susceptibility is characterized by a relative risk (RR) of less than 0.75. Other cutoffs, such as relative risk of less than 0.89, 0.88, 0.87, 0.86, 0.84, 0.83, 0.82, 0.81, 0.79, and so on, are also contemplated and are within scope of the invention.

The invention also relates to kits. In one such aspect, the invention relates to a kit for assessing susceptibility to cancer in a human individual, the kit comprising reagents necessary for selectively detecting at least one allele of at least one polymorphic marker selected from rs401681, rs2736100 and rs2736098, and markers in linkage disequilibrium therewith, in the genome of the individual, wherein the presence of the at least one allele is indicative of increased susceptibility to cancer. In another aspect, the invention relates to a kit for assessing susceptibility to cancer in a human individual, the kit comprising reagents for selectively detecting at least one allele of at least one polymorphic marker in the genome of the individual, wherein the polymorphic marker is selected from rs401681, rs2736100 and rs2736098, and wherein the presence of the at least one allele is indicative of a susceptibility to cancer. In one embodiment, the at least one polymorphic marker is selected from the markers set forth in Table 5, Table 6 and Table 7. In certain embodiments, the kit further comprises a collection of data comprising correlation data between the polymorphic markers assessed by the kit and susceptibility to the cancer.

Kit reagents may in one embodiment comprise at least one contiguous oligonucleotide that hybridizes to a fragment of the genome of the individual comprising the at least one polymorphic marker. In another embodiment, the kit comprises at least one pair of oligonucleotides that hybridize to opposite strands of a genomic segment obtained from the subject, wherein each oligonucleotide primer pair is designed to selectively amplify a fragment of the genome of the individual that includes one polymorphism, wherein the polymorphism is selected from the group consisting of the polymorphisms as defined in Table 5, Table 6 and Table 7, and wherein the fragment is at least 20 base pairs in size. In one embodiment, the oligonucleotide is completely complementary to the genome of the individual. In another embodiment, the kit further contains buffer and enzyme for amplifying said segment. In another embodiment, the reagents further comprise a label for detecting said fragment.

In one preferred embodiment, the kit comprises: a detection oligonucleotide probe that is from 5-100 nucleotides in length; an enhancer oligonucleotide probe that is from 5-100 nucleotides in length; and an endonuclease enzyme; wherein the detection oligonucleotide probe specifically hybridizes to a first segment of the nucleic acid whose nucleotide sequence is set forth in SEQ ID NO:1, and wherein the detection oligonucleotide probe comprises a detectable label at its 3′ terminus and a quenching moiety at its 5′ terminus; wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides; and wherein treating the nucleic acid with the endonuclease will cleave the detectable label from the 3′ terminus of the detection probe to release free detectable label when the detection probe is hybridized to the nucleic acid.

Kits according to the present invention may also be used in the other methods of the invention, including methods of assessing risk of developing at least a second primary tumor in an individual previously diagnosed with cancer, methods of assessing an individual for probability of response to a cancer therapeutic agent, and methods of monitoring progress of a treatment of an individual diagnosed with cancer and given a treatment for the disease.

In certain embodiments of the methods, uses, apparatus or kits of the invention, the at least one polymorphic marker that provides information about susceptibility to cancer is associated with the TERT gene. Being “associated with”, in this context, means that the at least one marker is in linkage disequilibrium with the TERT gene or its regulatory regions. Such markers can be located within the TERT gene, or its regulatory regions, or they can be in linkage disequilibrium with at least one marker within the TERT gene or its regulatory region that has a direct impact on the function of the gene. The functional consequence of the susceptibility variants associated with the TERT can be on the expression level of the TERT gene, the stability of its transcript or through amino acid alterations at the protein level, as described in more detail herein. Exemplary markers associated with the TERT gene are indicated in Table 8 herein, and certain embodiments relate to any one or more of those markers.

In certain other embodiments, the at least one polymorphic marker is associated with the CLPTM1L gene.

The skilled person will realize that the markers that are described herein to be associated with cancer can all be used in the various aspects of the invention, including the methods, kits, uses, apparatus, procedures described herein. In certain embodiments, the invention relates to markers associated with the human TERT gene. In certain embodiments, the invention relates to markers associated with the genomic region as set forth in SEQ ID NO:1 herein. In some embodiments, the invention relates to markers within the genomic region with the sequence as set forth in SEQ ID NO:1 herein. In certain other embodiments, the invention relates to the markers set forth in Table 5, Table 6, Table 7 and Table 8 herein. In certain embodiments, the invention relates to the markers set forth in Table 5. In certain embodiments, the invention relates to the markers set forth in Table 6. In certain embodiments, the invention relates to the markers set forth in Table 7. In certain embodiments, the invention relates to the markers set forth in Table 8. In certain other embodiments, the invention relates to any one of markers rs401681, rs2736100 and rs2736098, and markers in linkage disequilibrium therewith. In some other preferred embodiments, the invention relates to any one of rs401681, rs2736100 and rs2736098.

In certain embodiments, the at least one marker allele conferring increased risk of cancer is selected from rs401681 allele C (SEQ ID NO:2), rs2736100 allele G (SEQ ID NO:3) and rs2736098 allele A (SEQ ID NO:4). In these embodiments, the presence of the allele (the at-risk allele) is indicative of increased risk of cancer.

In certain embodiments, the at least one marker allele conferring a decreased risk of cancer is selected from rs401681 allele C, and marker alleles in linkage disequilibrium therewith, and wherein the cancer is melanoma cancer and/or colorectal cancer.

Certain embodiments of the invention relate to particular types of cancer. Thus, in certain embodiments, the cancer can be selected from any one of Basal Cell Carcinoma, Cutaneous Melanoma, Lung Cancer, Squamous Cell Carcinoma, Bladder Cancer, Prostate Cancer, Cervical Cancer, Thyroid Cancer, Colorectal Cancer and Endometrial Cancer. In certain other embodiments, any combinations of these cancers are contemplated, and such combinations are all within scope of the present invention.

In certain embodiments of the invention, linkage disequilibrium is determined using the linkage disequilibrium measures r² and |D′|, which give a quantitative measure of the extent of linkage disequilibrium (LD) between two genetic element (e.g., polymorphic markers). Certain numerical values of these measures for particular markers are indicative of the markers being in linkage disequilibrium, as described further herein. In one embodiment of the invention, linkage disequilibrium between marker (i.e., LD values indicative of the markers being in linkage disequilibrium) is defined as r²>0.1. In another embodiment, linkage disequilibrium is defined as r²>0.2. Other embodiments can include other definitions of linkage disequilibrium, such as r²>0.25, r²>0.3, r²>0.35, r²>0.4, r²>0.45, r²>0.5, r²>0.55, r²>0.6, r²>0.65, r²>0.7, r²>0.75, r²>0.8, r²>0.85, r²>0.9, r²>0.95, r²>0.96, r²>0.97, r²>0.98, or r²>0.99. Linkage disequilibrium can in certain embodiments also be defined as |D′|>0.2, or as |D′|>0.3, |D′|>0.4, |D′|>0.5, |D′|>0.6, |D′|>0.7, |D′|>0.8, |D′|>0.9, |D′|>0.95, |D′|>0.98 or |D′|>0.99. In certain embodiments, linkage disequilibrium is defined as fulfilling two criteria of r² and |D′|, such as r²>0.2 and |D′|>0.8. Other combinations of values for r² and |D′|, are also possible and within scope of the present invention, including but not limited to the values for these parameters set forth in the above.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein, as well as any particular cancer type (cancer at particular site), or combination of cancer types.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 A) shows a pair-wise correlation structure in a 200 kb interval (1.225-1.425 Mb, NCBI B36) on chromosome 5. The upper plot shows pair-wise D′ for 100 common SNPs (with minor allelic frequency >5%) from the HapMap (v22) CEU dataset. The lower plot shows the corresponding r² values; B) shows estimated recombination rates (saRR) in cM/Mb from the HapMap Phase II data (Frazer, K. A. et al. Nature 449, 851-61 (2007).); C) shows location of known genes in the region; D) shows chematic view of the association with basal cell carcinoma (BCC) in the Icelandic discovery sample set for directly genotyped SNPs (blue dots) and imputed SNPs (red dots).

FIG. 3 shows observed telomere length, as measured by quantitative PCR, as a function of SNP genotype for a) women born between 1925 and 1935 as a function of rs401681 genotype, b) women born between 1925 and 1935 as a function of rs2736098 genotype, c) women born between 1940 and 1950 as a function of rs401681 genotype, d) women born between 1940 and 1950 as a function of rs2736098 genotype.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein, including sequence listing, is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs401681” refers to the 3 allele of marker rs401681 being in the haplotype, and is equivalent to “rs401681 allele 3”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “cancer therapeutic agent” refers to an agent that can be used to ameliorate or prevent symptoms associated with cancer.

The term “cancer-associated nucleic acid”, as described herein, refers to a nucleic acid that has been found to be associated to cancer. This includes, but is not limited to, the markers and haplotypes described herein and markers and haplotypes in strong linkage disequilibrium (LD) therewith. In one embodiment, a cancer-associated nucleic acid refers to a genomic region, such as an LD-block, found to be associated with risk of cancer through at least one polymorphic marker located within the region or LD block.

The term “CLPTM1L” or “CLPTM1L gene”, as described herein, refers to the Cisplatin Resistance Related Protein on chromosome 5p13.3. The gene is also known as CLPTM1-like and CRR9p.

The term “TERT” or “TERT gene”, as described herein, refers to the Telomerase Reverse Transcriptase gene on chromosome 5p13.3.

The present inventors have discovered that variants on chromosome 5p13.3 associate with cancer at multiple sites. As shown in Tables 1-3 and 16 herein, the rs401681, rs2736100 and rs2736098 variants associate with risk of a variety of cancers, including Basal Cell Carcinoma, Lung Cancer, Bladder Cancer, Prostate Cancer, Cervical Cancer, Thyroid Cancer and Endometrial cancer. The most significant association was observed for Basal Cell Carcinoma (OR=1.27, P=7.96×10⁻¹¹ in Iceland for marker rs401681). The most significant site after BCC was lung cancer, reaching genome wide significance (OR=1.15, P=8.55×10⁻⁸ for rs401681) in the combined analysis of 4 populations from Iceland, the Netherlands and Spain in addition to the dataset from the IARC. Risk for the different cancers is comparable, ranging in most cases from 1.10-1.25.

The two known, genes in the region showing association to cancer, CLPTM1L and TERT, have both previously been studied in the context of cancer. CLPTM1L was identified in an ovarian cancer model as a gene that affected cisplatin-induced apoptosis but has not been extensively studied (Yamamoto, K., et al. Biochem Biophys Res Comm 280:1148-54 (2001). The TERT gene plays a leading role in maintenance of functional telomeres and has been firmly established as a key gene in cancer development. In particular, the well-documented association between telomere function and environmental insults such as radiation suggests a potential link between TERT and predisposition to BCC (reviewed in Ayouaz, A., et al. Biochimie 90:60-72 (2008)).

Based on these results, any one of the markers rs401681, rs2736100 and rs2736098 can be used to assess susceptibility to cancer, as described further herein. Furthermore, markers in linkage disequilibrium (LD) with these markers are equally useful in such applications. For example, the markers set forth in Tables 5, 6 and 7 represent markers in LD with the rs401681, rs2736100 and rs2736098 markers, respectively. Thus, in certain embodiments, markers in linkage disequilibrium with rs401681 are suitably selected from the group consisting of the markers listed in Table 5 herein. In certain embodiments, markers in linkage disequilibrium with rs2736100 are suitably selected from the group consisting of the markers listed in Table 6 herein. In certain embodiments, markers in linkage disequilibrium with rs2736098 are suitably selected from the group consisting of the markers listed in Table 7 herein.

Marker alleles that were found to be indicative of increased risk of several cancer types were found to be indicative of decreased risk of particular cancers, i.e. melanoma cancer and colorectal cancer. Thus, allele C of rs401681, which is indicative of increased risk of several cancers as shown herein was found to be indicative of decreased risk of melanoma cancer and colorectal cancer, i.e. the marker allele is protective for these particular cancers.

Telomeres are specific functional structures at the ends of eukaryotic chromosomes which are indispensable for chromosome protection and integrity (Collins, K. Curr Opin Cell Biol, 12: 378-83 (2000)). In proliferating cells lacking telomerase activity, telomeres progressively shorten with every cell division due to the end-replication problem and replication-associated erosion (Allsopp, R. C., et al. Proc Natl Acad Sci USA, 89: 10114-8 (1992)). Eventually, when telomeres are shortened and no longer protective, cells exit the cell cycle and enter a non-replicative state termed senescence (Campisi, J. Eur J Cancer, 33: 703-9 (1997)). Telomerase, a unique ribonucleoprotein with reverse transcriptase activity, catalyzes the de novo addition of telomeric repeat sequences onto the eroding chromosome ends and thereby counterbalances telomere-dependent replicative aging (Greider, C. W., and Blackburn, E. H. Cell, 43: 405-13 (1985)). Telomerase is repressed in most human somatic cells, but reactivated in more than 80% of all human cancers (reviewed in Deng, Y., and Chang, S. Lab Invest, 87: 1071-6 (2007))). Because of its involvement in carcinogenesis, telomerase is a promising candidate as both tumor marker and therapeutic target for telomerase inhibitors or antisense constructs (Harley, C. B. Nat Rev Cancer, 8: 167-79 (2008)).

The length of telomeric sequences is inversely related to age, reflecting the progressive shortening with each cell division. Thus, the average telomere size in peripheral blood cells and colorectal mucosa epithelia from older individuals was found to be shorter than that from younger individuals, corresponding to a rate of telomere loss of 33 bp/year (Hastie, N. D., et al. Nature, 346: 866-8 (1990)). However, telomere length also varies considerably between individuals in the same age group and it has been shown that this variation is to a large extent genetically determined (Slagboom, P. E., et al. Am J Hum Genet, 55: 876-82 (1994)). Recently, multiple studies have reported an association between short telomeres and increased risk of cancer at several sites, including lung, head and neck, bladder, kidney and breast (Wu, X., et al. J Natl Cancer Inst, 95: 1211-8 (2003); Shen, J., et al. Cancer Res, 67: 5538-44 (2007); Jang, J. S., et al. Cancer Sci, 99: 1385-9 (2008)). These findings suggest that the genetic factors that determine telomere length may also affect the risk for multiple types of cancer.

Telomeres are directly affected by several stimuli that are known risk factors for cancer. Telomere shortening is accelerated in response to oxidative stress caused by environmental factors such as radiation and cigarette smoke (Ayouaz, A., et al. Biochimie, 90: 60-72 (2008); McGrath, M., et al. Cancer Epidemiol Biomarkers Prev, 16: 815-9 (2007)) and chronic psychological stress has been associated with telomere shortening (Epel, E. S., et al. Proc Natl Acad Sci USA, 101: 17312-5 (2004)).

In light of this biological context, it is possible that the biological effect of the association to cancer described herein is through an effect on the TERT gene. Thus, markers within the gene, or markers in linkage disequilibrium with the gene (such as rs401681, rs2736100 and rs2736098) can be used to assess susceptibility to cancer. Furthermore, other markers within or in near proximity to the TERT gene, such as the markers set forth in Tables 8 and 9 herein, may represent variants with comparable or even more significant association to cancer (represented by larger OR values). Such variants are also useful for assessing susceptibility to cancer, and are within scope of the present invention.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions and inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 6 million SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007). A http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the markers described herein to be associated with cancer. Methods for detecting CNVs include comparative genomic hybridization (CGH) and genotyping, including use of genotyping arrays, as described by Carter (Nature Genetics 39:516-S21 (2007)). The Database of Genomic Variants (http://projects.tcag.ca/variation/) contains updated information about the location, type and size of described CNVs. The database currently contains data for over 15,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of relative risk), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In the present context, and individual who is at an increased susceptibility (i.e., increased risk) for a disease, is an individual in whom at least one specific allele at one or more polymorphic marker or haplotype conferring increased susceptibility (increased risk) for the disease is identified (i.e., at-risk marker alleles or haplotypes). The at-risk marker or haplotype is one that confers an increased risk (increased susceptibility) of the disease. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.1, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.08 is significant. In yet another embodiment, a risk of at least 1.10 is significant. In a further embodiment, a relative risk of at least 1.15 is significant. In another further embodiment, a significant increase in risk is at least 1.17 is significant. However, other cutoffs are also contemplated, e.g., at least 1.11, 1.12, 1.13, 1.14, 1.15, 1.16, 1.17, 1.18, 1.19, 1.20, 1.21, 1.22, 1.23, 1.24, 1.25, and so on, and such cutoffs are also within scope of the present invention. In other embodiments, a significant increase in risk is at least about 80%, including but not limited to about 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 10%. In other embodiments, a significant increase in risk is at least 15%, at least 17%, at least 18%, at least 19%, at least 20%, at least 25%, at least 30% and at least 40%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype as described herein is one where at least one allele of at least one marker or haplotype is more frequently present in an individual at risk for the disease (or trait) (affected), or diagnosed with the disease, compared to the frequency of its presence in a comparison group (control), such that the presence of the marker or haplotype is indicative of susceptibility to the disease. The control group may in one embodiment be a population sample, i.e. a random sample from the general population. In another embodiment, the control group is represented by a group of individuals who are disease-free. Such disease-free controls may in one embodiment be characterized by the absence of one or more specific disease-associated symptoms. Alternatively, the disease-free controls are those that have not been diagnosed with the disease. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors comprise at least one additional genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In certain embodiments, the marker is rs401681, wherein the presence of allele C is indicative of decreased risk of melanoma cancer and/or colorectal cancer. Alternatively, marker alleles in linkage disequilibrium with rs401681 allele C are indicative of decreased risk of melanoma cancer and/or colorectal cancer. In a preferred embodiment, the presence of allele C in rs401681, or a marker allele in linkage disequilibrium therewith, is indicative of a protection against melanoma cancer in the individual. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus, for rs401681, allele C is found to be indicative of protection against melanoma cancer and colorectal cancer. Therefore, the alternate allele, allele T, is an at-risk allele for melanoma cancer and colorectal cancer. Determination of the presence of this allele in individuals (in genotype datasets, samples containing DNA or in sequence data from individuals) is thus indicative of an increased risk of melanoma cancer and/or colorectal cancer in such individuals.

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., et al., Nat Genet 38:652-8 (2006); Haiman, C. A., et al., Nat Genet. 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with particular cancers may be assessed, including combinations of any one of the markers rs401681, rs2736100 and rs2736098, or markers in linkage disequilibrium therewith, with any other markes associated with risk of any one particular cancer. Such combinations may include any particular marker, or combination of markers, known to be associated with risk of the particular cancer.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet. 29:217-222 (2001); May, C. A., et al., Nature Genet. 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles or allelic combinations for each genetic element (e.g., a marker, haplotype or gene).

Many different measures have been proposed for assessing the strength of linkage disequilibrium (LD; reviewed in Devlin, B. & Risch, N., Genomics 29:311-22 (1995))). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 ('complete' disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, or at least 0.99. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, Japanese, Chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al., Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (such as <10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families to the study, first and second-degree relatives can be eliminated from the patient list. Furthermore, the test can be repeated for association correcting for any remaining relatedness among the patients, by extending a variance adjustment procedure previously described (Risch, N. & Teng, J. Genome Res., 8:1273-1288 (1998)) for sibships so that it can be applied to general familial relationships, and present both adjusted and unadjusted p-values for comparison. The method of genomic controls (Devlin, B. & Roeder, K. Biometrics 55:997 (1999)) can also be used to adjust for the relatedness of the individuals and possible stratification. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(f/pᵢ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study, and hence the less stringent the statistical measure that is applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold are a measure of a true genetic effect, and replication in additional cohorts is not necessarily from a statistical point of view. However, since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing a specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

As described herein, certain polymorphic markers and haplotypes comprising such markers are found to be useful for risk assessment of cancer. Risk assessment can involve the use of the markers for determining a susceptibility to cancer. Particular alleles of polymorphic markers (e.g., SNPs) are found more frequently in individuals with cancer, than in individuals without diagnosis of cancer. Therefore, these marker alleles have predictive value for detecting cancer, or a susceptibility to cancer, in an individual. Tagging markers in linkage disequilibrium with at-risk variants (or protective variants) described herein can be used as surrogates for these markers (and/or haplotypes). Such surrogate markers can be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that confers decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. The at-risk variant identified may not be the functional variant itself, but is in this instance in linkage disequilibrium with the true functional variant. The functional variant may for example be a tandem repeat, such as a minisatellite or a microsatellite, a transposable element (e.g., an Alu element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to the disease, or a susceptibility to the disease, in an individual. These tagging or surrogate markers that are in LD with the markers of the present invention can also include other markers that distinguish among haplotypes, as these similarly have predictive value for detecting susceptibility to the particular disease.

The present invention can in certain embodiments be practiced by assessing a sample comprising genomic DNA from an individual for the presence of variants described herein to be associated with cancer. Such assessment typically steps that detect the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of cancer. Detecting particular alleles of polymorphic markers can in certain embodiments be done by obtaining nucleic acid sequence data about a particular human individual, that identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker associated with a disease (or markers in linkage disequilibrium with at least one marker associated with the disease). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease, is indicative of the individual from which the dataset is derived is at increased susceptibility (increased risk) of the disease.

In certain embodiments of the invention, a polymorphic marker is correlated to a disease by referencing genotype data for the polymorphic marker to a look-up table that comprises correlations between at least one allele of the polymorphism and the disease. In some embodiments, the table comprises a correlation for one polymorhpism. In other embodiments, the table comprises a correlation for a plurality of polymorhpisms. In both scenarios, by referencing to a look-up table that gives an indication of a correlation between a marker and the disease, a risk for the disease, or a susceptibility to the disease, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers described herein, e.g., the markers presented in Tables 5, 6, 7, 8, and 9, e.g. rs401681, rs2736100 and rs2736098, may be useful for risk assessment and diagnostic purposes, either alone or in combination. Results of cancer risk based on the markers described herein can also be combined with data for other genetic markers or risk factors for cancer, to establish overall risk. Thus, even in cases where the increase in risk by individual markers is relatively modest, e.g. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in certain embodiments of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to cancer. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses or other methods known to the skilled person, may subsequently be used to determine the overall risk conferred based on the genotype status at the multiple loci. Assessment of risk based on such analysis may subsequently be used in the methods, uses and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined, as discussed in the above. Such markers and/or haplotypes may in those cases be also used as surrogate markers and/or haplotypes for the markers and/or haplotypes physically residing within the haplotype block as defined. As a consequence, markers and haplotypes in LD (typically characterized by inter-marker r² values of greater than 0.1, such as r² greater than 0.2, including r² greater than 0.3, also including markers correlated by values for r² greater than 0.4) with the markers and haplotypes of the present invention are also within the scope of the invention, even if they are physically located beyond the boundaries of the haplotype block as defined. This includes markers that are described herein (e.g., rs401681, rs2736100 and rs2736098), but may also include other markers that are in strong LD (e.g., characterized by r² greater than 0.1 or 0.2 and/or |D′|>0.8) with rs401681, rs2736100 and rs2736098 (e.g., the markers set forth in Table 5, 6 and 7).

For the SNP markers described herein, the opposite allele to the allele found to be in excess in patients (at-risk allele) is found in decreased frequency in cancer. These markers and haplotypes in LD and/or comprising such markers, are thus protective for cancer, i.e. they confer a decreased risk or susceptibility of individuals carrying these markers and/or haplotypes developing cancer. It is noteworthy that while allele C of rs401681 is predictive of increased risk of multiple cancers as shown herein, this allele is predictive of decreased risk of melanoma cancer and colorectal cancer, i.e. the allele is protective for these cancers.

Certain variants of the present invention, including certain haplotypes comprise, in some cases, a combination of various genetic markers, e.g., SNPs and microsatellites. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with cancer, (e.g., marker alleles as listed in Tables 1, 2 and 3) is one in which the marker allele or haplotype is more frequently present in an individual at risk for cancer (affected), compared to the frequency of its presence in a healthy individual (control), or in randombly selected individual from the population, wherein the presence of the marker allele or haplotype is indicative of a susceptibility to cancer. In other embodiments, at-risk markers in linkage disequilibrium with one or more markers shown herein to be associated with cancer (e.g., marker alleles as listed in Tables 1, 2 and 3) are tagging markers that are more frequently present in an individual at risk for cancer (affected), compared to the frequency of their presence in a healthy individual (control) or in a randomly selected individual from the population, wherein the presence of the tagging markers is indicative of increased susceptibility to cancer. In a further embodiment, at-risk markers alleles (i.e. conferring increased susceptibility) in linkage disequilibrium with one or more markers found to be associated with cancer, are markers comprising one or more allele that is more frequently present in an individual at risk for cancer, compared to the frequency of their presence in a healthy individual (control), wherein the presence of the markers is indicative of increased susceptibility to cancer.

### Study Population

In a general sense, the methods and kits of the invention can be utilized from samples containing nucleic acid material (DNA or RNA) from any source and from any individual, or from genotype data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing cancer, based on other genetic factors, biomarkers, biophysical parameters, history of cancer or related diseases, previous diagnosis of cancer, family history of cancer. A target population is in certain embodiments is a population or group with known radiation exposure, such as radiation exposure due to diagnostic or therapeutic medicine, radioactive fallout from nuclear explosions, radioactive exposure due to nuclear power plants or other sources of radiactivity, etc.

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of cancer in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Styrkarsdottir, U., et al. N Engl J Med Apr. 29 2008 (Epub ahead of print); Thorgeirsson, T., et al., Nature 452:638-42 (2008); Gudmundsson, J., et al. Nat. Genet. 40:281-3 (2008); Stacey, S. N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers of the present invention found to be associated with cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop a particular form of cancer. The variants described herein do however indicate increased and/or decreased likelihood that individuals carrying the at-risk or protective variants of the invention will develop cancer, such as cancer of the lung, bladder, prostate, cervix, endometrium, thyroid and/or basal cells of the skin. This information is however extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

Analysis of the functional role of the genetic cancer risk variants may provide information on the molecular pathways that lead to cancer development and/or disease progression. Thus, on one hand there will be true “predisposition” variants that affect mostly whether an individual develops a disease or not. On the other hand, other variants may also associate with a particular course of the disease by influencing subsequent genetic changes in the tumor. Characterization of these changes may lead to the development of treatment strategies that would be particularly suitable in individuals carrying the genetic risk variant.

### Genetic Testing for Predisposition to Multiple Cancers

In general, the knowledge of genetic variants that confer a risk of developing cancer offers the opportunity to apply a genetic test to distinguish between individuals with increased risk of cancer (i.e. carriers of the at-risk variants) and those with decreased risk of developing them (i.e. carriers of protective variants, and/or non-carriers of at-risk variants). The core value of genetic testing is the possibility of being able to diagnose disease, or a predisposition to disease, at an early stage and provide information to the clinician about prognosis/aggressiveness of the disease in order to be able to apply the most appropriate treatment.

The variants described herein show association to multiple forms of cancer. Thus it can be envisioned that they could have a utility in genetic testing for cancer predisposition in general. Notably, the variants show preferential association to cancer types that have a strong environmental component as well, such as UV radiation, smoking and exposure to industrial chemicals. Testing for the variants may be useful in a setting where individuals are exposed to these environmental agents. Individuals at high genetic risk could then be targeted for more frequent cancer screening. Also, intervention strategies that reduce or limit exposure to the environmental risk factors could be emphasized particularly in this group of individuals. An indication of possible intervention strategies for each cancer type are described below.

### Genetic Testing for Basal Cell Carcinoma

The strongest known risk factors for BCC include exposure to UV radiation, fair pigmentation traits and genetic factors. A positive family history is a risk factor for BCC and SCC (Hemminki, K. et al., Arch Dermatol, 139, 885 (2003); Vitasa, B. C. et al., Cancer, 65, 2811 (1990)) suggesting an inherited component to the risk of BCC. Several rare genetic conditions have been associated with increased risks of BCC, including Nevoid Basal Cell Syndrome (Gorlin's Syndrome), Xeroderma Pigmentosum (XP), and Bazex's Syndrome. XP is underpinned by mutations in a variety of XP complementation group genes. Gorlin's Syndrome results from mutations in the PTCH1 gene. In addition, variants in the CYP2D6 and GSTT1 genes have been associated with BCC (Wong, et al., BMJ, 327, 794 (2003)).

Fair pigmentation traits are known risk factors for BCC and are thought act, at least in part, through a reduced protection from UV irradiation. Thus, genes underlying these fair pigmentation traits have been associated with risk. MC1R, ASIP, and TYR have been shown to confer risk for BCC and/or SCC (Gudbjartsson, et. al., Nat. Gen. 40, 886 (2008); Bastiaens, et al., Am 3 Hum Genet, 68, 884 (2001); Han, et al., Int J Epidemiol, 35, 1514 (2006)).

Elucidation of genetic variants that affect risk of BCC, either though pigmentation traits or other mechanisms, can help identify individuals who have a high risk of developing these diseases. Thus, individuals who are at increased risk of BCC might be offered regular skin examinations to identify incipient tumours, and they might be counseled to avoid excessive UV exposure. Chemoprevention either using sunscreens or pharmaceutical agents (Bowden, Nat Rev Cancer, 4, 23 (2004)). might be employed. For individuals who have been diagnosed with BCC, knowledge of the underlying genetic predisposition may be useful in determining appropriate treatments and evaluating risks of recurrence and new tumors. Finally, screening for susceptibility to BCC or SCC might be important in planning the clinical management of transplant recipients and other immunosuppressed individuals.

### Genetic Testing for Melanoma.

Relatives of melanoma patients are themselves at increased risk of melanoma, suggesting an inherited predisposition [Amundadottir, et al., (2004), PLoS Med, 1, e65. Epub 2004 Dec. 28.]. A series of linkage based studies implicated CDKN2a on 9p21 as a major CM susceptibility gene [Bataille, (2003), Eur 3 Cancer, 39, 1341-7.]. CDK4 was identified as a pathway candidate shortly afterwards, however mutations have only been observed in a few families worldwide[Zuo, et al., (1996), Nat Genet, 12, 97-9.]. CDKN2a encodes the cyclin dependent kinase inhibitor p16 which inhibits CDK4 and CDK6, preventing G1-S cell cycle transit. An alternate transcript of CKDN2a produces p14ARF, encoding a cell cycle inhibitor that acts through the MDM2-p53 pathway. It is likely that CDKN2a mutant melanocytes are deficient in cell cycle control or the establishment of senescence, either as a developmental state or in response to DNA damage. Overall penetrance of CDKN2a mutations in familial CM cases is 67% by age 80. However penetrance is increased in areas of high melanoma prevalence [Bishop, et al., (2002), 3 Natl Cancer Inst, 94, 894-903].

Individual who are at increased risk of melanoma might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might be employed. For individuals who have been diagnosed with melanoma, knowledge of the underlying genetic predisposition may be useful in determining appropriate treatments and evaluating risks of recurrence and new primary tumours.

Endogenous host risk factors for CM are in part under genetic control. It follows that a proportion of the genetic risk for CM resides in the genes that underpin variation in pigmentation and nevi. The Melanocortin 1 Receptor (MC1R) is a G-protein coupled receptor involved in promoting the switch from pheomelanin to eumelanin synthesis. Numerous, well characterized variants of the MC1R gene have been implicated in red haired, pale skinned and freckle prone phenotypes. We and others have demonstrated the MC1R variants confer risk of melanoma (Gudbjartsson et. al., Nature Genetics 40:886-91 (2008)). Other pigmentation trait-associated variants, in the ASIP, TYR and TYRP1 genes have also been implicated in melanoma risk (Gudbjartsson et. al., Nature Genetics 40:886-91 (2008)). ASIP encodes the agouti signalling protein, a negative regulator of the melanocortin 1 receptor. TYR and TYRP1 are enzymes involved in melanin synthesis and are regulated by the MC1R pathway. Individuals at risk for BCC and/or SCC might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might, be employed. For individuals who have been diagnosed with BCC or SCC, knowledge of the underlying genetic predisposition may be useful in determining appropriate treatments and evaluating risks of recurrence and new primary tumours. Screening for susceptibility to BCC or SCC might be important in planning the clinical management of transplant recipients and other immunosuppressed individuals.

Genetic testing for Prostate cancer

Epidemiological studies suggest that the genetic component of the risk of prostate cancer is greater than in any other cancer (Lichtenstein et al, N Engl J Med 343, 78 (2000)). Despite strong evidence for genetic factors, highly penetrant susceptibility genes for prostate cancer have proven difficult to find. Analysis of data from large twin studies has suggested that the majority of genetic prostate cancer risk may be attributable to recessive and/or multiple interacting genetic variants (Risch, Cancer Epidemiol Biomarkers Prev 7, 733 (2001)). Recently, several common genetic variants have been identified that affect the risk of prostate cancer Amundadottir, et al, Nat Gen 38, 652 (2006); Gudmundsson, et al, Nat Gen 39, 631 (2007); Gudmundsson, et al., Nat Gen 39, 977 (2007); Gudmundsson, et al., Nat Gen 40, 281 (2008); Eeles, et al., Nat Gen 40, 316 (2008); Yeager, et al., Nature Gen 39, 645 (2007)).

The characterization of genetic risk variants for prostate cancer can be put to use in at least two ways. First, a genetic risk model can be incorporated into a screening protocol to aid in early detection of the disease when chances of cure are the highest. Second, genetic variants may be found that associate with progression of the disease and could be use to direct treatment selection.

### 1. Early Detection

Early diagnosis and treatment are key factors in determining the survival of certain sets of prostate cancer patients. The test most frequently used to screen for prostate cancer, the PSA blood test, is effective at detecting early stage prostate cancer but has limited specificity for the aggressive form of the disease, resulting in an extremely high rate of “over-diagnosis” of up to 50% (Draisma G, et al. 3 Natl Cancer Inst 95:868 (2003)). Consequently, prostate cancer incidence has risen rapidly in those European countries where opportunistic PSA screening is commonplace and, due to lack of prognostic tests, has led to excessive treatment of localized lesions that might never progress to symptomatic cancer. This over-treatment carries heavy costs, both financial and personal as side-effects of treatment can be considerable, including impaired urinary continence and sexual dysfunction. A genetic variant that is shown to associate with a clinically relevant for of the disease might be useful in increasing the sensitivity and specificity of the already generally applied Prostate Specific Antigen (PSA) test and Digital Rectal Examination (DRE). This can lead to lower rates of false positives (thus minimize unnecessary procedures such as needle biopsies) and false negatives, thereby increasing detection of occult disease and minimizing morbidity and mortality due to prostate cancer. Also, an individual determined to be a carrier of a risk allele for the development of prostate cancer will likely undergo more frequent PSA testing and have a lower threshold for needle biopsy in the presence of an abnormal PSA value.

### 2. Predicting Progression of Early-Stage Prostate Cancer

Today most men with screen-detected prostate cancer have localized disease at diagnosis. Many of these men may harbour clinically insignificant disease that will not impact their quality of life and life expectancy while in other men prostate cancer will progress to an advanced or lethal disease if left alone. Because of these uncertainties, and the lack of reliable prognostic markers, most men with localized disease are subjected to radical prostatectomy or radiotherapy which can adversely impact their urinary and sexual health. The reasons why some cancers are more aggressive than others remain poorly understood and the need for diagnostic resources to help differentiate between the two is immense. Identification of genetic markers that preferentially associate with an aggressive form of the disease could have important utility in guiding treatment selection. For example, if prostate cancer is diagnosed in an individual that is a carrier of an allele that confers increased risk of developing an aggressive form of prostate cancer, then the clinician would likely advise a more aggressive treatment strategy such as a prostatectomy instead of a less aggressive treatment strategy.

### Genetic Testing for Lung Cancer

Although the large majority of lung cancer cases can be attributed to smoking, the disease is also influenced by genetic factors (Jonsson et. al., JAMA 292, 2977 (2004); Amundadottir et. al., PLoS Med. 1, e65 (2004)). Recently, a genetic variant on chromosome 15q was identified that affects smoking behaviour and increases risk of lung cancer (Thorgeirsson et al., Nature 452, 638 (2008)).

Individuals with a family history of lung cancer and carriers of at-risk variants may benefit from genetic testing since the knowledge of the presence of genetic risk factors, or evidence for increased risk of being a carrier of one or more genetic risk factors, may provide incentive for implementing a healthier lifestyle, by avoiding or minimizing known environmental risk factors for lung cancer. For example, an individual who is a current smoker and is identified as a carrier of one or more of the variants shown herein to be associated with increased risk of lung cancer, may, due to his/her increased risk of developing the disease, choose to quit smoking.

Integration of Genetic Risk Models into Clinical Management of Lung Cancer:

Management of lung cancer currently relies on a combination of primary prevention (most importantly abstinence from smoking), early diagnosis and appropriate treatments. There are clear clinical imperatives for integrating genetic testing into several aspects of these management areas. Identification of cancer susceptibility genes may also reveal key molecular pathways that may be manipulated (e.g., using small or large molecular weight drugs) and may lead to more effective treatments.

### 1. Primary Prevention

Primary prevention options currently focus on avoiding exposure to tobacco smoke or other environmental toxins that have been associated with the development of lung cancer. Knowledge of the genetic risk for lung cancer may encourage individuals to abstain from smoking.

### 2. Early Diagnosis

Patients who are identified as being at high risk for lung cancer may be referred to have chest X-rays or sputum cytology examination. In addition, a spiral CT scan is a newly-developed procedure for lung cancer screening. Numerous lung cancer screening trials are currently taking place but presently, the U.S. Preventive Services Task Force (USPSTF) concludes that evidence is insufficient to recommend for or against screening asymptomatic persons for lung cancer with either low dose computerized tomography (LDCT), chest x-ray, sputum cytology, or a combination of these tests.

Many of the screening protocols being evaluated involve some form of radiation or and invasive procedure such as bronchoscopy. These protocols carry certain risks and may prove hard to implement due to the considerable costs involved. In light of the fact that only about 15% of lifetime smokers develop lung cancer, it is clear that the great majority of individuals at risk would be needlessly subjected to repeated screening tests with the associated costs and negative side-effects. The identification of genetic biomarkers that affect the risk of developing lung cancer could be used to help identify individuals should be offered extreme help in risk reduction programs such as smoking termination. In the case of failure to stop smoking, or in the case of ex-smokers, such genetic biomarkers could help in defining the subpopulation of individuals that would benefit the most from screening.

Less than 10% of lung cancer cases arise in individuals that have never smoked. Genetic biomarkers that predict the risk of lung cancer would be particularly useful in this group. The genetic component of this form of the disease is likely to be even stronger than in tobacco-related lung cancer. If genetic variants that affect the risk of non-smoking lung cancer were known, it might be possible to identify individuals at high risk for this disease and subject them to regular screening tests.

### Genetic Testing for Urinary Bladder Cancer (UBC)

Cigarette smoking and occupational exposure to specific carcinogens are the strongest known risk factors for UBC. Familial clustering of UBC cases suggests that there is a genetic component to the risk of the disease (Aben, K. K. et al. Int J Cancer 98, 274 (2002); Amundadottir, L. T. et al. PLoS Med 1, e65 (2004); Murta-Nascimento, C. et al. Cancer Epidemiol Biomarkers Prev 16, 1595 (2007)). Segregation analyses have suggested that this component consists of many genes, each conferring a small risk (Aben, K. K. et al., Eur 3 Cancer 42, 1428 (2006)). Epidemiological studies have evaluated potential associations between sequence variants in candidate genes and UBC but the results have in many cases been difficult to replicate.

Identification of genetic variants that confer a risk of developing UBC offers the opportunity to distinguish between individuals with increased risk of developing UBC (i.e. carriers of the at-risk variant) and those with decreased risk of developing UBC (i.e. carriers of the protective variant). In the case of increased genetic risk, an individual may be offered more frequent screening for the disease or be advised to take extra steps to avoid known environmental risk factors. The polymorphic markers of the present invention can be used alone or in combination, as well as in combination with other factors, including other genetic risk factors or biomarkers, for risk assessment of an individual for UBC. Many factors known to affect the predisposition of an individual towards developing risk of developing UBC are known to the person skilled in the art and can be utilized in such assessment. These include, but are not limited to, age, gender, smoking status and/or smoking history, family history of cancer, and of UBC in particular. Methods known in the art can be used for such assessment, including multivariate analyses or logistic regression.

Current clinical treatment options for UBC include different surgical procedures, depending on the severity of the cases, e.g. whether the cancer is invasive into the muscle wall of the bladder. Treatment options also include radiation therapy, for which a proportion of patients experience adverse symptoms. One application of genetic risk markers for UBC includes the use of such markers to assess response to these therapeutic options, or to predict the efficacy of therapy using any one of these treatment options. Thus, genetic profiling can be used to select the appropriate treatment strategy based on the genetic status of the individual, or it may be used to predict the outcome of the particular treatment option, and thus be useful in the strategic selection of treatment options or a combination of available treatment options. Again, such profiling and classification of individuals is supported further by first analysing known groups of patients for marker and/or haplotype status, as described further herein.

Genetic profiling based on the markers described herein, and model building based on such markers, can thus be useful in various aspects of bladder cancer risk management, including prediction of lifetime risk, management of disease at its various stages, and selection of appropriate treatment regimens.

### Genetic Testing for Cervical Cancer

Cervical cancer is invariably associated with an infection with an oncogenic subtype of human papillomavirus (HPV). Infection with HPV is very common but in the great majority of cases, infection is cleared by the immune system and does not develop into a malignant state. It has been shown that genetic factors play a substantial role in the development of cervical cancer (Czene, K. et al., Int J Cancer 99, 260 (2002); Hemminki, K., and Chen, B. Cancer Epidemiol Biomarkers Prev 15, 1413 (2006); Couto, E., and Hemminki, K Int J Cancer 119, 2699 (2006)). Some of these genetic factors may affect mechanisms that help clear the viral infection and indeed, several polymorphisms in immune response genes have been associated with susceptibility to chronic HPV infection and cancer development (Hildesheim and Wang, Virus Res 89, 229 (2002).

Integration of Genetic Risk Models into Clinical Management of Cervical Cancer:

Management of cervical cancer currently focuses on early diagnosis through PAP test-based screening and appropriate treatments of dysplastic lesions/invasive cancer. There are clear clinical imperatives for integrating genetic testing into several aspects of these management areas. Identification of cancer susceptibility genes may also reveal key molecular pathways that may be manipulated (e.g., using small or large molecular weight drugs) and may lead to more effective treatments.

### Primary Prevention

Young women who have not been infected with HPV can get vaccinated against the most common subtypes of the virus. However, vaccination has not proven to prevent cancer in women who have already been infected with an oncogenic subtype of the virus and may not provide protection against rare virus types that are not included in the vaccine.

The most important prevention strategy against cervical cancer for the great majority of women is avoiding infection with HPV by limiting the number of sexual partners and using condoms during sexual intercourse. This strategy also limits exposure to other sexual transmitted diseases which may act as co-factors in cervical cancer development. A person who is carries genetic risk factors for cervical cancer might be encouraged to apply all the preventive measures available.

Finally, cervical cancer occurs at a higher rate in immunosuppressed individuals. Screening for susceptibility to CC might be useful in planning the clinical management of transplant recipients and other immunosuppressed individuals.

### Early Diagnosis

Excluding the future effect of vaccinations, the most effective strategy in the fight against cervical cancer is regular screening in order to detect the disease before it becomes invasive. Screening for cervical cancer varies widely between countries, ranging from the organized, population-based screening programs (e.g. the Nordic countries), to ad hoc screening (e.g. the United States). The frequency of screening visits also varies between locations but commonly it is recommended that a woman gets a PAP test performed every year or every two years between age 22 and 70. Considerable evidence suggests that this screening regime is unnecessarily intensive and that a woman who has had 2 consecutive negative tests could be told to come every 5 years, greatly reducing the cost of the screening effort. However, considering the severity of the disease if not caught early, there is reluctance in changing these recommendations until further evidence is provided to support the safety of the alternative schedule. Assessment of genetic risk could be a tool to help determine the appropriate intervals between screening.

While cytological examination of PAP smears is highly effective in detecting dysplastic lesions and early stage CC which can be effectively treated by cone operation, a fraction of cases present with a persistent infection or re-infection which may progress to invasive cancer (Schiffman, M., et al., Lancet 370, 890 (2007)). These cases often need to be followed for years and subjected to repeated biopsies. There is an unmet clinical need to identify women with persistent or recurring infections that have the greatest risk of progressing to invasive CC. Such individuals might be subjected to more rigorous follow-up protocols or advised on how to reduce the risk by lifestyle changes. Knowledge of the underlying genetic predisposition might be useful in evaluating risks of progression.

### Genetic Testing for Thyroid Cancer

The primary known risk factor for thyroid cancer is radiation exposure. Thyroid cancer incidence within the US has been rising for several decades, which may be attributable to increased detection of sub-clinical cancers, as opposed to an increase in the true occurrence of thyroid cancer (Davies, L. and Welch, H. G., Jama, 295, 2164 (2006)). The introduction of ultrasonography and fine-needle aspiration biopsy in the 1980s improved the detection of small nodules and made cytological assessment of a nodule more routine (Rojeski, M. T. and Gharib, H., N Engl J Med 313, 428 (1985); Ross, D. S., J Clin Endocrinol Metab, 91, 4253 (2006)). This increased diagnostic scrutiny may allow early detection of potentially lethal thyroid cancers. However, several studies report thyroid cancers as a common autopsy finding (up to 35%) in persons without a diagnosis of thyroid cancer (Bondeson, L. and Ljungberg, O., Cancer, 47, 319 (1981); Harach, H. R., et al., Cancer, 56, 531 (1985); Solares, C. A., et al., Am 3 Otolaryngol, 26, 87 (2005); Sobrinho-Simoes, M. A. et al., Cancer, 43, 1702 (1979)). This suggests that many people live with sub-clinical forms of thyroid cancer which are of little or no threat to their health.

Individuals with a family history of thyroid cancer and carriers of at-risk variants may benefit from genetic testing since the knowledge of the presence of a genetic risk factor, or evidence for increased risk of being a carrier of one or more risk factors, may provide increased incentive for implementing a healthier lifestyle, by avoiding or minimizing known environmental risk factors for the disease. Genetic testing of patients diagnosed with thyroid cancer may furthermore give valuable information about the primary cause of the disease and can aid the clinician in selecting the best treatment options and medication for each individual.

The knowledge of underlying genetic risk factors for thyroid cancer can be utilized in the application of screening programs for thyroid cancer. Thus, carriers of at-risk variants for thyroid cancer may benefit from more frequent screening than do non-carriers. Homozygous carriers of at-risk variants are particularly at risk for developing thyroid cancer. Also, carriers may benefit from more extensive screening, including ultrasonography and/or fine needle biopsy. The goal of screening programs is to detect cancer at an early stage. Knowledge of genetic status of individuals with respect to known risk variants can aid in the selection of applicable screening programs. In certain embodiments, it may be useful to use the at-risk variants for thyroid cancer described herein together with one or more diagnostic tool selected from Radioactive Iodine (RAI) Scan, Ultrasound examination, CT scan (CAT scan), Magnetic Resonance Imaging (MRI), Positron Emission Tomography (PET) scan, Fine needle aspiration biopsy and surgical biopsy.

### Methods

Methods for cancer risk assessment and risk management are described herein and are encompassed by the invention. The invention also encompasses methods of assessing an individual for probability of response to therapeutic agents, methods for predicting the effectiveness of therapeutic agents, nucleic acids, polypeptides and antibodies and computer-implemented aspects of the invention. Kits for use in the various methods presented herein are also encompassed by the invention.

### Diagnostic and Screening Methods

In certain embodiments, the present invention pertains to methods of determining a susceptibility to cancer, by detecting particular alleles at genetic markers that appear more frequently in subjects diagnosed with cancer or subjects who are susceptible to cancer. In particular embodiments, the invention is a method of determining a susceptibility to cancer by detecting at least one allele of at least one polymorphic marker (e.g., the markers described herein). In other embodiments, the invention relates to a method of determining a susceptibility to cancer by detecting at least one allele of at least one polymorphic marker. The present invention describes methods whereby detection of particular alleles of particular markers or haplotypes is indicative of a susceptibility to cancer. Such prognostic or predictive assays can also be used to determine prophylactic treatment of a subject based on determination of the genetic risk of cancer for the subject.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait (such as cancer). The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of determining a decreased susceptibility to cancer, by detecting particular genetic marker alleles or haplotypes that appear less frequently in patients with cancer than in individuals not diagnosed with cancer, or in the general population.

As described and exemplified herein, particular marker alleles or haplotypes (e.g. markers on chromosome 5p13.3, e.g. rs401681, rs2736100 and rs2736098, and markers in linkage disequilibrium therewith) are associated with cancer. In one embodiment, the marker allele or haplotype is one that confers a significant risk or susceptibility to cancer. In another embodiment, the invention relates to a method of determining a susceptibility to cancer in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 2. In another embodiment, the invention pertains to methods of determining a susceptibility to cancer in a human individual, by screening for at least one marker selected from rs401681, rs2736100 and rs2736098. In another embodiment, the marker allele or haplotype is more frequently present in a subject having, or who is susceptible to, cancer (affected), as compared to the frequency of its presence in a healthy subject (control, such as population controls). In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to cancer. These diagnostic methods involve determining whether particular alleles or haplotypes that are associated with risk of cancer are present in particular individuals. The haplotypes described herein include combinations of alleles at various genetic markers (e.g., SNPs, microsatellites or other genetic variants). The detection of the particular genetic marker alleles that make up particular haplotypes can be performed by a variety of methods described herein and/or known in the art. For example, genetic markers can be detected at the nucleic acid level (e.g., by direct nucleotide sequencing, or by other genotyping means known to the skilled in the art) or at the amino acid level if the genetic marker affects the coding sequence of a protein (e.g., by protein sequencing or by immunoassays using antibodies that recognize such a protein). The marker alleles or haplotypes of the present invention correspond to fragments of a genomic segments (e.g., genes) associated with cancer. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.2 and/or |D′|>0.8).

In one embodiment, determination of a susceptibility to cancer can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to cancer, a hybridization sample can be formed by contacting the test sample containing an cancer-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of SEQ ID NO:1, as described herein, optionally comprising at least one allele of a marker described herein, or at least one haplotype described herein, or the probe can be the complementary sequence of such a sequence. The nucleic acid probe can also comprise all or a portion of the nucleotide sequence of the TERT gene. In a particular embodiment, the nucleic acid probe is a portion of the nucleotide sequence of SEQ ID NO:1, as described herein, optionally comprising at least one allele of at least one of the polymorphic markers set forth in Tables 5, 6, 7 and 8 herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to cancer.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with cancer. Hybridization of the PNA probe is thus diagnostic for cancer or a susceptibility to cancer.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s). Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid that contains a polymorphic marker or haplotype, and the presence of specific alleles can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet. 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of cancer or a determination of a susceptibility to cancer can be made by examining expression and/or composition of a polypeptide encoded by a nucleic acid associated with cancer in those instances where the genetic marker(s) or haplotype(s) of the present invention result in a change in the composition or expression of the polypeptide. Thus, determination of a susceptibility to cancer can be made by examining expression and/or composition of one of these polypeptides, or another polypeptide encoded by a nucleic acid associated with cancer, in those instances where the genetic marker or haplotype of the present invention results in a change in the composition or expression of the polypeptide. The markers of the present invention that show association to cancer may play a role through their effect on one or more of these nearby genes. In certain embodiments, the markers show an effect on the FoxE1 gene. Possible mechanisms affecting these genes (e.g., the TERT or CLPTM1L genes) include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

Thus, in another embodiment, the variants (markers or haplotypes) presented herein affect the expression of the TERT gene and/or the CLPTM1L gene. It is well known that regulatory element affecting gene expression may be located far away, even as far as tenths or hundreds of kilobases away, from the promoter region of a gene. Variants within such regions may thus affect the expression of distant genes affected by the regulatory region. Thus, by assaying for the presence or absence of at least one allele of at least one polymorphic marker within such regions, it is thus possible to assess the expression level of affected genes. It is thus contemplated that the detection of the markers as described herein, or haplotypes comprising such markers, can be used for assessing and/or predicting the expression of the TERT gene and/or the CLPTM1L gene, or another nearby gene associated with any one of the markers shown herein to confer risk of cancer.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a particular nucleic acid. An alteration in expression of a polypeptide encoded by the nucleic acid can be, for example, an alteration in the quantitative polypeptide expression (i.e., the amount of polypeptide produced). An alteration in the composition of a polypeptide encoded by the nucleic acid is an alteration in the qualitative polypeptide expression (e.g., expression of a mutant polypeptide or of a different splicing variant). In one embodiment, diagnosis of a susceptibility to cancer is made by detecting a particular splicing variant encoded by a nucleic acid associated with cancer, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, cancer. In one embodiment, the control sample is from a subject that does not possess a marker allele or haplotype associated with cancer, as described herein. Similarly, the presence of one or more different splicing variants in the test sample, or the presence of significantly different amounts of different splicing variants in the test sample, as compared with the control sample, can be indicative of a susceptibility to cancer. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with cancer can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of a polypeptide in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, determination of a susceptibility to cancer is made by detecting at least one marker or haplotype of the present invention, in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with cancer, means for analyzing the nucleic acid sequence of a nucleic acid associated with cancer, means for analyzing the amino acid sequence of a polypeptide encoded by a nucleic acid associated with cancer, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays for cancer.

In one embodiment, the invention pertains to a kit for assaying a sample from a subject to detect a susceptibility to cancer in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with cancer risk. In one such embodiment, the polymorphism is selected from the group consisting of the polymorphisms as set forth in Tables 5, 6, 7 and 8 herein. In another embodiment, the polymorphism is selected from rs401681, rs2736100 and rs2736098, and markers in linkage disequilibrium therewith. In another embodiment, the polymorphism is selected from rs401681, rs2736100 and rs2736098. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with risk of cancer. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers set forth in Tables 5, 6, 7 and 8. In another embodiment, the marker or haplotype to be detected comprises one or more markers, two or more markers, three or more markers, four or more markers or five or more markers selected from the group consisting of the markers rs401681, rs2736100 and rs2736098, and markers in linkage disequilibrium therewith. In another embodiment, the marker to be detected is selected from marker rs401681, rs2736100 and rs2736098.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one such embodiment, determination of the presence of the marker or haplotype is indicative of a susceptibility (increased susceptibility or decreased susceptibility) to cancer. In another embodiment, determination of the presence of the marker or haplotype is indicative of response to a therapeutic agent for cancer. In another embodiment, the presence of the marker or haplotype is indicative of prognosis of cancer. In yet another embodiment, the presence of the marker or haplotype is indicative of progress of cancer treatment. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents and Methods

Treatment options for cancer include current standard treatment methods and those that are in clinical trials. Aspects of the invention relating to the use of risk markers for cancer for predicting therapeutic outcome of a particular treatment module, or aspects relating to the application of certain treatment modules for the particular cancer, are contemplated to be useful in the context any therapeutic agents and methods of treating cancer.

Current treatment options for cancer include:

Treatment by Surgery. In principle, non-hematological cancers can be cured if entirely removed by surgery, but this is not always possible. When the cancer has metastasized to other sites in the body prior to surgery, complete surgical excision is usually impossible. In the Halstedian model of cancer progression, tumors grow locally, then spread to the lymph nodes, then to the rest of the body. This has given rise to the popularity of local-only treatments such as surgery for small cancers. Even small localized tumors are increasingly recognized as possessing metastatic potential.

Examples of surgical procedures for cancer include mastectomy for breast cancer and prostatectomy for prostate cancer. The goal of the surgery can be either the removal of only the tumor, or the entire organ. A single cancer cell is invisible to the naked eye but can regrow into a new tumor, a process called recurrence. For this reason, the pathologist will examine the surgical specimen to determine if a margin of healthy tissue is present, thus decreasing the chance that microscopic cancer cells are left in the patient.

In addition to removal of the primary tumor, surgery is often necessary for staging, e.g. determining the extent of the disease and whether it has metastasized to regional lymph nodes. Staging is a major determinant of prognosis and of the need for adjuvant therapy.

Occasionally, surgery is necessary to control symptoms, such as spinal cord compression or bowel obstruction. This is referred to as palliative treatment.

Treatment by Radiation therapy. Also called radiotherapy, X-ray therapy, or irradiation, radiation therapy is the use of ionizing radiation to kill cancer cells and shrink tumors. Radiation therapy can be administered externally via external beam radiotherapy (EBRT) or internally via brachytherapy. The effects of radiation therapy are localised and confined to the region being treated. Radiation therapy injures or destroys cells in the area being treated (the “target tissue”) by damaging their genetic material, making it impossible for these cells to continue to grow and divide. Although radiation damages both cancer cells and normal cells, most normal cells can recover from the effects of radiation and function properly. The goal of radiation therapy is to damage as many cancer cells as possible, while limiting harm to nearby healthy tissue.

Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma. Radiation dose to each site depends on a number of factors, including the radiosensitivity of each cancer type and whether there are tissues and organs nearby that may be damaged by radiation.

Treatment by Chemotherapy. Chemotherapy is the treatment of cancer with drugs (“anticancer drugs”) that can destroy cancer cells. In current usage, the term usually refers to cytotoxic drugs which affect rapidly dividing cells in general, in contrast with targeted therapy. Chemotherapy drugs interfere with cell division in various possible ways, e.g. with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can. Hence, chemotherapy has the potential to harm healthy tissue, especially those tissues that have a high replacement rate (e.g. intestinal lining). These cells usually repair themselves after chemotherapy.

Because some drugs work better together than alone, two or more drugs are often given at the same time. This is called “combination chemotherapy”; most chemotherapy regimens are given in a combination.

The treatment of some leukaemia's and lymphomas requires the use of high-dose chemotherapy, and total body irradiation (TBI). This treatment ablates the bone marrow, and hence the body's ability to recover and repopulate the blood. For this reason, bone marrow, or peripheral blood stem cell harvesting is carried out before the ablative part of the therapy, to enable “rescue” after the treatment has been given. This is known as autologous stem cell transplantation. Alternatively, hematopoietic stem cells may be transplanted from a matched unrelated donor (MUD).

Treatment by Targeted Therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors imatinib and gefitinib.

Monoclonal antibody therapy is another strategy in which the therapeutic agent is an antibody which specifically binds to a protein on the surface of the cancer cells. Examples include the anti-HER2/neu antibody trastuzumab (Herceptin) used in breast cancer, and the anti-CD20 antibody rituximab, used in a variety of B-cell malignancies.

Targeted therapy can also involve small peptides as “homing devices” which can bind to cell surface receptors or affected extracellular matrix surrounding the tumor. Radionuclides which are attached to this peptides (e.g. RGDs) eventually kill the cancer cell if the nuclide decays in the vicinity of the cell. Especially oligo- or multimers of these binding motifs are of great interest, since this can lead to enhanced tumor specificity and avidity.

Photodynamic therapy (PDT) is a ternary treatment for cancer involving a photosensitizer, tissue oxygen, and light (often using lasers). PDT can be used as treatment for basal cell carcinoma (BCC) or lung cancer; PDT can also be useful in removing traces of malignant tissue after surgical removal of large tumors.

Treatment by Immunotherapy. Cancer immunotherapy refers to a diverse set of therapeutic strategies designed to induce the patient's own immune system to fight the tumor. Contemporary methods for generating an immune response against tumours include intravesical BCG immunotherapy for superficial bladder cancer, and use of interferons and other cytokines to induce an immune response in renal cell carcinoma and melanoma patients. Vaccines to generate specific immune responses are the subject of intensive research for a number of tumours, notably malignant melanoma and renal cell carcinoma. Sipuleucel-T is a vaccine-like strategy in late clinical trials for prostate cancer in which dendritic cells from the patient are loaded with prostatic acid phosphatase peptides to induce a specific immune response against prostate-derived cells.

Allogeneic hematopoietic stem cell transplantation (“bone marrow transplantation” from a genetically non-identical donor) can be considered a form of immunotherapy, since the donor's immune cells will often attack the tumor in a phenomenon known as graft-versus-tumor effect. For this reason, allogeneic HSCT leads to a higher cure rate than autologous transplantation for several cancer types, although the side effects are also more severe.

Treatment by Hormonal Therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

Treatment by Angiogenesis inhibitors. Angiogenesis inhibitors prevent the extensive growth of blood vessels (angiogenesis) that tumors require to survive. Some, such as bevacizumab, have been approved and are in clinical use.

Symptom control. Although the control of the symptoms of cancer is not typically thought of as a treatment directed at the cancer, it is an important determinant of the quality of life of cancer patients, and plays an important role in the decision whether the patient is able to undergo other treatments. Although doctors generally have the therapeutic skills to reduce pain, nausea, vomiting, diarrhea, hemorrhage and other common problems in cancer patients, the multidisciplinary specialty of palliative care has arisen specifically in response to the symptom control needs of this group of patients.

Pain medication, such as morphine and oxycodone, and antiemetics, drugs to suppress nausea and vomiting, are very commonly used in patients with cancer-related symptoms.

Improved antiemetics such as ondansetron and analogues, as well as aprepitant have made aggressive treatments much more feasible in cancer patients.

Chronic pain due to cancer is almost always associated with continuing tissue damage due to the disease process or the treatment (i.e. surgery, radiation, chemotherapy). Although there is always a role for environmental factors and affective disturbances in the genesis of pain behaviours, these are not usually the predominant etiologic factors in patients with cancer pain. Furthermore, many patients with severe pain associated with cancer are nearing the end of their lives and palliative therapies are required. The typical strategy for cancer pain management is to get the patient as comfortable as possible using opioids and other medications, surgery, and physical measures.

The variants disclosed herein to confer increased risk of cancer can also be used to identify novel therapeutic targets for cancer. For example, genes containing, or in linkage disequilibrium with, one or more of these variants, or their products (e.g., the TERT gene, the CLPTM1L gene and their gene products), as well as genes or their products that are directly or indirectly regulated by or interact with these genes or their products, can be targeted for the development of therapeutic agents to treat cancer, or prevent or delay onset of symptoms associated with cancer. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein (e.g., the markers and haplotypes set forth in Table 2) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9: R440-442 (1999).

A genetic defect leading to increased predisposition or risk for development of a disease, such as cancer, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat cancer. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid. In certain embodiments, the agent or compound modulates the activity or expression of the TERT gene and/or the CLPTMIL gene. The agents or compounds may also inhibit or alter the undesired activity or expression of the encoded nucleic acid product, i.e. the TERT and/or CLPTM1L protein product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating cancer can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site as described herein, e.g. rs401681, rs2736100 and/or rs2736098, or markers in linkage disequilibrium therewith, is indicative of a different response, e.g. a different response rate, to a particular treatment modality. This means that a patient diagnosed with cancer, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. The treatment may include any of the treatment options described in more detail in the above under Therapeutic Agents and Methods. For example, for a newly diagnosed patient, the presence of a marker of the present invention may be assessed (e.g., through testing DNA derived from a blood sample, as described herein). If the patient is positive for a marker allele or haplotype (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

Any of the treatment methods and compounds described in the above under Therapeutic agents and Methods can be used in such methods. I.e., a treatment for cancer using any of the compounds or methods described or contemplated in the above may, in certain embodiments, benefit from screening for the presence of particular alleles for at least one of the polymorphic markers described herein, wherein the presence of the particular allele is predictive of the treatment outcome for the particular compound or method.

In certain embodiments, a therapeutic agent (drug) for treating cancer is provided together with a kit for determining the allelic status at a polymorphic marker as described herein (e.g., rs965513, or markers in linkage disequilibrium therewith). If an individual is positive for the particular allele or plurality of alleles being tested, the individual is more likely to benefit from the particular compound than non-carriers of the allele. In certain other embodiments, genotype information about the at least one polymorphic marker predictive of the treatment outcome of the particular compound is predetermined and stored in a database, in a look-up table or by other suitable means, and can for example be accessed from a database or look-up table by conventional data query methods known to the skilled person. If a particular individual is determined to carry certain alleles predictive of positive treatment outcome of a particular compound or drug for treating cancer, then the individual is likely to benefit from administration of the particular compound.

The present invention also relates to methods of monitoring progress or effectiveness of a treatment for cancer. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for cancer as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention may be more likely to respond favorably to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with cancer when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of lifestyle changes and administration of particular treatment, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

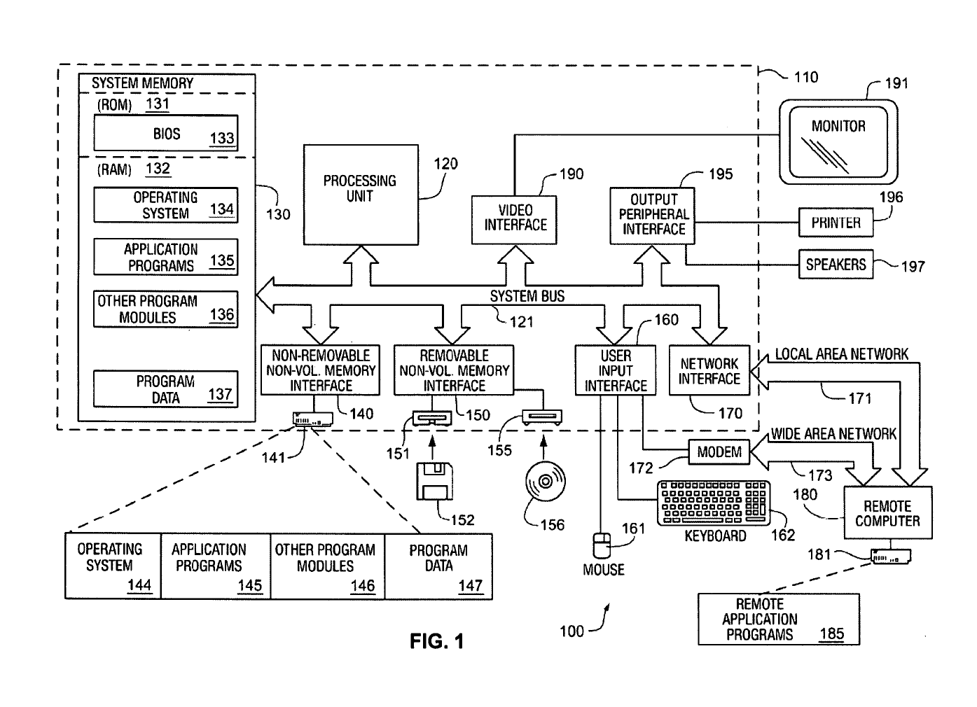

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the cancer, and reporting results based on such comparison.

In general terms, computer-readable media has capabilities of storing (i) identifier information for at least one polymorphic marker or a haplotype, as described herein; (ii) an indicator of the frequency of at least one allele of said at least one marker, or the frequency of a haplotype, in individuals with cancer; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

The markers and haplotypes described herein to be associated with increased susceptibility (e.g., increased risk) of cancer, are in certain embodiments useful for interpretation and/or analysis of genotype data. Thus in certain embodiments, an identification of an at-risk allele for cancer, as shown herein, or an allele at a polymorphic marker in LD with any one of the markers shown herein to be associated with cancer, is indicative of the individual from whom the genotype data originates is at increased risk of cancer. In one such embodiment, genotype data is generated for at least one polymorphic marker shown herein to be associated with cancer, or a marker in linkage disequilibrium therewith. The genotype data is subsequently made available to a third party, such as the individual from whom the data originates, his/her guardian or representative, a physician or health care worker, genetic counselor, or insurance agent, for example via a user interface accessible over the Internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk varians in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of SEQ ID NO. 1, or a nucleotide sequence comprising, or consisting of, the complement of the nucleotide sequence of SEQ ID NO:1, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene Surf'ZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention (e.g., a TERT or CLPTM1L polypeptide) by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorpic marker of the invention, can be used to identify individuals that require modified treatment modalities.

Antibodies can furthermore be useful for assessing expression of variant proteins (e.g., TERT and/or CLPTM1L) in disease states, such as in active stages of a disease, or in an individual with a predisposition to a disease related to the function of the protein, in particular cancer. Antibodies specific for a variant protein of the present invention that is encoded by a nucleic acid that comprises at least one polymorphic marker or haplotype as described herein can be used to screen for the presence of the variant protein, for example to screen for a predisposition to cancer as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting examples.

### Example 1

Sequence Variants on Chromosome 5p13.3 that Associate with Cancer at Multiple Sites

Recently, genome-wide association studies of several cancers have identified common genetic variants that associate with increased cancer risk (Gudmundsson, J., et al. Nat Genet 39:631-637 (2007); Stacey, S. N., et al., Nat. Genet. 39:865-69 (2007); Yeager, M. et al. Nat Genet. 39:645-649 (2007); Gudmundsson, J., et al. Nat Genet 39:977-983 (2007); Haiman, C. A., et al. Nat Genet 39:638-644 (2007); Eason, D. F., et al. Nature 447:1087-1093 (2007); Tomlinson, I., et al. Nat Genet 39:984-988 (2007); Gudbjartsson, D. F., et al. Nat Genet. 40:886-891 (2008); Stacey, S. N., et al. Nat Genet 40:703-706 (2008); Thorgeirsson, T. E., et al. Nature 452:638-642 (2008); Gudmundsson, 3., et al. Nat Genet 40:281-283 (2008); Eeles, R. A., et al. Nat Genet 40:316-321 (2008); Hung, R. J., et al. Nature 452:633-637 (2008); Amos, C. I., et al. Nat Genet 40:616-622 (2008); Thomas, G., et al. Nat Genet. 40:310-315 (2008)). Notably, in most cases the reported variants seem to be specific to the particular cancer type under study. This tissue specificity even holds true in the region on chromosome 8q24, which has been found to associate with several different types of cancer. Independent variants in the 8q24 region have been found that associate with risk of prostate, breast and bladder cancer. Only one of the prostate cancer variants has been shown also to associate with risk of another cancer, i.e. colorectal cancer (Tomlinson, I., et al. Nat Genet. 39:984-988 (2007)). We now show that variants on chromosome 5p13.3 associate with cancer at multiple sites.

### Cohorts

The cohorts are described in the following:

Prostate cancer: Described in Gudmundsson 3, et al. Nature Genetics 39, 631-637 (2007).
Lung cancer: Described in Thorgeirsson T E, et al. Nature 452, 638-642
Bladder cancer: Described in Kiemeney L A, et al. Nature Genetics (in press)
BCC: Described in Stacey S N, et al. Nature Genetics (in press)
Cervical cancer and Thyroid cancer: The cervical cancer program at deCODE genetics is a part of a larger program termed the Icelandic Cancer Project. The major hypothesis of the Icelandic Cancer Project is that cancer is a group of related disorders that have common genetic causes and can be viewed as a single phenotype. The projects have been approved by the Data Protection Authority of Iceland and the National Bioethics Committee. Cancer cases were identified based on records from the nation-wide Icelandic Cancer Registry (ICR; www.krabbameinsskra.is) which include information on the year and age at diagnosis, year of death, SNOMED code and ICD-10 classification. All participants are recruited by trained nurses through special recruitment clinics where they donate a blood sample and answer a lifestyle questionnaire. Clinical information on cancer patients were extracted from medical records at treatment centers. Written informed consent was obtained from all participants. Personal identifiers associated with medical information and blood samples were encrypted with a third-party encryption system in which the Data Protection Authority maintains the code. A total of 803 women were diagnosed with cervical cancer between Jan. 1, 1955 and Dec. 31, 2007. Samples from approximately 300 women were available for genotyping and genotypes of additional 150 women could be imputed.

### Genotyping

All cases and controls were assayed using genotyping systems and specialized software from Illumine (Human Hap300 and HumanCNV370-duo Bead Arrays, Illumine). Furthermore, all Dutch bladder cancer cases and controls have been genotyped with the HumanCNV370-duo Bead Arrays. These chips provide about 75% genomic coverage in the Utah CEPH (CEU) HapMap samples for common SNPs at r2>0.8 (Barrett, J. C. and Cardon, L. R., Nat Genet. 38:659-662 (2006)). SNP data were discarded if the minor allele frequency in the combined case and control was <0.001 or had less than 95% yield or showed a very significant distortion from Hardy-Weinberg equilibrium in the controls (P<1×10⁻¹⁰). Any chips with a call rate below 98% of the SNPs were excluded from the genome-wide association analysis.

All single SNP genotyping was carried out applying the Centaurus (Nanogen) platform (Kutyavin, I. V., et al. Nucleic Acids Res 34:e128 (2006)). The quality of each Centaurus SNP assay was evaluated by genotyping each assay in the CEU HapMap samples and comparing the results with the HapMap publicly released data. Assays with >1.5% mismatch rate were not used and a linkage disequilibrium (LD) test was used for markers known to be in LD. Approximately 10% of the Icelandic case samples that were genotyped on the Illumina platform were also genotyped using the Centaurus assays and the observed mismatch rate was lower than 0.5%. All genotyping was carried out at the deCODE Genetics facility.

### Statistical Analysis

Association analysis. A likelihood procedure described in a previous publication (Yeager, M., et al. Nat Genet 39:645-649 (2007)) and implemented in the NEMO software was used for the association analyses. An attempt was made to genotype all individuals and all SNPs reported, and for each of the SNPs, the yield was higher than 95% in every study group. The SNPs rs4645960 and rs16901979 are not a part of the Human Hap300/HumanCNV370-duo chips. For these SNPs, a subset of the large Icelandic control set as well as all Icelandic cases and all individuals from the replication study groups were genotyped by single track assays. We tested the association of an allele to UBC using a standard likelihood ratio statistic that, if the subjects were unrelated, would have asymptotically a X² distribution with one degree of freedom under the null hypothesis. Allelic frequencies rather than carrier frequencies are presented for the markers in the main text. Allele-specific ORs and associated P values were calculated assuming a multiplicative model for the two chromosomes of an individual (Gudmundsson, J., et al. Nat Genet 39:977-983 (2007)). Results from multiple case-control groups were combined using a Mantel-Haenszel model (Haiman, C. A., et al. Nat Genet. 39:638-644 (2007)) in which the groups were allowed to have different population frequencies for alleles, haplotypes and genotypes but were assumed to have common relative risks.

Correction of the GWA studies by genomic control. To adjust for possible population stratification and the relatedness amongst individuals, we divided the X² test statistics from the individual scans using the method of genomic control (13), i.e. the 302,140 X² test statistics were divided by their means, which were 1.04 and 1.075 for Iceland and the Netherlands, respectively.

### Results

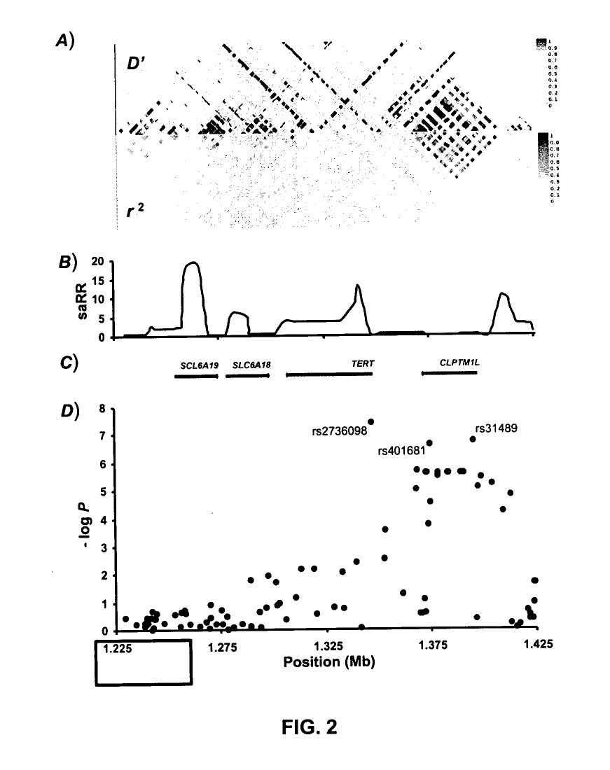

In a genome-wide association study of Basal Cell Carcinoma, we identified two signals on Chr1p and one on Chr1q that reached genome-wide significance in analysis of Icelandic and European sample sets (Stacey 2008, submitted). Subsequently, we followed up the initial GWA scan, increasing the effective sample size by chip-genotyping additional Icelandic BCC cases (total 1011 cases) and imputing genotypes from un-typed BCC cases, using the genealogy database of all Icelanders as previously described (Gudbjartsson, D. F., et al. Nat Genet. 40:609-15 (2008)). The results were adjusted for relatedness between individuals and for potential population stratification using the method of genomic control. The third strongest signal from the GWA was located on chromosome 5p15.3 and represented by three SNPs, rs31489, rs401681 and rs4975616 (minimum P=1.9×10⁻⁷). Those three markers are strongly correlated for all pairwise LD tests, D′>0.9 and r2>0.75 (from 0.75 to 0.87), and belong to an area of high linkage disequilibrium (LD) (FIG. 2). This area overlaps with two genes, CLPTM1L (encoding cisplatin resistance related protein CRR9p) and TERT (encoding human telomerase reverse transcriptase).

To further confirm the association with 5p15.3, we genotyped rs401681 by single track assays in additional 731 BCC cases from Iceland and 525 BCC cases and 525 controls from Eastern Europe. The association of allele C of rs401681 to BCC in the combined analysis of the Icelandic and Eastern Europe reached genome-wide significance (P=3.13×10⁻¹¹, OR=1.25) (Table 1A). We did not observe heterogeneity of the ORs between the Icelandic and East European groups. Results from all groups combined demonstrated that the association of rs401681 allele C to BCC did not deviate from the multiplicative model (P>0.05).

The two genes in the LD region, CLPTM1L and TERT have both been studied in the context of cancer. CLPTM1L was identified in an ovarian cancer model as a gene that affected cisplatin-induced apoptosis but has not been extensively studied (Yamamoto, K. et al. Biochem Biophys Res Comm 280:1148-54 (2001)). The TERT gene plays a leading role in maintenance of functional telomeres and has been firmly established as a key gene in cancer development. In particular, the well-documented association between telomere function and environmental insults such as radiation suggests a potential link between TERT and predisposition to BCC (reviewed in Ayouaz, A., et al. Biochimie 90:60-72 (2008)). Given the relevance of this genomic region to general cancer biology, we assessed the association of rs401681 allele C to 16 cancer types in individuals of European ancestry, making use of samples and data collected through the Icelandic Cancer Project (Rafnar, T., et al. Nat Rev Cancer 4:488-92 (2004)) and by a number of collaborators in Europe. This Icelandic sample collection includes cases with various types of cancer, ascertained through the nation-wide Icelandic Cancer Registry. We have previously genotyped over 38,000 Icelandic individuals, using the HumanHap300/HumanCNV370-duo BeadChips. In addition to the chip data, we used single track genotyping on available cases that had not been genotyped on the chip and imputations of un-genotyped individuals using a database containing the genealogy of all Icelanders. We also genotyped all non-Icelandic case control sets by single-track genotyping and combined all genotype information with result summaries of public GWA datasets (CGEMS, ICR, IARC). In total, we assessed the association of rs401681allele C to 16 individual cancer sites using approximately 30,000 cases and 40,000 controls.

In total, 6 cancer sites showed nominal positive association (P<0.05) with rs401681 (Table 1A). The most significant site after BCC was lung cancer, reaching genome wide significance (OR=1.15, P=8.55×10⁻⁸) in the combined analysis of 4 populations from Iceland, the Netherlands and Spain in addition to the dataset from the IARC, made publically available in 2008 (20). The ORs were very similar in the 4 groups, ranging from 1.13 to 1.19. We divided the lung cancer cases in the Icelandic Dutch and Spanish populations into 4 groups based on histology (small cell, squamous cell, adenocarcinoma, others) and assessed the frequency of rs401681allele C in these different types. In all three populations, we observed a higher frequency of the risk variant among cases with squamous cell carcinoma compared to the other histologies (combined OR 1.29, P=0.0019). Comparing squamous cell carcinomas to the control group, the effect was even stronger with an OR of 1.41. We observed an association between rs401681 allele C and bladder cancer in 9 European case control groups (combined OR=1.12, P=4.05×10⁻⁵). While some groups were small, all groups showed an effect in the same direction, ranging from 1.02 to 1.23. For prostate cancer, we were able to analyse data from 5 groups (over 9,000 cases), including the public CGEMS dataset, and demonstrated a significant effect which was consistent between populations (combined OR=1.07, P=4.45×10⁻⁴). Finally, we detected a significant association between rs401681allele C and cervical cancer in Iceland (OR=1.31, P=7.48×10⁻⁴). In this group of cases, a trend towards a stronger association was noted in cases with the squamous cell carcinoma than cases with adenocarcinoma.

Imputation of un-genotyped SNPs in the area was performed using the HapMap CEU database and the genotyped SNPs on the chip. This analysis showed that multiple markers in the area are also associated with BCC, including rs2736100 (OR 1.13, P=1.7×10⁻⁶) and rs2736098 (OR 1.27; P=3.9×10⁻⁸), the latter being a synonymous coding SNP (A305A) in the second exon of the TERT gene (FIG. 2). The SNP rs2736098 is correlated with rs31489, rs401681 and rs4975616 (for all pairwise LD tests D′ is over 0.9 and the r2>0.3 (from 0.33 to 0.39)) with rs401681 being the best correlated marker.

Follow-up analysis revealed that markers rs2736100 and rs2736098 are both associated strongly with cancer at multiple sites (Table 3 and Table 4). It is noteworthy that the risk for rs2736098 is even higher than for rs401681 (Table 4). The rs2736098 marker is correlated to rs401681 and rs2736100 by r²-values of 0.39 and 0.12, respectively (HapMap CEU sample, Release 22). Markers rs401681 and rs2736100 are however in poor LD (r2<0.05). While the signal for rs2736098 is strongest of these three markers, it appears not to fully explain the association to the other two markers. Thus, there may be another, not yet identified, identified genetic variant in LD with these three markers that has an even stronger association to cancer. Alternatively, the association signal in the region is more complex, with multiple unrelated association signals.

It is of interest that 4 of the 5 cancers associated with the risk variants are cancer types that have strong environmental contribution to risk, i.e. smoking and occupational exposures for lung and bladder cancer, UV irradiation for BCC and infection with human papillomavirus for cervical cancer. The majority of cancers in these organs arise in the epithelial layer that is in closest contact with the environment. Although no strong environmental risk factors are currently known for prostate cancer, several external factors such as diet, physical activity and inflammation may have an effect on disease risk. Although telomere length is partly inherited (Slagboom, P E, et al. Am J Hum Genet. 55:876-82 (1994)) various environmental factors such as smoking and radiation also affect telomere length (Valdes, I P, et al. Nat Genet. 40:623-30 (2008)).

TABLE 1A
Association of rs401681 allele C on Chr 5p15.3 to basal cell carcinoma and cancers of
the lung, bladder, prostate, cervix and endometrium in Iceland.

| Study | Number | | Frequency | | | | |
|---|---|---|---|---|---|---|---|
| population | Cases | Controls | Cases | Controls | OR | 95% CI | P value |
| Basal cell | 2,032^{a} | 28,787 | 0.603 | 0.545 | 1.27 | 1.18-1.36 | 7.96 × 10−11 |
| carcinoma | | | | | | | |
| Lung cancer | 1,381^{a} | 28,787 | 0.576 | 0.545 | 1.13 | 1.04-1.23 | 4.21 × 10−3 |
| Bladder | 823^{a} | 28,787 | 0.585 | 0.545 | 1.17 | 1.06-1.30 | 2.87 × 10−3 |
| cancer | | | | | | | |
| Prostate | 2,294^{a} | 28,787 | 0.569 | 0.545 | 1.10 | 1.03-1.18 | 5.69 × 10−3 |
| cancer | | | | | | | |
| Cervical | 369^{a} | 28,787 | 0.611 | 0.545 | 1.31 | 1.12-1.53 | 7.48 × 10−4 |
| cancer | | | | | | | |
| Endometrial | 470^{a} | 28,787 | 0.592 | 0.545 | 1.21 | 1.06-1.38 | 5.50 × 10−3 |
| cancer | | | | | | | |

All P values shown are two-sided. Shown are the corresponding numbers of cases and controls (N), allelic frequencies of variants in affected and control individuals, the allelic odds-ratio (OR) with P values based on the multiplicative model.
^{a}The number reflects the effective sample size obtained by combining results from genotyped individuals and imputation of un-genotyped individuals. See supplementary material.

TABLE 1B
Association to Basal Cell Carcinoma for markers in 200 Kb interval
on chromosome 5p13.3. Genotypes for markers in the HapMap
data set were imputed, as described under Methods.

| Marker 1 | Marker 2 | P value | Position build 36 |
|---|---|---|---|
| rs401681 | rs4246740 | 0.75 | 1239086 |
| rs401681 | rs6554634 | 0.56 | 1239121 |
| rs401681 | rs4640842 | 0.56 | 1240074 |
| rs401681 | rs4460173 | 0.61 | 1240180 |
| rs401681 | rs4072529 | 0.56 | 1240281 |
| rs401681 | rs4975599 | 0.37 | 1241837 |
| rs401681 | rs7736074 | 0.21 | 1242456 |
| rs401681 | rs7719875 | 0.87 | 1243088 |
| rs401681 | rs6884486 | 0.4 | 1243440 |
| rs401681 | rs10060236 | 0.4 | 1244061 |
| rs401681 | rs10061926 | 0.25 | 1244336 |
| rs401681 | rs6872717 | 0.6 | 1248209 |
| rs401681 | rs4975607 | 0.79 | 1255817 |
| rs401681 | rs12520454 | 0.2 | 1257868 |
| rs401681 | rs10072823 | 0.25 | 1258636 |
| rs401681 | rs11133684 | 0.54 | 1268474 |
| rs401681 | rs4975629 | 0.89 | 1269775 |
| rs401681 | rs6554665 | 0.12 | 1270223 |
| rs401681 | rs6554666 | 0.12 | 1270471 |
| rs401681 | rs4975628 | 0.64 | 1273032 |
| rs401681 | rs9418 | 0.35 | 1278121 |
| rs401681 | rs7704882 | 0.96 | 1278434 |
| rs401681 | rs7728646 | 0.96 | 1278564 |
| rs401681 | rs7728667 | 0.96 | 1278626 |
| rs401681 | rs4975623 | 0.61 | 1285491 |
| rs401681 | rs6554677 | 0.84 | 1289291 |
| rs401681 | rs6554679 | 0.017 | 1289690 |
| rs401681 | rs7447815 | 0.24 | 1293757 |
| rs401681 | rs6554684 | 0.87 | 1293848 |
| rs401681 | rs12513872 | 0.021 | 1301354 |
| rs401681 | rs6554691 | 0.13 | 1301873 |
| rs401681 | rs10078761 | 0.11 | 1302594 |
| rs401681 | rs2853691 | 0.43 | 1305950 |
| rs401681 | rs2736118 | 0.0068 | 1313195 |
| rs401681 | rs2075786 | 0.0067 | 1319310 |
| rs401681 | rs4246742 | 0.29 | 1320356 |
| rs401681 | rs10069690 | 0.0089 | 1332790 |
| rs401681 | rs2242652 | 0.18 | 1333028 |
| rs401681 | rs2736098 | 3.90E−08 | 1347086 |
| rs401681 | rs2735845 | 0.00029 | 1353584 |
| rs401681 | rs4975615 | 1.00E−05 | 1368343 |
| rs401681 | rs6554759 | 0.28 | 1370102 |
| rs401681 | rs1801075 | 0.28 | 1370949 |
| rs401681 | rs7727912 | 0.09 | 1371960 |
| rs401681 | rs451360 | 0.26 | 1372680 |
| rs401681 | rs421629 | 2.50E−06 | 1373136 |
| rs401681 | rs380286 | 2.50E−06 | 1373247 |
| rs401681 | rs10073340 | 3.00E−05 | 1374873 |
| rs401681 | rs466502 | 3.40E−06 | 1378767 |
| rs401681 | rs465498 | 2.50E−06 | 1378803 |
| rs401681 | rs452932 | 2.50E−06 | 1383253 |
| rs401681 | rs452384 | 2.50E−06 | 1383840 |
| rs401681 | rs467095 | 2.50E−06 | 1389221 |
| rs401681 | rs31484 | 2.50E−06 | 1390906 |
| rs401681 | rs11948616 | 0.46 | 1396682 |
| rs401681 | rs31490 | 7.90E−06 | 1397458 |
| rs401681 | rs27070 | 3.60E−06 | 1399303 |
| rs401681 | rs37008 | 6.50E−06 | 1404538 |
| rs401681 | rs37005 | 6.30E−05 | 1409450 |
| rs401681 | rs27064 | 1.50E−05 | 1412938 |
| rs401681 | rs27063 | 0.63 | 1413125 |
| rs401681 | rs2292024 | 0.72 | 1417242 |
| rs401681 | rs409932 | 0.22 | 1420736 |
| rs401681 | rs506156 | 0.31 | 1421678 |
| rs401681 | rs461193 | 0.45 | 1421997 |
| rs401681 | rs10075239 | 0.46 | 1423300 |
| rs401681 | rs6873098 | 0.022 | 1424093 |
| rs401681 | rs881618 | 0.022 | 1424719 |

TABLE 1C
Association to Basal Cell Carcinoma for markers in 200 Kb interval on
chromosome 5p13.3. Results are based on a combination of
genotyped cases on the Illumina Hap300 chip and
un-genotyped, imputed cases, as described under Methods.

| Marker 1 | Marker 2 | P value | Position build 36 |
|---|---|---|---|
| rs401681 | rs4975536 | 0.36 | 1229601 |
| rs401681 | rs4975603 | 0.63 | 1234556 |
| rs401681 | rs4246736 | 0.38 | 1239853 |
| rs401681 | rs4975596 | 0.96 | 1242347 |
| rs401681 | rs11747247 | 0.27 | 1253573 |
| rs401681 | rs13159461 | 0.23 | 1256437 |
| rs401681 | rs6554660 | 0.61 | 1260527 |
| rs401681 | rs7727745 | 0.75 | 1265357 |
| rs401681 | rs6554667 | 0.38 | 1270494 |
| rs401681 | rs4975542 | 0.19 | 1275480 |
| rs401681 | rs10060827 | 0.64 | 1276062 |
| rs401681 | rs4975625 | 0.84 | 1281215 |
| rs401681 | rs7445640 | 0.73 | 1289212 |
| rs401681 | rs4075202 | 0.17 | 1296475 |
| rs401681 | rs4073918 | 0.012 | 1297425 |
| rs401681 | rs2736122 | 0.072 | 1310621 |
| rs401681 | rs4975605 | 0.16 | 1328528 |
| rs401681 | rs2736100 | 0.0041 | 1339516 |
| rs401681 | rs2853676 | 0.93 | 1341547 |
| rs401681 | rs2853668 | 0.003 | 1353025 |
| rs401681 | rs4635969 | 0.055 | 1361552 |
| rs401681 | rs4975616 | 2.20E−06 | 1368660 |
| rs401681 | rs402710 | 0.00018 | 1373722 |
| rs401681 | rs401681 | 2.30E−07 | 1375087 |
| rs401681 | rs31489 | 1.90E−07 | 1395714 |
| rs401681 | rs27061 | 0.93 | 1415793 |
| rs401681 | rs2963265 | 0.11 | 1423832 |

TABLE 2
Association of rs401681 allele C on Chr 5p15.3 to basal cell carcinoma and cancers of
the lung, bladder, prostate, cervix, thyroid and endometrium in Iceland and other populations.

| | Number | | Frequency | | | | |
|---|---|---|---|---|---|---|---|
| Study population | Cases | Controls | Cases | Controls | OR | 95% CI | P value |
| Basal cell carcinoma | | | | | | | |
| Iceland | 2,032^{a} | 28,787 | 0.603 | 0.545 | 1.27 | 1.18-1.36 | 7.96 × 10−11 |
| Eastern Europe | 525 | 525 | 0.616 | 0.573 | 1.16 | 0.97-1.39 | 0.098 |
| All combined^{b} | 2,557 | 29,312 | 0.610 | 0.560 | 1.25 | 1.17-1.34 | 3.13 × 10−11 |
| Lung cancer | | | | | | | |
| Iceland | 1,381^{a} | 28,787 | 0.576 | 0.545 | 1.13 | 1.04-1.23 | 4.21 × 10−3 |
| The Netherlands | 529 | 1,832 | 0.610 | 0.570 | 1.18 | 1.02-1.35 | 0.021 |
| Spain | 367 | 1,427 | 0.582 | 0.538 | 1.19 | 1.01-1.41 | 0.034 |
| IARC | 1,920 | 2,517 | 0.617 | 0.586 | 1.16 | 1.06-1.27 | 8 × 10−4 |
| All combined^{b} | 4,197 | 34,563 | 0.596 | 0.560 | 1.15 | 1.10-1.22 | 8.55 × 10−8 |
| Bladder cancer | | | | | | | |
| Iceland | 823^{a} | 28.787 | 0.585 | 0.545 | 1.17 | 1.06-1.30 | 2.87 × 10−3 |
| The Netherlands | 1,277 | 1,832 | 0.584 | 0.570 | 1.06 | 0.96-1.17 | 0.27 |
| UK | 707 | 506 | 0.564 | 0.514 | 1.23 | 1.04-1.44 | 0.014 |
| Italy-Torino | 329 | 379 | 0.550 | 0.545 | 1.02 | 0.84-1.24 | 0.84 |
| Italy-Brescia | 122 | 156 | 0.574 | 0.564 | 1.04 | 0.74-1.46 | 0.82 |
| Belgium | 199 | 378 | 0.603 | 0.554 | 1.22 | 0.95-1.56 | 0.11 |
| Eastern Europe | 214 | 515 | 0.619 | 0.575 | 1.20 | 0.96-1.51 | 0.12 |
| Sweden | 346 | 905 | 0.545 | 0.521 | 1.10 | 0.92-1.31 | 0.30 |
| Spain | 173 | 1,427 | 0.546 | 0.538 | 1.03 | 0.83-1.29 | 0.78 |
| All combined^{b} | 4,190 | 34,885 | 0.578 | 0.535 | 1.12 | 1.06-1.18 | 4.05 × 10−5 |
| Prostate cancer | | | | | | | |
| Iceland | 2,294^{a} | 28,787 | 0.569 | 0.545 | 1.10 | 1.03-1.18 | 5.69 × 10−3 |
| The Netherlands | 994 | 1,832 | 0.576 | 0.570 | 1.02 | 0.92-1.14 | 0.67 |
| Chicago, US | 635 | 693 | 0.581 | 0.568 | 1.06 | 0.90-1.23 | 0.49 |
| Spain | 459 | 1,427 | 0.559 | 0.538 | 1.09 | 0.94-1.26 | 0.27 |
| CGEMS | 5,109 | 5,059 | 0.558 | 0.543 | 1.06 | 1.00-1.11 | 0.036 |
| All combined^{b} | 9,491 | 37,798 | 0.569 | 0.553 | 1.07 | 1.03-1.11 | 4.45 × 10−4 |
| Cervical cancer | | | | | | | |
| Iceland | 369^{a} | 28,787 | 0.611 | 0.545 | 1.31 | 1.12-1.53 | 7.48 × 10−4 |
| Thyroid cancer | | | | | | | |
| Iceland | 528^{a} | 28,787 | 0.538 | 0.545 | 0.97 | 0.85-1.10 | 0.644 |
| Endometrial cancer | | | | | | | |
| Iceland | 470^{a} | 28,787 | 0.592 | 0.545 | 1.21 | 1.06-1.38 | 5.50 × 10−3 |

All P values shown are two-sided. Shown are the corresponding numbers of cases and controls (N), allelic frequencies of variants in affected and control individuals, the allelic odds-ratio (OR) with P values based on the multiplicative model.
^{a}The number reflects the effective sample size obtained by combining results from genotyped individuals and imputation of un-genotyped individuals.
^{b}For the combined study populations, the reported control frequency was the average, unweighted control frequency of the individual populations, while the OR and the P value were estimated using the Mantel-Haenszel model.

TABLE 3
Association of rs2736100 allele G on Chr 5p15.3 to basal cell carcinoma,
cancers of the lung, bladder, prostate, cervix, thyroid and endometrium.

| | Number | | Frequency | | | |
|---|---|---|---|---|---|---|
| Study population | Cases | Controls | Cases | Controls | OR | P value |
| Basal cell carcinoma | | | | | | |
| Iceland | 1,820 | 28,777 | 0.531 | 0.501 | 1.13 | 4.38 × 10−4 |
| Lung cancer | | | | | | |
| Iceland | 1,377^{a} | 28,777 | 0.521 | 0.501 | 1.08 | 0.043 |
| The Netherlands | 508 | 1,831 | 0.558 | 0.519 | 1.17 | 0.0265 |
| Spain | 358 | 1,336 | 0.536 | 0.510 | 1.11 | 0.219 |
| IARC | | | | | 1.19 | 8.5 × 10−5 |
| All combined^{b} | | | | | 1.13 | 1.7 × 10−6 |
| Bladder cancer | | | | | | |
| Iceland | 790^{a} | 28,777 | 0.478 | 0.501 | 0.91 | 0.066 |
| The Netherlands | 1,278 | 1,831 | 0.513 | 0.519 | 0.98 | 0.645 |
| UK | 459 | 339 | 0.512 | 0.509 | 1.01 | 0.901 |
| Italy-Torino | 271 | 293 | 0.528 | 0.558 | 0.88 | 0.307 |
| Italy-Brescia | 159 | 170 | 0.556 | 0.541 | 1.06 | 0.691 |
| Belgium | 161 | 352 | 0.553 | 0.523 | 1.13 | 0.370 |
| Eastern Europe | 152 | 374 | 0.542 | 0.505 | 1.16 | 0.270 |
| Sweden | 292 | 424 | 0.489 | 0.489 | 1.00 | 0.99 |
| Spain | 171 | 1,336 | 0.526 | 0.510 | 1.07 | 0.581 |
| All combined^{b} | | | | | 0.98 | 0.48 |
| Prostate cancer | | | | | | |
| Iceland | 2,245^{a} | 28,777 | 0.505 | 0.501 | 1.02 | 0.59 |
| The Netherlands | 982 | 1,831 | 0.522 | 0.519 | 1.02 | 0.827 |
| Chicago, US | 612 | 688 | 0.497 | 0.488 | 1.03 | 0.670 |
| Spain | 436 | 1,336 | 0.493 | 0.31 | 0.93 | 0.373 |
| CGEMS | 1,176 | 1,104 | 0.481 | 0.505 | 0.90 | 0.11 |
| All combined^{b} | | | | | 0.99 | 0.82 |
| Cervical cancer | | | | | | |
| Iceland | 275 | 28,777 | 0.538 | 0.501 | 1.16 | 0.085 |
| Thyroid cancer | | | | | | |
| Iceland | 182 | 28,777 | 0.577 | 0.501 | 1.36 | 0.00381 |
| Endometrial cancer | | | | | | |
| Iceland | 376 | 28,777 | 0.525 | 0.501 | 1.10 | 0.186 |

All P values shown are two-sided. Shown are the corresponding numbers of cases and controls (N), allelic frequencies of variants in affected and control individuals, the allelic odds-ratio (OR) with P values based on the multiplicative model.
^{a}The number reflects the effective sample size obtained by combining results from genotyped individuals and imputation of un-genotyped individuals. See supplementary material.
^{b}For the combined study populations, the reported control frequency was the average, unweighted control frequency of the individual

TABLE 4
Association of rs2736098 allele A on Chr 5p15.3 to basal cell carcinoma,
cancers of the lung, bladder, prostate and cervix.

| | Number | | Frequency | | | |
|---|---|---|---|---|---|---|
| Study population | Cases | Controls | Cases | Controls | OR | P value |
| Basal cell carcinoma | | | | | | |
| Iceland | 1,653 | 1,709 | 0.327 | 0.263 | 1.36 | 1.018 × 10−8 |
| Lung cancer | | | | | | |
| Iceland | 700 | 1,709 | 0.304 | 0.263 | 1.22 | 0.00405 |
| The Netherlands | 528 | 532 | 0.328 | 0.258 | 1.40 | 4.60 × 10−4 |
| Spain | 366 | 1,387 | 0.269 | 0.229 | 1.24 | 0.0244 |
| All combined^{a} | | | | | 1.27 | 8.8 × 10−7 |
| Bladder cancer | | | | | | |
| Iceland | 463 | 1,709 | 0.284 | 0.263 | 1.11 | 0.209 |
| The Netherlands | 1,067 | 532 | 0.309 | 0.258 | 1.28 | 0.00299 |
| UK | 334 | 263 | 0.284 | 0.260 | 1.13 | 0.356 |
| Italy-Torino | 76 | 85 | 0.283 | 0.265 | 1.10 | 0.715 |
| Italy-Brescia | 159 | 169 | 0.270 | 0.237 | 1.20 | 0.320 |
| Belgium | 133 | 148 | 0.286 | 0.250 | 1.2 | 0.339 |
| Eastern Europe | 206 | 234 | 0.323 | 0.288 | 1.38 | 0.0300 |
| Sweden | 252 | 440 | 0.300 | 0.228 | 1.45 | 0.00366 |
| Spain | 173 | 1,387 | 0.249 | 0.229 | 1.11 | 0.417 |
| All combined^{a} | | | | | 1.21 | 2.8 × 10−6 |
| Prostate cancer | | | | | | |
| Iceland | 1,834 | 1,709 | 0.288 | 0.263 | 1.13 | 0.021 |
| The Netherlands | 982 | 532 | 0.318 | 0.258 | 1.34 | 5.44 × 10−4 |
| Chicago, US | 642 | 673 | 0.298 | 0.265 | 1.18 | 0.0595 |
| Spain | 450 | 1,387 | 0.251 | 0.229 | 1.13 | 0.174 |
| All combined^{a} | | | | | 1.18 | 9.9 × 10−6 |
| Cervical cancer | | | | | | |
| Iceland | 247 | 1,709 | 0.296 | 0.263 | 1.17 | 0.133 |

All P values shown are two-sided. Shown are the corresponding numbers of cases and controls (N), allelic frequencies of variants in affected and control individuals, the allelic odds-ratio (OR) with P values based on the multiplicative model.
^{a}For the combined study populations, the reported control frequency was the average, unweighted control frequency of the individual populations, while the OR and the P value were estimated using the Mantel-Haenszel model.

TABLE 5
Markers in linkage disequilibrium (LD) with rs401681. The markers were
selected from the Caucasian HapMap dataset. Shown are marker
names, values for the LD measures D′ and r2 to rs401681,
the corresponding p-value, the location of the marker in NCBI Build 36,
and reference to the sequence ID for flanking sequence of the marker.

| | | | | Pos in | Pos. Seq |
|---|---|---|---|---|---|
| Marker | D′ | r2 | P value | Bld 36 | ID NO: 1 |
| rs2736098 | 0.94371 | 0.394285 | 2.80E−12 | 1347086 | 51091 |
| rs2735845 | 0.812051 | 0.153473 | 0.000023 | 1353584 | 57589 |
| rs4635969 | 1 | 0.361702 | 2.79E−13 | 1361552 | 65557 |
| rs4975615 | 0.96143 | 0.772861 | 4.56E−24 | 1368343 | 72348 |
| rs4975616 | 0.964609 | 0.86912 | 1.61E−28 | 1368660 | 72665 |
| rs6554759 | 1 | 0.218769 | 1.31E−08 | 1370102 | 74107 |
| rs1801075 | 1 | 0.218769 | 1.53E−08 | 1370949 | 74954 |
| rs451360 | 1 | 0.373833 | 1.48E−13 | 1372680 | 76685 |
| rs421629 | 1 | 1 | 2.78E−37 | 1373136 | 77141 |
| rs380286 | 1 | 1 | 2.78E−37 | 1373247 | 77252 |
| rs402710 | 1 | 0.667674 | 6.62E−23 | 1373722 | 77727 |
| rs10073340 | 1 | 0.201183 | 4.21E−08 | 1374873 | 78878 |
| rs401681 | 1 | 1 | — | 1375087 | 79092 |
| rs466502 | 1 | 0.966555 | 2.61E−35 | 1378767 | 82772 |
| rs465498 | 1 | 1 | 1.36E−37 | 1378803 | 82808 |
| rs452932 | 1 | 1 | 5.57E−37 | 1383253 | 87258 |
| rs452384 | 1 | 1 | 1.36E−37 | 1383840 | 87845 |
| rs467095 | 1 | 1 | 3.15E−37 | 1389221 | 93226 |
| rs31484 | 1 | 1 | 1.36E−37 | 1390906 | 94911 |
| rs31489 | 1 | 0.871795 | 7.70E−31 | 1395714 | 99719 |
| rs31490 | 1 | 0.966555 | 4.56E−35 | 1397458 | 101463 |
| rs27070 | 0.896536 | 0.777014 | 8.09E−25 | 1399303 | 103308 |
| rs37008 | 0.89493 | 0.748554 | 9.41E−24 | 1404538 | 108543 |
| rs37005 | 0.929849 | 0.804816 | 9.41E−26 | 1409450 | 113455 |
| rs27064 | 1 | 0.126214 | 3.66E−06 | 1412938 | 116943 |
| rs409932 | 0.550831 | 0.107998 | 0.00078 | 1420736 | 124741 |

TABLE 6
Markers in linkage disequilibrium (LD) with rs2736100. The markers were
selected from the Caucasian HapMap dataset. Shown are marker names,
values for the LD measures D′ and r2 to rs401681, the corresponding
p-value, the location of the marker in NCBI Build 36, and
reference to the sequence ID for flanking sequence of the marker

| | | | | Pos in | Pos. Seq |
|---|---|---|---|---|---|
| Marker | D′ | r2 | P value | Bld 36 | ID NO: 1 |
| rs2736118 | 0.553481 | 0.133602 | 0.000352 | 1313195 | 17200 |
| rs10069690 | 1 | 0.254032 | 2.01E−10 | 1332790 | 36795 |
| rs2242652 | 1 | 0.144385 | 9.12E−07 | 1333028 | 37033 |
| rs2736100 | 1 | 1 | — | 1339516 | 43521 |
| rs2853676 | 0.70042 | 0.152251 | 0.000062 | 1341547 | 45552 |
| rs2736098 | 0.512301 | 0.12432 | 0.000519 | 1347086 | 51091 |
| rs2853668 | 0.795545 | 0.221923 | 4.17E−07 | 1353025 | 57030 |
| rs2735845 | 0.746929 | 0.138924 | 0.000033 | 1353584 | 57589 |

TABLE 7
Markers in linkage disequilibrium (LD) with rs2736098. The markers were
selected from the Caucasian HapMap dataset. Shown are marker names,
values for the LD measures D′ and r2 to rs401681, the corresponding
p-value, the location of the marker in NCBI Build 36, and
reference to the sequence ID for flanking sequence of the marker.

| | | | | Pos in | Pos. Seq |
|---|---|---|---|---|---|
| Marker | D′ | r2 | P value | Bld 36 | ID NO: 1 |
| rs2736100 | 0.512301 | 0.12432 | 0.000519 | 1339516 | 43521 |
| rs2736098 | 1 | 1 | — | 1347086 | 51091 |
| rs2735845 | 0.707257 | 0.262957 | 7.91E−08 | 1353584 | 57589 |
| rs4635969 | 0.877743 | 0.123373 | 0.00015 | 1361552 | 65557 |
| rs4975615 | 0.932366 | 0.320072 | 7.44E−10 | 1368343 | 72348 |
| rs4975616 | 0.941731 | 0.366745 | 9.75E−12 | 1368660 | 72665 |
| rs451360 | 1 | 0.16287 | 1.13E−07 | 1372680 | 76685 |
| rs421629 | 0.941112 | 0.388022 | 1.51E−11 | 1373136 | 77141 |
| rs380286 | 0.942602 | 0.389248 | 3.78E−12 | 1373247 | 77252 |
| rs402710 | 0.92164 | 0.246097 | 5.20E−08 | 1373722 | 77727 |
| rs401681 | 0.94371 | 0.394285 | 2.80E−12 | 1375087 | 79092 |
| rs466502 | 0.94235 | 0.380001 | 7.48E−12 | 1378767 | 82772 |
| rs465498 | 0.94371 | 0.394285 | 2.80E−12 | 1378803 | 82808 |
| rs452932 | 0.942602 | 0.389248 | 3.78E−12 | 1383253 | 87258 |
| rs452384 | 0.943012 | 0.393703 | 5.60E−12 | 1383840 | 87845 |
| rs467095 | 0.943263 | 0.389464 | 4.37E−12 | 1389221 | 93226 |
| rs31484 | 0.94371 | 0.394285 | 2.80E−12 | 1390906 | 94911 |
| rs31489 | 0.937846 | 0.339478 | 1.24E−10 | 1395714 | 99719 |
| rs31490 | 0.941868 | 0.388643 | 7.56E−12 | 1397458 | 101463 |
| rs27070 | 0.779095 | 0.277985 | 2.25E−08 | 1399303 | 103308 |
| rs37008 | 0.784065 | 0.291201 | 9.63E−09 | 1404538 | 108543 |
| rs37005 | 0.782316 | 0.297432 | 9.94E−09 | 1409450 | 113455 |
| rs27064 | 0.804444 | 0.184486 | 6.31E−06 | 1412938 | 116943 |
| rs2963260 | 0.776843 | 0.106335 | 0.000333 | 1430021 | 134026 |
| rs2963258 | 0.765568 | 0.1038 | 0.000525 | 1430857 | 134862 |
| rs4975544 | 0.647026 | 0.101441 | 0.000786 | 1432743 | 136748 |
| rs12516758 | 0.594292 | 0.137518 | 0.000361 | 1443349 | 147354 |
| rs2962043 | 0.484372 | 0.175515 | 0.000067 | 1561837 | 265842 |
| rs2963284 | 0.512451 | 0.157993 | 0.000108 | 1562930 | 266935 |

TABLE 8
Known SNP markers within the TERT and 10 kb flanking the gene.
Shown are the position of the marker on Chr 5 in NCBI
Build 36, marker names, and reference to the
sequence ID for flanking sequence of the marker.

| C05 Bld 36 | | Pos SEQ |
|---|---|---|
| Location | Marker | ID NO: 1 |
| 1296296 | rs34614851 | 301 |
| 1296427 | rs13361701 | 432 |
| 1296475 | rs4075202 | 480 |
| 1296699 | rs35661976 | 704 |
| 1296847 | rs4994840 | 852 |
| 1296867 | rs35948576 | 872 |
| 1297286 | rs7448994 | 1291 |
| 1297425 | rs4073918 | 1430 |
| 1297575 | rs35952163 | 1580 |
| 1298073 | rs10623391 | 2078 |
| 1298074 | rs33970920 | 2079 |
| 1298102 | rs6871519 | 2107 |
| 1298363 | rs4975540 | 2368 |
| 1298588 | rs7716467 | 2593 |
| 1299972 | rs13176158 | 3977 |
| 1299995 | rs6883980 | 4000 |
| 1300873 | rs4975620 | 4878 |
| 1301354 | rs12513872 | 5359 |
| 1301690 | rs34985696 | 5695 |
| 1301775 | rs12656500 | 5780 |
| 1301873 | rs6554691 | 5878 |
| 1302047 | rs4583925 | 6052 |
| 1302353 | rs4507531 | 6358 |
| 1302356 | rs35988686 | 6361 |
| 1302594 | rs10078761 | 6599 |
| 1303056 | rs6870915 | 7061 |
| 1303292 | rs6875445 | 7297 |
| 1303463 | rs2853693 | 7468 |
| 1303545 | rs2853692 | 7550 |
| 1304712 | rs34288233 | 8717 |
| 1304901 | rs4975539 | 8906 |
| 1305108 | rs35870082 | 9113 |
| 1305111 | rs10710089 | 9116 |
| 1305127 | rs34378183 | 9132 |
| 1305163 | rs35355672 | 9168 |
| 1305364 | rs35430833 | 9369 |
| 1305398 | rs34097921 | 9403 |
| 1305463 | rs4975618 | 9468 |
| 1305475 | rs13179246 | 9480 |
| 1305534 | rs4975617 | 9539 |
| 1305709 | rs35096542 | 9714 |
| 1305765 | rs34344863 | 9770 |
| 1305950 | rs2853691 | 9955 |
| 1305972 | rs35535053 | 9977 |
| 1306592 | rs34527601 | 10597 |
| 1306744 | rs2853690 | 10749 |
| 1306780 | rs5031049 | 10785 |
| 1306918 | rs35033501 | 10923 |
| 1307021 | rs35196264 | 11026 |
| 1307098 | rs35749463 | 11103 |
| 1307201 | rs35699764 | 11206 |
| 1307247 | rs34506158 | 11252 |
| 1307251 | rs34996780 | 11256 |
| 1307287 | rs2853689 | 11292 |
| 1307451 | rs34742644 | 11456 |
| 1307594 | rs35719940 | 11599 |
| 1307753 | rs35080081 | 11758 |
| 1307844 | rs35523995 | 11849 |
| 1307873 | rs34321948 | 11878 |
| 1307943 | rs35013447 | 11948 |
| 1308052 | rs34630753 | 12057 |
| 1308361 | rs35970923 | 12366 |
| 1308520 | rs33954691 | 12525 |
| 1308622 | rs35083412 | 12627 |
| 1308844 | rs35387865 | 12849 |
| 1309228 | rs35976105 | 13233 |
| 1309237 | rs35548585 | 13242 |
| 1309256 | rs34539509 | 13261 |
| 1309283 | rs2853688 | 13288 |
| 1309393 | rs34042051 | 13398 |
| 1309545 | rs35412895 | 13550 |
| 1309569 | rs34468758 | 13574 |
| 1309585 | rs2853687 | 13590 |
| 1309604 | rs35354089 | 13609 |
| 1309652 | rs34240934 | 13657 |
| 1309809 | rs35295542 | 13814 |
| 1309906 | rs34927774 | 13911 |
| 1310011 | rs34821059 | 14016 |
| 1310024 | rs34044586 | 14029 |
| 1310175 | rs36115594 | 14180 |
| 1310183 | rs36107545 | 14188 |
| 1310215 | rs35082932 | 14220 |
| 1310288 | rs35041195 | 14293 |
| 1310335 | rs35738432 | 14340 |
| 1310563 | rs34153812 | 14568 |
| 1310587 | rs34439046 | 14592 |
| 1310620 | rs34461542 | 14625 |
| 1310621 | rs2736122 | 14626 |
| 1310700 | rs35526657 | 14705 |
| 1310813 | rs35167723 | 14818 |
| 1310904 | rs34452728 | 14909 |
| 1310905 | rs34599610 | 14910 |
| 1310920 | rs5865366 | 14925 |
| 1311231 | rs35689290 | 15236 |
| 1311559 | rs35867091 | 15564 |
| 1311667 | rs35092866 | 15672 |
| 1311974 | rs35300412 | 15979 |
| 1312060 | rs2736121 | 16065 |
| 1312063 | rs13182885 | 16068 |
| 1312073 | rs13182892 | 16078 |
| 1312079 | rs9764053 | 16084 |
| 1312088 | rs2736120 | 16093 |
| 1312108 | rs13165630 | 16113 |
| 1312112 | rs2736119 | 16117 |
| 1313033 | rs28576270 | 17038 |
| 1313053 | rs2853686 | 17058 |
| 1313195 | rs2736118 | 17200 |
| 1313331 | rs34018970 | 17336 |
| 1313401 | rs35880956 | 17406 |
| 1313449 | rs34238050 | 17454 |
| 1313450 | rs34693615 | 17455 |
| 1313461 | rs35703455 | 17466 |
| 1313510 | rs35727757 | 17515 |
| 1313513 | rs35735738 | 17518 |
| 1313514 | rs35311994 | 17519 |
| 1313515 | rs35973242 | 17520 |
| 1313715 | rs34062885 | 17720 |
| 1313837 | rs35605907 | 17842 |
| 1313846 | rs34555789 | 17851 |
| 1313877 | rs35550096 | 17882 |
| 1313957 | rs34026153 | 17962 |
| 1313961 | rs36049021 | 17966 |
| 1313982 | rs35621472 | 17987 |
| 1314051 | rs34041736 | 18056 |
| 1314052 | rs11133715 | 18057 |
| 1314220 | rs36077395 | 18225 |
| 1314314 | rs2736117 | 18319 |
| 1314317 | rs34524651 | 18322 |
| 1314460 | rs34060726 | 18465 |
| 1314749 | rs35412024 | 18754 |
| 1314803 | rs34853903 | 18808 |
| 1314809 | rs35348962 | 18814 |
| 1314866 | rs33987455 | 18871 |
| 1315002 | rs35122668 | 19007 |
| 1315004 | rs36121240 | 19009 |
| 1315344 | rs35901859 | 19349 |
| 1315404 | rs34074935 | 19409 |
| 1315492 | rs34864919 | 19497 |
| 1315667 | rs34653167 | 19672 |
| 1316016 | rs34714150 | 20021 |
| 1316042 | rs34909002 | 20047 |
| 1316053 | rs36119674 | 20058 |
| 1316322 | rs35228187 | 20327 |
| 1316339 | rs36077524 | 20344 |
| 1316378 | rs2736116 | 20383 |
| 1316408 | rs34529095 | 20413 |
| 1316471 | rs35617524 | 20476 |
| 1316477 | rs35999328 | 20482 |
| 1316587 | rs34895517 | 20592 |
| 1316650 | rs34289611 | 20655 |
| 1316987 | rs35209454 | 20992 |
| 1317068 | rs2736115 | 21073 |
| 1317145 | rs34555101 | 21150 |
| 1317152 | rs2853685 | 21157 |
| 1317218 | rs34057268 | 21223 |
| 1317290 | rs34656059 | 21295 |
| 1317483 | rs34458182 | 21488 |
| 1317587 | rs34528119 | 21592 |
| 1318204 | rs2736114 | 22209 |
| 1318373 | rs2736113 | 22378 |
| 1318664 | rs7730303 | 22669 |
| 1318723 | rs2736112 | 22728 |
| 1318853 | rs34864337 | 22858 |
| 1318935 | rs2736111 | 22940 |
| 1319141 | rs34823760 | 23146 |
| 1319223 | rs9282869 | 23228 |
| 1319226 | rs2853684 | 23231 |
| 1319310 | rs2075786 | 23315 |
| 1319361 | rs35596904 | 23366 |
| 1319425 | rs36070059 | 23430 |
| 1319919 | rs34033712 | 23924 |
| 1320059 | rs35971139 | 24064 |
| 1320202 | rs3891054 | 24207 |
| 1320213 | rs34194491 | 24218 |
| 1320356 | rs4246742 | 24361 |
| 1320497 | rs11956330 | 24502 |
| 1320659 | rs35359768 | 24664 |
| 1320736 | rs34476748 | 24741 |
| 1320747 | rs35706685 | 24752 |
| 1320751 | rs34246010 | 24756 |
| 1320805 | rs34673480 | 24810 |
| 1320848 | rs35973454 | 24853 |
| 1320881 | rs35812074 | 24886 |
| 1320944 | rs17402061 | 24949 |
| 1320967 | rs34285898 | 24972 |
| 1321239 | rs35949937 | 25244 |
| 1321294 | rs35667898 | 25299 |
| 1321446 | rs34181584 | 25451 |
| 1321464 | rs35243220 | 25469 |
| 1321580 | rs33988305 | 25585 |
| 1321596 | rs34881188 | 25601 |
| 1321836 | rs34923115 | 25841 |
| 1321847 | rs6899038 | 25852 |
| 1321944 | rs6863310 | 25949 |
| 1322006 | rs6863494 | 26011 |
| 1322066 | rs35883631 | 26071 |
| 1322132 | rs6863697 | 26137 |
| 1322148 | rs34948922 | 26153 |
| 1322175 | rs35333551 | 26180 |
| 1322244 | rs35218116 | 26249 |
| 1322278 | rs34020702 | 26283 |
| 1322316 | rs11951776 | 26321 |
| 1322365 | rs6882077 | 26370 |
| 1322923 | rs35209375 | 26928 |
| 1322945 | rs35958533 | 26950 |
| 1322974 | rs35278007 | 26979 |
| 1323358 | rs34701155 | 27363 |
| 1323389 | rs35657226 | 27394 |
| 1323434 | rs35029914 | 27439 |
| 1323539 | rs34860744 | 27544 |
| 1323546 | rs7447741 | 27551 |
| 1323547 | rs35440658 | 27552 |
| 1323872 | rs34146029 | 27877 |
| 1323877 | rs34002187 | 27882 |
| 1323983 | rs11742908 | 27988 |
| 1324069 | rs34554161 | 28074 |
| 1324101 | rs34503345 | 28106 |
| 1324524 | rs11133719 | 28529 |
| 1324585 | rs35664326 | 28590 |
| 1324661 | rs13172201 | 28666 |
| 1324714 | rs35442442 | 28719 |
| 1324793 | rs34980560 | 28798 |
| 1324849 | rs35884863 | 28854 |
| 1324861 | rs35928703 | 28866 |
| 1324878 | rs34297995 | 28883 |
| 1324879 | rs35082205 | 28884 |
| 1325191 | rs35929262 | 29196 |
| 1325197 | rs34031216 | 29202 |
| 1325654 | rs10700998 | 29659 |
| 1325688 | rs10700999 | 29693 |
| 1325701 | rs6885542 | 29706 |
| 1325722 | rs10701000 | 29727 |
| 1325740 | rs6860512 | 29745 |
| 1325813 | rs3134645 | 29818 |
| 1325884 | rs6860815 | 29889 |
| 1325886 | rs2853682 | 29891 |
| 1325900 | rs10701001 | 29905 |
| 1325975 | rs35507727 | 29980 |
| 1325986 | rs4484476 | 29991 |
| 1326020 | rs34767663 | 30025 |
| 1326075 | rs34958852 | 30080 |
| 1326102 | rs34894456 | 30107 |
| 1326101 | rs5865363 | 30106 |
| 1326213 | rs36063319 | 30218 |
| 1326270 | rs34084612 | 30275 |
| 1326648 | rs5865362 | 30653 |
| 1326649 | rs5865361 | 30654 |
| 1326861 | rs2736123 | 30866 |
| 1327445 | rs35517815 | 31450 |
| 1327646 | rs35963133 | 31651 |
| 1327727 | rs35192176 | 31732 |
| 1327830 | rs34774976 | 31835 |
| 1327983 | rs35768726 | 31988 |
| 1328528 | rs4975605 | 32533 |
| 1328857 | rs13156167 | 32862 |
| 1328887 | rs13156282 | 32892 |
| 1328914 | rs13170634 | 32919 |
| 1328915 | rs13156298 | 32920 |
| 1328925 | rs13170637 | 32930 |
| 1328936 | rs13170644 | 32941 |
| 1328952 | rs13156311 | 32957 |
| 1328953 | rs13170656 | 32958 |
| 1330275 | rs35096965 | 34280 |
| 1330488 | rs35072952 | 34493 |
| 1330577 | rs33961405 | 34582 |
| 1330637 | rs11750711 | 34642 |
| 1330728 | rs35811757 | 34733 |
| 1330729 | rs35577391 | 34734 |
| 1330759 | rs7737938 | 34764 |
| 1330803 | rs7719661 | 34808 |
| 1330976 | rs7724028 | 34981 |
| 1331092 | rs35438621 | 35097 |
| 1331125 | rs34140705 | 35130 |
| 1331570 | rs34682571 | 35575 |
| 1331584 | rs2075785 | 35589 |
| 1331629 | rs34049290 | 35634 |
| 1332224 | rs35241335 | 36229 |
| 1332306 | rs33951489 | 36311 |
| 1332391 | rs34108128 | 36396 |
| 1332439 | rs33963617 | 36444 |
| 1332505 | rs33956095 | 36510 |
| 1332523 | rs34625402 | 36528 |
| 1332627 | rs28428579 | 36632 |
| 1332639 | rs34795236 | 36644 |
| 1332701 | rs35247701 | 36706 |
| 1332790 | rs10069690 | 36795 |
| 1332813 | rs34227159 | 36818 |
| 1332815 | rs35278664 | 36820 |
| 1332837 | rs35656989 | 36842 |
| 1332904 | rs35045715 | 36909 |
| 1332964 | rs10054203 | 36969 |
| 1333022 | rs10078991 | 37027 |
| 1333028 | rs2242652 | 37033 |
| 1333128 | rs7734992 | 37133 |
| 1333152 | rs34859168 | 37157 |
| 1333189 | rs34197543 | 37194 |
| 1333252 | rs34471035 | 37257 |
| 1333258 | rs35695689 | 37263 |
| 1333263 | rs33948291 | 37268 |
| 1333387 | rs34170122 | 37392 |
| 1333411 | rs33959226 | 37416 |
| 1333477 | rs13167280 | 37482 |
| 1333573 | rs35868315 | 37578 |
| 1333589 | rs35659884 | 37594 |
| 1333830 | rs4975538 | 37835 |
| 1333938 | rs6897196 | 37943 |
| 1333948 | rs34002450 | 37953 |
| 1333950 | rs11278847 | 37955 |
| 1334079 | rs35079836 | 38084 |
| 1334418 | rs35071105 | 38423 |
| 1334429 | rs35818299 | 38434 |
| 1334529 | rs34989691 | 38534 |
| 1334693 | rs6866456 | 38698 |
| 1334743 | rs6881768 | 38748 |
| 1334890 | rs6554743 | 38895 |
| 1334975 | rs6554744 | 38980 |
| 1335020 | rs36002710 | 39025 |
| 1335159 | rs6867141 | 39164 |
| 1335165 | rs6867270 | 39170 |
| 1335194 | rs7722143 | 39199 |
| 1335220 | rs35300318 | 39225 |
| 1335319 | rs7726159 | 39324 |
| 1335407 | rs34301490 | 39412 |
| 1335414 | rs7725218 | 39419 |
| 1335452 | rs34399181 | 39457 |
| 1335654 | rs35809415 | 39659 |
| 1335746 | rs34846301 | 39751 |
| 1336104 | rs35888851 | 40109 |
| 1336312 | rs7713218 | 40317 |
| 1336334 | rs2853681 | 40339 |
| 1336339 | rs2853680 | 40344 |
| 1336371 | rs2853679 | 40376 |
| 1336375 | rs3134644 | 40380 |
| 1336399 | rs2853678 | 40404 |
| 1336486 | rs7717443 | 40491 |
| 1336841 | rs6420019 | 40846 |
| 1337046 | rs6420020 | 41051 |
| 1337135 | rs4449583 | 41140 |
| 1337151 | rs34785213 | 41156 |
| 1337389 | rs11951797 | 41394 |
| 1337421 | rs35903242 | 41426 |
| 1337484 | rs13189814 | 41489 |
| 1337507 | rs11960793 | 41512 |
| 1337517 | rs11960795 | 41522 |
| 1337525 | rs11954060 | 41530 |
| 1337532 | rs13188694 | 41537 |
| 1337535 | rs11951801 | 41540 |
| 1337554 | rs13175402 | 41559 |
| 1337568 | rs6898599 | 41573 |
| 1337576 | rs13188816 | 41581 |
| 1337580 | rs13189988 | 41585 |
| 1337597 | rs13171544 | 41602 |
| 1337619 | rs13190026 | 41624 |
| 1337628 | rs13175540 | 41633 |
| 1337631 | rs13171555 | 41636 |
| 1337705 | rs28374414 | 41710 |
| 1337749 | rs2736101 | 41754 |
| 1337767 | rs3134643 | 41772 |
| 1337789 | rs34170979 | 41794 |
| 1337795 | rs35318915 | 41800 |
| 1337806 | rs36105933 | 41811 |
| 1337976 | rs35029535 | 41981 |
| 1338162 | rs10866498 | 42167 |
| 1338681 | rs11334193 | 42686 |
| 1338694 | rs11323794 | 42699 |
| 1338974 | rs7705526 | 42979 |
| 1339293 | rs35135137 | 43298 |
| 1339477 | rs34363858 | 43482 |
| 1339516 | rs2736100 | 43521 |
| 1339532 | rs35116243 | 43537 |
| 1339846 | rs35490698 | 43851 |
| 1340002 | rs34829399 | 44007 |
| 1340194 | rs2853677 | 44199 |
| 1340209 | rs35402043 | 44214 |
| 1340290 | rs35838177 | 44295 |
| 1340340 | rs2736099 | 44345 |
| 1340505 | rs7710703 | 44510 |
| 1340623 | rs11291391 | 44628 |
| 1340626 | rs34211134 | 44631 |
| 1341151 | rs34790490 | 45156 |
| 1341303 | rs35086922 | 45308 |
| 1341547 | rs2853676 | 45552 |
| 1341883 | rs34677523 | 45888 |
| 1342286 | rs2853675 | 46291 |
| 1342287 | rs2853674 | 46292 |
| 1342300 | rs2853673 | 46305 |
| 1342463 | rs11950844 | 46468 |
| 1342510 | rs35467152 | 46515 |
| 1342535 | rs35260354 | 46540 |
| 1342552 | rs35121132 | 46557 |
| 1342553 | rs35849820 | 46558 |
| 1342579 | rs34209796 | 46584 |
| 1342594 | rs35252439 | 46599 |
| 1342617 | rs34450169 | 46622 |
| 1342619 | rs35994758 | 46624 |
| 1342997 | rs34582601 | 47002 |
| 1343240 | rs11414507 | 47245 |
| 1345191 | rs34253063 | 49196 |
| 1345299 | rs35334674 | 49304 |
| 1345351 | rs36006348 | 49356 |
| 1345446 | rs34052286 | 49451 |
| 1345626 | rs34006362 | 49631 |
| 1345635 | rs34952664 | 49640 |
| 1345643 | rs33951612 | 49648 |
| 1345649 | rs35746398 | 49654 |
| 1345714 | rs7713080 | 49719 |
| 1345810 | rs35291888 | 49815 |
| 1345983 | rs2853672 | 49988 |
| 1346291 | rs2853671 | 50296 |
| 1346339 | rs35127005 | 50344 |
| 1346368 | rs35691354 | 50373 |
| 1346570 | rs35459373 | 50575 |
| 1346767 | rs34094720 | 50772 |
| 1347086 | rs2736098 | 51091 |
| 1347338 | rs35837567 | 51343 |
| 1347429 | rs11952056 | 51434 |
| 1348204 | rs2735943 | 52209 |
| 1348208 | rs2853670 | 52213 |
| 1348216 | rs35733142 | 52221 |
| 1348243 | rs35550267 | 52248 |
| 1348274 | rs34654879 | 52279 |
| 1348307 | rs35148557 | 52312 |
| 1348322 | rs34233268 | 52327 |
| 1348340 | rs35265333 | 52345 |
| 1348349 | rs2853669 | 52354 |
| 1348373 | rs35226131 | 52378 |
| 1348452 | rs35161420 | 52457 |
| 1348456 | rs34328523 | 52461 |
| 1348459 | rs34764648 | 52464 |
| 1348514 | rs35596874 | 52519 |
| 1348617 | rs10462697 | 52622 |
| 1348682 | rs33958877 | 52687 |
| 1348701 | rs2735942 | 52706 |
| 1348716 | rs34768248 | 52721 |
| 1348735 | rs13181701 | 52740 |
| 1348803 | rs34685900 | 52808 |
| 1349072 | rs7712562 | 53077 |
| 1349090 | rs33958769 | 53095 |
| 1349255 | rs3215401 | 53260 |
| 1349259 | rs5865365 | 53264 |
| 1349260 | rs33923311 | 53265 |
| 1349421 | rs36081204 | 53426 |
| 1349451 | rs2735941 | 53456 |
| 1349456 | rs2736110 | 53461 |
| 1349486 | rs2735940 | 53491 |
| 1349653 | rs33985695 | 53658 |
| 1349727 | rs33977403 | 53732 |
| 1349751 | rs35638571 | 53756 |
| 1349758 | rs33987166 | 53763 |
| 1349759 | rs2736109 | 53764 |
| 1350078 | rs10548207 | 54083 |
| 1350082 | rs36044608 | 54087 |
| 1350258 | rs6554754 | 54263 |
| 1350474 | rs10618795 | 54479 |
| 1350488 | rs2736108 | 54493 |
| 1350854 | rs2736107 | 54859 |
| 1350918 | rs7449190 | 54923 |
| 1351645 | rs3888705 | 55650 |
| 1351733 | rs13190087 | 55738 |
| 1351782 | rs2736106 | 55787 |
| 1351806 | rs34736137 | 55811 |
| 1352213 | rs2735948 | 56218 |
| 1352379 | rs2735846 | 56384 |
| 1352388 | rs35821362 | 56393 |
| 1352392 | rs2735947 | 56397 |
| 1352756 | rs2736105 | 56761 |
| 1352859 | rs13174814 | 56864 |
| 1352862 | rs13174919 | 56867 |
| 1352980 | rs35266184 | 56985 |
| 1353025 | rs2853668 | 57030 |
| 1353070 | rs2853667 | 57075 |
| 1353310 | rs4975612 | 57315 |
| 1353401 | rs2736103 | 57406 |
| 1353429 | rs2735946 | 57434 |
| 1353439 | rs11749061 | 57444 |
| 1353497 | rs34868693 | 57502 |
| 1353580 | rs36037576 | 57585 |
| 1353584 | rs2735845 | 57589 |
| 1353643 | rs35535864 | 57648 |
| 1354874 | rs6880140 | 58879 |
| 1355115 | rs34218850 | 59120 |
| 1355144 | rs2736102 | 59149 |
| 1355914 | rs2853666 | 59919 |
| 1356580 | rs4975613 | 60585 |
| 1356901 | rs2735945 | 60906 |
| 1357238 | rs10052815 | 61243 |
| 1357432 | rs2735944 | 61437 |
| 1357445 | rs10070025 | 61450 |

TABLE 9
SNP markers in the coding and promoter sequences of the TERT gene. Shown are
SNPs in exons of the TERT gene and one promoter SNP that has been associated
with TERT expression (Matsubara, et al., 2006, BBRC 341, 128-131)

| | Pos in Bld | | | | | |
|---|---|---|---|---|---|---|
| Region | 36 | Db SNP rs# | Function | dbSNP | Protein | Amino acid |
| exon 16 | 1306918 | rs35033501 | synonymous | A | Pro [P] | 1108 |
| | | | contig reference | G | Pro [P] | 1108 |
| exon 15 | 1307594 | rs35719940 | missense | A | Thr [T] | 1062 |
| | | | contig reference | G | Ala [A] | 1062 |
| exon 14 | 1308520 | rs33954691 | synonymous | T | His [H] | 1013 |
| | | | contig reference | C | His [H] | 1013 |
| exon 12 | 1313715 | rs34062885 | missense | A | Arg [R] | 948 |
| | | | contig reference | C | Ser [S] | 948 |
| exon 11 | 1317587 | rs34528119 | synonymous | T | His [H] | 925 |
| | | | contig reference | C | His [H] | 925 |
| exon 5 | 1332439 | rs33963617 | synonymous | T | Ala [A] | 699 |
| | | | contig reference | C | Ala [A] | 699 |
| | 1332505 | rs33956095 | synonymous | T | Gly [G] | 677 |
| | | | contig reference | C | Gly [G] | 677 |
| | 1332523 | rs34625402 | synonymous | A | Arg [R] | 671 |
| | | | contig reference | G | Arg [R] | 671 |
| exon 4 | 1333387 | rs34170122 | synonymous | G | Ala [A] | 612 |
| | | | contig reference | C | Ala [A] | 612 |
| | 1333411 | rs33959226 | synonymous | G | Ala [A] | 604 |
| | | | contig reference | A | Ala [A] | 604 |
| exon 3 | 1335654 | rs35809415 | synonymous | T | Val [V] | 553 |
| | | | contig reference | C | Val [V] | 553 |
| exon 2 | 1346570 | rs35459373 | synonymous | G | Leu [L] | 477 |
| | | | contig reference | C | Leu [L] | 477 |
| | 1346767 | rs34094720 | missense | T | Tyr [Y] | 412 |
| | | | contig reference | C | His [H] | 412 |
| | 1347086 | rs2736098 | synonymous | A | Ala [A] | 305 |
| | | | contig reference | G | Ala [A] | 305 |
| | 1347338 | rs35837567 | synonymous | A | Ala [A] | 221 |
| | | | contig reference | G | Ala [A] | 221 |
| | 1347429 | rs11952056 | missense | C | Thr [T] | 191 |
| | | | contig reference | G | Ser [S] | 191 |
| exon 2 | 1347166 | rs61748181 | missense | C | Ala [A] | 279 |
| | | | | T | Thr [T] | |
| Promoter | 1349486 | rs2735940 | May affect | C | | |
| | | | | T | | |

### Example 2

The C allele of marker rs401681 was found to be associated with a protection against cutaneous melanoma and colorectal cancer. Thus a significant association between rs401681(C) and protection against cutaneous melanoma (OR=0.88, P=8.0×10⁻⁴) in a sample set consisting of 2,443 melanoma cases and 30,839 controls from Iceland, Sweden and Spain. We note that a recently published study of telomere length in individuals with skin cancers showed that while short telomeres are associated with increased risk of BCC, long telomeres are associated with increased risk of melanoma (Han, J. et al. J Invest Dermatol 129, 415-21 (2009)). The rs401681(C) variant was also marginally associated with protection against colorectal cancer (OR=0.95, P=8.4×10³) although this was not significant after taking into account the number of cancer sites tested.

### Example 3

We examined the joint effect of rs401681(C) and rs2736098 (A), for 5 cancers, using only samples typed for both SNPs (Table 10). After adjusting for rs2736098 (A), the association of rs401681(C) remained significant in all except prostate cancer. After adjusting for rs401681(C), rs2736098 (A) remained significant for 3 cancers, lung, bladder and prostate. Overall, these results indicate that neither rs401681(C) nor rs2736098 (A) can, by themselves, fully account for the association observed between sequence variants in this region and the 5 cancer types. This suggests that a unique variant capturing the effect of both rs401681(C) and rs2736098(A) remains to be discovered or, alternatively, that the region contains more than one variant that predisposes to cancers at the same or different sites, analogous to the region on 8q24 where independent variants have been found that associate with different cancer types. We analyzed the association between 27 SNPs surrounding rs401681 and rs2736098 and the 17 cancer types studied using the Icelandic sample sets and found that 15 sites showed an association with one or more of these SNPs at the P<0.05 level (Table 11).

TABLE 10
Joint analysis of rs401681(C) and rs2736098 (A) of BCC and
cancers of the lung, bladder, prostate and cervix.

| | | rs401681(C) adjusted for | | | rs2736098(A) adjusted for | | |
|---|---|---|---|---|---|---|---|
| | # | rs2736098(A) | | | rs401681(C) | | |
| Cancer type | populations | OR | 95% CI | P value | OR | 95% CI | P value |
| Basal cell carcinoma | 2 | 1.20 | 1.10-1.31 | 7.8 × 10⁻⁵ | 1.09 | 0.99-1.21 | 0.091 |
| Lung cancer | 3 | 1.11 | 1.01-1.21 | 0.024 | 1.14 | 1.03-1.25 | 0.010 |
| Bladder cancer | 9 | 1.07 | 1.00-1.16 | 0.036 | 1.12 | 1.04-1.20 | 0.0034 |
| prostate cancer | 4 | 1.01 | 0.95-1.08 | 0.68 | 1.13 | 1.05-1.21 | 0.0015 |
| Cervical cancer | 1 | 1.27 | 1.03-1.55 | 0.022 | 0.97 | 0.77-1.22 | 0.80 |

TABLE 11
SNPs in the region depicted in FIG. 1 with a P value <0.05 for
one or more of 17 cancer sites, using chip genotyped and
in silico genotyped cases and controls in Iceland

| SNP | DISEASE | D′ | R2 |
|---|---|---|---|
| rs10060827 | SCC | 0.261966 | 0.029914 |
| rs13159461 | bladder | 0.059183 | 0.001709 |
| rs13159461 | cervix | 0.059183 | 0.001709 |
| rs13159461 | pancreas | 0.059183 | 0.001709 |
| rs2736100 | BCC | 0.139999 | 0.018319 |
| rs2736100 | lung | 0.139999 | 0.018319 |
| rs2736100 | thyroid | 0.139999 | 0.018319 |
| rs2736122 | colorectal | 0.244406 | 0.015226 |
| rs2736122 | prostate | 0.244406 | 0.015226 |
| rs2736122 | thyroid | 0.244406 | 0.015226 |
| rs2853668 | BCC | 0.298694 | 0.02924 |
| rs2853668 | thyroid | 0.298694 | 0.02924 |
| rs2853676 | bladder | 0.148461 | 0.010933 |
| rs2853676 | lung | 0.148461 | 0.010933 |
| rs2963265 | endometrium | 0.359744 | 0.08954 |
| rs31489 | BCC | 1 | 0.871795 |
| rs31489 | bladder | 1 | 0.871795 |
| rs31489 | cervix | 1 | 0.871795 |
| rs31489 | endometrium | 1 | 0.871795 |
| rs31489 | lung | 1 | 0.871795 |
| rs31489 | prostate | 1 | 0.871795 |
| rs401681 | BCC | NA | NA |
| rs401681 | bladder | NA | NA |
| rs401681 | cervix | NA | NA |
| rs401681 | endometrium | NA | NA |
| rs401681 | lung | NA | NA |
| rs401681 | prostate | NA | NA |
| rs402710 | BCC | 1 | 0.667674 |
| rs402710 | endometrium | 1 | 0.667674 |
| rs402710 | lung | 1 | 0.667674 |
| rs402710 | prostate | 1 | 0.667674 |
| rs4073918 | BCC | 0.001157 | 0.000001 |
| rs4073918 | bladder | 0.001157 | 0.000001 |
| rs4073918 | thyroid | 0.001157 | 0.000001 |
| rs4246736 | bladder | 0.054681 | 0.001421 |
| rs4246736 | kidney | 0.054681 | 0.001421 |
| rs4246736 | pancreas | 0.054681 | 0.001421 |
| rs4635969 | bladder | 1 | 0.361702 |
| rs4635969 | cervix | 1 | 0.361702 |
| rs4975536 | breast | 0.247364 | 0.032948 |
| rs4975542 | colorectal | 0.324438 | 0.005749 |
| rs4975596 | breast | 0.178476 | 0.0158 |
| rs4975605 | melanoma | 0.019886 | 0.00038 |
| rs4975605 | multi_myeloma | 0.019886 | 0.00038 |
| rs4975605 | thyroid | 0.019886 | 0.00038 |
| rs4975616 | BCC | 0.964609 | 0.86912 |
| rs4975616 | bladder | 0.964609 | 0.86912 |
| rs4975616 | cervix | 0.964609 | 0.86912 |
| rs4975616 | endometrium | 0.964609 | 0.86912 |
| rs4975616 | lung | 0.964609 | 0.86912 |
| rs4975625 | lung | 0.215441 | 0.027142 |
| rs4975625 | SCC | 0.215441 | 0.027142 |
| rs6554667 | endometrium | 0.230769 | 0.002143 |
| rs6554667 | prostate | 0.230769 | 0.002143 |
| rs7445640 | lung | 0.287008 | 0.044994 |
| rs7727745 | bladder | 0.025954 | 0.000063 |
| rs7727745 | lymphoma | 0.025954 | 0.000063 |
| rs7727745 | multi myeloma | 0.025954 | 0.000063 |

D′ and R2 with reference to rs401681
NA = not applicable

### Example 4

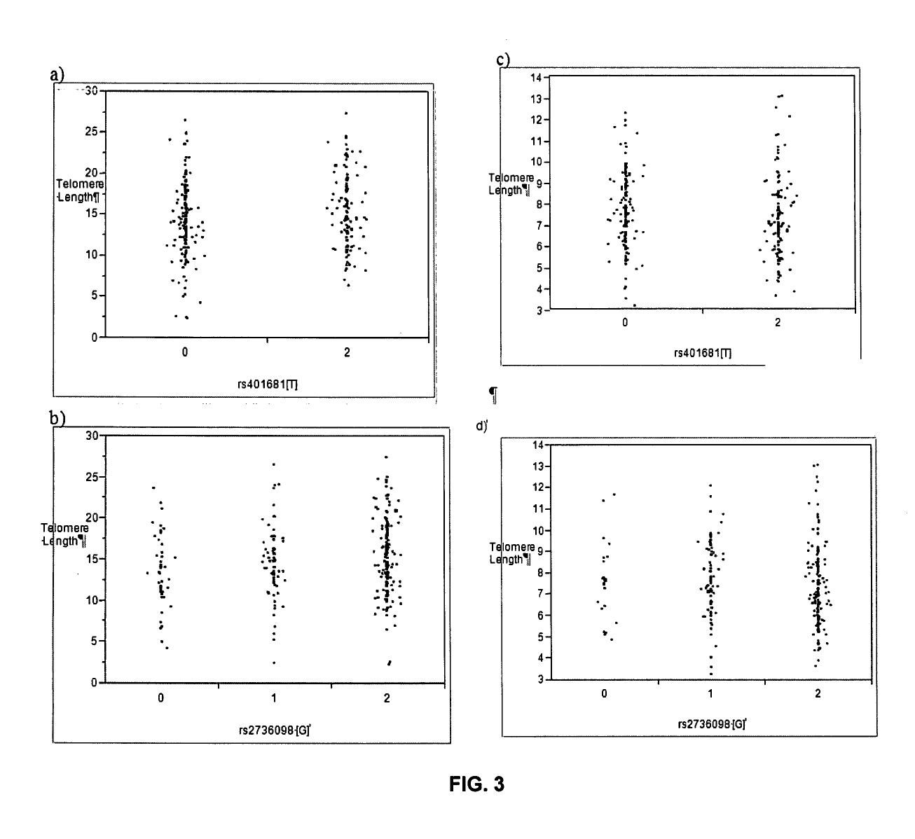

We postulated that the cancer-associated sequence variants in the TERT gene might be associated with shorter telomeres. In order to test this hypothesis, we examined the association between rs401681 and rs2736098 and telomere length in DNA from whole blood, using a quantitative PCR assay. To limit variability, we took into account several factors that have been reported to affect telomere length, including age, gender and smoking status (Valdes, A M, et al., Lancet 366:662-664 (2005); Frenck, R. W Jr., et al. Proc Natl Acad Sci USA 95:5607-10 (1998)) and selected from our database 276 females born between 1925 and 1935 who reported to have never smoked and who had not been diagnosed with cancer. To maximize the contrast, only women homozygous for allele C or allele T at rs401681 were included in the test. In these subjects, rs401681(C) and rs2736098(A) were associated with shorter telomeres with nominal significance (P=0.017 and 0.027, respectively) (FIG. 3, Table 12). However, when we tested telomere length in a group of 260 younger women (selected by the same criteria regarding smoking and cancer, but born between 1940 and 1950), there was no association between telomere length and the risk alleles. Indeed, the effect estimates, while insignificant (P=0.08 and 0.28 for rs401681 and rs2736098, respectively) were in the opposite direction. These results suggest that the variants may lead to an increase in the gradual shortening of telomeres over time, the effect only becoming apparent after a certain age.

TABLE 12
Multiple linear regression for log (telomerase/RNAseP)

| | | | |
|---|---|---|---|
| Variable | Estimate | Standard error | P-value |

Women born 1925-1935

| | | | |
|---|---|---|---|
| (Intercept) | 2.67 | 0.632 | 3.31E−05 |
| age | −0.001 | 0.008 | 0.891 |
| plate 2 | −0.061 | 0.064 | 0.343 |
| plate 3 | 0.071 | 0.099 | 0.471 |
| rs401681 allele C | −0.053 | 0.022 | 0.017 |
| (Intercept) | 2.607 | 0.634 | 5.31E−05 |
| age | −0.001 | 0.008 | 0.921 |
| plate 2 | −0.069 | 0.064 | 0.285 |
| plate 3 | 0.068 | 0.099 | 0.49 |
| rs2736098 allele A | −0.064 | 0.029 | 0.027 |

Women born 1940-1950

| | | | |
|---|---|---|---|
| (Intercept) | 2.29 | 0.367 | 1.78E−09 |
| age | −0.005 | 0.006 | 0.441 |
| plate 5 | −0.012 | 0.046 | 0.79 |
| plate 6 | −0.05 | 0.065 | 0.444 |
| rs401681 allele C | 0.028 | 0.016 | 0.081 |
| (Intercept) | 2.292 | 0.371 | 2.56E−09 |
| age | −0.004 | 0.006 | 0.451 |
| plate 5 | −0.005 | 0.046 | 0.921 |
| plate 6 | −0.044 | 0.065 | 0.504 |
| rs2736098 allele A | 0.025 | 0.024 | 0.283 |

### Example 5

We assessed the association of rs401681(C) and rs2736098(A) with the major histological types of lung cancer (Table 13). For all histological types except carcinoids, the frequency of the risk variants was higher than in controls with the highest frequencies found in squamous cell carcinomas.

TABLE 13
Frequencies of rs401681(C) and rs2736098(A) in different histological
subtypes of lung cancer

| | Iceland | | Spain | | Netherlands | |
|---|---|---|---|---|---|---|
| | 28,890 | | 1,427 | | 1,832 | |
| | controls, | | controls, | | controls, | |
| Histology | freq. 0.545 | | freq. 0.538 | | freq. 0.570 | |
| rs401681 (C) | N | freq. | N | freq. | N | freq. |
| Adenocarcinoma | 305 | 0.582 | 83 | 0.596 | 184 | 0.595 |
| Squamous cell | 178 | 0.624 | 132 | 0.617 | 208 | 0.637 |
| carcinoma | | | | | | |
| Small cell carcinoma | 104 | 0.577 | 70 | 0.528 | 74 | 0.601 |
| Carcinoma NOS | 79 | 0.551 | 40 | 0.589 | 10 | 0.700 |
| Large cell carcinoma | 31 | 0.661 | 25 | 0.440 | 16 | 0.531 |
| Other (incl. Carcinoid) | 56 | 0.508 | 2 | 1.000 | 32 | 0.578 |
| | 3,667 | | 1,384 | | 1,740 | |
| | controls, | | controls, | | controls, | |
| | freq. 0.272 | | freq. 0.229 | | freq. 0.286 | |
| rs2736098 (A) | N | freq. | N | freq. | N | freq. |
| Adenocarcinoma | 305 | 0.305 | 84 | 0.238 | 183 | 0.325 |
| Squamous cell | 153 | 0.281 | 134 | 0.299 | 209 | 0.316 |
| carcinoma | | | | | | |
| Small cell carcinoma | 95 | 0.316 | 69 | 0.268 | 73 | 0.288 |
| Carcinoma NOS | 68 | 0.397 | 45 | 0.267 | 10 | 0.300 |
| Large cell carcinoma | 30 | 0.300 | 24 | 0.167 | 16 | 0.468 |
| Other (incl. Carcinoid) | 46 | 0.239 | 2 | 0.750 | 32 | 0.437 |

### Example 6

### Study Populations and Methods

### Icelandic Study Populations

All projects at deCODE Genetics have been approved by the National Bioethics Committee and the Data Protection Authority of Iceland. Cancer cases were identified based on records from the nation-wide Icelandic Cancer Registry (ICR; www.krabbameinsskra.is) which includes information on the year and age at diagnosis, age at death, SNOMED code and ICD-10 classification. The ICR has been in operation since 1954, covers the entire population of Iceland and determines incidence of cancer by site. The registry receives information from all pathology and cytology laboratories in Iceland, in addition to hematology laboratories, hospital wards, private medical practitioners and other individual health care workers. Approximately 94.5% of diagnoses in the ICR have histological confirmation (Tulinius, H., et al. Cancer Epidemiol Biomarkers Prev 6:863-73 (1997)). All participants are recruited by trained nurses through special recruitment clinics where they donate a blood sample and answer a lifestyle questionnaire. Clinical information on cancer patients were extracted from medical records at treatment centers. Written informed consent was obtained from all participants. Personal identifiers associated with medical information and blood samples were encrypted with a third-party encryption system in which the Data Protection Authority of Iceland maintains the code.

### Controls

The 28,890 controls (41.7% males, 58.3% females; mean age 61 years; SD=21) used in this study consisted of individuals from other ongoing genome-wide association studies at deCODE. The controls had not been diagnosed with any of the cancers under study according to nation-wide lists from the Icelandic Cancer Registry (ICR). No individual disease group accounts for more than 10% of the total control group. If we include all 36,139 chip-genotyped individuals in our control group (also those who have been diagnosed with cancer), the frequency of rs401681 (C) is 0.547 which is very similar to the frequency of 0.545 in the control group (N=28,890) containing individuals that have not been diagnosed with a cancer.

### Skin Cancer Cases (BCC, SCC and Melanoma)

A detailed description of the skin cancer populations can be found in previous reports (Gudbjartsson, D F et al. Nat Genet 40:886-91 (2008); Stacey, S N et al. Nat Genet 40:1313-18 (2008)). The ICR has maintained records of BCC diagnoses since 1981. The records contain all new occurrences of histologically verified BCC, sourced from all the pathology laboratories in the country that deal with such lesions. Diagnoses of BCC made up to the end of 2007 were included and were identified by ICD10 code C44 with a SNOMED morphology code indicating BCC. The ICR has recorded histologically confirmed diagnoses of squamous cell carcinoma (SCC) of the skin since 1955. SCC diagnoses made up to the end of 2007 were included and were identified by ICD10 code C44 with a SNOMED morphology code indicating SCC. Invasive cutaneous melanoma (CM) was identified through ICD10 code C43. Metastatic melanoma (where the primary lesion had not been identified) was identified by a SNOMED morphology code indicating melanoma with a /6 suffix, regardless of the ICD10 code. Ocular melanoma (OM) and melanomas arising at mucosal sites were not included.

### Breast Cancer Cases

A detailed description of the breast cancer population is given by Stacey et al. (2007) (Stacey S N et al. Nat Genet 39:865-9 (2007)). In brief, all prevalent cases living in Iceland who had a diagnosis entered into the ICR up to the end of December 2006 were eligible to participate in the study. The ICR contained the records of 4,785 individuals diagnosed during this period. Consent, samples and successful genotypes were obtained from approximately 1,945 patients. The median age at diagnosis for genotyped cases was 56 years as compared to 61 years for all breast cancer cases in the ICR.

### Cervical Cancer Cases

A total of 803 women were diagnosed with invasive cervical cancer between Jan. 1, 1955 and Dec. 31, 2007. Samples from 276 women were available for direct genotyping and an equivalent of 93 women could be genotyped in silico. The median age at diagnosis for directly genotyped cases was 42 years (range 19-91 years) as compared to 46.5 years for all cervical cancer cases in the ICR.

### Lung Cancer Cases

The Icelandic lung cancer study population has been described previously (Thorgeirsson, T E et al. Nature 452:638-42 (2008)). Briefly, a total of 3,665 lung cancer patients were diagnosed from Jan. 1, 1955, to Dec. 31, 2007. Recruitment of both prevalent and incident cases was initiated in the year 1998. Samples from 797 cases were available for genotyping and an equivalent of 652 cases were genotyped in silico. The lung cancer patients participating in the genetic study answer a lifestyle questionnaire that includes questions on smoking status (never, former, current), and the quantity and duration of smoking. The median age at diagnosis for directly genotyped cases was 67 years (range 16-91 years) as compared to 68 years for all lung cancer cases in the ICR.

### Prostate Cancer Cases

A detailed description of the prostate cancer study population has been published previously (Amundadottir, L T et al Nat Genet 38:652-8 (2006)). Briefly, a total of 4,457 Icelandic prostate cancer patients were diagnosed from Jan. 1, 1955, to Dec. 31, 2007. The Icelandic prostate cancer sample included 1,754 directly genotyped patients and an equivalent of 522 cases genotyped in silico. The mean age at diagnosis for directly genotyped patients was 71 years (median 71 years) and the range was from 40 to 96 years, while the mean age at diagnosis was 73 years for all prostate cancer cases in the ICR.

### Urinary Bladder Cancer (UBC) Cases

A description of Icelandic UBC cases has been published previously (Kiemeney, L A et al. Nat Genet. 40:1307-12 (2008)). The ICR contains records of 1,642 Icelandic UBC patients diagnosed from Jan. 1, 1955 to Dec. 31, 2006. Recruitment started in the year 2001 and both prevalent and incident cases were included. The mean participation rate for newly diagnosed cases was 65%. Samples from 578 cases (76% males; diagnosed from December 1974 to June 2006) were available for direct genotyping and an equivalent of 202 cases were genotyped in silico. The median age at diagnosis for directly genotyped cases was 67 years (range 22-94 years) as compared to 68.5 years for all UBC cases in the ICR.

### Colorectal Cancer

A total of 3,615 individuals were diagnosed with colorectal cancer between Jan. 1, 1955 and Dec. 31, 2007. Samples from 1,044 cases were available for direct genotyping and an equivalent of 529 cases were genotyped in silico. The median age at diagnosis for directly genotyped cases was 68 years as compared to 72 years for all colorectal cancer cases in the ICR.

### Endometrial Cancer

A total of 889 women were diagnosed with endometrial cancer between Jan. 1, 1955 and Dec. 31, 2007. Samples from 387 women were available for genotyping and an equivalent of 83 women were genotyped in silico. The median age at diagnosis for directly genotyped cases was 60 years as compared to 63 years for all endometrial cancer patients in the ICR.

### Kidney Cancer

A total of 1,472 individuals were diagnosed with renal cell cancer between Jan. 1, 1955 and Dec. 31, 2007. Samples from 422 cases were available for genotyping and an equivalent of 203 cases were genotyped in silico. The median age at diagnosis for all directly genotyped cases was 65 years, or the same as for all renal cell cancer cases in the ICR.

### Lymphoma

A total of 1,137 individuals were diagnosed with lymphoma between Jan. 1, 1955 and Dec. 31, 2007. Samples from 178 cases were available for genotyping and an equivalent of 70 cases were genotyped in silico. The median age at diagnosis for directly genotyped cases was 49 years as compared to 56 years for all lymphoma cases in the ICR

### Multiple Myeloma

A total of 483 individuals were diagnosed with multiple myeloma between Jan. 1, 1955 and Dec. 31, 2007. Samples from 64 cases were available for genotyping and an equivalent of 62 cases were genotyped in silico. The median age at diagnosis for directly genotyped cases was 68 years as compared to 69 years for all multiple myeloma cases in the ICR

### Ovarian Cancer

A total of 1,072 women were diagnosed with ovarian cancer between Jan. 1, 1955 and Dec. 31, 2007. Samples from 363 women were available for genotyping and an equivalent of 134 women were genotyped in silico. The median age at diagnosis for all directly genotyped cases was 51 years as compared to 60 years for all ovarian cancer cases in the ICR. Pancreatic cancer

A total of 1,134 individuals were diagnosed with pancreatic cancer between Jan. 1, 1955 and Dec. 31, 2007. Samples from 75 cases were available for genotyping and an equivalent of 226 cases were genotyped in silico. The median age at diagnosis for directly genotyped cases was 70 years as compared to 71 years for all pancreatic cancer cases in the ICR

### Stomach Cancer

A total of 3,210 individuals were diagnosed with stomach cancer between Jan. 1, 1955 and Dec. 31, 2007. Samples from 277 cases were available for genotyping and an equivalent of 485 cases were genotyped in silico. The median age at diagnosis for directly genotyped cases was 68 years as compared to 71 years for all stomach cancer cases in the ICR.

### Thyroid Cancer

A detailed description of the thyroid cancer population can be found in Gudmundson et al. (Gudmundsson, J et al. submitted (2008)). A total of 1,110 individuals were diagnosed with thyroid cancer between Jan. 1, 1955 and Dec. 31, 2007. Samples from 413 cases were available for direct genotyping and an equivalent of 115 cases were genotyped in silico. The median age at diagnosis for directly genotyped cases was 44 years as compared to 56 years for all thyroid cancer cases in the ICR.

### Dutch Study Populations

### Controls

The 1,832 cancer-free control individuals (46% males) were recruited within a project entitled “Nijmegen Biomedical Study” (NBS). The details of this study were reported previously (Wetzels, J F et al. Kidney Int 72:632-7 (2007)). Briefly, this is a population-based survey conducted by the Department of Epidemiology and Biostatistics and the Department of Clinical Chemistry of the Radboud University Nijmegen Medical Centre (RUNMC), in which 9,371 individuals participated from a total of 22,500 age- and sex stratified, randomly selected inhabitants of Nijmegen. Control individuals from the NBS were invited to participate in a study on gene-environment interactions in multi-factorial diseases, such as cancer. The 1,832 controls is a subsample of all the participants to the NBS, frequency-age-matched to a series of breast cancer and a series of prostate cancer patients. All the 1,832 participants are of self-reported European descent and were fully informed about the goals and the procedures of the study. The study protocols of the NBS were approved by the Institutional Review Board of the RUNMC and all study subjects signed a written informed consent form.

### Prostate Cancer Cases

The details of the recruitment of prostate cancer cases was reported previously (Gudmundsson, 3 et al. Nat Genet 39:631-7 (2007)). In short, the case series (994 genotyped cases) was comprised of two recruitment-sets; Group-A was comprised of hospital-based cases recruited from January 1999 to June 2006 at the Urology Outpatient Clinic of the Radboud University Nijmegen Medical Centre (RUNMC); Group-B consisted of cases recruited from June 2006 to December 2006 through a population-based cancer registry held by the Comprehensive Cancer Centre IKO that serves a region of 1.3 million inhabitants in the eastern part of the Netherlands (www.ikcnet.nl). This recruitment took place within the EU 6^{th} Framework POLYGENE project, a project on the identification of susceptibility genes for prostate and breast cancer (www.polygene.eu)). Both A and B groups were of self-reported European descent. The average age at diagnosis for patients in Group-A was 63 years (median 63 years) and the range was from 43 to 83 years. The average age at diagnosis for patients in Group-B was 65 years (median 66 years) and the range was from 43 to 75 years. The study protocol was approved by the Institutional Review Board of Radboud University and all study subjects gave written informed consent.

### Bladder Cancer Cases

The Dutch bladder cancer population has been described in a previous publication (Kiemeney, L A et al. Nat Genet 40:1307-12 (2008)). Briefly, patients were recruited for the Nijmegen Bladder Cancer Study (NBCS) (see http://dceg.cancer.gov/icbc/membership.html). As with the recruitment of the prostate cancer patients, the NBCS identified patients through the population-based regional cancer registry held by the Comprehensive Cancer Centre East, Nijmegen. Patients diagnosed between 1995 and 2006 under the age of 75 years were selected and their vital status and current addresses updated through the hospital information systems of the 7 community hospitals and one university hospital (RUNMC) that are covered by the cancer registry. All patients still alive on Aug. 1, 2007 were invited to the study by the Comprehensive Cancer Center on behalf of the patients' treating physicians. In case of consent, patients were sent a lifestyle questionnaire to fill out and blood samples were collected by Thrombosis Service centers which hold offices in all the communities in the region. 1,651 patients were invited to participate. Of all the invitees, 1,082 gave informed consent (66%): 992 filled out the questionnaire (60%) and 1016 (62%) provided a blood sample. The number of participating patients was increased with a non-overlapping series of 376 bladder cancer patients who were recruited previously for a study on gene-environment interactions in three hospitals (RUNMC, Canisius Wilhelmina Hospital, Nijmegen, and Streekziekenhuis Midden-Twente, Hengelo, the Netherlands). Ultimately, completed questionnaires and blood samples were available for 1,276 and 1,392 patients, respectively. All the patients that were selected for the analyses (N=1,277) were of self-reported European descent. The median age at diagnosis was 62 (range 25-93) years and 82% of the participants were males. Data on tumor stage and grade were obtained through the cancer registry. The study protocols of the NBCS were approved by the Institutional Review Board of the RUNMC and all study subjects gave written informed consent.

### Lung Cancer Cases

The collection of patients with lung cancer took place as an extension of the prostate, breast, and bladder cancer studies within the framework of the EU 6^{th} framework POLYGENE project. Patients with lung cancer were identified through the population-based cancer registry of the Comprehensive Cancer Center IKO, Nijmegen, the Netherlands. Patients who were diagnosed in one of three hospitals (Radboud University Nijmegen Medical Center and Canisius Wilhelmina Hospital in Nijmegen and Rijnstate Hospital in Arnhem) and who were still alive at April 15^{th}, 2008 were recruited for a study on gene-environment interactions in lung cancer. 458 patients gave informed consent and donated a blood sample. This case series was increased with 94 patients to a total of 552 by linking three other studies to the population-based cancer registry in order to identify new occurrences of lung cancer among the participants of these other studies. All three other studies (i.e., POLYGENE, the Nijmegen Biomedical Study, and the RUNMC Urology Outpatient Clinic Epidemiology Study) were initiated to study genetic risk factors for disease and participants to these studies gave general informed consent for DNA-related research and linkage with disease registries. Information on histology, stage of disease, and age at diagnoses was obtained through the cancer registry.

### Kidney Cancer Cases

The Dutch patients with kidney cancer were recruited through the outpatient urology clinic of the Radboud University Nijmegen Medical Center. From January 1999 onwards, blood samples and lifestyle data have been collected from patients visiting the outpatient clinic for studies into gene-environment interactions for urological diseases. The 8,000 patients who participated in this study gave informed consent for the study and for linking their data with disease registries. The study was linked with the population-based cancer registry in order to identify patients who were diagnosed with renal cell cancer. 362 patients were identified.

### Spanish Study Populations

### Controls

The 1,427 Spanish control samples are from individuals that attended the University Hospital in Zaragoza, Spain, for diseases other than cancer between November 2001 and May 2007. The controls were of both genders and median age was 52 years. Controls were questioned to rule out prior cancers before the blood sample was collected. All patients and controls were of self-reported European descent. Study protocols were approved by the Institutional Review Board of Zaragoza University Hospital. All subjects gave written informed consent

### Bladder Cancer Cases

The patients were recruited from the Urology and Oncology Departments of Zaragoza Hospital between September 2007 and February 2008. A total of 173 patients with histologically-proven urothelial cell carcinoma of the bladder were enrolled (response 77%). The median time interval from bladder cancer diagnosis to collection of blood samples was 9 months (range 1 to 29 months). Clinical information including age at onset, grade and stage was obtained from medical records. The median age at diagnosis for the patients was 65 years (range 27 to 94) and 87% were males. Study protocols were approved by the Institutional Review Board of Zaragoza University Hospital. All subjects gave written informed consent.

### Lung Cancer Cases

The patients were recruited from the Oncology Department of Zaragoza Hospital in Zaragoza, from June 2006 to June 2008. During the 24 month interval of recruitment, 367 patients were enrolled (88% participation rate). Clinical information including age at onset and histology were collected from medical records. Study protocols were approved by the Institutional Review Board of Zaragoza University Hospital. All subjects gave written informed consent.

### Prostate Cancer Cases

The study population consisted of 560 prostate cancer cases of which 459 (82%) were successfully genotyped. The cases were recruited from the Oncology Department of Zaragoza Hospital in Zaragoza, Spain, from June 2005 to September 2006. All patients were of self-reported European descent. Clinical information including age at onset, grade and stage was obtained from medical records. The average age at diagnosis for the patients was 69 years (median 70 years) and the range was from 44 to 83 years. Study protocols were approved by the Institutional Review Board of Zaragoza University Hospital. All subjects were gave written informed consent.

### The Eastern Europe Study Population

The details of this study population have been described previously (Thurumaran, R K et al. Carcinogenesis 27:1676-81 (2006)). Cases and controls were recruited as part of a study designed to evaluate the risk of various cancers due to environmental arsenic exposure in Hungary, Romania and Slovakia between 2002 and 2004. The recruitment was carried out in the counties of Bacs, Bekes, Csongrad and Jasz-Nagykun-Szolnok in Hungary; Bihor and Arad in Romania; and Banska Bytrica and Nitre in Slovakia. The BCC cases (525), bladder cancer cases (N=214) and controls (N=525) were of Hungarian, Romanian and Slovak nationalities. BCC and bladder cancer cases were invited on the basis of histopathological examinations by pathologists. Hospital-based controls were included in the study, subject to fulfillment of a set of criteria. All general hospitals in the study areas were involved in the process of control recruitment. The controls were frequency matched with cases for age, gender, country of residence and ethnicity. Controls included general surgery, orthopedic and trauma patients aged 30-79 years. Patients with malignant tumors, diabetes and cardiovascular diseases were excluded as controls. The median age for the bladder cancer patients was 65 years (range 36-90) and 83% of the patients were males. The median age at diagnosis for BCC cases was 67 years (range 30-85) and the median age for the controls was 61 years (range 28-83). 51% of the controls were males. The response rates among cases and controls were ˜70%. Clinicians took venous blood from cases and controls after consent forms had been signed. Cases and controls recruited to the study were interviewed by trained personnel and completed a general lifestyle questionnaire. Ethnic background for cases and controls was recorded along with other characteristics of the study population. Local ethical boards approved the study.

Leeds Bladder Cancer Study, United Kingdom Details of the Leeds Bladder Cancer Study have been reported previously (Sak, S C et al. Br J Cancer 92:2262-65 (2005)). In brief, patients from the urology department of St James's University Hospital, Leeds were recruited from August 2002 to March 2006. All those patients attending for cystoscopy or transurethral resection of a bladder tumor (TURBT) who had previously been found, or were subsequently shown, to have urothelial cell carcinoma of the bladder were included. Exclusion criteria were significant mental impairment or a blood transfusion in the past month. All non-Caucasians were excluded from the study leaving 764 patients. The median age at diagnosis of the patients was 73 years (range 30-101). 71% of the patients were male and 61% of all the patients had a low risk tumor (pTaG1/2). Genotyping was successful in 707 patients. The controls were recruited from the otolaryngology outpatients and ophthalmology inpatient and outpatient departments at St James's Hospital, Leeds, from August 2002 to March 2006. All controls of appropriate age for frequency matching with the cases were approached and recruited if they gave their informed consent. As for the cases, exclusion criteria for the controls were significant mental impairment or a blood transfusion in the past month. Also, controls were excluded if they had symptoms suggestive of bladder cancer, such as haematuria. 2.8% of the controls were non-Caucasian leaving 530 Caucasian controls for the study. 71% of the controls were male. Data were collected by a health questionnaire on smoking habits and smoking history (non- ex- or current smoker, smoking dose in pack-years), occupational exposure history (to plastics, rubber, laboratories, printing, dyes and paints, diesel fumes), family history of bladder cancer, ethnicity and place of birth, and places of birth of parents. The response rate of cases was approximately 99%, that among the controls approximately 80%. Ethical approval for the study was obtained from Leeds (East) Local Research Ethics Committee, project number 02/192.
Torino Bladder Cancer Case Control Study, Italy The source of cases for the Torino bladder cancer study are two urology departments of the main hospital in Torino, the San Giovanni Battista Hospital (Matullo, G. et al. Cancer Epidemiol Biomarkers Prev 14:2569-78 (2005)). Cases are all Caucasian men, aged 40 to 75 years (median 63 years) and living in the Torino metropolitan area. They were newly diagnosed between 1994 and 2006 with a histologically confirmed, invasive or in situ, bladder cancer. The sources of controls are urology, medical and surgical departments of the same hospital in Torino. All controls are Caucasian men resident in the Torino metropolitan area. They were diagnosed and treated between 1994 and 2006 for benign diseases (such as prostatic hyperplasia, cystitis, hernias, heart failure, asthma, and benign ear diseases). Controls with cancer, liver or renal diseases and smoking related conditions were excluded. The median age of the controls was 57 years (range 40 to 74). Data were collected by a professional interviewer who used a structured questionnaire to interview both cases and controls face-to-face. Data collected included demographics (age, sex, ethnicity, region and education) and smoking. For cases, additional data were collected on tumor histology, tumor site, size, stage, grade, and treatment of the primary tumor. The response rates were 90% for cases and 75% for controls. Genotyping was successful for 329 cases and 379 controls. Ethical approval for the study was obtained from Comitato Etico Interaziendale, A.O.U. San Giovanni Batista—A.). C.T.O./Maria Adelaide.

### The Brescia Bladder Cancer Study, Italy

The Brescia bladder cancer study is a hospital-based case-control study. The study was reported in detail previously (Shen M. et al. Cancer Epidemiol Biomarkers Prev 12:1234-40 (2003)). In short, the catchment area of the cases and controls was the Province of Brescia, a highly industrialized area in Northern Italy (mainly metal and mechanical industry, construction, transport, textiles) but also with relevant agricultural areas. Cases and controls were enrolled in 1997 to 2000 from the two main city hospitals. The total number of eligible subjects was 216 cases and 220 controls. The response rate (enrolled/eligible) was 93% (N=201) for cases and 97% (N=214) for controls. Genotyping was successful in 122 cases and 156 controls. Only males were included. All cases and controls had Italian nationality and were of Caucasian ethnicity. All cases had to be residents of the Province of Brescia, aged between 20 and 80, and newly diagnosed with histologically confirmed bladder cancer. The median age of the patients was 63 years (range 22-80). Controls were patients admitted for various urological non-neoplastic diseases and were frequency matched to cases on age, hospital and period of admission. The study was formally approved by the ethical committee of the hospital where the majority of subjects were recruited. A written informed consent was obtained from all participants. Data were collected from clinical charts (tumor histology, site, grade, stage, treatments, etc.) and by means of face-to-face interviews during hospital admission, using a standardized semi-structured questionnaire. The questionnaire included data on demographics (age, ethnicity, region, education, residence, etc.), and smoking. ISCO and ISIC codes and expert assessments were used for occupational coding. Blood samples were collected from cases and controls for genotyping and DNA adducts analyses.

### The Belgian Case Control Study of Bladder Cancer

The Belgian study has been reported in detail (Kellen, E. et al. Int J Cancer 118:2572-78 (2006)). In brief, cases were selected from the Limburg Cancer Registry (LIKAR) and were approached through urologists and general practitioners. All cases were diagnosed with histologically confirmed urothelial cell carcinoma of the bladder between 1999 and 2004, and were Caucasian inhabitants of the Belgian province of Limburg. The median age of the patients was 68 years. 86% of all the patients were males. For the recruitment of controls, a request was made to the “Kruispuntbank” of the social security for simple random sampling, stratified by municipality and socio-economic status, among all citizens above 50 years of age of the province. The median age of the controls was 64 years; 59% of the controls were males. Three trained interviewers visited cases and controls at home. Information was collected through a structured interview and a standardized food frequency questionnaire. In addition, biological samples were collected. Data collected included medical history, lifetime smoking history, family history of bladder cancer and a lifetime occupational history. Informed consent was obtained from all participants and the study was approved by the ethical review board of the Medical School of the Catholic University of Leuven, Belgium.

### The Swedish Bladder Cancer Study

The Swedish patients come from a population-based study of urinary bladder cancer patients diagnosed in the Stockholm region in 1995-1996 (Larsson, P. et al. Scand J Urol Nephrol 37:195-201 (2003)). Blood samples from 346 patients were available out of a collection of 538 patients with primary urothelial carcinoma of the bladder. The average age at onset for these patients is 69 years (range 32-97 years) and 67% of the patients are males. Clinical data, including age at onset, grade and stage of tumor, were prospectively obtained from hospitals and urology units in the region. The control samples came from blood donors in the Stockholm region and were from cancer free individuals of both genders. The regional ethical committee approved of the study and all participants gave informed consent.

### Prostate Cancer Study, Chicago

The Chicago study population consisted of 680 prostate cancer cases of which 635 (93%) were successfully genotyped. The cases were recruited from the Pathology Core of Northwestern University's Prostate Cancer Specialized Program of Research Excellence (SPORE) from May 2002 to May 2007. The average age at diagnosis for the patients was 60 years (median 59 years) and the range was from 39 to 87 years. The 693 controls were recruited as healthy control subjects for genetic studies at the University of Chicago and Northwestern University Medical School, Chicago, US. All individuals from Chicago included in this report were of self-reported European descent. Study protocols were approved by the Institutional Review Boards of Northwestern University and the University of Chicago. All subjects gave written informed consent.

### IARC's Dataset on Lung Cancer

The International Agency for Research on Cancer (IARC), Lyon, France, the CEPH and the CNG, have conducted a genome-wide association study of lung cancer consisting of 1,926 lung cancer cases and 2,522 controls from five eastern European countries (Hung, R J et al. Nature 452:633-37 (2008)). The results and data from the genome-wide phase of the study (310,023 SNPs) have been made available on the IARC website for other groups to use for meta-analyses and other studies. (http://www.cng.fr/prog_cancergenomics/lung_cancer.html).

### ICR's Dataset on Colorectal Cancer

The Institute of Cancer Research (ICR) has generated genotype data for 547,487 SNPs in 922 individuals with colorectal neoplasia and 927 controls ascertained through the Colorectal Tumour Gene Identification (CoRGI) consortium (Tomlionson I P et al. Nat Genet 40:623-30 (2008)). In order to facilitate the identification of additional loci predisposing to colorectal cancer, the genotype count data and allelic test results from the genome-wide phase of this study have been made available to other groups for meta-analyses and further studies. (http://www.icr.ac.uk/research/research_sections/cancer_genetics/cancer_genetics_teams/mole cular_and_population_genetics/software_and_databases/index.shtml)

### GCEMS Dataset on Prostate and Breast Cancer

The Cancer Genetics Markers of Susceptibility (CGEMS) initiative of the National Cancer Institute is conducting GWA studies with follow-up replication studies to identify common, inherited gene variations for cancers of the prostate and breast. Publicly available data from the GWA scans were retrieved from the projects website (https://caintegrator.nci.nih.gov/cgems).

### Genotyping

Whole-genome association studies have been performed on the following cancers in the Icelandic population; prostate cancer, breast cancer, lung cancer, BCC, melanoma, urinary bladder cancer and colorectal cancer (Stacey, S N et al Nat Genet 40:1313-18 (2008)); Stacey S N et al Nat Genet. 39:865-9 (2007)); Thorgeirsson, T E et al Nature 452:638-42 (2008)); Kiemeney, L A et al. Nat Genet 40:1307-12 (2008)); Gudmundsson, J. et al. Nat Genet 39:631-37 (2007)). All cases and controls were assayed using genotyping systems and specialized software from Illumina (Human Hap300 and HumanCNV370-duo Bead Arrays, Illumina). Furthermore, all Dutch bladder cancer cases and controls have been genotyped with the HumanCNV370-duo Bead Arrays. These chips provide about 75% genomic coverage in the Utah CEPH (CEU) HapMap samples for common SNPs at r2>0.8 (Barrett, J C & Cardon, L R Nat Genet 38:659-62 (2006)). SNP data were discarded if the minor allele frequency in the combined case and control was <0.001 or had less than 95% yield or showed a very significant distortion from Hardy-Weinberg equilibrium in the controls)(P<1×10⁻¹°. Any chips with a call rate below 98% of the SNPs were excluded from the genome-wide association analysis.

All single SNP genotyping was carried out applying the Centaurus (Nanogen) platform (Kutyavin, I V et al. Nucleic Acids Res 34:e128 (2006)). The quality of each Centaurus SNP assay was evaluated by genotyping each assay in the CEU HapMap samples and comparing the results with the HapMap publicly released data. Assays with >1.5% mismatch rate were not used and a linkage disequilibrium (LD) test was used for markers known to be in LD. Approximately 10% of the Icelandic case samples that were genotyped on the Illumina platform were also genotyped using the Centaurus assays and the observed mismatch rate was lower than 0.5%. All genotyping was carried out at deCODE Genetics.

### Assessment of Telomere Length

We selected whole blood as the tissue for analyzing telomere length for its accessibility but studies have shown that the length of telomeres is very similar within different tissues of the same individual but vary significantly between individuals (Marten U M et al. Nat Genet 18:76-80 (2998)). Telomeres were measured utilizing quantitative Taqman® PCR as described by Cawthon (Cawthon, R M Nucleic Acids Res 30:e47 (2002)). RNAseP endogenous control assay (Cat.no. 4316844) (Applied Biosystems Inc.) was used to correct for DNA input. This quantitative PCR method has been shown to give consistent results as Southern blot and FISH based telomere measurements (Schwob, A E et al. Mol Biol Cell 19:1548-60 (2008)). All reactions were run on ABI7900TH real time PCR system (Applied Biosystems Inc.). All assays were done in duplicate and repeated in an independent experiment. The use of RNAseP is a standard procedure in gene dosage measurements with real time qRT-PCR (Schwob, A E et al. Mol Biol Cell 19:1548-60 (2008); Writzl, K. et al. Hum Reprod 21:753-4 (2006)). The main limitation of the method is that it measures relative telomere length rather than actual telomere length. For us, the relative telomere length is sufficient for determining if there is a difference in telomere length between individuals depending on their genotype.

### Regression Analysis of Telomere Length Data

A total of 528 females were analyzed in two batches, each batch done with 3 plates, batch 1 included 268 women with a mean age at blood sampling of 72.8 (SD 5.0) years, batch 2 included 260 women with a mean age at blood sampling of 57.8 (SD 4.6) years. The relationship between the SNPs showing association and telomerase length was analyzed by multiple regression. The logarithm of the ratio between telomerase and RNAseP was taken as dependent variable, and the covariates age at blood sampling and plate were included in the models. SNPs showing association were analyzed using multiple linear regression. The experiments were carried out at two different points in time and were analyzed separately.

### Association Analysis

A likelihood procedure described in a previous publication (Gretarsdottir, S. et al. Nat Genet. 35:131-8 (2003)) and implemented in the NEMO software was used for the association analyses. An attempt was made to genotype all individuals and all SNPs reported, and for each of the SNPs, the yield was higher than 95% in every study group. We tested the association of an allele to cancer using a standard likelihood ratio statistic that, if the subjects were unrelated, would have asymptotically a X²distribution with one degree of freedom under the null hypothesis. Allelic frequencies rather than carrier frequencies are presented for the markers in the main text. Allele-specific ORs and associated P values were calculated assuming a multiplicative model for the two chromosomes of an individual (Falk C T & Rubinstein P Ann Hum Genet. 51(Pt3):227-33 (1987). Results from multiple case-control groups were combined using a Mantel-Haenszel model (Mantel N & Haenszel W J Natl cancer Inst 22:719-48 (1959)) in which the groups were allowed to have different population frequencies for alleles, haplotypes and genotypes but were assumed to have common relative risks. All P values are reported as two-sided.

For the analysis of the Icelandic samples, the same set of cancer free controls used in the BCC discovery analysis was used for all other cancer types, introducing a potential bias. However, due to the lack of association with common cancers like breast and colorectal cancer and also because of the modest effect sizes for the cancers associating with rs401681(C), the frequency of the variant is not substantially different in the Icelandic cancer free controls (0.545) compared to the whole group of Icelanders (N=36,139) genotyped with the BeadChips (0.547) which includes all cancer cases. Therefore, the potential bias introduced into the estimation of the association of the sixteen cancers with rs401681(C) is small. Furthermore, this effect is confined to the Icelandic part of our study.

### Test of Un-Genotyped Hapmap Markers

To test for SNP that are in the CEU section of the Hapmap database, but that are absent on the Illumina chip, we use a method based on haplotypes of two markers on the chip. We used a method we have previously employed (Styrkarsdottir, U et al. N Eng J Med 358:2355-65 (2008)), that is an extension of the two-marker haplotype tagging method (Pe'er, I et al. Nat Genet. 38:663-7 (2006)) and is similar in spirit to two other proposed methods (Nicolae, D L Genet Epidemiol 30:718-27 (2006); Zaitlen N. et al. Am J Hum Genet. 80:683-91 (2007)). We computed associations with a linear combination of the different haplotypes chosen to act as surrogates to HapMap markers in the regions. In the 5p13.33 region displayed in FIG. 2 (corresponding to a 200 kb interval), we tested with this method 95 markers in addition to the ones on the chip. These calculations were based on 1,025 BCC cases and 28,890 controls genotyped on chip. Of those markers, rs2736098 had the most significantly association with BCC.

### Genomic Control and Inflation Factors

To adjust for possible population stratification and the relatedness amongst individuals, we divided the X² statistics from the initial scan of basal cell carcinoma in Iceland, using the method of genomic control, i.e. the 304 thousand test statistics were divided by their means, which was 1.22. In the cases where the method of genomic control is not directly applicable (i.e. if the genome wide association results are not available for the same groups), we used the genealogy to estimate the inflation factor. Since some of the Icelandic patients and controls are related to each other, both within and between groups, the X² statistics have a mean>1. We estimated the inflation factor by simulating genotypes through the Icelandic genealogy, as described previously, and corrected the X² statistics for Icelandic OR's accordingly. The estimated inflation factor for different analyses is presented in Table 15.

### In Silico Genotyping of Un-Genotyped Individuals

We extend the classical SNP case-control association study design by including un-genotyped cases with genotyped relatives. For every un-genotyped case, we calculate the probability of the genotypes of its relatives given its four possible phased genotypes (see Figure below for an example). In practice we have chosen to include only the genotypes of the case's parents, children, siblings, half-siblings (and the half-sibling's parents), grand-parents, grand-children (and the grand-children's parents) and spouses. We assume that the individuals in the small sub-pedigrees created around each case are not related through any path not included in the pedigree. We also assume all alleles that are not transmitted to the case have the same frequency—the population allele frequency. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually false. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin B. et al. Nat Genet 36:1129-30 (2004)) has proven to be successful at adjusting case-control test statistics for relatedness. We therefore apply the method of genomic control to account for the dependence between the terms in our pseudolikelihood and produce a valid test statistic.

Fisher's information was used to estimate the effective sample size of the part of the pseudolikelihood due to un-genotyped cases. Breaking the total fisher information, I, into the part due to genotyped cases, I_{g}, and the part due to ungenotyped cases, Iᵤ, I=I_{g}+Iᵤᵢ and denoting the number of genotyped cases with N, the effective sample size due to the un-genotyped cases is estimated by

$$\frac{{I}_{u}}{{I}_{g}} N .$$

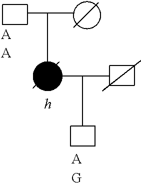

| Transmitted (h) | | |
|---|---|---|
| Paternally | Maternally | Prob(genotypes \| h)^{a} |
| A | A | f |
| A | G | ½ |
| G | A | 0 |
| G | G | 0 |

An example of how the genotypes of relatives are used to obtain information about the genotypes of an un-genotyped case. Un-genotyped individuals are indicated by a strike-through. The case's father is homozygous for the A allele and the case's son is heterozygous AG. Therefore, the case must have received an A allele from her father and either transmitted an A or a G allele to her son, the probability of which depends upon the population frequency of A (denoted by f). ^{a}Probabilities are given up to a normalization constant.

TABLE 14
Frequency of rs401681 in cancer cases and controls genotyped directly by
chip or single track assays and cancer cases genotyped in silicoin Iceland.

| | | | Genotyped in silico | | | |
|---|---|---|---|---|---|---|
| | | | effective N | | | |
| | Genotyped | | cases^{a} | | Total | |
| | N | | (available N | | N | |
| Cancer site | cases | Freq. | cases)^{b} | Freq. | cases | Freq. |
| BCC | 1,769 | 0.602 | 271 (959) | 0.619 | 2040 | 0.604 |
| Lung | 797 | 0.584 | 652 (2,092) | 0.565 | 1,449 | 0.575 |
| Bladder | 578 | 0.583 | 202 (718) | 0.582 | 780 | 0.583 |
| Prostate | 1,754 | 0.564 | 522 (1,778) | 0.589 | 2,276 | 0.569 |
| Cervix | 276 | 0.611 | 93 (388) | 0.611 | 369 | 0.611 |
| Breast | 1,945 | 0.543 | 556 (1863) | 0.533 | 2,501 | 0.541 |
| Colorectal | 1,044 | 0.538 | 529 (1,716) | 0.533 | 1,573 | 0.536 |
| Cutaneous | 577 | 0.523 | 62 (204) | 0.500 | 639 | 0.520 |
| melanoma | | | | | | |
| Endometrium | 387 | 0.580 | 83 (332) | 0.636 | 470 | 0.592 |
| Kidney | 422 | 0.585 | 203 (770) | 0.547 | 625 | 0.572 |
| Lymphoma | 178 | 0.497 | 70 (206) | 0.544 | 248 | 0.510 |
| Multiple | 64 | 0.617 | 62 (193) | 0.564 | 126 | 0.591 |
| myeloma | | | | | | |
| Ovary | 363 | 0.541 | 134 (447) | 0.541 | 497 | 0.541 |
| Pancreas | 75 | 0.513 | 226 (712) | 0.553 | 301 | 0.543 |
| SCC (skin) | 547 | 0.578 | ND | ND | 547 | 0.578 |
| Stomach | 277 | 0.528 | 485 (1,975) | 0.540 | 762 | 0.536 |
| Thyroid | 413 | 0.551 | 115 (384) | 0.496 | 528 | 0.538 |

^{a}Effective sample size from cases genotyped in silico
^{b}Available for in silico genotyping (having a 1^{st} or 2^{nd} degree relative)
ND = not done

TABLE 15
Inflation factors used for correction of chi-square statistics in different
analyses for relatedness and Genomic Control

| | rs401681 | | | | | | | | |
|---|---|---|---|---|---|---|---|---|---|
| | | | | genotyped directly | | | rs401681 and rs2736098 | | |
| | genotyped directly^{a} | | | and in silico^{b} | | | genotyped directly^{a} | | |
| | N | N | Corr. | N | N | Corr. | N | N | Corr. |
| Cancer | cases | controls | factor | cases | controls | factor | cases | controls | Factor |
| Basal cell | 1,769 | 28,890 | 1.11 | 2,040 | 28,890 | 1.16 | 1,600 | 3,667 | 1.17 |
| carcinoma | | | | | | | | | |
| Lung cancer | 797 | 28,890 | 1.06 | 1,449 | 28,890 | 1.18 | 687 | 3,667 | 1.08 |
| Bladder cancer | 578 | 28,890 | 1.04 | 780 | 28,890 | 1.07 | 460 | 3,667 | 1.05 |
| Prostate cancer | 1,754 | 28,890 | 1.09 | 2,276 | 28,890 | 1.19 | 1,640 | 3,667 | 1.17 |
| Cervix cancer | 276 | 28,890 | 1.00 | 369 | 28,890 | 1.02 | 249 | 3,667 | 1.03 |

^{a}Inflation factor calculated through a simulation of genealogy
^{b}Inflation factor calculated by the method of genomic control, using the 300,000 markers from the chip

### Example 7

The association of rs401681 with cutaneous melanoma was analyzed further. The initial discovery that allele C of this marker confers protection against melanoma was confirmed when expanding the analysis. As shown in Table 16, the association was assessed in additional cohorts from Sweden, Spain, Holland, Austria and Italy. All cohorts independently indicated a protective effect of the C allele, with overall OR value of 0.86 and the overall p-value of 5.0×10⁻⁸ (Table 16). These results confirm that the C allele of rs401681 confers protection against cutaneous melanoma, and as a consequence the alternate allele T of rs401681 is a risk allele of cutaneous melanoma, with an OR value of 1.16.

TABLE 16
Association of rs401681 with cutaneous melanoma (CM) in several populations.

| | Risk | Number | | Frequency | | | | |
|---|---|---|---|---|---|---|---|---|
| Sample Group | Allele | Cases | Controls | Cases | Controls | OR | 95% CI | P |
| Iceland CM | C | 591 | 34,998^{a} | 0.52 | 0.55 | 0.90 | (0.80, 1.01) | 7.9 × 10⁻² |
| Sweden CM | C | 1,056 | 2,631 | 0.49 | 0.54 | 0.85 | (0.77, 0.94) | 1.2 × 10⁻³ |
| Spain CM | C | 748 | 1,758 | 0.51 | 0.54 | 0.90 | (0.80, 1.02) | 9.4 × 10⁻² |
| Holland CM | C | 736 | 1,832 | 0.53 | 0.57 | 0.83 | (0.73, 0.94) | 3.9 × 10⁻³ |
| Austria CM | C | 152 | 376 | 0.53 | 0.53 | 0.98 | (0.75, 1.27) | 0.88 |
| Italy CM | C | 560 | 368 | 0.49 | 0.56 | 0.74 | (0.62, 0.89) | 1.2 × 10⁻³ |
| All CM Combined | C | 3,843 | 41,963 | NA | NA | 0.86 | (0.81, 0.91) | 5.0 × 10⁻⁸ |

^{a}Skin cancer-free controls.

1. A method for determining a susceptibility to cancer in a human individual, comprising determining whether at least one allele of at least one polymorphic marker is present in a nucleic acid sample obtained from the individual, wherein the at least one polymorphic marker is selected from the group consisting of rs401681, rs2736100 and rs2736098, and markers in linkage disequilibrium therewith, wherein the linkage disequilibrium is characterized by a value for r² of at least 0.2,
determining a susceptibility to cancer in the subject from the presence or absence of the at least one allele, and wherein the presence of the at least one allele is indicative of a susceptibility to cancer for the individual.

2. The method according to claim 1, wherein the at least one polymorphic marker is selected from the markers set forth in Table 5, Table 6 and Table 7.

3. The method according to claim 1, wherein the at least one polymorphic marker is selected from rs401681, rs2736100 and rs2736098.

4. The method according to claim 1, further comprising assessing the frequency of at least one haplotype in the individual.

5. The method of claim 1, wherein the susceptibility conferred by the presence of the at least one allele is increased susceptibility.

6. The method according to claim 5, wherein the presence of allele C in marker rs401681, allele G in marker rs2736100 and/or allele A in marker rs2736098 is indicative of increased susceptibility to cancer in the individual.

7. The method according to claim 5, wherein the presence of the at least one allele or haplotype is indicative of increased susceptibility to cancer with a relative risk (RR) or odds ratio (OR) of at least 1.10.

8. The method according to claim 5, wherein the presence of the at least one allele or haplotype is indicative of increased susceptibility with a relative risk (RR) or odds ratio (OR) of at least 1.15.

9. The method according to claim 1, wherein the susceptibility conferred by the presence of the at least one allele or haplotype is decreased susceptibility.

10. The method of claim 1, further comprising determining whether at least one at-risk allele of at least one at-risk variant for cancer not in linkage disequilibrium with any one of the markers set forth in any one of Table 5, Table 6 and Table 7 is present in a sample comprising genomic DNA from a human individual or a genotype dataset derived from a human individual.

11. A method of determining a susceptibility to cancer in a human individual, the method comprising determining whether at least one allele of at least one polymorphic marker is present in a nucleic acid sample obtained from the individual, wherein the at least one polymorphic marker is associated with the TERT gene, and wherein the presence of the at least one allele is indicative of a susceptibility to cancer for the individual.

12. A method of determining a susceptibility to cancer in a human individual, the method comprising:
obtaining nucleic acid sequence data about a human individual identifying at least one allele of at least one polymorphic marker selected from the group consisting of rs401681, rs2736100 and rs2736098, and markers in linkage disequilibrium therewith, wherein the linkage disequilibrium is characterized by a value for r² of at least 0.2, and wherein different alleles of the at least one polymorphic marker are associated with different susceptibilities to cancer in humans, and
determining a susceptibility to cancer from the nucleic acid sequence data.

13. The method of claim 12, comprising obtaining nucleic acid sequence data about at least two of said polymorphic markers selected from the group consisting of rs401681, rs2736100 and rs2736098, and markers in linkage disequilibrium therewith.

14. The method of claim 12, wherein determination of a susceptibility comprises comparing the nucleic acid sequence data to a database containing correlation data between the at least one polymorphic marker and susceptibility to cancer.

15. (canceled)

16. (canceled)

17. The method of claim 12, wherein obtaining nucleic acid sequence data comprises obtaining a biological sample from the human individual and analyzing sequence of the at least one polymorphic marker in nucleic acid in the sample.

18. (canceled)

19. (canceled)

20. The method of claim 12, further comprising reporting the susceptibility to at least one entity selected from the group consisting of the individual, a guardian of the individual, a genetic service provider, a physician, a medical organization, and a medical insurer.

21. (canceled)

22. (canceled)

23. The method of claim 1, wherein the at least one polymorphic marker is associated with the TERT gene.

24. A method of identification of a marker for use in assessing susceptibility to cancer, the method comprising:
a. identifying at least one polymorphic marker in linkage disequilibrium with at least one of rs401681, rs2736100 and rs2736098, wherein the linkage disequilibrium is characterized by a value for r² of at least 0.2;
b. determining the genotype status of a sample of individuals diagnosed with, or having a susceptibility to, cancer; and
c. determining the genotype status of a sample of control individuals; and
d. identifying the at least one polymorphic marker for use in assessing susceptibility to thyroid cancer from (b) and (c),
wherein a significant difference in frequency of at least one allele in at least one polymorphism in individuals diagnosed with, or having a susceptibility to, cancer, as compared with the frequency of the at least one allele in the control sample is indicative of the at least one polymorphism being useful for assessing susceptibility to cancel;
wherein an increase in frequency of the at least one allele in the at least one polymorphism in individuals diagnosed with, or having a susceptibility to, cancer, as compared with the frequency of the at least one allele in the control sample is indicative of the at least one polymorphism being useful for assessing increased susceptibility to cancer, and
wherein a decrease in frequency of the at least one allele in the at least one polymorphism in individuals diagnosed with, or having a susceptibility to, cancer, as compared with the frequency of the at least one allele in the control sample is indicative of the at least one polymorphism being useful for assessing decreased susceptibility to, or protection against, cancer.

25. (canceled)

26. (canceled)

27. A method of genotyping a nucleic acid sample obtained from a human individual comprising determining whether at least one allele of at least one polymorphic marker is present in a nucleic acid sample from the individual sample, wherein the at least one marker is selected from rs401681, rs2736100 and rs2736098, and markers in linkage disequilibrium therewith, wherein the linkage disequilibrium is characterized by a value for r² of at least 0.2, and wherein determination of the presence of the at least one allele in the sample is indicative of a susceptibility to cancer in the individual.

28. (canceled)

29. The method according to claim 27, wherein genotyping comprises amplifying a segment of a nucleic acid that comprises the at least one polymorphic marker by Polymerase Chain Reaction (PCR), using a nucleotide primer pair flanking the at least one polymorphic marker.

30. The method according to claim 27, wherein genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, single-stranded conformation analysis and micro array technology.

31. (canceled)

32. The method according to claim 30, wherein the process is a microarray technology.

33. (canceled)

34. (canceled)

35. (canceled)

36. (canceled)

37. (canceled)

38. The method of claim 1, further comprising analyzing non-genetic information to make cancer risk assessment, diagnosis, or prognosis of the individual.

39. The method of claim 38, wherein the non-genetic information is selected from age, gender, ethnicity, socioeconomic status, previous disease diagnosis, medical history of subject, family history of cancer, biochemical measurements, and clinical measurements.

40. The method of claim 38, further comprising calculating combined risk.

41. (canceled)

42. (canceled)

43. (canceled)

44. (canceled)

45. (canceled)

46. (canceled)

47. (canceled)

48. A computer-readable medium having computer executable instructions for determining susceptibility to cancer in a human individual, the computer readable medium comprising: data indicative of at least one polymorphic marker;
a routine stored on the computer readable medium and adapted to be executed by a processor to determine risk of developing cancer in an individual for the at least one polymorphic marker;
wherein the at least one polymorphic marker is selected from rs401681, rs2736100 and rs2736098, and markers in linkage disequilibrium therewith, wherein the linkage disequilibrium is characterized by a value for r² of at least 0.2.

49. The computer readable medium of claim 48, wherein the computer readable medium contains data indicative of at least two polymorphic markers.

50. The computer readable medium of claim 48, wherein the data indicative of at least one polymorphic marker comprises parameters indicative of susceptibility to cancer for the at least one polymorphic marker, and wherein risk of developing cancer in an individual is based on the allelic status for the at least one polymorphic marker in the individual.

51. The computer readable medium of claim 48, wherein said data indicative of at least one polymorphic marker comprises data indicative of the allelic status of said at least one polymorphic marker in the individual.

52. The computer readable medium of claim 48, wherein said routine is adapted to receive input data indicative of the allelic status of said at least one polymorphic marker in said individual.

53. (canceled)

54. (canceled)

55. (canceled)

56. An apparatus for determining a genetic indicator for cancer in a human individual, comprising:
a processor a computer readable memory having computer executable instructions adapted to be executed on the processor to analyze marker and/or haplotype information for at least one human individual with respect to at least one polymorphic marker selected from rs401681, rs2736100 and rs2736098, and markers in linkage disequilibrium therewith, wherein the linkage disequilibrium is characterized by a value for r² of at least 0.2, and
generate an output based on the marker or haplotype information, wherein the output comprises a risk measure of the at least one marker or haplotype as a genetic indicator of cancer for the human individual.

57. (canceled)

58. The apparatus according to claim 56, wherein the computer readable memory further comprises data indicative of the risk of developing cancer associated with at least one allele of at least one polymorphic marker or at least one haplotype, and wherein a risk measure for the human individual is based on a comparison of the at least one marker and/or haplotype status for the human individual to the risk of cancer associated with the at least one allele of the at least one polymorphic marker or the at least one haplotype.

59. (canceled)

60. The apparatus according to claim 56, wherein the at least one marker or haplotype comprises at least one marker selected from the markers set forth in Table 5, Table 6 and Table 7.

61. (canceled)

62. (canceled)

63. (canceled)

64. (canceled)

65. The method according to claim 1, wherein the human individual is of an ancestry that includes European ancestry.

66. The method according to claim 1, wherein the cancer is selected from Basal Cell Carcinoma, Lung Cancer, Bladder Cancer, Prostate Cancer, Cervical Cancer, Thyroid Cancer and Endometrial Cancer.

67. The method of claim 9, wherein the at least one marker allele is allele C of marker rs401681, or a marker allele in linkage disequilibrium therewith, wherein the linkage disequilibrium is characterized by a value for r² of at least 0.2.

68. The method of claim 67, wherein the cancer is melanoma cancer and/or colorectal cancer.
